(19) United States
(12) Patent Application Publication
(10) Pub. No.: US 2025/0312469 A1
(43) Pub. Date: Oct. 9, 2025

(54) FL118 COMPLEXES FOR TARGETING DDX5, UbE2T or USP2a

(71) Applicant: CANGET BIOTEKPHARMA, Buffalo, NY (US)

(72) Inventors: Xiang Ling, Buffalo, NY (US); Fengzhi Li, Buffalo, NY (US)

(21) Appl. No.: 19/043,126

(22) Filed: Jan. 31, 2025

Related U.S. Application Data
(63) Continuation of application No. PCT/US25/14017, filed on Jan. 31, 2025.
(60) Provisional application No. 63/550,284, filed on Feb. 6, 2024.

(51) Int. Cl. A61K 47/68; A61K 9/00; A61K 9/16; A61K 45/06; A61K 47/64; A61K 47/69; A61P 35/00

(52) U.S. Cl. CPC A61K 47/6803; A61K 9/0053; A61K 9/1682; A61K 45/06; A61K 47/643; A61K 47/6951; A61P 35/00

(57) ABSTRACT

A method of targeting DDX5, UbE2T or USP2a in a subject having cancer by administering a composition comprising an effective amount of complex comprising FL118 or a FL118-based analogue. The method may include combining the administration of one cancer therapeutic approach selected from either chemotherapy, targeted therapy, and/or immunotherapy. The disclosure includes methods of manufacturing complex comprising FL118 or an FL118-based compound as well as the methods used for testing various properties of FL118-complex or a FL118-based compound-complex.

### CROSS-REFERENCE TO RELATED APPLICATIONS

This application is a continuation of Patent Cooperation Treaty Application PCT/US25/14017, filed Jan. 31, 2025, which claims priority from a U.S. Provisional Application, Ser. No. 63/550,284, filed Feb. 6, 2024, which incorporates by reference in its entirety the subject matter of the previously mentioned U.S. provisional patent application and the PCT international patent application number PCT/US25/14017, entitled “FL118 COMPLEXES FOR TARGETING DDX5, UbE2T or USP2a.”

### BACKGROUND

The novel small molecule, FL118 possesses high efficacy against metastatic and treatment-resistant colorectal cancer (CRC) (Ling, et al. PLOS ONE. 2012; 7: e45571 and Zhao J, et al. Mol Pharmaceutics. 2014; 11:457-67) and pancreatic ductal adenocarcinoma (PDAC) (Ling, et al. J Exp Clin Cancer Res. 2018; 37:240 and Ling, et al. Clin Transl Med. 2022; 12: e45571) as well as other types of cancers including osteosarcoma and prostate cancer (Ling, et al. Journal of Pharmaceutical Analysis. 2024 May; 101001). FL118 inhibits the expression of survivin, Mcl-1, XIAP and cIAP2 (Ling, et al. PLOS ONE. 2012; 7: e45571). The mechanism by which FL118 inhibits the expression of multiple oncogenic and antiapoptotic proteins (e.g., survivin, Mcl-1, XIAP, cIAP2, etc.) continues to be explored.

### BRIEF SUMMARY

One aspect of the disclosure relates to a method of targeting DEAD-box RNA helicase 5 (DDX5), ubiquitin-conjugating enzyme E2 T (UbE2T) and/or ubiquitin specific protease 2a (USP2a) in a subject having cancer, the method comprising administering a composition comprising an effective amount of a complex comprising a compound of the formula:

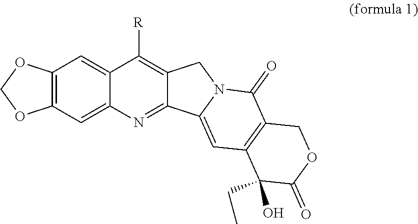

wherein R are selected from the group consisting of: H,

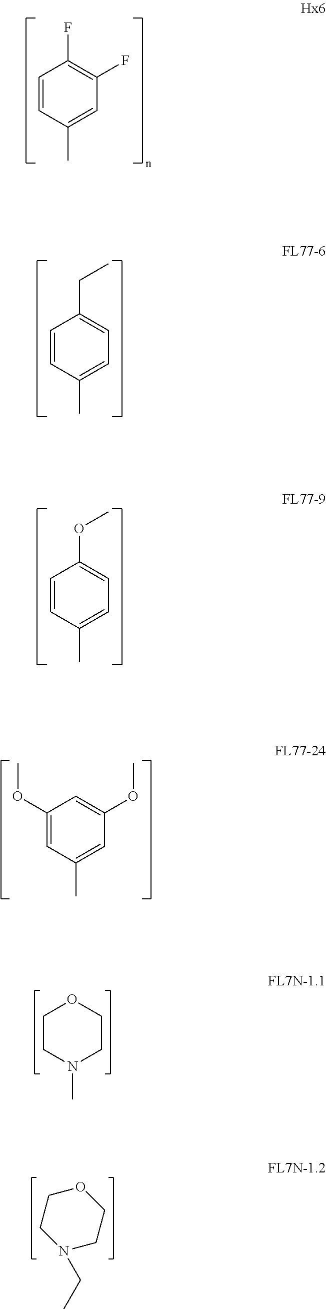

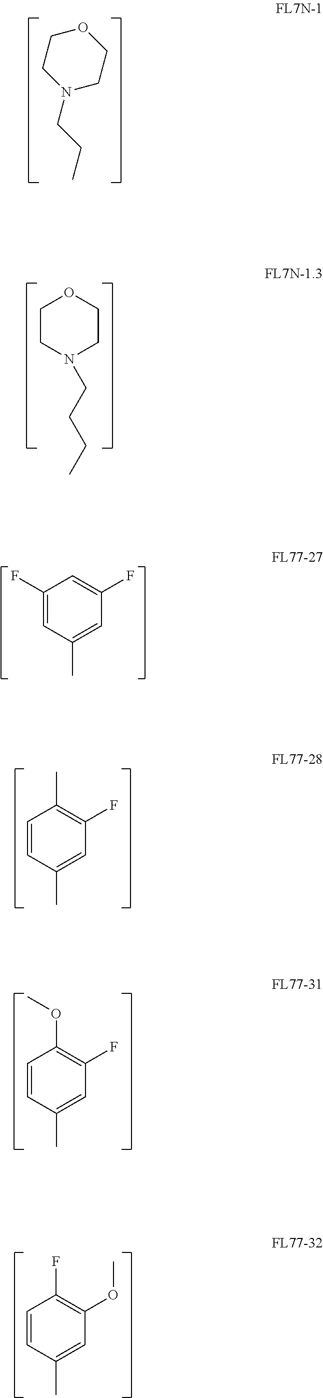

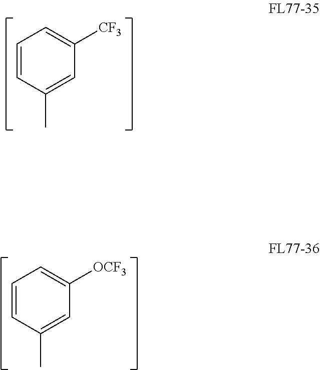

and a pharmaceutically acceptable salt.

In an aspect, the complex encapsulates or partially encapsulates the compound.

In an aspect, administration of the complex degrades and/or decreases the activity or expression of DDX5, UbE2T and USP2a.

In an aspect, the complex further comprises a human protein or a humanized protein or a humanized protein fragment, wherein the compound is non-covalently formulated in a process with the human protein to form a non-covalent protein-compound complex.

In an aspect, the human protein comprises human serum albumin (HSA), human fibrinogen, or human globulin.

In an aspect, the human globulin comprises alpha globulin, beta globulin, or gamma globulin or a combination thereof.

In an aspect, the human globulin comprises a human immunoglobulin (Ig).

In an aspect, the human Ig comprises a human IgG antibody, a human IgA antibody, a human IgM antibody, a human IgE antibody or a human IgD antibody.

In an aspect, the human Ig antibody comprises a human or humanized monoclonal antibody (mAb).

In an aspect, the human Ig antibody comprises a human or humanized single-chain variable fragment (scFv).

In an aspect, the complex comprises a cyclodextrin selected from the group consisting of β-cyclodextrin (βCD), hydroxypropyl-β-cyclodextrin (HPβCD), sulfobutylether-β-cyclodextrin (SBEβCD), methyl-β-cyclodextrin (meβCD), and derivatives thereof.

In an aspect, the complex comprises HPβCD and is formulated for oral administration.

In an aspect, the complex is administered intravenously (i.v.), intraperitoneally (i.p.) or orally (per oral/p.o.) administered in a daily, weekly to biweekly dosage from about 0.1 mg/kg to about 20 mg/kg.

In an aspect, the complex is administered at a dosage between about 0.5 mg/kg/wk and about 10 mg/kg/wk of the complex, alternatively between about 1.0 mg/kg/wk and about 8 mg/kg/wk of the complex, or alternatively between about 2.5 mg/kg/wk and about 7.5 mg/kg/wk of the complex.

In an aspect, the subject has drug resistant cancer.

In an aspect, the subject has cancerous desmoplasia.

In an aspect, the method further comprises administering the complex in combination with at least one cancer therapeutic approach.

In an aspect, at least one cancer therapeutic approach is selected from the group consisting of chemotherapy, targeted therapy, and immunotherapy.

One aspect of the disclosure is a method of synthesizing a complex comprising: dissolving the compound of formula 1 in at least one organic solvent to form a first solution; dissolving the human protein or the humanized protein or the humanized protein fragment in an aqueous solution to form a second solution; combining the first solution and the second solution to form a third solution; and, removing the organic and aqueous components from the third solution to produce a complex.

In an embodiment, the organic solvent comprises dissolved cyclodextrin.

In an aspect, the organic solvent includes solvents that are miscible with water or partially dissolved in water.

In an aspect, the ratio of the compound of formula 1 to the human protein or the humanized protein or the humanized protein fragment is in a ratio of about 4:1 to a ratio of about 12:1.

One aspect of the disclosure is a method of manufacturing a cyclodextrin-drug complex comprising: dissolving a cyclodextrin into at least one organic solvent forming a cyclodextrin master solution; dissolving the compound of formula 1 into the cyclodextrin master solution forming a cyclodextrin-drug complex suspension; homogenizing the cyclodextrin-drug complex suspension; spray-drying the cyclodextrin-drug complex suspension to produce a cyclodextrin-drug complex powder; and drying the cyclodextrin-drug complex powder.

In an aspect, the organic solvent is selected from the group consisting of methanol, ethanol, formic acid (FA), a salt formate, glacial acetic acid, a salt acetate, glyoxal, ethylene glycol (EG), propylene glycol (PG), formamide (FAD), (N,N,N′,N′)-tetramethyl-ethylenediamine (TEMED), ethanolamide (EA), 2-mercaptoethanol (MercE), and a combination thereof.

In an aspect, the cyclodextrin-drug complex further comprises a cyclodextrin selected from the group consisting of β-cyclodextrin (βCD), hydroxypropyl-β-cyclodextrin (HPβCD), sulfobutylether-β-cyclodextrin (SBEβCD), methyl-β-cyclodextrin (meβCD), and derivatives thereof.

In an aspect, the ratio of the compound of formula 1 to cyclodextrin is in the ratio of about 1:1 to 1:2.

In an aspect, the cyclodextrin-drug complex comprises HPβCD and is formulated for oral administration.

These and other advantages, aspects, and novel features of the present disclosure, as well as details of illustrated embodiments thereof, will be more fully understood from the following description and drawings.

### BRIEF DESCRIPTION OF THE DRAWINGS AND TABLES

Embodiments of the present disclosure will now be described, by way of example only, with reference to the attached Figures, wherein:

FIGS. 1A-F show that FL118 affinity purification identified that the oncogenic protein DDX5 is the physical and functional direct biochemical target for FL118. FIG. 1A shows a FL118 affinity column purification identified a ˜70 kD protein. FL118 was directly coupled to a beaded resin. Then, SW620 protein lysates were purified with the FL118 affinity column through steps of stringent washing, elution with 8M urea, de-urea, sample concentration to 20-30 μl, which was displayed on 5-20% SDS PAGE gel. FIG. 1B shows the ˜70 kD protein in (FIG. 1A) was analyzed via mass spectrometry (MS). The protein band was digested in gel and ten peptides were isolated and used for the searching protein database. The 10 peptides (red letters) fully matched the DDX5 (also called p68) protein sequence. FIGS. 1C and 1E show a FL118 treatment for 6h eliminates DDX5 phosphorylation on tyrosine (Y) residues in both Mia Paca-2 cells (FIG. 1C) and SW620 cells (FIG. 1E). FIGS. 1D and 1F show a FL118 treatment for 24h not only maintains DDX5 in a dephosphorylation status, but also inhibits DDX5 proteins in both Mia Paca-2 cells (FIG. 1D) and SW620 cells (FIG. 1F). PDAC Mia Paca-2 cells and CRC SW620 cells with and without FL118 treatment for 6h (FIGS. 1C and 1E) or 24h (FIGS. 1D and 1F) as shown; cells were then analyzed by immunoprecipitation (IP) with anti-DDX5 antibodies (αDDX5) or control IgG antibodies, followed by Western blots with anti-phospho-tyrosine-specific antibodies (αp-Y) and αDDX5, respectively.

FIGS. 2A-D show the small molecule drug binding to protein determined by using the isothermal titration calorimetry (ITC) technology. Representative ITC analysis results are shown. The KD values presented within the figure were the mean±SD from two ITC analyses. The y-axis, with different labelling scales is for result pattern visibility and dot inclusion. Purified Flag-tagged DDX5 (Flag-DDX5) (FIG. 2A and FIG. 2D), Flag-Topl (FIG. 2B) and BSA (FIG. 2C) were loaded into a 96 DeepWell PP plate, and then FL118 (FIG. 2A-2C) or topotecan (FIG. 2D) was automatically titrated stepwise into the protein cell by 20 injections in 60 min (one injection per 3 min) on the MicroCal-Malvern Auto-ITC200.

FIGS. 3A-D show that FL118 inhibits Agrin mRNA and protein as well as Agrin downstream target, YAP1, while DDX5 KO mimics FL118 treatment. FIG. 3A shows FL118 inhibits Agrin mRNA. Panc-1 and MiaPaca2 cells were treated with vehicle (control) and FL118 at 10, 100, and 500 nM, respectively, for 24h as shown. Cells were then analyzed using quantitative real-time RT-PCR. Data are the mean±SD from three independent tests. ** p<0.01; *** p<0.001. FIG. 3B shows FL118 inhibits Agrin and YAP1 proteins. Panc-1 cells treated with and without FL118 as shown, cells were then analyzed using Agrin and YAP1 antibody via Western blots. FIG. 3C shows Panc-1 cell clones with DDX5 knockout (KO) express very low/no Agrin and YAP1. Control Panc-1 cells in parallel with DDX5 KO Panc-1 cell clones B1-2, A1-2 and C2-1 were analyzed for Agrin and YAP1 expression using Western blots FIG. 3D shows Mia Paca-2 cell clones with DDX5 KO express low Agrin and YAP1. Control Mia Paca-2 cells in parallel with DDX5 KO Mia Paca-2 cell clones A8 and A9 were analyzed for Agrin and YAP1 expression using Western blots. GAPDH in FIG. 3B and FIG. 3C and Actin in FIG. 3D are the internal control of total protein loading.

FIG. 4 shows the inhibitory effects of FL118 on the protein expression of Agrin and its downstream targets in pancreatic cancer cell lines. Mia Paca-2, Panc-1 and BxPC-3 cells treated with and without FL118 (0, 10, 100 and 500 nM) as shown, cells were then lysed and analyzed using the corresponding antibodies for DDX5, Agrin, YAP1, p-FAK, FAK, Integrin and Actin via Western blots. Actin expression is the internal control of total protein loading.

FIG. 5 shows the inhibitory effects of FL118 on the protein expression of Agrin and its downstream targets in pancreatic cancer patient-derived xenograft (PDX) tumors in vivo. Mice bearing pancreatic cancer PDX14244 or PDX17624 tumors were orally treated with vehicle and with FL118 (5 mg/kg), and then tumors were collected on day 1, day 2 and day 3 after oral treatment as shown. The collected tumor tissues were then homogenized with untrasound and analyzed using the corresponding antibodies for DDX5, Agrin, YAP1, p-FAK, FAK, Integrin and Actin via Western blots. Actin expression is the internal control of total protein loading.

FIGS. 6A-B show that DDX5 knockout (KO) Panc-1 A2-1C clone cells exhibited cell attachment defect, which can be rescued by adding soluble Agrin (sAgrin) in the cultural media. FIG. 6A shows Agrin (5 μg/mL) can rescue DDX5 KO cell attachment defect (6 h after seeding, stained). FIG. 6B shows non-attached cell number statistics calculated from the corresponding experiment in FIG. 6A.

FIGS. 7A-B show that DDX5 KO Panc-1 C2-1 clone cells exhibited cell attachment defect, which can be rescued by adding sAgrin in the cultural media. FIG. 7A shows Agrin (5 μg/mL) can rescue DDX5 KO cell attachment defect (4 h after seeding, stained). FIG. 7B shows non-attached cell number statistics calculated from the corresponding experiment in FIG. 7A.

FIGS. 8A-B show that DDX5 KO MiaPaca2 A8 clone cells exhibited cell attachment defect, which can be rescued by adding sAgrin in the cultural media. FIG. 8A shows Agrin (5 μg/mL) can rescue DDX5 KO cell attachment defect (16 h after seeding, stained). FIG. 8B shows non-attached cell number statistics calculated from the corresponding experiment in FIG. 8A.

FIGS. 9A-B show that DDX5 KO MiaPaca2 A9 clone cells exhibited cell attachment defect, which can be rescued by adding sAgrin in the cultural media. FIG. 9A shows Agrin (5 μg/mL) can rescue DDX5 KO cell attachment defect (16 h after seeding, stained). FIG. 9B shows non-attached cell number statistics calculated from the corresponding experiment in FIG. 9A.

FIGS. 10A-B show that addition of sAgrin in the cultural media can rescue DDX5 KO Panc-1 C2-1 cell wound healing defect. FIG. 10A shows DDX5 KO Panc-1 C2-1 cell wound healing defect can be rescued by Agrin. FIG. 10B shows relative wound healing area statistics calculated from Experiment FIG. 10A.

FIGS. 11A-B show that addition of sAgrin in the cultural media can rescue DDX5 KO MiaPaca-2 A8 cell wound healing defect. FIG. 11A shows DDX5 KO MiaPaca-2 A8 cell wound healing defect can be rescued by Agrin. FIG. 11B shows percentage of wounded area statistics calculated from Experiment FIG. 11A.

FIGS. 12A-B show that FL118 pretreatment mimics DDX5 KO and induces Panc-1 cell attachment defect and blocks Panc-1 cell attachment. FIG. 12A shows Panc-1 cell attachment. FL118 pretreatment for 24 h and then reseeding the FL118 pre-treated Panc-1 cells for 6 h without FL118 presence in parallel with Panc-1 cell without FL118 pretreatment (control). FIG. 12B shows attached Panc-1 ell number statistics calculated from the Experiment in FIG. 12A.

FIGS. 13A-B show that FL118 pretreatment mimics DDX5 KO and induces BxPC-3 cell attachment defect and blocks BxPC-3 cell attachment. FIG. 13A shows BxPC-3 cell attachment. FL118 pretreatment for 24 h and then the FL118 pre-treated BxPC-3 cells were reseeded for 6 h without FL118 presence in parallel with Panc-1 cell without FL118 pretreatment (control). FIG. 13B shows attached Panc-1 ell number statistics calculated from the Experiment in FIG. 13A.

FIGS. 14A-B show that cancer cell attachment blocked by FL118 pretreatment can be rescued by Agrin by the addition of Agrin. FIG. 14A shows pretreatment of BxPC-3 cell by FL118 (10 nM, 24 h)-resulted cell attachment defect can be rescued in the presence of Agrin during seeding. FIG. 14B shows pretreatment of BxPC-3 cell by FL118 (100 nM, 24 h)-resulted cell detachment can be rescued by addition of Agrin.

FIGS. 15A-B show that FL118 treatment mimics DDX5 KO and blocks Panc-1 cell migration wound healing. FIG. 15A shows FL118 dose-dependent inhibition of cell migration (wound healing). FIG. 15B shows percentage wound healing area statistics calculated from the Experiment in FIG. 15A.

FIGS. 16A-B show that FL118 treatment mimics DDX5 KO and blocks BxPC-3 cell migration wound healing. FIG. 16A shows FL 118 dose-dependent inhibition of cell migration (wound healing). FIG. 16B shows percentage wound healing area statistics calculated from Experiment A.

FIGS. 17A-B show that FL118 24h pretreatment-inhibited BxPC-3 cell migration wound healing can be rescued by Agrin. FIG. 17A shows FL118-induced BxPC-3 cell wound healing damage can be rescued by Agrin. FIG. 17B shows percentage wound healing area statistics derived from Experiment A

FIGS. 18A-B show that FL118 treatment mimics DDX5 KO and blocks MiaPaca-2 cell migration wound healing. FIG. 18A shows FL118 dose-dependent inhibition of cell migration (wound healing). FIG. 18B shows percentage wound healing area statistics calculated from Experiment A.

FIGS. 19A-B show that FL118 24h pretreatment-inhibited MiaPaca-2 cell migration wound healing can be rescued by Agrin. FIG. 19A shows FL118-induced MiaPaca-2 cell wound healing damage can be rescued by Agrin. FIG. 19B shows Percentage wound healing area statistics derived from Experiment A.

FIGS. 20A-B show that FL118 treatment inhibits Panc-1 cell 3D culture spheroid formation. FIG. 20A shows morphology of Panc-1 cell spheroid formation with or without FL118 treatment. FIG. 20B shows Panc-1 cell viability statistics measured from Experiment A.

FIGS. 21A-B show that FL118 treatment inhibits MiaPaca-2 cell 3D culture spheroid formation. FIG. 21A shows morphology of MiaPaca-2 cell spheroid formation with or without FL118 treatment. FIG. 21B shows MiaPaca-2 cell viability statistics measured from Experiment A.

FIGS. 22A-B show that the DDX5 KO MiaPaca-2 cells exhibited 3D culture spheroid formation defect (much smaller spheroid size), which can be rescued by adding Agrin in the cell culture medium (7 days). FIG. 22A shows 3D spheroid size examples in various treatment conditions as shown for MiaPaca-2 cells (wild type cells version DDX5 KO cell clones A8 and A9). FIG. 22B shows 3D spheroid size statistics measured from Experiment A for wild type cells version DDX5 KO cell clones, A8 and A9).

FIGS. 23A-B show that the DDX5 KO Panc-1 cells exhibited 3D culture spheroid formation defect (much smaller spheroid size), which can be rescued by adding Agrin in the cell culture medium (7 days). FIG. 23A shows 3D spheroid size examples in various treatment conditions as shown for Panc-1 cells (wild type cells version DDX5 KO cell clones A2-1C and C2-1). FIG. 23B shows 3D spheroid size statistics measured from Experiment A for wild type cells version DDX5 KO cell clones A2-1C and C2-1).

FIG. 24 shows that FL118 inhibited pancreatic cancer cell and stroma spreading from the ex vivo-cultured pancreatic tumor tissues, which can be restored by adding Agrin in the ex vivo culture medium. Human pancreatic cancer PDX tissues isolated from severe combined immunodeficiency (SCID) mice were cut into ≤2 mm3 size and cultured as explants in cell cultural dish treated with or without FL118 and in the presence or absence of Agrin as shown for 7 days. Then, the status of cell and stroma spreading from the tumor edge were photographed. Three independent examples were shown.

FIG. 25 shows that FL118 induces the loosing of the dense stroma in the pancreatic cancer tissues, which can be restored by adding Agrin in the ex vivo culture medium. Human pancreatic cancer PDX tissues cultured as explants with or without FL118 and/or Agrin treatment for 7 days. Then, the ex vivo-cultured tissues were cut into tissue slide sections for H&E staining to observe desmoplasia changes. Three independent examples of H&E staining tissue slide sections were shown.

FIGS. 26A-B show the FL118 binding-based individual protein dot distributions displayed by using each protein Z-score as the X axis versus the FL118 binding signal as the Y axis. FIG. 26A shows protein dot distributions of the 4815 proteins with a Z-score >0 from the >9400 proteins on the ProtoArray are shown. FIG. 26B shows protein dot distributions of the 64 proteins with a Z-score >0 from the 112 ubiquitin-relevant proteins on the ProtoArray are shown. In addition to USP2a and UbE2T, a function-unknown ubiquitin-like protein 7 (UBL7) also had a Z-score >3; this molecule may act as a co-factor during the regulation of the protein ubiquitination (Ub)/deUb process.

FIGS. 27A-C shows the binding affinity of FL118 to USP2a and UbE2T in Isothermal Titration calorimetry (ITC) testing. ITC tests were performed with a MicroCal-Malvern Auto-ITC200. Purified Flag-tagged USP2a (FIG. 27A) and UbE2T (FIG. 27B) in parallel with the control protein bovine serum albumin (BSA) (FIG. 27C) were loaded into a 96-DeepWell PP plates, respectively. FL118 was then titrated stepwise into the protein sample cell through 20 injections (2 μL per injection) within 60 minutes (one injection every 3 min).

FIGS. 28A-I show the effects of FL118 on USP2a activity and expression: FIG. 28A shows FL118 inhibits USP2a deubiquitinase activity in vitro. Flag-tagged MdmX (0.1 μM) was mixed with 0.1 μM Mdm2 for 60 min at 30° C. in the presence of UbE1, UbE2D3, ubiquitin, and ATP in a buffer. Then, 0.1 μM USP2a, pre-incubated with or without 50 nM FL118 on ice for 30 min, was added into the reactions (lanes 2, 3) followed by continuous incubation at 30° C. for 60 min. Reactions were then immunoblotted with Flag antibody. Signals were detected with enhanced chemiluminescence (ECL). FIG. 28B shows immunoprecipitation (IP) with MdmX mAb and Western blotting (WB) with polyUb antibody. FIG. 28C shows direct WB with poly Ub antibody. FIG. 28D, FIG. 28E, FIG. 28F, FIG. 28G show effects of FL118 on USP2a mRNA (FIG. 28D, real-time RT-PCR) and protein (FIG. 28E, FIG. 28F, FIG. 28G, WB) in DU145, PC3, and LNCaP cells. Each bar in FIG. 28D is the mean±SD from 3 independent analyses. GAPDH in FIG. 28E, FIG. 28F, and FIG. 28G is the internal control. FIG. 28H and FIG. 281 show forced overexpression of USP2a in LNCaP (FIG. 28H) and DU145 (FIG. 281) cells enhances the inhibition of cell growth by FL118. Cells stably transfected with pcDNA3 (control) or pcDNA3-USP2a in 96-well plates were treated with and without FL118 as shown for 72 h; cell growth was then analyzed using MTT assays. Each bar is the mean±SD from 3 independent analyses; the t-test was used for P-values: * P≤0.5; ** P≤0.01; *** P≤0.001.

FIGS. 29A-D show that FL118 inhibits UbE2T protein but not its mRNA, which happens independently of p53 status. Results showed that FL118 destabilizes UbE2T proteins (FIG. 29A, FIG. 29C, FIG. 29D) without inhibitory effects on its mRNA (FIG. 29B). Prostate cancer (PCa) cells were treated with and without FL118 as shown. Cells were then lysed to determine UbE2T protein expression (WB) and mRNA (real-time RT-PCR). GAPDH in FIG. 29A, FIG. 29C, and FIG. 29D is the internal control for total protein loading. Each bar in FIG. 29B is the mean±SD from 3 independent analyses.

FIGS. 30A-B show that USP2a and UbE2T control the expression of multiple FL118-inhibited proteins. FIG. 30A. Forced expression of USP2a increases the expression of survivin, Mcl-1, XIAP, and cIAP2. LNCaP cells were transfected with empty vector or USP2a vectors (OriGene). The G418-selected cells were then subjected to Western blotting (WB) analyses using their antibodies. FIG. 30B shows lentiviral UbE2T shRNA silencing of UbE2T decreases the expression of survivin, Mcl-1, XIAP, and cIAP2 with cell apoptosis (cleaved PARP). UbE2T-highly expressing DU145 cells were infected with the lentiviral particles containing control shRNA or UbE2T shRNA (#2, #5). The puromycin-selected cells were then analyzed with WB to determine their expression and PARP cleavage. GAPDH in FIG. 30A and FIG. 30B is the internal control.

FIGS. 31A-D show that FL118 activates p53 and Mdm2, inhibits AR, AR-Vs, c-Myc, and MdmX, and induces apoptosis (PARP cleavage). PCa (C4-2, 22Rv1, LNCaP) cells were treated with or without FL118 as shown. Cells were then lysed to determine the expression of AR, AR-Vs, p53, p21, Mdm2, MdmX, and c-Myc as well as PARP cleavage (apoptosis hallmark) using their corresponding antibodies by WB. GAPDH in FIG. 31A, FIG. 31B, and FIG. 31C or actin in FIG. 31D are the internal controls for total protein loading.

FIGS. 32A-D shows that the ubiquitin-mediated proteasome degradation pathway may be involved in FL118-mediated inhibition of AR and AR-Vs. FIG. 32A, FIG. 32B, FIG. 32C show FL118-mediated inhibition of AR and AR-Vs can be at least partially restored in the presence of proteasome inhibitor MG132 in C4-2, LNCaP, and 22Rv1 cells. Cells and treatment are as shown. GAPDH is the internal control. FIG. 32D shows AR and AR-Vs mRNA is inhibited to different degrees by FL118 in LNCaP cells versus in 22Rv1 castration resistant prostate cancer (CRPC) cells. Each bar is the mean±SD from 3 independent analyses. * P-value ≤0.5.

FIGS. 33A-F show that FL118 promotes rapid degradation of UbE2T, survivin, Mcl-1, c-Myc, FASN, and AR in p53 mutant/null PCa cells, which can be reversed by MG132. FIG. 33A shows DU145 cells were treated with or without FL118 in the presence or absence of MG132 for 16 h. Expression of UbE2T, c-Myc, survivin, and PARP was determined by WB. FIG. 33B shows DU145 cells were treated with or without FL118 as shown. FASN and Mcl-1 expression was determined by WB. FIG. 33C shows PC-3 cells were treated with or without FL118 or MG132 as shown. Survivin and Mcl-1 expression was determined by WB. FIG. 33D shows PC-3 cells were treated with or without FL118 or MG132 as shown. UbE2T and AR expression was determined by WB. FIG. 33E shows DU145 cells were treated with and without FL118 as shown. AR expression was determined by WB analysis. FIG. 33F shows PC-3 with AR overexpression by transfection of AR expression vector or pcDNA3 empty vector. Actin in FIG. 33A, FIG. 33B, FIG. 33C and GAPDH in FIG. 33D, FIG. 33E, FIG. 33F are internal controls for total protein loading.

FIGS. 34A-F show FL118 anti-prostate cancer efficacy and toxicity from in vivo studies. FIG. 34A shows the expression of USP2a, UbE2T and DDX5 in the 3 commonly used PCa cell lines for USP2a and UbE2T expression. GAPDH is an internal control. FIG. 34B shows growth inhibition of LNCaP, DU145, and PC-3 cells in response to FL118 treatment. Subconfluent PCa cells in 96-well plates were treated with and without FL118 in a series of concentrations as shown for 72 h. Cell growth/viability was then analyzed using MTT assays. Data curves are the mean±SD from 3 independent assays. FIG. 34C shows antitumor efficacy of FL118 on DU145 xenografts. SCID mice were orally administered the vehicle or FL118 (8 mg/kg, weekly×4) as arrowed. FIG. 34D shows antitumor efficacy of FL118 and enzalutamide (Enza) on CRPC LAPC9 tumors in SCID mice. Mice were treated with Enza (10 mg/kg, intraperitoneally (i.p.) 3 times per week for 6 times over 2 weeks (green arrows) or with the vehicle or FL118 (8 mg/kg) orally once per week for 2 times (black arrows) for comparison with the Enza treatment. FIG. 34E shows antitumor efficacy of FL118 on the 22Rv1 CRPC tumors. SCID mice were orally administered the vehicle or FL118 (5 mg/kg, weekly×4, arrowed). Tumor model setup in FIG. 34C, FIG. 34D, FIG. 34E; xenograft tumors were first established from PCa cells on SCID mice. The tumors were then inoculated on mice at the flank area. Treatment began when tumors reached 150-200 mm3. FIG. 34F shows body weight changes after vehicle or FL118 treatment from FIG. 34E. Data curves are the mean±SD from 5 tumor sizes (FIG. 34C, FIG. 34D, FIG. 34E) or 5 mouse body weights (FIG. 34F).

FIGS. 35A-B shows the association of USP2a and UbE2T expression with prostate cancer patient outcomes from clinical specimens. FIG. 35A shows USP2a, UbE2T, and AR expression. Thirty μg protein lysates from normal prostate tissues (Normal 1, no matched tumor; Normal 2 matches PCa-8611; Normal 3 matches 11703), and PCa specimens were analyzed for the expression of USP2a, UbE2T, and AR using Western blotting (WB). GAPDH is the internal control. PCa tissues marked with “*” were from either metastatic sites or primary sites with metastatic disease. FIG. 35B shows summary of clinical information from the patients related to the 19 PCa tissues. The full name of all abbreviations used in FIG. 35B can be found in the footnote to the “FIG. 35B” section.

FIGS. 36A-B shows the hERG Test for FL118's effects on hERG activity (FIG. 36A) and the positive control quinidine's effect on hERG activity (FIG. 36B). The experiment was performed on an IonWorks™ HT instrument (Molecular Devices Corporation), which automatically performs electrophysiology measurements in 48 single cells simultaneously in a specialized 384-well plate (PatchPlate™).

FIGS. 37A-C shows that FL118 exhibits favorable PK profiles after i.v. injection or p.o . . . . A. FL118 i.v. PK results. SCID mice were subcutaneously implanted with human FaDu and SW620 tumors. After tumor grew to 800-1000 mm3, FL118 was i.v.-injected for one time at 1.5 mg/kg. Then, blood and tumor tissues were collected at 10 min, 1 h, 4 h, 12 h, 24, and 48 h (The data “A” was reported in Ling et al., Am J Transl Resb7: 1765-81, 2015). FIG. 37B and FIG. 37C shows FL118 p.o. PK results. Tumor mouse setup was the same as in A. FL118 was orally administered for one time at 1.5 mg/kg via p.o. routes. Then, blood and tumor tissues were collected at 30 min, 1 h, 2 h, 4 h, 12, and 24 h. Three SCID mice at each time point were used (FIG. 37A, FIG. 37B, FIG. 37C). Standard deviation (SD) was analyzed using Excel software.

FIGS. 38A-E show the different prostate cancer cell models with DDX5 knockout (KO) or with UbE2T KO and their regulation relationship. Vector-free Crispr-Cas9-mediated gene KO technology was used to knock out DDX5 and UbE2T in various PCa cell lines as shown (FIG. 38A, FIG. 38B, FIG. 38C and FIG. 38D). Multiple DDX5 KO cell clones were generated from 22Rv1 (FIG. 38A), LNCaP (FIG. 38B) and Du145 (FIG. 38C) cell lines. Multiple UbE2T KO cell clones were generated from the Du145 cell line (FIG. 38D). Individual cell clones along with the corresponding control cells were harvested and lysed to determine DDX5 and UbE2T protein expression using Western blots as shown. GAPDH is the internal total protein loading control. FIG. 38E shows use of DDX5 KO in LNCaP and Du145 cells as well as the use of UbE2T KO in Du145 cells to study the gene expression relationship of DDX5, UbE2T and USP2a using Western blots. Actin is the internal total protein loading control.

FIGS. 39A-C show that the functional analysis of the DDX5 KO DU145 cells. FIG. 39A shows DDX5 KO DU145 cell growth in complete cell culture medium over 7 days. Cells were seeded in 96-well plates and grown for 7 days (medium was changed every 3 days) and then cell growth was analyzed using MTT assays. Each time point is the mean±SD from 3 independent analyses. FIG. 39B shows effects of FL118 treatment for 72h on the growth inhibition of DU145 cells with and without DDX5 KO. Cells were seeded in 96-well plates and treated with and without FL118 as shown for 72 h. Cell growth was then analyzed using MTT assays. Each bar is the mean±SD from 3 independent analyses. FIG. 39C shows Morphological changes comparison of DU145 cells with versus without DDX5 KO. Cells were seeded in 12-well plates overnight and then cell morphology photos were digitally taken. Represent images are shown.

FIGS. 40A-B show the antitumor activity and toxicity (body weight changes) of FL118 formulated with methanol-HPβCD solution. Human colorectal cancer SW620 xenograft tumors were first generated through implanting SW620 cancer cells at the flank area of SCID mice. Then, the tumors were isolated and individual experimental mice were subcutaneously implanted with 30-50 mg non-necrotic tumor masses at the flank area of individual mice. Seven to 10 days after tumor transplantation at which the implanted xenograft tumors were grown to100-200 mm3 (defined as day 0), mice were randomly divided into the required groups for treatment vial oral administration of the formulated FL118 or vehicles with a schedule of weekly×4 (arrowed). FIG. 40A shows FL118 efficacy on SW620 cancer cell-established xenograft tumors in SCID mice. The tumor growth curves are shown. FIG. 40B shows SCID mouse body weight changes after treatment with vehicle or with the formulated FL118. The mouse body weight curves are shown.

FIGS. 41A-B show the antitumor activity and toxicity (body weight changes) of FL118 formulated with ethanol-HPβCD solution. Experimental human colorectal cancer SW620 xenograft tumor SCID mice set up and follow-up oral treatment are the same as described in FIGS. 40A-B. FIG. 41A shows FL118 efficacy on SW620 xenograft tumors in SCID mice. The tumor growth curves are shown. FIG. 41B shows SCID mouse body weight changes after treatment with the formulated FL118 or with vehicle. The mouse body weight curves are shown.

FIGS. 42A-B show the antitumor activity and toxicity (body weight changes) of FL118 formulated with formic acid (FA)-HPβCD solution. Experimental human colorectal cancer SW620 xenograft tumor SCID mice set up and follow-up oral treatment are the same as described in FIGS. 40A-B. FIG. 42A shows FL118 efficacy on SW620 xenograft tumors in SCID mice. The tumor growth curves are shown. FIG. 42B shows SCID mouse body weight changes after treatment with the formulated FL118 or with vehicle. The mouse body weight curves are shown.

FIGS. 43A-B shows the antitumor activity and toxicity (body weight changes) of FL118 formulated with glacial acetic acid HPβCD solution. Experimental human colorectal cancer SW620 xenograft tumor SCID mice set up and follow-up oral treatment are the same as described in FIGS. 40A-B. FIG. 43A shows FL118 efficacy on SW620 xenograft tumors in SCID mice. The tumor growth curves are shown. FIG. 43B shows SCID mouse body weight changes after treatment with the formulated FL118 or with vehicle. The mouse body weight curves are shown.

FIGS. 44A-B show the antitumor activity and toxicity (body weight changes) of FL118 formulated with zinc acetate (ZA)-HPβCD solution. Experimental human colorectal cancer SW620 xenograft tumor SCID mice set up and follow-up oral treatment are the same as described in FIGS. 40A-B. FIG. 44A shows FL118 efficacy on SW620 xenograft tumors in SCID mice. The tumor growth curves are shown. FIG. 44B shows SCID mouse body weight changes after treatment with the formulated FL118 or with vehicle. The mouse body weight curves are shown.

FIGS. 45A-B show the antitumor activity and toxicity (body weight changes) of FL118 formulated with glyoxal-HPβCD solution. Experimental human colorectal cancer SW620 xenograft tumor SCID mice set up and follow-up oral treatment are the same as described in FIGS. 40A-B. FIG. 45A shows FL118 efficacy on SW620 xenograft tumors in SCID mice. The tumor growth curves are shown. FIG. 45B shows SCID mouse body weight changes after treatment with the formulated FL118 or with vehicle. The mouse body weight curves are shown.

FIGS. 46A-B show the antitumor activity and toxicity (body weight changes) of FL118 formulated with ethylene glycol (EG)-HPβCD solution. Experimental human colorectal cancer SW620 xenograft tumor SCID mice set up and follow-up oral treatment are the same as described in FIGS. 40A-B. FIG. 46A shows FL118 efficacy on SW620 xenograft tumors in SCID mice. The tumor growth curves are shown. FIG. 46B shows SCID mouse body weight changes after treatment with the formulated FL118 or with vehicle. The mouse body weight curves are shown.

FIGS. 47A-B show the antitumor activity and toxicity (body weight changes) of FL118 formulated with propylene glycol (PG)-HPβCD solution. Experimental human colorectal cancer SW620 xenograft tumor SCID mice set up and follow-up oral treatment are the same as described in FIGS. 40A-B. FIG. 47A and FL118 efficacy on SW620 xenograft tumors in SCID mice. The tumor growth curves are shown. FIG. 47B shows SCID mouse body weight changes after treatment with the formulated FL118 or with vehicle. The mouse body weight curves are shown.

FIGS. 48A-B show the antitumor activity and toxicity (body weight changes) of FL118 formulated with formamide (FAD)-HPβCD solution. Experimental human colorectal cancer SW620 xenograft tumor SCID mice set up and follow-up oral treatment are the same as described in FIGS. 40A-B. FIG. 48A shows FL118 efficacy on SW620 xenograft tumors in SCID mice. The tumor growth curves are shown. FIG. 48B shows SCID mouse body weight changes after treatment with the formulated FL118 or with vehicle. The mouse body weight curves are shown.

FIGS. 49A-B show the antitumor activity and toxicity (body weight changes) of FL118 formulated with N,N,N′,N′-tetramethyl ethylene diamine (TEMED)-HPβCD solution. Experimental human colorectal cancer SW620 xenograft tumor SCID mice set up and follow-up oral treatment are the same as described in FIGS. 40A-B. FIG. 49A shows FL118 efficacy on SW620 xenograft tumors in SCID mice. The tumor growth curves are shown. FIG. 49B shows SCID mouse body weight changes after treatment with the formulated FL118 or with vehicle. The mouse body weight curves are shown.

FIGS. 50A-B shows the antitumor activity and toxicity (body weight changes) of FL118 formulated with ethanolamide (EA)-HPβCD solution. Experimental human colorectal cancer SW620 xenograft tumor SCID mice set up and follow-up oral treatment are the same as described in FIGS. 40A-B. FIG. 50A shows FL118 efficacy on SW620 xenograft tumors in SCID mice. The tumor growth curves are shown. FIG. 50B shows SCID mouse body weight changes after treatment with the formulated FL118 or with vehicle. The mouse body weight curves are shown.

FIGS. 51A-B show the antitumor activity and toxicity (body weight changes) of FL118 formulated with mercaptoethanol (MercE)-HPβCD solution. Experimental human colorectal cancer SW620 xenograft tumor SCID mice set up and follow-up oral treatment are the same as described in FIGS. 40A-B. FIG. 51A shows FL118 efficacy on SW620 xenograft tumors in SCID mice. The tumor growth curves are shown. FIG. 51B shows SCID mouse body weight changes after treatment with the formulated FL118 or with vehicle. The mouse body weight curves are shown.

FIG. 52 shows the potential impurity chemical structure possibly in the final FL118 active pharmaceutical ingredient (API).

FIG. 53 shows the X-ray powder diffractometer (XRPD) pattern of FL118 API. The FL118 XRPC pattern indicated that FL118 has a crystal status.

FIG. 54 shows that the Differential Scanning calorimetry (DSC) curve of FL118 API. The FL118 DSC curve indicated that FL118 has no melting point.

FIG. 55 shows the X-ray powder diffractometer (XRPD) overlay of the HPβCD-FL118 complex powder product. The XRPD results indicate that HPβCD-FL118 complex powder products are all amorphous. Of note, the HPβCD-FL118 complex powder used is FR00535-01-190708.

FIG. 56 shows the Modulated Differential Scanning calorimetry (mDSC) profile of the HPβCD-FL118 complex powder product at the Condition 1. Of note, the HPβCD-FL118 complex powder used at Condition 1 is FR00535-01-190708.

FIG. 57 shows the Modulated Differential Scanning calorimetry (mDSC) profile of the HPβCD-FL118 complex powder product at Condition 2. Of note, the HPβCD-FL118 complex powder used at Condition 2 is FR00535-01-190708.

FIG. 58 shows the Modulated Differential Scanning calorimetry (mDSC) profile of the HPβCD-FL118 complex powder product at Condition 3. Of note, the HPβCD-FL118 complex powder at Condition 3 is FR00535-01-190708.

FIG. 59 shows the Modulated Differential Scanning calorimetry (mDSC) profile of the HPβCD excipient for comparison.

FIG. 60 shows the X-ray powder diffractometer (XRPD) overlay of HPβCD-FL118 complex (FR00535-02-190904-01). The FL118 XRPC pattern indicated that HPβCD-FL118 complex powder product is amorphous.

FIG. 61 shows the Modulated Differential Scanning calorimetry (mDSC) profile of the HPβCD-FL118 complex powder product (FR00535-02-190904-01). The mDSC results indicate that there is no glass transition temperature for HPβCD-FL118 complex product.

FIG. 62 shows the X-ray powder diffractometer (XRPD) overlay of the 10-day stability at two conditions. The XRPD results indicate that the HPβCD-FL118 complex samples are very stable in both conditions.

FIG. 63 shows the High-Performance Liquid Chromatography (HPLC) overlay of the 10-day stability at two conditions. The HPLC results indicate that the HPβCD-FL118 complex samples are very stable in both conditions.

FIG. 64 shows the X-ray powder diffractometer (XRPD) overlay of the HPβCD-FL118 complex. The XRPD results indicate that HPβCD-FL118 complex is amorphous. Of note, the HPβCD-FL118 complex batch used is FR00535-5-191104-01.

FIGS. 65A-B show the FL118-HPβCD complex particle size distributions. FIG. 65A shows particle size distributions prior to microfluidizer processing and FIG. 65B shows after being processed using the microfluidizer equipment.

FIGS. 66A-B show the analysis of FL118-HPβCD complex powder products (batch: 2020-252-23). FIG. 66A shows X-ray powder diffractometer (XRPD) overlay of the HPβCD-FL118 complex (red curve) with naked FL118 API (blue curve). The XRPD results indicate that HPβCD-FL118 complex is amorphous with a portion of the FL118 DP in a cocrystal status. FIG. 66B shows the modulated Differential Scanning calorimetry (mDSC) profile of the HPβCD-FL118 complex powder product with weak melting point around 250° C. (i.e., a weak glass transition temperature).

FIG. 67 shows the analysis of FL118-HPβCD complex powder products (batch: 2020-252-18). X-ray powder diffractometer (XRPD) overlay of the HPβCD-FL118 complex (blue curve) with naked FL118 API (red curve). The XRPD results indicate that HPβCD-FL118 complex is amorphous with a portion of DP in a cocrystal status.

FIG. 68 shows the ethanol-processed FL118 products (Lot No.: FR00535-5-191104-01) improved FL118 efficacy with better oral bioavailability. SCID mice were implanted subcutaneously with 25-50 mg non-necrotic SW620 xenograft tumors (having mutations in Kras, p53 and APC genes) at the flank area of mice. When tumors reached 100-200 mm³ (defined day 0), FL118 at doses of 6, 5, 4, 3, 2 and 1 mg/kg was orally administered via weekly×4 (arrowed) to each mouse (5 mice per group per dose), respectively. FIG. 68A shows FL118 at doses of 6 mg/kg. FIG. 68B shows FL 118 at doses of 5 mg/kg. FIG. 68C shows FL118 at doses of 4 mg/kg FIG. 68D shows FL118 at doses of 3 mg/kg. FIG. 68E shows FL 118 at doses of 2 mg/kg. FIG. 68F shows FL 118 at doses of 1 mg/kg. The vehicle-treated tumor curve is the mean±SD derived from five mice. FL118-treated individual tumor curves from 5 mice in each group are shown.

FIGS. 69A-D show the glacial acetic acid (GAA)/ethanol solvent-processed FL118 products (Lot No.: CGT SDC LOT 2020-252-23) further improved the FL118 efficacy-toxicity ratio. Severe combined immunodeficiency (SCID) mice were implanted subcutaneously with 25-50 mg non-necrotic SW620 xenograft tumors (having triple mutations in Kras, p53, and APC genes) at the flank area of mice. When tumors reached 100-200 mm³ (defined day 0), FL118 at the doses of 10, 7.5, 5, and 2.5 mg/kg was orally administered weekly 4 times (arrowed) to each group of mice (3 female and 2 male mice per group per dose), respectively. FIG. 69A, FIG. 69B, FIG. 69C shows the vehicle-treated tumor curve is the mean±SD from 5 SCID mice; and the FL118-treated individual tumor curves from 5 mice (3 female and 2 male) in each dosage group are shown. FIG. 69D shows the mouse body weight change curves derived from the new FL118 product are the mean body weight+SD from each group (5 mice per group).

FIGS. 70A-B show that FL118 effectively eliminates human OS tumor in severe combined immunodeficiency (SCID) mice. FIG. 70A shows FL118 eliminates OS tumor but not DOX or Doxil (liposome-formulated DOX). The SK-ES-1 OS cells (2×10⁶ per tumor site) mixed with 50% Matrigel were subcutaneously injected into 2-3 SCID mice in the flank area to establish xenograft tumors. The established OS tumors were implanted into experimental SCID mice for drug efficacy studies. Treatment with vehicle, DOX, Doxil, or glacial acetic acid formulated, clinically compatible FL118 at the indicated doses was started when tumors grew to the size of 150-200 mm³. The schedule and route were weekly×4 via oral (p.o.) administration (arrowed). Each tumor curve is the mean tumor size+SD from 2-4 SCID mice. FIG. 70B shows mouse body weight change curves after vehicle, DOX, Doxil, or FL118 treatment. Each body weight change curve is the mean body weight change+SD from 2-4 SCID mice.

FIGS. 71A and 71B show the female rat body weight and food consumption with oral FL118 on Days 1 and 8. Normal Sprague Dawley female rats were used in 4 groups with vehicle or different levels of FL118 oral administration on Day 1 and Day 8. FIG. 71A shows female rat mean body weight changes over times after FL118 oral administration on Days 1 and 8. FIG. 71B shows female rat mean food consumption over times after FL118 oral administration on Days 1 and 8.

FIGS. 72A-B show the male rat body weight and food consumption with oral FL118 on Days 1 and 8. Normal Sprague Dawley male rats were used in 4 groups with vehicle or different levels of FL118 oral administration on Day 1 and Day 8. FIG. 72A shows male rat mean body weight changes over times after FL118 oral administration on Days 1 and 8. FIG. 72B shows male rat mean food consumption over times after FL118 oral administration on Days 1 and 8.

FIGS. 73A-B shows the female dog body weight and food consumption with oral FL118 on Days 1 and 8. Normal female beagle dogs were used in 4 groups with vehicle or different levels of FL118 products oral administration on Day 1 and Day 8. FIG. 73A shows Female dog mean body weight changes over times after FL118 oral administration on Days 1 and 8. FIG. 73B shows female dog mean food consumption over times after FL118 oral administration on Days 1 and 8.

FIGS. 74A-B show the male beagle body weight and food consumption with oral FL118 on Days 1 and 8. Normal male rats were used in 4 groups with vehicle or different levels of FL118 oral administration on Day 1 and Day 8. FIG. 74A shows the male dog mean body weight changes over times after FL118 oral administration on Day 1 and Day 8. FIG. 74B shows the male dog mean food consumption over times after FL118 oral administration on Days 1 and 8.

FIG. 75 shows the test article FL118's effect on hERG activity (FIG. 75A) and the positive control of quinidine's effect on hERG activity (FIG. 75B).

FIG. 76 shows the test article FL118's serum and tumor concentration over time (i.e., pharmacokinetics/PK) after oral administration.

FIG. 77 shows FL118's spectral wavelength versus absorbance changes in four different solvent content buffer conditions (PBS, PBS with 4% HSA, PBS with salt and PBS with 4% HSA+salt).

FIG. 78 shows the comparison of FL118's spectral wavelength versus absorbance changes in 2% IgG in PBS buffer, in PBS buffer only and in 4% HSA in PBS buffer. The data demonstrated that IgG could increase FL118 absorbance while 4% human serum albumin (HSA, of note, this is a concentration that mimics human blood HSA concentration) could decrease FL118's absorbance in comparison with FL118 in PBS.

FIG. 79 shows the comparison of FL118's spectral wavelength versus absorbance changes with high salts (NaCl) in PBS buffer only, in 2% IgG in PBS buffer, and in 4% HSA in PBS buffer. The data demonstrated that in the presence of a high salt, IgG could decrease FL118 absorbance while 4% human serum albumin (HSA, of note, this is a concentration that mimics human blood HSA concentration) could further decrease FL118's absorbance in comparison with FL118 in salt of PBS.

FIG. 80 shows the comparison of the wavelength versus absorbance change of FL118 in three conditions of (1) in PBS buffer only, (2) in 4% HSA in PBS buffer, and (3) FL118 non-covalently formulated with HSA in PBS buffer.

FIG. 81 shows the antitumor efficacy and toxicity (animal body weight loss) of SPEFL118 in a range of different concentrations administrated via intraperitoneal routes (i.p.) with a weekly×2 schedule in SW620 tumor models. FIG. 81A. The efficacy curve of vehicle and a series of SPEFL118 dose levels via i.p. routes. SW620 tumors were initially established by subcutaneous injection of 2×10⁶ SW620 cells at the flank area of SCID mice. Experimental tumor mice were prepared by subcutaneously implanting 25-50 mg non-necrotic SW620 tumors (at the flank area) isolated from the human SW620 tumor maintaining SCID (severe combined immunodeficiency) mice. When tumors reached 100-200 mm³ (defined day 0), vehicle and a series of SPEFL118 were administered via i.p. weekly×2 as shown (arrowed). Each tumor growth curve from vehicle-treated and different SPEFL118 dose-treated mice is shown. FIG. 81B. Mouse body weight change curves: The mouse body weight change curve from vehicle-treated and different SPEFL 118 dose-treated mice is shown.

FIG. 82 shows the antitumor efficacy and toxicity (animal body weight loss) of SPEFL118 in a range of different concentrations with one-time oral administration (p.o.) in human SW620 colorectal cancer tumor models. FIG. 82A. The efficacy curve of vehicle and a series of SPEFL 118 dosing levels via p.o. (oral routes) with one-time p.o. routes. Tumor mouse setup is the same as in FIG. 81A. Tumor growth curve from vehicle-treated and different SPEFL118 dose-treated mice is shown. FIG. 82B. Mouse body weight change curves: The mouse body weight change curve from vehicle-treated and different SPEFL 118 dose-treated mice is shown.

FIG. 83 shows the antitumor efficacy and toxicity (animal body weight loss) of SPEFL118 versus Doxorubicin (DOX) via intravenous administration (i.v.) in the fibrosarcoma HT1080 (CCL-121) tumor models from female SCID mice. FIG. 83A. The efficacy curve of vehicle, DOX (5 mg/kg, weekly ×3) and SPEFL118 (2.5 mg/kg and 5 mg/kg, weekly×4, arrowed) via i.v . . . . Tumor mouse setup is the same as in FIG. 81A and the weekly×3 (DOX) or weekly×4 (SPEFL118) were used in this study. Each tumor growth curve from vehicle-, DOX- and SPEFL 118-treated mice is the mean tumor size+SD derived from 3 tumors on 3 female mice. FIG. 83B. Mouse body weight change curves: The mouse body weight change curve from vehicle-, DOX- and SPEFL 118-treated mice is the mean body weight+SD derived from 3 female mice.

FIG. 84 shows the antitumor efficacy and toxicity (animal body weight loss) of SPEFL118 versus Doxorubicin (DOX) via i.v. administration in the fibrosarcoma HT1080 (CCL-121) tumor models from male SCID mice. FIG. 84A. The efficacy curve of vehicle, DOX (5 mg/kg, weekly ×3) and SPEFL 118 (2.5 mg/kg and 5 mg/kg, weekly×4, arrowed) via i.v . . . . Tumor mouse setup is the same as in FIG. 81A and the weekly×3 (DOX) or weekly×4 (SPEFL 118) were used for this study. Each tumor growth curve from vehicle-, DOX- and SPEFL 118-treated mice is the mean tumor size+SD derived from 3 tumors on 3 male mice. FIG. 84B. Mouse body weight change curves: The mouse body weight change curve from vehicle-, DOX- and SPEFL 118-treated mice is the mean body weight+SD derived from 3 male mice.

FIG. 85 shows the antitumor efficacy and toxicity (animal body weight loss) of SPEFL118 at different dosing levels via weekly one-time i.v., followed by 3-time i.p. administration in the fibrosarcoma HT1080 (CCL-121) tumor models from female SCID mice.

FIG. 85A. The efficacy curve of vehicle and different SPEFL 118 dose-treated tumor mice via weekly i.v. (1 time) and p.o. (3 times) as shown (Arrowed). Tumor mouse setup is the same as in FIG. 81A. Each tumor growth curve from vehicle-treated and different SPEFL118 dose-treated mice is the mean tumor size+SD der.ed from 3-4 tumors on 3-4 female mice. FIG. 85B. Mouse body weight change curves: The mouse body weight change curve from vehicle-treated and different SPEFL 118 dose-treated mice is the mean body weight+SD derived from 5 female mice in each different group.

FIG. 86 shows the antitumor efficacy and toxicity (animal body weight loss) of vehicle and 4-dose SPEFL 118 with the schedule of weekly×4 via i.v. administration in the fibrosarcoma HT1080 (CCL-121) tumor models in female SCID mice. FIG. 86A. The efficacy curve of vehicle and 4-defferent SPEFL 118 doses via i.v. using weekly×4 schedule (arrowed). Tumor mouse setup is the same as in FIG. 81A. Each tumor growth curve from vehicle-treated and the 4-different SPEFL 118 dose-treated mice is the mean tumor size+SD derived from 5 female mice of each dose group. Of note, all 5 mice for SPEFL118 at a dose of 12.5 mg/kg via i.v. had more than 20% body weigh lose on the day 3 after first treatment. Therefore, this SPEFL118 12.5 mg/kg group was withdrawn. FIG. 86B. Mouse body weight change curves: The mouse body weight change curve derived from vehicle-treated and each of the 4 SPEFL118 dose-treated mice (arrowed) is the mean body weight+SD derived from 5 female mice in each different group.

FIG. 87 shows the antitumor efficacy and toxicity (animal body weight loss) of SPEFL118 (10 mg/kg) with the schedule of bi-weekly×2 (Q2w×2) via i.v. administration in the human fibrosarcoma HT1080 (CCL-121) tumor animal models in female SCID mice. FIG. 87A. The efficacy curve of vehicle and SPEFL 118 at the dose of 10 mg/kg via i.v . . . . Tumor mouse setup is the same as in FIG. 81A. Each tumor growth curve from vehicle-treated and SPEFL 118-treated mice is the mean tumor size+SD derived from 5 female mice. FIG. 87B. Mouse body weight change curves: The mouse body weight change curve from vehicle-treated and SPEFL 118-treated mice (arrowed) is the mean body weight+SD derived from 5 female mice.

FIG. 88 shows the antitumor efficacy and toxicity (animal body weight loss) of vehicle and 4-dose SPEFL 118 with the schedule of weekly×4 (Qw×4) via p.o. administration in the fibrosarcoma HT1080 (CCL-121) tumor models in female SCID mice. FIG. 88A. The efficacy curve of vehicle and 4-defferent SPEFL 118 doses via p.o. using the schedule of weekly ×4 (arrowed). Tumor mouse setup is the same as in FIG. 81A Each tumor growth curve from vehicle-treated and the 4-different SPEFL 118 dose-treated mice is the mean tumor size+SD derived from 5 female mice of each dose group. Of note, the toxicity of SPEFL 118 at15 mg/kg is very high. One mouse died on day 3 and two mice died on day 14 after the first treatment. Therefore, The SPEFL118 at 15 mg/kg group was withdrawn from further studies. FIG. 88B. Mouse body weight change curves: The mouse body weight change curve derived from vehicle-treated and each of the 4 SPEFL 118 dose-treated mice (arrowed) is the mean body weight+SD derived from 5 female mice of each different group

FIG. 89 shows the antitumor efficacy and toxicity (animal body weight loss) of SPEFL118 (10 mg/kg) with the schedule of bi-weekly×2 (Q2w×2) via p.o. administration in the human fibrosarcoma HT1080 (CCL-121) tumor animal models in female SCID mice. FIG. 89A. The efficacy curve from vehicle-treated and SPEFL118 (10 mg/kg)-tumor SCID mice via p.o. routes. Tumor mouse setup is the same as in FIG. 81A. Each tumor growth curve from vehicle-treated and SPEFL 118-treated mice is the mean tumor size+SD derived from 5 female mice. FIG. 89B. Mouse body weight change curves: The mouse body weight change curve from vehicle-treated and SPEFL 118-treated mice is the mean body weight+SD derived from 5 female mice.

FIG. 90 shows the antitumor efficacy and toxicity (animal body weight loss) of vehicle and 4-dose SPEFL 118 with the schedule of weekly×4 (Qw×4) via p.o. administration in the human colorectal cancer SW620 tumor animal models in female SCID mice. FIG. 90A. The efficacy curve of vehicle and 4-defferent SPEFL 118 doses via p.o. using the schedule of weekly×4 (arrowed). Tumor mouse setup is the same as in FIG. 81A. Each tumor growth curve from vehicle-treated and the 4-different SPEFL 118 dose-treated tumor mice is the mean tumor size+SD derived from 5 female mice of each dose group. Of note, in the 15 mg/kg group, there are three mice died (one mouse (two mice) at the end of the second week because of moribund (one mouse) and body weight lose more than 20% (two mice). Therefore, the SPEFL 118 at 15 mg/kg group was withdrawn without further studies. FIG. 90B. Mouse body weight change curves: The mouse body weight change curve derived from vehicle-treated and each of the 4 SPEFL118 dose-treated mice (arrowed) is the mean body weight+SD derived from 5 female mice of each different group.

FIG. 91 shows the antitumor efficacy and toxicity (animal body weight loss) of SPEFL118 (10 mg/kg) with the schedule of bi-weekly×2 (Q2w×2) via p.o. administration in the human colorectal cancer SW620 tumor animal models in female SCID mice. FIG. 91A. The efficacy curve from vehicle-treated and SPEFL118 (10 mg/kg)-tumor SCID mice via p.o. routes. Tumor mouse setup is the same as in FIG. 81A. Each tumor growth curve from vehicle-treated and SPEFL 118-treated mice is the mean tumor size+SD derived from 5 female mice. FIG. 91B. Mouse body weight change curves: The mouse body weight change curve from vehicle-treated and SPEFL 118-treated mice is the mean body weight+SD derived from 5 female mice.

Table 1: Protein microarray (ProtoArray, Invitrogen) identified the interaction of FL118 with USP2a and UbE2T using tritium (3H)-labeled FL118 as a probe.

Table 2: Tritium (3H)-labeled FL118 binding to USP2a, UbE2T and bovine serum albumin (BSA, negative control) tested with NANOSEP 3K OMEGO device.

Table 3: Pharmacokinetics (PK) parameters of FL118 in human tumor and mouse plasma following single-dose intravenous (i.v.) administration of 1.5 mg/kg FL118.

Table 4: Pharmacokinetics (PK) parameters of FL118 in human tumor and mouse plasma following single-dose oral (p.o.) administration of 1.5 mg/kg FL118.

Table 5: FL118 drug substance (DS) manufacturing and chemical stability test plan outline.

Table 6: FL118 chemical stability results from the stress test at 60° C. for up to 30 days.

Table 7: FL118 chemical stability results from the stress test at 25° C. with high relative humility (92.5% RH) for up to 30 days.

Table 8: FL118 chemical stability results from the 10-day light stress test at 25° C./60% RH.

Table 9: FL118 chemical stability results from the accelerated test condition (40° C./75% RH) for the batch of P12211-005-P1 for up to 6 months.

Table 10: FL118 chemical stability results from the accelerated test condition (40° C./75% RH) for the batch of P12211-006-P1 for up to 6 months.

Table 11: FL118 chemical stability results from the accelerated test condition (40° C./75% RH) for the batch of C180402127-BF18001-P1 for up to 6 months.

Table 12: FL118 chemical stability results from the long-term condition (25° C./60% RH) for the batch of P12211-005-P1 for up to 24 months.

Table 13: FL118 chemical stability results from the long-term condition (25° C./60% RH) for the batch of P12211-006-P1 for up to 24 months.

Table 14: FL118 chemical stability results from the long-term condition (25° C./60% RH) for the batch of C180402127-BF18001-P1 for up to 24 months.

Table 15: Spray drying parameters for HPβCD-FL118 complex preparation.

Table 16: Characterization for HPβCD-FL118 complex prepared through spray drying.

Table 17: Spray drying parameters for HPβCD-FL118 complex preparation at 2g scale.

Table 18: Characterization for HPβCD-FL118 complex prepared via spray drying.

Table 19: HPβCD-FL118 complex 10 days stability study design.

Table 20: HPβCD-FL118 complex 10 days stability test results.

Table 21: Relative Retention Time (RRT) results of 10 days stability at two conditions.

Table 22: Results of HPβCD-FL118 complex (Batch No.: FR00535-02-190904-01) dissolution test in two mediums.

Table 23: A modified process development for HPβCD-FL118 complex preparation.

Table 24: Spray drying parameters for HPβCD-FL118 complex preparation at 10g scale.

Table 25: Characterization for the scale-up HPβCD-FL118 complex prepared via spray drying at the 10-gram level of the FL118 API.

Table 26: Summary of the mean body weights by group.

Table 27: Effects of FL118 versus vehicle on rat hematological parameters on day 10.

Table 28: Effects of FL118 versus vehicle on rats' serum biochemical parameters on day 10.

Table 29: FL118 oral treatment groups for toxicology testing with a limited number of rats.

Table 30: Effects of FL118 versus vehicle on rats' hematological parameters.

Table 31: Effects of FL118 versus vehicle on rats' serum biochemical parameters.

Table 32: FL118 oral treatment groups for toxicology testing with the standard numbers of rats.

Table 33: FL118-related changes in hematology on Day 15 (percent difference in mean values from concurrent controls).

Table 34: Effects of FL118 (Group 2, 3, 4) versus vehicle (group 1) on male rats' hematological parameters (WuXi AppTec).

Table 35: Effects of FL118 (Group 2, 3, 4) versus vehicle (group 1) on female rats' hematological parameters (WuXi AppTec).

Table 36: FL118-related changes in serum chemistry on Day 15 (percent difference in mean values from concurrent controls).

Table 37: Effects of FL118 (Group 2, 3, 4) versus vehicle (Group 1) on male rats' serum chemical parameters (WuXi AppTec).

Table 38: Effects of FL118 (Group 2, 3, 4) versus vehicle (Group 1) on female rats' serum chemical parameters (WuXi AppTec).

Table 39: FL118 oral treatment groups for toxicology testing with the FDA requirement-matched standard numbers of dogs.

Table 40: FL118 treatment-related hematology changes (M: male; F: female).

Table 41 (−1, −2): Effects of FL118 (Group 2, 3, 4) versus vehicle (group 1) on male dogs' hematological parameters (WuXi AppTec).

Table 42 (−1, −2): Effects of FL118 (Group 2, 3, 4) versus vehicle (group 1) on female dogs' hematological parameters (WuXi AppTec).

Table 43: FL118 treatment-related serum chemistry changes.

Table 44 (−1, −2): Effects of FL118 (Group 2, 3, 4) versus vehicle (Group 1) on male dogs' serum chemical parameters (WuXi AppTec).

Table 45 (−1, −2): Effects of FL118 (Group 2, 3, 4) versus vehicle (Group 1) on female dogs' serum chemical parameters (WuXi AppTec).

Table 46: FL118 whole human blood partitioning results.

Table 47: FL118 Caco-2 cell permeability results.

Table 48: FL118 human plasma protein binding results. More than 99% of FL118 bound to human blood proteins. Of note, ≥50% of human blood protein is human serum albumin (HSA).

Table 49a: FL118 half-life results in cryopreserved human hepatocytes.

Table 49b: Testosterone half-life results in cryopreserved human hepatocytes.

Table 50a: FL118 stability results from liver microsome.

Table 50b: Control compound testosterone results parameters from liver microsome.

Table 51a: FL118 stability results from liver S9 fraction.

Table 51b: The results parameters from liver S9 fraction using control compounds testosterone and 7-hydroxycoumarin.

Table 52: FL118 stability results from human plasma and whole human blood.

Table 53: FL118 is not a substrate of ABCG2/BCRP.

Table 54: FL118 does not inhibit ABCG2/BCRP to take other substrates.

Table 55: FL118 does not inhibit P-gp to take other substrates.

Table 56: CYP probe substrates and metabolites.

Table 57: FL118 does not inhibit CYP P450 (CYP1A2, CYP2B6, CYP3A) activity.

Table 58a: Positive inducers used for FL118 CYP induction studies.

Table 58b: Human hepatocytes used for FL118 CYP induction studies.

Table 59: No induction of CYP1A2 mRNA in human hepatocytes.

Table 60: No induction of CYP2B6 mRNA in human hepatocytes.

Table 61: A minor induction (5-8%) of CYP3A4 in one of the three human hepatocyte donors.

Table 62: Cell viability after CYP Induction treatment in human hepatocytes.

Table 63: Summary of experimental conditions for hERG inhibition.

Table 64a: FL118's effects on hERG Inhibition hERG inhibition-Individual Data are shown.

Table 64b: Summary of FL118's effects on hERG inhibition.

Table 65: FL118 does not induce the TA97A bacterial strain mutation.

Table 66: FL118 does not induce the TA98 bacterial strain mutation.

Table 67: FL118 does not induce the TA100 bacterial strain mutation.

Table 68: FL118 does not induce the TA1535 bacterial strain mutation: a, Bacterial tester strain TA1535 results with the Test article FL118; and b, Bacterial tester strain TA1535 results with the Test article FL118 (partial repeat).

Table 69: FL118 does not induce the WP2-uvrA bacterial strain mutation.

Table 70: MLA suspension growth after treatment with vehicle (DMSO), methyl cyclophosphamide (CP) and test article FL118 for 4h.

Table 71: Results derived from MLA cells treated with vehicle (DMSO), cyclophosphamide (CP) and the test article FL118 for 4h.

Table 72: Summary of MLA results derived from MLA cells treated with vehicle (DMSO), cyclophosphamide (CP) and the test article FL118 for 4h.

Table 73: MLA suspension growth after treatment with vehicle (DMSO), methyl methanesulfonate (MMS) and test article FL118 for 24h.

Table 74: Results derived from MLA cells treated with vehicle (DMSO), methyl methanesulfonate (MMS) and the test article FL118 for 24h.

Table 75: A summary of MLA results derived from MLA cells treated with vehicle (DMSO), methyl methanesulfonate (MMS) and the test article FL118 for 24h.

Table 76: FL118 pharmacokinetic (PK) Parameters obtained from mouse plasma and human tumor tissue by using single-dose oral administration of 1.5 mg/kg FL118.

Table 77: Mouse plasma and human tumor tissue FL118 concentrations (ng/ml and ng/g, respectively) over time (hours) derived from a single dose oral administration of 1.5 mg/kg FL118.

### DETAILED DESCRIPTION OF THE DISCLOSURE

The present disclosure relates to the new discovery that FL118 targets the protein DDX5. Reported in May 2022 Clinical and Translational Medicine (CTM), FL118 binds to and then dephosphorylates and degrades the oncogenic protein, DDX5 (also called p68), which control its downstream targets, survivin, Mcl-1, XIAPJ, cIAP2, etc. DDX5 encodes a member of the DEAD box family of RNA helicases that are involved in a variety of cellular processes. As a result of its role as an adaptor molecule, DDX5 promotes interactions with a large number of other factors. This protein is involved in pathways that include the alteration of RNA structures, plays a role as a coregulator of transcription, a regulator of splicing, and in the processing of small noncoding RNAs.

The present disclosure describes the role that the oncogenic protein DDX5 plays a role in the formation of dense and stiff stroma extra cellular matrix (ECM) structure, known as desmoplasia. Desmoplasia is the growth of fibrous connective tissue secondary to an insult or around a malignant neoplasm, usually causing dense fibrosis around tumors. By directly binding to and degrading DDX5, FL118 interferes with desmoplasia formation.

It is believed that FL118 affects the desmoplastic reaction via inhibiting the expression of the proteoglycan Agrin, a proteoglycan that promotes cancer stroma extracellular matrix (ECM) stiffness (i.e., desmoplasia). DDX5 knockout or knockdown inhibits Agrin mRNA, Agrin protein, as well as Agrin downstream target, YAP1. YAP1 protein acts as a transcription coregulator that promotes transcription of genes involved in desmoplasia promotion, cellular proliferation and suppressing apoptotic genes.

FL118 also targets ubiquitin specific peptidase 2 (USP2a). This gene encodes for an ubiquitin-specific protease that can deubiquitinates polyubiquitinated target proteins such as fatty acid synthase, murine double minute 2 (MDM2), MDM4/MDMX and cyclin D1. MDM2 and

MDM4 are negative regulators of the p53 tumor suppressor and cyclin DI is required for cell cycle G1/S transition. Moreover, USP2a is required for TNF-alpha (tumor necrosis factor alpha)-induced Nuclear factor-kappa (NF-kB) signaling. Tumor necrosis factor-α (TNF-α) play a vital role in regulation of the inflammatory process in tumor development. Nuclear factor-kappa B (NF-κB) is one of the key transcription factors which regulate processes in tumor promotion. The studies demonstrated that FL118 inhibits USP2a deubiquitinase activity in vitro and in vivo.

FL118 also targets ubiquitin conjugating enzyme E2 T (UbE2T). UbE2T is the E2 ubiquitin-conjugating enzyme of the Fanconi anemia DNA repair pathway and it is overexpressed in several cancers. FL118 or a FL118-based compound inhibits UbE2T protein but not its mRNA. Silencing of UbE2T results in the downregulation of survivin, Mcl-1, XIAP, and cIAP2 in parallel with cell apoptosis induction

The present disclosure further relates to the use of DDX5, USP2a and UbE2T as the cancer therapeutic targets for FL118 or a FL118-based compound in one or more defined formulations to treat cancer alone and mechanistically in combination with other cancer therapeutic approaches (e.g., chemotherapy, targeted therapy, or immunotherapy). Likewise, methods for the defined formulation of FL118 as well as the mechanistically based FL118 formulation in combination with the defined other therapeutics for the treatment of human cancers are disclosed herein.

The present disclosure further relates to the novel pharmaceutical formulation composition and process of FL118 or a FL118-based compound for its use alone or mechanistically in combination with other cancer therapeutics.

The present disclosure describes the newly expanded formulation of the anticancer drug FL118 or a FL118-based compound using a single protein (human protein or humanized protein) to encapsulate FL118 to form a protein encapsulated FL118 or a FL118-based compound in a non-covalent manner. The detailed content of an example of this invention is described below.

In one embodiment, the human protein described in this invention is the human serum albumin (HSA) to be used for the formulation of FL118 or a FL118-based compound to form a stable HSA-FL118 or a FL118-based compound non-covalent complex. The stable HSA-FL118 or a FL118-based compound non-covalent complex means that FL118 or a FL118-based compound is stably encapsulated within the HSA protein through such as hydrophobic effects, van der Waals forces, hydrogen bonds for FL118 to tightly intact with HSA via HSA encapsulation of FL118 or a FL118-based compound small molecule inside. In another embodiment, only part of the FL118 or a FL118-based compound molecule is encapsulated by HSA.

In another embodiment, the human protein described in this invention is the human globulin to be used for the encapsulation of FL118 or a FL118-based compound to form a stable globulin encapsulated FL118 (non-covalent nature). The stable globulin encapsulated FL118 or a FL118-based compound means that FL118 or a FL118-based compound is stably encapsulated within the globulin protein through non-covalent bonds such as hydrophobic effects, van der Waals forces, hydrogen bonds for FL118 or a FL118-based compound to tightly intact with globulin via globulin encapsulation of FL118 or a FL118-based compound small molecule inside. In another embodiment, only part of the FL118 or a FL118-based compound molecule is encapsulated by globulin.

In the compositions of this invention, the HSA or human globulin is not present in the form of a polymer or an aggregate status such as nanoparticles. Therefore, the tightly bound FL118 or a FL118-based compound is encapsulated within the protein superstructure of HSA or the protein superstructure of human globulin, but not within pores of their possible nanoparticle.

The term “human globulin” includes alpha globulin, beta globulin, and gamma globulin. Non-limiting examples of various globulins include human clotting proteins, complement, many acute phase proteins, immunoglobulins, and lipoproteins. In one embodiment, the globulin is recombinant human globulin. In one embodiment, the human globulin is human immunoglobulin (Ig), including, but not limited to, human IgG, IgA, IgM, IgE or IgD antibody.

Human antibody also includes the humanized mouse monoclonal antibody (mAb) or a human/humanized mAb fragment that targets a cancer-associated cell surface antigen such as EGFR. Specifically, the term “antibody” includes a single-chain variable fragment (scFv or “nanobody”), humanized, fully human or chimeric antibodies, single-chain antibodies, diabodies, and antigen-binding fragments of antibodies that do not contain the Fc region (e.g., Fab fragments). In certain embodiments, the antibody is a human antibody or a humanized antibody. A “humanized” antibody contains only the three CDRs (complementarity determining regions) and sometimes a few carefully selected “framework” residues (the non-CDR portions of the variable regions) from each donor antibody variable region recombinantly linked onto the corresponding frameworks and constant regions of a human antibody sequence. A “fully humanized antibody” is created in a hybridoma from mice genetically engineered to have only human-derived antibody genes or by selection from a phage-display library of human-derived antibody genes.

In another embodiment, the term “fibrinogen” includes any human fibrinogen. Human fibrinogen is a soluble glycoprotein present in human blood plasma, from which fibrin is produced by the action of the enzyme thrombin. In one embodiment, the fibrinogen is a recombinant human fibrinogen.

In the current invention, the encapsulation of FL118 or a FL118-based compound by a human protein including the use of various “polar organic solvent”. The term of polar organic solvent includes solvents that are miscible with water or partially dissolved in water. Specifically, these polar organic solvent includes (1) Water soluble alcohols: methanol, ethanol, isopropanol, butanol, pentanol, t-butanols, etc. (2) Water soluble diols and triols, tetraols: ethylene glycol, propylene glycol, glycerol, etc. (3) Water soluble aldehydes and ketones: acetone, butanone, pentanones, hexanones, acetaldehyde, formyl aldehyde, propionaldehyde, butyraldehyde, etc. (4) Water soluble nitriles: acetonitrile, propionitrile, butanitrile, etc. (5) Water soluble polymers with low molecular weight: polyethylene glycols, polypropylene glycols, etc. (6) Water soluble amides: DMF, dimethylacetamide, dimethylpropanamide, etc. (7) Water soluble ethers: diethyl ether, THF, dioxanes, etc. (8) All Other water soluble organic solvents: DMSO, etc.

In the current invention, the encapsulation of FL118 or a FL118-based compound by a human protein may or may not include an excipient that was dissolved in a type of “polar organic solvent” for the human protein to formulate/encapsulate FL118 or a FL118-based compound into a protein-drug-excipient complex.

In one embodiment, the excipient including the human protein formulation/encapsulation process is a type of cyclodextrin (CD) including, buy may not be limited to, β-cyclodextrin (β-CD), hydroxypropyl-β-cyclodextrin (HPβCD), sulfobutylether-β-cyclodextrin (SBEβCD). These types of CDs could be completely dissolved into most if not all “polar organic solvents). Thus, both a human protein in a “polar organic solvent” with or without a type of CD will effectively encapsulate the hydrophobic FL118 API (active pharmaceutical ingredient).

In another embodiment, HSA encapsulates FL118. HSA is well-known for its conformation changes when its environment is altered. It has been reported that HSA displayed different confirmations in acidic, neutral and basic conditions. HSA's conformation in a polar organic solvent or a cosolvent, such as ethanol, or methanol/ethanol with water, or 1, 4-dioxane with water, or 2-butanone/ethanol with water, or acetone with water, or formic acid with water or acetic acid with water or DMSO with water, is dramatically different from the pure water. For example, studies in the literature shows that suspending HSA in the water/organic cosolvents is accompanied by two main processes, (1) the water desorption-sorption, (2) the non-sorption that is attributed to rupture of protein-protein contact, depending on the nature of organic solvent and water content. Furthermore, the prepared HSA solution in the water/organic cosolvents results in the increase in the accessible surface areas, which has capacity to change the water sorption and calorific properties of the intended HSA suspension. HSA in the water/organic cosolvents is no longer in its natural state; it is partially denatured. Due to the fact that relative polarity of the cosolvent is lower than the pure water's, the resulting conformation changes of HSA in the desired organics/water mixture would allow some of the hydrophobic pockets to be opened up, and thus allowing the hydrophobic FL118 or a FL118-based compound with or without a type of CD to be tightly/encapsulated into these hydrophobic pockets. Surprisingly, FL118 or a FL118-based compound formulated using HSA with or without a type of CD can be administrated not only by i.v. or i.p., but also through the p.o. route with equivalent to or even better than the antitumor efficacy of these using i.v. or i.p. routes.

As shown in the invention examples, the single protein (HSA)-encapsulated FL118 or a FL118-based compound product exhibited high efficacy to inhibit human tumors in human tumor animal models with low toxicity. One reason is because the encapsulated FL118 or a FL118-based compound has a higher affinity to the cancer cells and will have fewer interactions with the normal cells.

Surprisingly, as shown in the examples, the single protein non-covalently encapsulated FL118 formulation product is orally available with even lower toxicity than in i.v. or i.p. routes, while oral administration of HSA-encapsulated FL 118 product could obtain similar or even better antitumor activity in comparison with those from i.v. and i.p. routes. It is well known that p.o. routes have advantages over the drugs that can only be administrated via i.v. or i.p. routes.

In one embodiment, the single protein is dissolved in a polar organic solvent with water or a co-solvent with water containing at least one water soluble organic solvent that helps the pharmaceutical agent to be able for being encapsulated into the single protein with or without the presence of a type of CD. The encapsulation process is monitored by UV or other instruments. Once the desired percent of single protein-encapsulated FL118 or a FL118-based compound is achieved, the encapsulation process is terminated and the final FL118 or a FL118-based compound product is prepared. After the filtration (e.g., through 0.22 um membrane or high-speed centrifugation or other sterilization procedure), the concentrations of FL118 can be quantified by UV spectrometer, HPLC or other methods after organic solvent extraction through the protein precipitation. After quantification, the single protein-encapsulated FL118 solutions can be lyophilized to powder products for storage. In addition, in some embodiments, the single protein-encapsulated FL118 can be further purified via running through Sephadex G25 column, in which the large molecule, single protein-encapsulated FL118 come out the first, followed by the un-capsulated FL118 (if any). In short, this invention provides novel methods to prepare single protein-encapsulated FL118 (SPEFL118) or a FL118-based compound without chemically modifying structures of the single protein by FL118 or a FL118-based compound in the presence or absence of a type of CD.

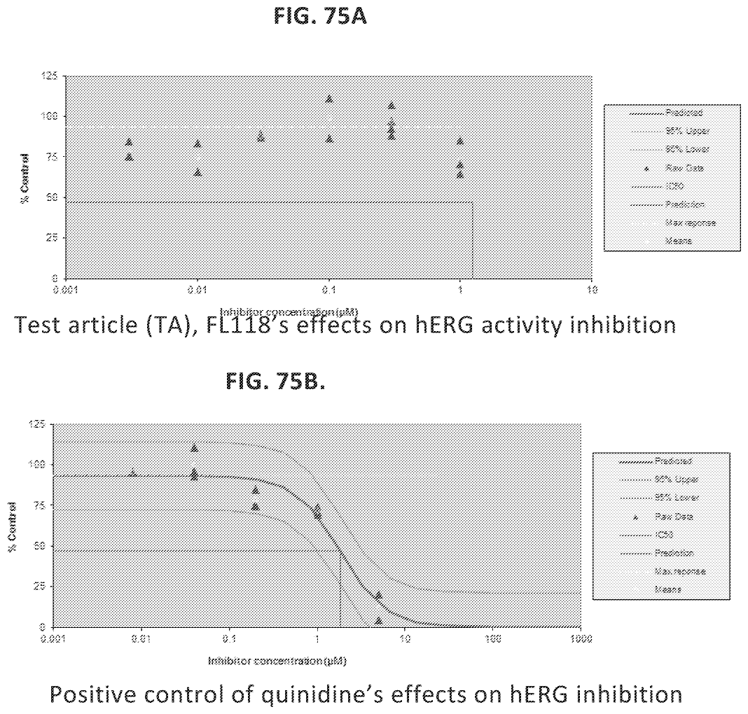

The single protein in this invention is HSA. HSA is a biopolymer, with a molecular weight at about 66K with a particle size at about 10 nm determined by dynamic light scattering (DLS). In the compositions of this invention the particle size of the albumin having one or more molecules of FL118 (with MW 392) or a FL118-based compound encapsulated therein does not change, see FIGS. 75-79. The HSA having the FL118 tightly bound therein is soluble and stable in water or saline at a pH in the range of from about 4 to 6.5.

In this invention, the formulated project compositions include a human protein-encapsulated the hydrophobic drug FL118 or a FL118-based compound with or without a type of CD in an aqueous solution with a pH 4 to <7. The aqueous composition solution can be prepared from any form of the SPEFL118 including the lyophilized SPEFL118 format. The obtained aqueous composition solution can be in turn administered orally, intravenously or intraperitoneally via weekly with every 4-week as a course, or bi-weekly with every 4-6 week as a course or daily for five times per week with every other week per course.

The obtained aqueous composition solution will be administrated at a dose range of from 0.1 mg/kg to about 10 mg/kg, which will depend on animal species and the drug administrative routes.

The single protein encapsulation of FL118 described in this invention is a novel approach to deliver FL118 for the better efficacy without toxic organic solvents. The antitumor efficacy testing with human tumor animal models obtained surprising outcomes (high efficacy, low toxicity with increase maximum tolerated dose (MTD)). This is because single Protein Encapsulation can be particularly useful with hydrophobic pharmaceutical agents like FL118 that is not water-soluble.

In one embodiment, the invention provides a method comprising: a polar organic solvent with water in the presence or absence of a type of CD for a protein to formulate FL118 or a FL118-based compound into a non-covalent protein-FL118 complex, which may or may not need to provide a third polar organic solvent with water for formulation. In one embodiment, the invention further provides sterilizing the third solution via a filtration to remove microorganisms.

In one embodiment, the FL118 or a FL118-based compound molecule is completely encapsulated within the single protein. the protein-encapsulated FL118 or a FL118-based compound product is administrated via oral routes (p.o.), intravenous routes (i.v.) or intraperitoneal routes (i.p.) through weekly schedules, bi-weekly schedules or daily for five times per week.

In one embodiment, the invention-provided single protein-formulated protein-FL118 or a FL118-based compound complex in a non-covalent or covalent state is applied for various types of cancer treatment alone or in combination with other type of existed cancer treatment approaches including chemotherapy, immunotherapy, or radiotherapy.

Use of HSA and IgG as single protein for FL118 or a FL118-based compound formulation has additional considerations. The long half-life of HSA or IgG is maintained mainly by the neonatal Fc receptor (FcRn) or the Brambell receptor, which is expressed in a wide variety of tissues and organs. This major histocompatibility complex class I-related receptor was originally discovered to play an important role in the delivery of IgGs from the mother to the young, regulate serum IgG concentration, and maintain.

The long half-life of IgGs in the serum. It was later discovered that FcRn can bind both IgG and HSA at different sites and is responsible for the long half-lives of both IgGs and HSA. As expected, FcRn mutation was found to cause familial hypercatabolic hypoproteinemia (Wani, M. A., et el., Proc Natl Acad Sci USA, 2006, 103, 5084-5089). The mechanism of HSA rescue and recycling involves: (1) FcRn binding HSA in the endosome due to high affinity at acidic pH. (2) The resulting FcRn-HSA complex (1:1 ratio) is sent back to the bloodstream. (3) The FcRn-HSA complex dissociates due to low affinity at pH 7.4, releasing HSA back in the circulation. In cells that express low levels of FcRn, HSA would be endocytosed to lysosomes, where it is degraded to amino acids. As a result, this FcRn-mediated recycling pathway is a major factor contributing to the long half-life of both IgGs and HSA in human and has significant pathophysiological and therapeutic implications.

A human antibody or a humanized mouse monoclonal antibody (mAb) or a human/humanized mAb fragment that targets a cancer-associated cell surface antigens are non-covalently and covalently formulated with FL118 or a FL118-based compound for various types of cancer treatment alone or in combination with other type of existed cancer treatment approaches such as chemotherapy, immunotherapy, or radiotherapy via oral routes (po), intravenous routes (i.v.) or intraperitoneal routes (i.p.) through weekly schedules, bi-weekly schedules or daily for five times per week.

The definitions of certain terms as used in this invention are provided below. Unless defined otherwise, all technical and scientific terms used herein generally have the same meaning as commonly understood by one with ordinary skill in the art to which this disclosure belongs. As used in this invention specification and the appended claims, the singular forms “a”, “an” and “the” include plural referents unless the content clearly dictates otherwise. For example, reference to “a type”, “a class” includes a combination of two or more types, classes and the like.

### Terminology

As used herein, the term “desmoplasia” refers to a dense and stiff stroma ECM structure associated with tumors and cancer (e.g., pancreatic cancer), and may prevent therapeutics (e.g., chemotherapies, targeted therapies, or immunotherapies) access the tumor cells.

As used herein, the term “cancer therapeutics” refers to any approaches that are used for treating cancer including, but should not be limited to, various types of chemotherapies, targeted therapies including antibody therapy and immunotherapies including small molecules, biologicals, checkpoint inhibitors and various types of immune cell therapies.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. The term “about” as used in connection with a numerical value throughout the specification and the claims denotes an interval of accuracy, familiar and acceptable to a person skilled in the art. In general, unless otherwise indicated, the interval of accuracy is +/−10%. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

As used herein, the “administration” of an agent or drug, e.g., one or more compounds or other types of anticancer agents, to a subject or subjects includes any route of introducing or delivering to a subject a compound to perform its intended function. Administration can be carried out by any suitable route, including orally, intranasally, by inhalation, parenterally (intravenously, intramuscularly, intraperitoneally, or subcutaneously), rectally, or topically.

Administration includes self-administration and the administration by another. It is also to be appreciated that the various modes of treatment or prevention of medical conditions as described are intended to mean “substantial”, which includes total but also less than total treatment/prevention, and wherein some biologically or medically relevant result is achieved.

As used herein, the terms “assessing,” “assaying,” “determining,” and “measuring” are used interchangeably and include both quantitative and qualitative determinations. These terms refer to any form of measurement, and include determining if a characteristic, trait, or feature is present or not. Assessing may be relative or absolute. “Assessing the presence of” includes determining the amount of something present and/or absent.

As used herein, the term “clinical factors” refers to any data that a medical practitioner may consider in determining a diagnosis, prognosis, or therapeutic regimen for treating or preventing a disease or diseases. Such factors include, but are not limited to, the patient's medical history, a physical examination of the patient, complete blood count, examination of blood cells or bone marrow cells, cytogenetics, pulmonary health, vascular indications of disease, and immunophenotyping of cells.

As used herein, the terms “comparable” or “corresponding” in the context of comparing two or more samples, responses to treatment, or drugs, refer to the same type of sample, response, treatment, and drug respectively used in the comparison. In some embodiments, comparable samples may be obtained from the same individual at different times. In other embodiments, comparable samples may be obtained from different individuals, e.g., different patients or even healthy individuals such as a blood sample. In general, comparable samples are normalized by a common factor for control purposes.

As used herein, the term “composition” refers to a product with specified ingredients in the specified amounts, as well as any product which results, directly or indirectly, from combination of the specified ingredients in the specified amounts.

As used herein, the term “diagnosis” means detecting a disease or disorder or determining the stage or degree of a disease or disorder. Typically, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease, i.e., there may be differential diagnoses that can be inferred from a diagnostic factor or symptom. Likewise, there may be instances where a factor or symptom that is indicative of a particular disease is present in an individual that does not have the particular disease. The term “diagnosis” also encompasses determining the therapeutic effect of a drug therapy, or predicting the pattern of response to a drug therapy. The diagnostic methods may be used independently, or in combination with other diagnosing and/or staging methods known in the medical art for a particular disease or disorder.

As used herein, the terms “drug,” “compound,” “active agent,” “agent,” “actives,” “pharmaceutical composition,” “pharmaceutical formulation,” and “pharmacologically active agent” or “active pharmacological agent” are used interchangeably and refer to any chemical compound, complex or composition, charged or uncharged, that is suitable for administration and that has a beneficial biological effect, suitably a therapeutic effect in the treatment of a disease or abnormal physiological condition, although the effect may also be prophylactic in nature. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent,” and “API” (active pharmaceutical ingredient) are used, then, or when a particular active agent is specifically identified, it is to be understood that applicants intend to include the active agent per se as well as pharmaceutically acceptable, pharmacologically active salts, esters, amides, prodrugs, conjugates, metabolites, analogs, etc.

As used herein, the terms “effective amount” or “pharmaceutically effective amount” or “therapeutically effective amount” of a composition, is a quantity sufficient to achieve a desired therapeutic and/or prophylactic effect, e.g., an amount which results in the prevention of, or a decrease in, the symptoms associated with a disease that is being treated. The amount of a composition of the disclosure administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions of the present disclosure can also be administered in combination with one or more additional therapeutics.

As used herein, the term “neoplastic disease” refers to cancers of any kind and origin and precursor stages thereof. Accordingly, the term “neoplastic disease” includes the subject matter identified by the terms “neoplasia”, “neoplasm”, “cancer”, “pre-cancer” and “tumor.” A neoplastic disease generally manifests by abnormal cell division resulting in an abnormal level of a particular cell population. Likewise, the monoclonal expansion of endothelial cells may refer to a “neoplasm” of the pulmonary arteriolar endothelial cells. The abnormal cell division underlying a neoplastic disease, moreover, is typically inherent in the cells and not a normal physiological response to infection or inflammation. In some embodiments, neoplastic diseases for diagnosis using methods provided herein include carcinoma. By “carcinoma,” it is meant a benign or malignant epithelial tumor.

As used herein, the term “pharmaceutically acceptable salt” includes a salt with an inorganic base, organic base, inorganic acid, organic acid, or basic or acidic amino acid. As salts of inorganic bases, the disclosure includes, for example, alkali metals such as sodium or potassium; alkaline earth metals such as calcium and magnesium or aluminum; and ammonia. As salts of organic bases, the disclosure includes, for example, trimethylamine, triethylamine, picoline, ethanolamine, diethanolamine, and triethanolamine. As salts of inorganic acids, the instant disclosure includes, for example, hydrochloric acid, hydroboric acid, nitric acid, sulfuric acid, and phosphoric acid. As salts of organic acids, the instant disclosure includes, for example, formic acid, acetic acid, trifluoroacetic acid, fumaric acid, oxalic acid, tartaric acid, maleic acid, lactic acid, citric acid, succinic acid, malic acid, methanesulfonic acid, benzenesulfonic acid, and p-toluenesulfonic acid. As salts of basic amino acids, the instant disclosure includes, for example, arginine, lysine and ornithine. Acidic amino acids include, for example, aspartic acid and glutamic acid.

As used herein, the term “prognosis” refers to a prediction of the probable course and outcome of a clinical condition or disease. A prognosis is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. The terms “favorable prognosis” and “positive prognosis,” or “unfavorable prognosis” and “negative prognosis” as used herein are relative terms for the prediction of the probable course and/or likely outcome of a condition or a disease. A favorable or positive prognosis predicts a better outcome for a condition than an unfavorable or negative prognosis. In a general sense, a “favorable prognosis” is an outcome that is relatively better than many other possible prognoses that could be associated with a particular condition, whereas an unfavorable prognosis predicts an outcome that is relatively worse than many other possible prognoses that could be associated with a particular condition. Typical examples of a favorable or positive prognosis include a better than average cure rate, a lower propensity for metastasis, a longer than expected life expectancy, differentiation of a benign process from a cancerous process, and the like. For example, a positive prognosis is one where a patient has a 50% probability of being cured of a particular cancer after treatment, while the average patient with the same cancer has only a 25% probability of being cured.

As used herein, the term “reference level” refers to a level of a substance which may be of interest for comparative purposes. In some embodiments, a reference level may be a specified composition dosage as an average of the dose level from samples taken from a control subject. In other embodiments, the reference level may be the level in the same subject at a different time, e.g., a time course of administering the composition, such as the level determined between 1 minute (min) and 10 min, etc.

As used herein, the terms “sample” or “test sample” refer to any liquid or solid material containing collected from a subject. In suitable embodiments, a test sample is obtained from a biological source, i.e., a “biological sample,” such as cells in culture or a tissue sample from an animal, most preferably, a murine subject, mammal or human subject.

As used herein, the terms “subject” or “individual,” refer to a mammal, such as a mouse, rat, or human, but can also be another animal such as a domestic animal, e.g., a dog, cat, or the like, a farm animal, e.g., a cow, a sheep, a pig, a horse, or the like, or a laboratory animal, e.g., a monkey, a rat, a mouse, a rabbit, a guinea pig, or the like. The term “patient” refers to a “subject” who is, or is suspected to be, afflicted with a disease.

As used herein, the terms “treating” or “treatment” or “alleviation” refer to both therapeutic treatment and prophylactic or preventative measures, wherein the objective is to prevent or slow down (lessen) the targeted pathologic condition or disorder. A subject is successfully “treated” for a disorder if, after receiving a therapeutic agent according to the methods of the present disclosure, the subject shows observable and/or measurable reduction in or absence of one or more signs and symptoms of a particular disease or condition.

Generally, the method includes dissolving the hydrophobic molecule FL118 or a FL118-based compound in a suitable organic solvent to form a first solution. In an embodiment, cyclodextrin has been dissolved in the suitable organic solvent. In another embodiment the suitable organic solvent is cyclodextrin-free. Then, dissolving a human protein or a humanized protein or a humanized protein fragment in an aqueous solution forms a second solution. The first solution and second solution are combined to form a third solution, and the organic and aqueous components are separated/removed from the third solution to produce a complex. Then, before application of the protein-FL118 or a FL118-based compound for cancer treatment, the protein-FL118 or a FL118-based compound complex product is dissolved in an aqueous solution such as sterile ddH2O water, saline, PBS or a commercialized aqueous solution like ORA-Plus or ORA-Blend etc. (SpecializedRx). The organic solvent of the first solution includes one or more of the organic solvents described elsewhere. For example, the organic compound acetone solvent may be a ketone-containing compound, such as an aliphatic ketone that includes 3-5 carbon atoms (i.e., a C3-C5 ketone), such as, but not limited to, acetone, methyl ethyl ketone, 2-pentanone, and 3-pentanone.

In some embodiments, the compound of formula 1 (hydrophobic FL118 or a FL118-based compound) is soluble in one or more organic solvents. In an embodiment, the compound of formula 1 can be dissolved in a solvent in which cyclodextrin has been dissolved. In another embodiment, compound of formula 1 can be dissolved in a solvent which is free of cyclodextrin. The organic solvent should be more volatile or close to the volatility point of water and miscible with other organic solvents such as ketone and/or ketone derivative solvents. In the case that two organic solvents are used, the second organic solvent may include, but may not be limited to, methanol, ethanol, dichloromethane, acetonitrile, benzene, n-butanol, butyl acetate, carbon tetrachloride, chloroform, cyclohexane, 1,2-dichloroethane, dioxane, ethyl acetate, diethyl ether, heptane, hexane, methyl-t-butyl ether, 2-butanone, pentane, n-propanol, isopropanol, diisopropyl ether, tetrahydrofuran, toluene, trichloroethylene, and combinations thereof. In some cases, the hydrophobic FL118 or FL118-based compound is initially dissolved in a second organic solvent containing one or more of the second organic solvents. Then, the second organic solvent is combined with the C3-C5 ketone-containing compound, e.g., acetone or acetone-derived organic solvents.

The ratio of the first organic solvent to the second organic solvent may be any suitable ratio to facilitate the hydrophobic FL118 or FL118-based compound to be encapsulated into HSA to become HSA-FL118 or HSA-FL118-based compound non-covalent complex. In an embodiment, wherein two organic solvents are used to form a solution, the ratio of organic solvents may be combined in any ratio between about 1:99 to between about 9:1. In an embodiment, the molar ratio of a compound of formula 1 (FL118 or a FL118-based compound) to protein may be about 4:1 to about 12:1.

The amount of FL118 in the first solution may vary, depending on the desired outcome in the presence or absence of a type of CD. In some cases, FL118 or a FL118-based compound in the first solution is at a concentration range of 0.1-10 mg/mL

The second solution may be an aqueous solution with or without other organic solvent in any percentage and may include HSA in any suitable amount and percentage, and may include additional components, such as pH controlling agents to control pH<7. These pH controlling agents include, but may not be limited to, phosphate, bicarbonate, citrate, tris (hydroxymethyl) aminomethane (Tris), N-Tris (hydroxymethyl)methyl-3-aminopropanesulfonic acid (TAPS), bicine, tricine, 4-(2-hydroxyethyl)-1-piperazineethanesulfonic acid (HEPES), 3-(N-morpholino)-propanesulfonic acid (MOPS), 2-(N-morpholino)-ethanesulfonic acid (MES) and piperazine-N,N′-bis(2-ethanesulfonic acid) (PIPES). Other suitable additional components include, without limitation, minerals/salts, antioxidants (such as, e.g., ascorbic acid), chelating agents (such as, e.g., ethylenediaminetetraacetic acid (EDTA) or glutathione), amino acids (such as, e.g., glycine), proteins, preservatives, tonicity controlling agents, and the like. In some cases, Solution 2 includes phosphate-buffered saline (PBS). In some cases, the second solution includes a compound that modulates albumin conformation such as hydrophobic free radical scavengers (e.g. 2,6-di-tert-butyl-4-methylphenol, Vitamin E).

In some cases, the second solution may include an antioxidant, e.g., ascorbic acid. The antioxidant may be present in any suitable amount.

In illustrative embodiments, the process formulation-resulted protein-FL118 (or a FL118-based compound) complex product is administered in a daily, weekly to biweekly dosage from about 0.1 mg/kg to about 20 mg/kg via orally or i.v. routes.

### Overview

There are several causes to treatment resistance in cancer. A nearly universal mechanism that causes treatment resistance is tumor desmoplasia. Tumor desmoplasia can block access tumor cells for various treatment and different therapeutics. The desmoplasia-based tumor microenvironment (TME) can create a protective niche that facilitates tumor proliferation, survival, immunosuppression, and therapy resistance (Meads MB et al. Environment-mediated drug resistance: a major contributor to minimal residual disease. Nat Rev Cancer. 2009, 9:665-674; de Haart S J et al. Accessory cells of the microenvironment protect multiple myeloma from T-cell cytotoxicity through cell adhesion-mediated immune resistance. Clin Cancer Res. 2013, 19:5591-5601). Therefore, new strategies to disrupt the tumor desmoplasia are needed.

One aspect of the present disclosure shows that FL118 is a cancer desmoplasia disruptor which can help other cancer therapeutics (e.g., chemotherapies, targeted therapies, or immunotherapies) to more effectively killing cancer cells. The present disclosure also demonstrates that tumor desmoplasia depends on the DDX5-controlled Agrin signaling pathway. The disclosure shows that the DDX5-promoted Agrin signaling pathway can be inhibited by FL118 and provides a mechanism of action for FL118 to disrupt tumor desmoplasia—by FL118 targeting DDX5. By disrupting tumor desmoplasia, other cancer therapeutics (e.g., chemotherapies, targeted therapies, or immunotherapies) have a chance of working; breaking down tumor defenses or gaining access to tumor cells to kill or stop tumor growth. This FL118-associated novel strategy to overcome desmoplasia-associated treatment resistance is further strengthened by other unexpected discoveries disclosed herein.

High heterogeneity in cancer presents another type of major mechanism of treatment resistance (Swanton C: Intratumor heterogeneity: evolution through space and time, Cancer research 2012, 72:4875-4882). For example, the gene-expression signature of favorable versus unfavorable prognosis can be detected in different regions of the same tumor, and a significant percentage of somatic mutations may not be detected across every tumor region of the same tumor (Gerlinger M, et al.: Intratumor heterogeneity and branched evolution revealed by multiregion sequencing, The New England Journal of Medicine 2012, 366:883-892). This extensive intra-tumor heterogeneity presents another difficult challenge with respect to personalized cancer treatment (personalized medicine) and biomarker development.

The present disclosure describes the discovery of two new oncogenic proteins, USP2a and UbE2T, which FL118 targets functionally and directly. FL118 disrupts desmoplasia through interactions with DDX5 and thus to inhibit the DDX5-Agrin signaling pathway and USP2a/UbE2T signaling pathways. Therefore, FL118 overcomes various mechanisms of resistance by targeting DDX5/USP2a/UbE2T signaling pathways demonstrating FL118 acting as a Triple oncogene Target Inhibitor (TTi).

This disclosure also presents a new method of manufacturing FL118-cyclodextrin complex by using the two or more organic solvents in different composition ratios for the formulation of FL118 with a type of CD (e.g., HPβCD) is disclosed. In an embodiment, the method comprises using organic solvents such as glacial acetic acid (GAA) and ethanol, and further comprises using a microfluidizer and spray drying dispersion for the production of FL118 products. The present disclosure describes exemplary embodiments of new formulations of FL118 composition and process with unique organic solvent-containing strategies as well as the organic solvent-free formulation of the FL118-HPβCD complex into a powder, allowing an aqueous suspension format for intraperitoneal, intravenous or oral administration, powder capsule format or tablet format for oral administration.

### Pharmaceutical Compositions

Pharmaceutical compositions, applicants add the newly developed and expanded disclosures of various identified organic solvents including the glacial acetic acid-microfluidizer-processed formulation of FL118 with a type of cyclodextrin (CD).

The excipients used to formulate FL118 can be any type of CD including β-cyclodextrin (βCD), hydroxypropyl-β-cyclodextrin (HPβCD), sulfobutylether-β-cyclodextrin (SBEβCD) or another type of cyclodextrin derivative.

In one recipe, methanol-HPβCD-FL118 complex or ethanol-HPβCD-FL118 complex suspension goes through a spray-dry process or a lyophilization process to remove methanol or ethanol to produce an HPβCD-FL118 complex powder format.

In another recipe, formic acid-(FA)-HPβCD-FL118 complex suspension, glacial acetic acid (GAA)-HPβCD-FL118 complex suspension, zinc acetate-(ZA)-HPβCD-FL118 complex suspension or glyoxal-HPβCD-FL118 complex suspension goes through a spray-dry process or a lyophilization process to remove these organic solvents to produce an HPβCD-FL118 complex powder format.

In the third aspect, the ethylene glycol (EG)-HPβCD-FL118 complex suspension, propylene glycol (PG)-HPβCD-FL118 complex suspension, formamide-(FAD)-HPβCD-FL118 complex suspension, (N,N,N′,N′)-tetramethyl-ethylenediamine (TEMED)-HPβCD-FL118 complex suspension, ethanolamide (EA)-HPβCD-FL118 complex suspension or 2-mercaptoethanol (MercE)-HPβCD-FL118 complex suspension goes through a lyophilization process or any other method to remove organic solvents to produce an HPβCD-FL118 complex solid format.

In the next aspect, HPβCD-FL118 complex powder/solid format is directly diluted with saline containing 0-5% propylene glycol (PG) and 0-5% polyethylene glycol 400 or 300 (PEG400 or PEG300). In some cases, the HPβCD-FL118 complex powder format is directly diluted with saline containing 2.5% PG and 2.5% PEG400 to become an aqueous suspension for administration.

In some other cases such as in the case of using glacial acetic acid (GAA)-involved formulation-processed FL118 products, the HPβCD-FL118 complex powder format is directly diluted with saline to become an aqueous suspension for administration alone or mechanistically in combination with one or more types of other cancer therapeutics (e.g., chemotherapies, targeted therapies, or immunotherapies).

In the next aspect, the HPβCD-FL118 complex powder is alternatively formulated into a tablet format, which includes the pharmaceutical composition of fillers/binders/diluents (e.g., celluloses/cellulose derivatives, starches/starch derivatives, and/or lactose), disintegrant (e.g., colloidal silicone dioxide, croscarmellose sodium, or crosspovidone), glidant (e.g., dibasic calcium phosphate or colloidal silicone dioxide), lubricants (e.g., magnesium stearate, stearic acid, polyethylene glycol, or Talc), antimicrobials/preservative (e.g., propylene glycol, propylene paraben, methyl paraben, or glycerin).

The HPβCD-FL118 complex powder can be formulated with for example, microcrystalline cellulose (MCC, 30%-80%), corn starch (0%-40%), lactose (10%-25%), colloidal silicone dioxide (0%-3%), dibasic calcium phosphate (1%-10%), and/or magnesium stearate (0.2%-3%) for making a FL118 tablet.

Pharmaceutical compositions are typically formulated to be compatible with an intended route of administration. The composition format product described above can be accordingly administered orally, intraperitoneally, or intravenously.

The pharmaceutical composition and method of the present disclosure for FL118 will be used alone or mechanistically in combination with a type of other cancer therapeutics (e.g., chemotherapies, targeted therapies, or immunotherapies) by using FL118 to disrupt tumor desmoplasia resistance to treatment.

### Pharmaceutical Formulation Process

The formulation of FL118 in this disclosure is the further to the development of related disclosures. See, PCT/US15/22095 (Use of the FL118 core chemical structure platform to generate FL118 derivatives for treatment of human disease). All of which are hereby incorporated by reference in their entirety.

The formulation of FL118 in this disclosure is in the process of first making a FL118-HPβCD formulation complex in the following process with appropriate organic solvents:

(1) Dissolving a type of cyclodextrin (CD) such as HPβCD into an appropriate organic solvent including, but may not be limited to, methanol, ethanol, formic acid (FA), glacial acetic acid, zinc acetate (ZA), glyoxal, ethylene glycol (EG), propylene glycol (PG), formamide (FAD), (N,N,N′,N′)-tetramethyl-ethylenediamine (TEMED), ethanolamide (EA) or 2-mercaptoethanol (MercE), respectively, to make a master solution of methanol-HPβCD, ethanol-HPβCD, FA-HPβCD, glacial acetic acid-HPβCD, ZA-HPβCD, glyoxal-HPβCD, EG-HPβCD, PG-HPβCD, FAD-HPβCD, TEMED-HPβCD, EA-HPβCD or MereE-HPβCD in an appropriate concentration of HPβCD at 10-40% (w/v: HPβCD 10-40g/100 mL) for formulating FL118 into a concentration of 10 mg/mL to 40 mg/mL, in turn. In this disclosure, the fixed ratio of FL118: HPβCD is 1:10 (w/w), which is roughly equivalent to one FL118 molecule to 1.5 molecules of HPβCD. Dissolution of HPβCD into methanol, ethanol, FA, glacial acetic acid, ZA, glyoxal, EG, PG, FAD, TEMED, EA or MercE should be achieved by vortex and/or shaking until all HPβCD is completely dissolved into methanol, ethanol, FA, glacial acetic acid, ZA, glyoxal, EG, PG, FAD, TEMED, EA or MercE, respectively, to become a clear solution of methanol-HPβCD, ethanol-HPβCD, FA-HPβCD, glacial acetic acid-HPβCD, ZA-HPβCD, glyoxal-HPβCD, EG-HPβCD, PG-HPβCD, FAD-HPβCD, TEMED-HPβCD, EA-HPβCD or 2MercE-HPβCD, respectively.

(2) Dispersing the FL118 API completely into the solution of methanol-HPβCD, ethanol-HPβCD, FA-HPβCD, glacial acetic acid-HPβCD, ZA-HPβCD, glyoxal-HPβCD, EG-HPβCD, PG-HPβCD, FAD-HPβCD, TEMED-HPβCD, EA-HPβCD or 2MercE-HPβCD, respectively, by a process of homogenization using a dispersion homogenizer (or other disperse methods such as vortex) to form methanol-HPβCD-FL118 complex suspension, ethanol-HPβCD-FL118 complex suspension, FA-HPβCD-FL118 complex solution, ZA-HPβCD-FL118 complex suspension, glyoxal-HPβCD-FL 118 complex suspension, PG-HPβCD-FL118 complex suspension, FAD-HPβCD-FL118 complex suspension, TEMED-HPβCD-FL118 complex suspension, EA-HPβCD-FL118 complex suspension or 2MercE-HPβCD-FL118 complex suspension, respectively. The particle size distribution in these complex suspensions after homogenization can have an approximate range of 60-800 μm.

(3) The use of a microfluidizer can reduce the particle sizes in these complex suspensions to the a range of about 60 μm to about 800 μm; alternatively, from about 30 μm to about 400 um; alternatively, from about 15 μm to about 200 um; alternatively, from about 7 μm to about 100 um; alternatively, from about 3 μm to about 50 um; alternatively, from about 1 μm to about 25 um; alternatively, from about 0.5 μm to about 15 μm; or, alternatively from about 0.1 um-11.3 um.

(4) For the resultant suspension or solution (in the FA case) of methanol-HPβCD-FL118 complex, ethanol-HPβCD-FL118 complex, FA-HPβCD-FL118 complex, glacial acetic acid-HPβCD-FL118 complex, ZA-HPβCD-FL118 complex or glyoxal-HPβCD-FL118 complex, a spray-dry process is used to remove methanol, ethanol, FA, glacial acetic acid, ZA or glyoxal, respectively, in a closed loop spray drying system with solvent recovery and an inert gas (e.g., nitrogen) to make an inert atmosphere during spray drying process. The resultant suspension of EG-HPβCD-FL118 complex, PG-HPβCD-FL118 complex, FAD-HPβCD-FL118 complex, TEMED-HPβCD-FL118 complex, EA-HPβCD-FL118 complex or MercE-HPβCD-FL118 complex should go through a lyophilization process or other approaches to remove the organic solvent of EG, PG, FAD, TEMED, EA or 2MercE, respectively, as many as possible.

(5) The resulted HPβCD-FL118 complex above can be then resuspended with clinical saline in the presence of 0-5% propylene glycol (PG) and 0-5% polyethylene glycol 400 (PEG400 in a concentration of FL118 ranging from 0.1 mg/mL to 5 mg/mL for oral administration).

A clinically compatible and most updated FL118 formulation by using the mixed organic solvents of glacial acetic acid and ethanol is provided below as a scaleup example. The principle of this process was used for manufacturing non-GLP GMP FL118 product for toxicology studies in rats and dogs and the formulation principle is currently in the process scaleup for manufacturing clinically used FL 118 products by using GMP FL118 in the GLP manufacturing condition.

1) Dissolve 20g HPβCD in 100 mL glacial acetic acid/ethanol-mixed organic solvents (glacial acetic acid: ethanol ratio=1 (10%): 9 (90%) to make a glacial acetic acid/ethanol-HPβCD solution through vortex or shaking until all HPβCD is dissolved into the glacial acetic acid/ethanol-mixed organic solvent.

2) Dissolve 2g FL118 (i.e., API: HPβCD=1:10) into the glacial acetic acid/ethanol-HPβCD solution to make the glacial acetic acid/ethanol-HPβCD-FL118 complex suspension by using a microfluidizer (e.g., LM-10) set at 5000 PSI for 3-5 cycles, which will reduce at least one order of the particle distribution sizes in the suspension.

3) Using a spray-drying dispersion process with the equipment that has a closed loop spray-dry system with solvent recovery and an inert gas (e.g., nitrogen) to make an inert atmosphere during the spray-dry process to remove glacial acetic acid and ethanol. Of note, since the glacial acetic acid/ethanol-HPβCD-FL118 complex is a suspension, the container of the glacial acetic acid/ethanol-HPβCD-FL118 complex suspension should have a magnetic bar inside on a magnetic plate for well suspension during the spray-drying process. The resultant HPβCD-FL118 complex powder product can be further dried over 30° C. overnight to get most of remaining glacial acetic acid and ethanol.

In the next aspect, FL118-HPβCD complex powder can be directly resuspended into clinical saline to a concentration 1-5 mg/mL of FL118 API for oral administration for patients.

In another aspect, FL118-HPβCD complex powder can be further made into capsule or tablet formats as follows for oral administration.

For making FL118 capsules, FL118-HPβCD complex powder can be directly made into capsules without adding additional ingredients.

For making FL118 tablets, the FL118-HPβCD complex powder (5-25%) is mixed with microcrystalline cellulose (MCC, 30%-80%), corn starch (0%-40%), lactose (10%-25%), colloidal silicone dioxide (0%-3%), dibasic calcium phosphate (1%-10%), and magnesium stearate (0.2%-3%). The excipient mixture will be further milled to let every ingredient be evenly distributed in the powder. This smooth powder is then pressed by a dry compression process to make the smooth powder into tablets.

The current disclosure is not to be limited in terms of any particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. For example, the solvents of methanol, ethanol, formic acid, or acetic acid can be alone or in an appropriate percentage mixture for dissolving a type of CD (e.g., HPβCD) as a strategy for the formulation process. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting. In addition, where features or aspects of the disclosure are described in terms of Markush groups, those skilled in the art will recognize that the disclosure is also thereby described in terms of any individual members or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member.

While various aspects and illustrative embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein are incorporated by reference herein in their entireties and for all purposes to the same extent as if each individual publication, patent, or patent application is specifically and individually incorporated by reference in its entirety for all purposes.

The foregoing summary is illustrative only and is not intended to be in any way limiting. In addition to the illustrative aspects, embodiments, and features described above, further aspects, embodiments, and features will become apparent by reference to the drawings and examples presented in the detailed description in the current disclosure.

### Materials and Methods Used

The present disclosure is further illustrated by many examples presented below, which should not be construed as limiting in any way. The following is a description of the materials and methods used throughout the examples.

Cell culture, cell lines, and reagents used: The human embryonic kidney 293T (HEK293T) cell line, human pancreatic cancer cell lines (Panc-1, MiaPaca-2. BxPC-3), human head and neck (H&N) cancer cell line (FaDu), human CRC cell lines (SW620, HT29) and human prostate cancer cell lines (LNCaP, C4-2. Du145, PC-3, 22Rv1, LAPC9) were originally purchased from ATCC. All these cell lines were maintained in either DMEM or RPMI 1640 medium supplied with 10% fetal bovine serum (FBS, Atlanta Biologicals, Lawrenceville, GA), penicillin (100 units/mL) and streptomycin (0.1 μg/mL) (Thermo Fisher Scientific/Invitrogen, Grand Island, NY). Cells were routinely sub-cultured twice a week and maintained in a humidified incubator with 5% CO2 at 37° C. Monoclonal anti-tubulin antibody, polyclonal anti-actin antibody and goat peroxidase-conjugated anti-rabbit IgG antibody were purchased from Sigma (St. Louis, MO) or Invitrogen/ThermoFisher Scientific (Waltham, MA). Antibodies for survivin (FL-142), c-Rel/NF-kB, Sp-1, c-Myc, Mcl-1, cyclin D1, normal IgG and GAPDH were from Santa Cruz (Santa Cruz, CA). DDX5 antibodies from R&D System (Minneapolis, MN) and Sino Biological (Wayne, PA). Antibodies for c-Myc, Mcl-1, cyclin DI were purchased from protein tech (Chicago, IL). Topl antibodies were purchased from TopoGen (TG2012-4, Lot12FB04) and from BD Biosciences (X-21, RUO). Antibodies from Kras, mKras, cIAP2, XIAP, Mcl-1, c-Myc, survivin, caspase-3 (cleaved and full length) and PARP (cleaved and full length) were purchased from Cell Signaling (Beverly, MA, USA). Antibodies for Agrin, YAP1, FAK, phosopho-FAK, integrin were purchased from Invitrogen/ThermoFisher Scientific, ABclonal (Woburn, MA) or Cell Signaling. MTT (3-[4,5-dimethylthiazol-2-yl]-2,5-diphenyltetrazolium bromide) and leupeptin were purchased from USB/affymetrix (Santa Clara, CA). Bovine serum albumin (BSA) was purchased from EMD (Billerica, MA). Topotecan, SN-38 and MG132 were purchased from Selleck Chemicals (Houston, TX). pCMV6-Entry-Myc-DDK (Flag)-DDX5, pCMV6-Entry-Myc-DDK-USP2, pCMV6-Entry-Myc-DDK-UbE2T and pCMV6-Entry-Myc-DDK-Topl expression vectors were purchased from OriGene (Rockville, MD). Ubiquitin-specific antibodies were purchased from UBPBio (Dallas, TX).

Purification of FL118-binding proteins using FL118 affinity column: The purification was based on the use of CarboxyLink™ Immobilization kit with UltraLink® Support (Part #53154) from Thermo Scientific (Rochford, IL). The process includes many experimental steps as described below.

FL118 ligand coupling to the resin slurry: (i) equilibrate two columns of diaminodipropylamine (DADPA) UltraLink Support and the bottle of Wash Buffer to room temperature, and then remove caps from the column and set the two warmed columns onto a home-made rack to allow the storage solution to drain from the column; (ii) equilibrate column by adding 2 ml of coupling buffer (CB) and allowing it to flow through resin bed and drain from column. Then, gradually equilibrate the resin with 3 ml (column volume is 2 ml) of each of the following solutions with gradual increased DMSO from 10% to 80% before adding FL118 in 80% DMSO: 20% CB. Discard flow-through from the collection test tube; (iii) add 5 ml control solution (4.5 ml DMSO+0.5 ml CB) to the column 1 and in parallel, add 5 ml FL118 solution [2.5 ml FL118 (1 mg/ml in DMSO)+2 ml DMSO+0.5 ml CB] to the column 2; (i.v.) resuspend the resin in the control or FL118 solution by gentle reverse up and down; (v) transfer the resin slurry to a 15 ml conical tube labeled with column 1 and column 2; (vi) add 200 μL PharmaLink Coupling Reagent (37% formaldehyde solution) in each 15 ml tube: 200 μL per 2-4 ml FL118 binding solution); (vii) cap the tube and incubate the control column 1 slurry and the column 2 FL118 slurry at 54° C. in swirling for ˜ 24 hours; (viii) then add 1000 μL CB plus 50 μL formaldehyde to each column swirling at 55° C. for 48 hours.

Affinity purification column setup: (i) transfer the control resin slurry and the FL118-coupled resin slurry into two columns, respectively; (ii) allow resin to settle and then open the bottom cap to allow the solution to flow out to drain all the reaction solution; (iii) washing the column with 2 ml CB, and then with 2 ml ultrapure water/DMSO solution (75:25). Then washing with 1.5 ml 100% DMSO to help remove non-reacted FL118; (i.v.) continuously washing the column with 5 ml CB to each column. Then washing with 4 ml tris washing buffer (0.1 M Tris, pH 8.0), and then continue to wash column with 9×4 ml tris washing buffer (0.1 M Tris, pH 8.0) to quench the active sites that were not sealed by FL118. On the final washing drained to ˜2 ml, recap column and put columns at oven at 32-37C for 45 min to facilitate quenching the active sites that were not sealed by FL118. Then additional 3×4 ml tris washing buffer (0.1 M Tris, pH 8.0).

Affinity column purification of FL118-binding proteins: Cancer cell lysates (2×5 mg) were passed through affinity column and control column in parallel, respectively. After extensive washing with washing buffer, the proteins binding on the column were eluted with 8M urea buffers.

Protein display in gel: After the eluted protein solution de-urea and concentration into 20-30 μL through 3K OMEGA NANOSEP 1.5 ml tube device (PALL Life Sciences, Ann Arbor, MI), the entirely resulted protein mixtures from the control column and the FL118 affinity column in parallel were displayed on a 5-20% gradient SDS PAGE gel, and the displayed unknown protein band was isolated for protein identification.

Identification of the unknown protein that binds to FL118 using mass spectrometry (MS): After the unknown protein bend in FL118 column and the corresponding gel area from the control column were isolated from the get display, in-gel digestion was performed by adding trypsin (sequencing grade, Promega, Madison, WI, 10 ng/μL in 10% (v/v) acetonitrile, 40 mM ammonium bicarbonate) to dehydrate gel slices and incubated at 37° C. for 16 h. After digestion, the tryptic products were extracted twice and dried; the dried peptides were then reconstituted in 10 μL of 2% (v/v) formic acid for analysis with Liquid Chromatography Tandem Mass Spectrometry (LC-ESI-MS/MS). LC-ESI-MS/MS analysis of peptides was performed using a nano-ACQUITY UPLC system (Waters Corporation, Milford, MA) coupled through a nano-spray ionization source to a Quadrupole-Time-of-Flight (Q-ToF) Premier mass spectrometer (Micromass, Waters Corporation). The reconstituted tryptic digests in formic acid were loaded and eluted with a gradient of 99% solvent A (0.1% (v/v) formic acid)/1% solvent B (0.1% (v/v) formic acid in acetonitrile) to 10% solvent A/90% solvent B at 0.8 μL per min. For fragmentation analysis, the Q-ToF mass spectrometer was programmed to select ions with a mass/charge (m/z) in the range of 300-1500 Da, and ions with+2 to +5 charges. All LC-MS/MS spectra were transformed to the Micromass Pick List (PKL) file formats using the ProteinLynx Global SERVER and the default parameters of MaxEnt3 algorithm. The PKL file was used to search against the human, on a local MASCOT server using the following search parameters (i) trypsin as the proteolytic enzyme with two possible missed cleavages; (ii) carbamidomethylation of cysteine as a fixed modification; (iii) oxidation of methionine as a variable modification; (i.v.) an allowable mass error of 100 ppm for peptides and 0.1 Da for fragment ions; (v) the peptide charge set to 2+ and 3+; and (vi) the instrument option set to ESI-QUAD-TOF. The criteria used to identify the proteins included ion scores equal to or greater than the threshold significant ion score (p<0.5), and similarity between the observed protein molecular mass from gel electrophoresis and the calculated molecular mass provided in the database. The eluate from a negative control column (no FL118) contained only keratins as expected.

Immunoprecipitation (IP) assays: Pertinent to this study, for IP with DDX5 or normal IgG antibodies, Mia Paca-2 or SW620 cells were treated with and without FL118 (100 nM, 500 nM) for 6 h and 24h, respectively. Up to 10⁷ cells for each time point/test were harvested with a scraper in cold PBS buffers and collected by centrifugation at 1000g×5 min and washed with cold PBS once. Cell pellets were lysed in 1 mL cold RIPA lysis buffer containing proteinase inhibitors (refer to Western blots). The cell lysates were precleared with centrifuging 14,000 rpm for 15 min. The resulting supernatants were transferred to a new 1.5 ml tube and incubated with up to 3 μg either DDX5 antibodies or normal IgG antibodies for 1 h at 4° C., followed by incubation with 20-40 μL 50% protein A/G-Agarose slurry overnight at 4° C. on a rocker platform. After centrifugation at 1000 g for 5 min at 4° C., the pellet was washed with RIPA buffer or PBS for 2-4 times and collected by centrifugation as above. The pellets were then resuspended in 20-40 μL 1× sample buffer, boiled for 2-4 min and stored on ice for Western blot analyses.

Western blot analyses: For Western blot analysis, cancer cells that were treated with and without FL118 (in some experiments in the presence of MG132) were lysed in RIPA buffer containing 150 mM NaCl, 1.0% IGEPAL CA-630, 0.5% sodium deoxycholate, 0.1% SDS, and 50 mM Tris, pH 8.0. For frozen prostate cancer tissue specimens and their adjacent non-tumor tissues (if any) obtained from Roswell Park pathology network, 30-50 mg of each tissue in a 1.5 Eppendorf tube containing 500 μL of 2×SDS lysis buffer containing Tris pH 6.8+4% SDS+10% Glycerol were homogenized through ultrasound treatment for 10-15 second per time with 3-6 times until tumor tissue was lysed. After all tissues were processed, 1.5 mL tubes were centrifuged at 13,000 rom in a microcentrifuge for 1-2 min and then the supernatant in each tube was transferred into a new 1.5 mL tube for protein concentration measurement (refer to the section of “Studies with Human patient tumor specimens” for more information). Twenty to fifty μg total protein from each sample (cell lysis or tumor tissue lysis) were heated at 95° C. for 5 minutes after mixing with equal volumes of 2× SDS loading buffer. Samples were separated on 10-15% SDS-polyacrylamide gel electrophoresis (SDS-PAGE) gels and electro-transferred to Pure Nitrocellulose Membranes with either 0.45 μm or 0.2 μm (Bio-Rad, Hercules, CA) based on the tested protein size. The membrane was then blocked in 5% skim milk in TBS-T buffer (20 mM Tris/HCl pH 7.5, 0.137 M NaCl, and 0.1% Tween 20) at room temperature for 2-3h. Next, the membrane was incubated with different primary antibodies in TBS-T containing 5% BSA overnight at 4° C. in the range of dilutions from 1:500 to 1:2000 based on the antibody dilution recommended by antibody firms. After washing with TBS-T, the membrane was incubated in TBS-T buffer containing 5% skim milk and corresponding secondary antibody (1:5000) for 45-60 minutes at room temperature with shaking. Proteins of interest were detected using Western Lightning ECL-Plus (Perkin Elmer, Waltham, MA) and visualized by various times (3-120 seconds) of exposure. Actin, tubulin and/or GAPDH were detected as the internal control to normalize total protein loading for each sample.

Purification of Flag-tagged DDX5 (Flag-DDX5), USP2a (Flag-USP2), UbE2T (Flag-(bE2T) and topoisomerase 1 (Top1) (Flag-Top1) proteins: Flag-DDX5, Flag-USP2, Flag-UbE2T and Flag-Topl proteins were purified from the protein expression vector-transfected HEK293T cells by using the FLAG® M Purification Kit for mammalian expression systems (CELLMM2-IKT, Sigma). Briefly, sub-confluent populations of HEK293T cells were transfected with expression vectors for Flag-DDX5, Flag-USP2, Flag-UbE2T and Flag-Top1 (OriGene) using Lipofectamine 2000 (Invitrogen) as per the manufacturer's instructions. After 72h of transfection, cells were lysed in Cell Lytic M lysis reagent (Sigma Cat #C2978) containing a protease inhibitor cocktail. Supernatants were collected by centrifugation at 13,000 rpm for 10 minutes at 4° C. The Flag fusion proteins from the cell lysates were purified with the ANTI-FLAG M2 affinity gel, which is a highly specific monoclonal antibody covalently attached to agarose resin. For this purification step, the required amount of ANTI-FLAG M2 resin was transferred to a fresh microcentrifuge tube and washed twice with 0.5 ml of 1× wash buffer (50 mM Tris-HCl, 150 mM NaCl, pH7.4). Of note, 10× wash buffers were provided in the kit. The washed resin was then added to the protein extract in a microcentrifuge tube and agitated in a roller shaker overnight. After this binding step, the resin was collected by centrifugation and washed three times with 1× wash buffer to remove non-specific proteins. The bound Flag-tagged proteins were eluted from the resin by competitive elution with the 3×Flag peptide (Catalog Number F4799) in 1× wash buffer.

Replacement of the Tris-HCl buffer with the PBS buffer for purified proteins for drug-protein binding analyses using isothermal titration calorimetry (ITC): After the purified proteins were eluted from the agarose resin with 3×flag peptide, the same purified protein samples from different batches were combined. Then, Flag-DDX5, Flag-USP2, Flag-UbE2T and Flag-Toplproteins in the 1× wash buffer (50 mM Tris-HCl, 150 mM NaCl, pH7.4) containing Flag peptide were replaced by using 1.5 mL size Nanosep 10K Omega device (PALL Life Sciences). Briefly, 200-300 μL purified proteins with a concentration of 1-2 μg/μL were pipetted into the sample reservoir of the Nanosep device and centrifuged at 8000 RPM for 15-20 min to remove the 1× wish buffer with Flag peptides (removing the flow-out after each time centrifugation). Then, the retained proteins were washed with 300-400 μL 1× phosphate-buffered saline (PBS) (137 mM NaCl, 10 mM Phosphate, 2.7 mM KCl at pH7.4) 4 times by centrifugation as above. The PBS-washed proteins were then resuspended in 200-300 μL 1× PBS (pH7.4) depending on the initial protein concentration. The PBS-resuspended protein samples were recovered from the reservoir and the protein concentrations were determined using the nano-drop reader (Bio-Rad) and BCA Protein Assay (Thermo/Pierce) for double-making sure the concentration. This buffer replacement is important for the next step of the isothermal titration calorimetry (ITC) to analyze FL118 binding affinity to the proteins.

FL118-DDX5 and FL118-topoisomerase 1 (Top1) binding affinity determination: The binding affinity of FL118 with DDX5 versus with Topl were determined using ITC, a state-of-the art technology for measuring small molecule-protein interactions to define the drug-protein affinity (KD) in in the following steps.

(1) Preparation of FL118 solution (>160 μL/sample, a required volume for testing on the MicroCal-Malvern Auto-ITC200 equipment) containing 100 μM FL118 in 1× Phosphate buffered saline (PBS, pH7.4) and 8% DMSO from 2 mM FL118 stock in DMSO (the same solution was also prepared for topotecan). The minimal DMSO concentration in the solution was determined in advance, and the 8% DMSO is the optimal concentration to use in order to balance the lowest concentration while there is no FL118 precipitation.

(2) Preparation of Flag-DDX5, Flag-USP2, Flag-UbE2T and Flag-Top1 protein solutions (>360 μL/sample), respectively, containing 10 μM of Flag-DDX5, Flag-USP2, Flag-UbE2T or Flag-Top1 proteins in 1×PBS (pH7.4) and 8% DMSO from protein stocks in 1× PBS (the same solution was also prepared for BSA).

(3) preparation of washing/diluting solution (>2.5 mL/sample) containing 1× PBS (pH7.4) and 8% DMSO.

(4) ITC assay was performed using the auto-iTC200 instrument (MicroCal, GE). A protein solution (Flag-DDX5, Flag-USP2, Flag-UbE2T, Flag-Topl or BSA) with defined concentration (10 μM) in a volume of 360 μL in 1×PBS (pH7.4) containing 8% DMSO was loaded into the calorimeter cell as the Macromolecule (M). Then, a small compound ligand (FL118 or topotecan) at a concentration of 10× protein concentration (100 μM) in the same buffer containing 8% DMSO was directly injected into the M cell at a volume of 2 μl per injection every 3 minutes for equilibrium in a total of 20 injections in one hour at 25° C. At each injection, the program calculates [M]t and [X]t (total protein and total ligand at that injection). The binding constant (KD)), binding stoichiometry (n), and thermodynamic parameters (AH and AS) were determined by fitting the titration curve to a one-site binding mode, using the Origin software provided by the manufacturer.

Preparation of FL118 for in vitro application: FL118 was synthesized in house with a purity ≥99%. For in vitro cell cultural studies, FL118 was initially dissolved in DMSO at a concentration of 1 mM as the stock solution for further dilution to variously needed concentrations in the appropriate medium for in vitro experiments. FL 118 has been also manufactured by Canget-hired CRO and the relevant disclosure has been provided elsewhere in this invention.

Real time quantitative (q) RT-PCR: Real time RT-PCR methods were described previously (9, 56). Pertinent to this study, total RNAs were extracted from cancer cells using TRI REAGENT RT (Molecular Research Center), and 5 μg per sample was converted to cDNA with anchored oligo (dT) primers using RevertAid First Strand cDNA Synthesis Kit (Thermo Fisher Scientific, Catalog No. K1622) following the manufacturer's instructions. Individual reverse transcription (RT) reactions of 20 μL were then diluted to 200 μL with sterile H2O. Five microliters of diluted RT reaction were used for real-time qPCR using Maxima SYBR Green/ROX qPCR Master Mix (2×, Thermo Fisher Scientific, Catalog No. K0223). The primers used in qPCR were as follows: 5′-GGC GCA CAG CAC AAG AGG-3′ (DDX5 qPCR-F, forward) and 5′-ATG GCA GGA AGC AAA TAA GAC AA-3′ (DDX5 qPCR-R, reverse); 5′-CTT CTG CTT CAA GGA GCT GGA AG-3′ (hsv5p2, survivin, forward) and 5′-GCA CTT TCT TCG CAG TTT CCT C-3′ (hvs3p2, survivin, reverse); 5′-CCA CAG CAA ACC TCC TCA CAG-3′ (myc5, c-Myc, forward) and 5′-GCA GGA TAG TCC TTC CGA GTG-3′ (myc3, c-Myc, reverse); 5′-GTC TCC TCT GAC TTC AAC AGC G-3′ (hGAPDH-5, forward) and 5′-ACC ACC CTG TTG CTG TAG CCA A-3′ (hGAPDH-3, reverse). GAPDH was used as an internal control. Triplicate qPCR reactions were performed for individual samples. The qPCR condition is 50° C. for 2 min and 95° C. for 10 min as a pre-denature step, followed by 40 cycles at 95° C. for 15 seconds and 60° C. for 1 min in a thin-wall 96-well PCR plate. The data were analyzed using the Applied Biosystems 7300 Real Time PCR System and normalized to GAPDH.

Vector-free CRISPR-Cas9 technology to knock out DDX5 and UbE2T in pancreatic cancer cells and or in prostate cancer cells: CRISPR-Cas9 technique was performed to knock out DDX5 gene in Panc-1 PDAC cells. To simplify the DDX5 knockout (KO) process, instead of using expression vectors in the classical approach, the DDX5 or UbE2T sgRNA-Cas9 enzyme protein ribonucleoprotein (RNP) complex was used directly through electroporation transfection for KO of the DDX5 gene or UbE2T gene in pancreatic cancer cells and/or in prostate cancer cells. Specifically, the process used the DDX5-Alt-R CRISPR-Cas9 crRNA or UbE2T-Alt-R CRISPR-Cas9 crRNA (a part of sgRNA), Alt-R CRISPR-Cas9 tracrRNA (another part of the sgRNA) with ATTO™ 550 (Cat #1075928), Alt-R® S.p. Cas9 Nuclease V3 (Cat #1081058), Alt-R® Genome Editing Detection Kit (Cat #1075932), IDTE pH 7.5 (1× TE Solution) (Cat #Nov. 1, 2002-02) and Alt-RR Cas9 Electroporation Enhancer (Cat #1075915) from Integrated DNA Technologies (IDT); and QuickExtract DNA extract solution (Cat #QE0905T) from Lucigene and NeonTransfection System 10 μL kit (Cat #MPK1096) from Thermo Fisher Scientific. Then, DDX5 sgRNAs or UbE2T sgRNAs were formed into RNP complex in room temperature in vitro with the Cas9 enzyme in a process with defined conditions using the ordered reagents. Pancreatic or prostate cancer cells harvested from 10 cm tissue culture dishes and washed with 5 mL PBS and collected by centrifugation to make 5×10⁵ cells in a 9 μL resuspension buffer R (from NeonTransfection System 10 μL kit). Then, such 9 μL cells+1 μL RNP complex from above+2 μL of Alt-R® Cas9 Electroporation Enhancer (18 μM) were mixed and 10 μL such mixture was used for electroporation at 1,600 V, 10 ms pulse width and 3 pulses. After electroporation, the cells were transferred to a 6-well plate containing 3 mL of complete DMEM cell culture medium and incubated at 37° C. with 5% CO2 for 72 h. Next, the positive transfection was validated through the observation of presence of fluorescence in the cells. Specifically, the Alt-R CRISPR-Cas9 tracrRNA labeled with ATTO™ 550 fluorescence allows detection and intracellular visualization of molecular components via fluorescent microscopy. The fluorescence signal was detected after 48 h of electroporation under fluorescent microscopy to verify the positive transfection. After 72 h of electroporation, cells were collected from the cell culture dish. Half of the cells were used for Alt-R genome editing detection and the other half were used for limited dilution-based single cell cloning.

To isolate single DDX5 KO cell clones or UbE2T KO cell clones from the electroporated pancreatic or prostate cancer cell pool, the electroporated cell pools were processed with a series of dilutions in wells containing 200 μL DMEM complete medium in 96 well plates to the diluted degree that would make many single well only contain a single cell. After observance of single cells forming colonies in a single well under the microscope, cell clones were transferred to a 6-well plate containing 3 ml DMEM complete medium for further culture. Cells were harvested at 80% confluence and 90% of the cells were used for detection of DDX5 KO by Western blotting and the rest cells were left for further growth in the 6-well plate. Western blot positive cell clones were further amplified and some of them were deposited in Liquid Nitrogen while others were used for functional analysis.

Cell attachment after seeding of pancreatic cancer cells with or without DDX5 KO: DDX5 KO cells in parallel with control cells without DDX5 KO were harvested by trypsin digestion and PBS wishing. The harvested cells were then resuspended in the DMEM complete medium with and without Agrin at a concentration from 2.5, 5 or 10 μg/mL. 0.1 million cells were added to each well in 24 well plates. Quantitative analysis of non-attached cell numbers and collecting of cell attachment images were carried out at 4-16 hours after seeding which was depending on cell types.

Cell monolayer wound healing test of DDX5 KO pancreatic cancer cells: When DDX5 KO pancreatic cancer cells in parallel with control cells without DDX5 KO almost reached the confluent monolayer, the monolayer was gently scratched using a pipette tip. After 4 times washing with PBS, DMEM complete medium with or without Agrin at the concentration of 5 μg/mL and/or 10 μg/mL was added to each well and replace cell plates back to the CO2 incubator for cell migration healing for 48 to 72 hours. Representative cell migration wound healing images were taken 48-72 hours after wound formation by gently scribing.

Cell attachment after seeding of pancreatic cancer cells with or without FL118 pretreatment: The attached wild-type cells with and without 24-hour FL118 pretreatment were harvested by trypsin digestion and PBS wishing. The harvested cells were then reseeded at an expected 30-60% confluence if all cell attached to the cell cultural dish. During the reseeding process, the cell cultural medium for the FL 118-pretreated cells may be in parallel with and without Agrin at a concentration of 5 and/or 10 μg/mL om the cell culture medium. Representative cell attachment images were taken 4-16 hours dependent on control cell attachment speed after cell seeding. Quantitative analysis of non-attached cell numbers was documented in parallel for each condition.

Cell monolayer wound healing test of pancreatic cancer cells in the presence and absence of FL118 and or Agrin: When wild-type pancreatic cancer cells almost reached the confluent monolayer, the monolayer was gently scratched with a pipette tip to form a wound and then the monolayer wound healing via cell migration was documented at 48 to 72 hours (after wound formation) with or without FL 118 treatment in the cell cultural medium in the presence and absence of exogenously adding Agrin at the concentration of 5 μg/mL and/or 10 μg/mL in the cell cultural medium. Representative cell migration wound healing images were taken at zero hour and wound healing for 48-72 hours.

MTT assay for determining cancer cell growth viability: The MTT assay was described previously (13). Pertinent to this study, CRC or PDAC cell growth after genetic overexpression DDX5 and/or FL118 treatment with different concentrations was determined by MTT cell growth/viability assay. Briefly, ˜2500 viable cells with or without genetic manipulation were plated in each well in 96-well plates. After being incubated overnight in a 5% CO₂ incubator at 37° C., cells were treated with and without FL118 at different concentrations and continuously incubated for 72 h. MTT, a colorimetric substrate, was then added to a final concentration of 0.4 mg/mL to each well. Cells in 96-well plates were further incubated in a 5% CO₂ incubator at 37° C. for 4 h, and then the medium was aspirated. The MTT metabolic product formazan was solubilized by adding 200 μl of DMSO to each well. Absorbance in the relevant wells was measured at 570 nm using an Ultra Microplate Reader (Bio-Tek Instruments).

3D) cell spheroid formation studies: Pancreatic cancer cells were cultured in the ultra-low attachment plates in the pancreatic cancer organoid culture medium. In some studies, to observe the spheroid formation, DDX5 KO pancreatic cancer cells in the presence and absence of Agrin at a concentration of 2.5 μg/mL, 5 μg/mL and/or 10 μg/mL for 6-7 days were cultured. In some other studies, pancreatic cancer cells were cultured and treated with and without FL118 in a series of concentrations (0.8-500 nM) for 6-7 days to observe the spheroid formation and determine the cell viability. Then pancreatic cancer cells were cultured and treated with and without 1-2 appropriate FL118 concentrations in the presence and absence of Agrin at a concentration of 2.5 μg/mL, 5 μg/mL and/or 10 μg/mL for 6-7 days to observe the spheroid formation. Representative images were take during this time and cell viability was determined by MTT assay.

Pancreatic cancer patient-derived xenograft (PDX) tumor (PDX14244) in vitro culture and studies: PDX14244 tumors were collected from SCID mice, and the isolated tumors were maintained in PBS solution. Then cut the fresh tumor tissues into many small pieces (e.g., 2×2×2 mm) and the left tumors were frozen for other use. The small pieces of tumor tissues were washed twice in PBS and then added onto the stiff gel prepared at the ratio of gel: PBS=1:1 in the 24-well plate (2-3 pieces per well). Then, 2-3 wells were treated with vehicle (500 μL culture medium+DMSO), 2-3 wells were treated with FL118 (500 μL medium+10 nM FL118), and 2-3 wells were treated with FL118+Agrin (500 μL medium+10 nM FL118+5 μg/mL Agrin). On day 3, replace the old medium content with freshly prepared same medium content. On day 7, the cultured PDX14244 tumor tissues were taken out and representative images were taken under microscopes, and the tumor tissues were transferred into a fresh 24-well plate and immediately fixed in 10% neutral buffered formalin (NBF) for 24 hours ad then replaced NBF with 70% ethanol for 1-4 days for paraffin embedding to make block cassettes for histology analysis. Tissue blocks were cut and H&E staining for tumor tissue stroma ECM stiffness (desmoplasia) observation.

FL118 labeling with tritium (3H): Preparation of tritium (3H)-labeled FL118 were done by Moravek Biochemicals, Inc. (Brea, CA) through service. (1) Generation of ³H-labeled FL118 (3H-FL118) with specificity of 5.6 Ci/mmol: The ³H labeling of FL118 using 3H/1H exchanging approaches. Specifically, a 50 mL round bottom flask was charged with 1 mg FL118, 100 mg PdBaSO4 5%, 60 Ci T2 Gas. The flask was then immersed in silicone oil bath at 180° C.-190° C. for 6 hours. The tritium gas was removed. The reaction mixture was dissolved in 1 mL of DMSO and back-exchanged 10× with 50% ethanol. Injected directly to CapCeLL PAK C-18 column (4.6×250 mm), mobile phase 30% CH3CN, 0.1% TFA, flow 1 mL/min, U.V. =200 nm, r.t. =40 min. After purification, total activity was 5 mCi (solid) with a specificity of 5.6 Ci/mmol at a radiochemical purity of >97% for ³H-FL118. (2) Generation of ³H-labeled FL118 with specificity of 16.5 Ci/mmol: The ³H labeling of FL118 using 3H/1H exchanging approaches. Specifically, a 50 mL round bottom flask was charged with 1 mg FL118, 120 mg PdBaSO4 5% solution and 90 Ci T2 Gas. The flask was then immersed in silicone oil bath at 190° C. for 24 h. The tritium gas was removed. The reaction mixture was dissolved in 1 mL of DMSO and back-exchanged 10× with 50% ethanol and injected directly to CapCeLL Pak C-18 column (4.6×250 mm), mobile phase 30% CH3CN, 0.1% TFA, flow 6 ml/min, U.V. =200 nm, r.t. =40 min. After purification total activity was 5 mCi (solid) with a specificity of 16.5 Ci/mmol at a radiochemical purity of ≥81% for ³H-FL118.

Use of ³H-FL118 as a radioactive probe to search FL118-binding proteins through screening of human protein microarray (ProtoArray R with 9,400 human proteins, Invitrogen): The study process includes the following steps.

1. Blocking the ProtoArray: (i) Immediately place the mailer containing the Human Protein Microarray v5.0 at 4° C. upon removal from storage at −20° C. and equilibrate the mailer at 4° C. for at least 15 minutes prior to use. (ii) Place Human Protein Microarrays with the barcode facing up in the bottom of a 4-chamber incubation tray such that the barcode end of the microarray is near the tray end containing an indented numeral. (iii) Using a sterile pipette, add 5 mL FL118 Assay Buffer into each chamber. Avoid pipetting buffer directly onto the array surface. (iv) Incubate the tray for 1 hour at 4° C. on a shaker set at 50 rpm (circular shaking). (v) After incubation, remove Protein Microarrays from FL118 Assay Buffer. To remove array from the 4-chamber incubation tray, insert the tip of forceps into the indented numeral end and gently pry the array upward. Using a gloved hand, pick up the microarray by holding the array by its edges only. Tap to remove excess liquid from slide surface. (vi) Proceed immediately to Probing the Array.

2. Probing the ProtoArray: (i) Place each ProtoArray horizontally in a separate sterile 50 mL conical tube with about ⅓ of the array extended outside of the tube. The barcoded end of the array should protrude from the tube (face up). (ii) For each ProtoArray, add 100 μL of probing mixture including ³H-FL118 (final concentration of ³H-FL118 at 0.1 μM and 0.5 μM, respectively on different ProtoArray) and the positional mapping reagent ³H-estradiol and pipet the mixture gently onto the surface of the ProtoArray. (iii) Gently place a coverslip over the surface of the ProtoArray using forceps to avoid capturing bubbles. (iv) Position the ProtoArray with coverslip within the conical tube with the printed side of the array facing up. Cap the tube. Place the tube on a flat surface such that the printed side of the array is facing up and the tube is as level as possible. If needed, tape the conical tube on the flat surface to avoid any accidental disturbances. (v) Incubate the array at 4° C. for 90 minutes without shaking. (vi) Remove conical tube containing ProtoArray from incubator and add 40 mL Tritium SMI Assay Buffer to the tube. (vii) Incubate the array in buffer for 30 seconds at room temperature. The glass coverslip will float off. Do not remove the coverslip with forceps if it is not dislodged from the array. (viii) Using forceps, carefully remove the dislodged coverslip without touching the array surface. Discard the coverslip appropriately as radioactive waste. (ix) Decant the Tritium SMI Assay Buffer (be sure to dispose of the radioactive waste properly). (x) Add 40 mL of fresh Tritium SMI Assay Buffer to the tube. Incubate the array for 30 seconds at room temperature. Decant buffer. Repeat wash step one more time (be sure to dispose of the radioactive waste properly). (xi) If Tritium SMI Assay Buffer with NaCl is used, complete one additional wash with Tritium SMI buffer lacking NaCl.

3. Drying and exposing the ProtoArray: (i) Remove the array from the chamber at the end of the probing procedure. Tap one edge of the array gently on a laboratory wipe for a few seconds to drain any buffer. (ii) Place each array in a slide holder (or a sterile 50 ml conical tube, if you do not have a slide holder). Ensure the array is properly placed and is secure in the holder to prevent any damage to the array during centrifugation. (iii) Centrifuge the array in the slide holder or 50 mL conical tube at 200 Å˜ g for 1 minute in a centrifuge (equipped with a plate rotor, if you are using the slide holder) at room temperature. Verify the array is completely dry. (iv) Using transparent tape, adhere the slides to an 8×10 Exeter Conservation Board (or thick filter paper of similar size). Only tape the top and bottom edges of the slide without covering any array area. The adhesion helps to prevent unwanted movement during the long exposure time and also helps to prevent the tritium from transferring on to the screen. Place ProtoArray in X-ray film cassette and directly overlay with a tritium-sensitive phosphor screen. (v) Expose ProtoArray to the phosphor screen for 16 days.

4. Image acquisition and analysis: GenePix Pro v7 (Molecular Devices Corporation) and/or ScanArray. Acquisition Software (PerkinElmer, Inc.) was used for image acquisition and analysis.

Confirmation of FL118-USP2a and FL118-UbE2T binding by alternatively using Nanosep Device: The binding of FL118 to USP2a or UbE2T was alternatively determined using the low protein-binding polypropylene Omega™ membrane Nanosep 3K Centrifugal Devices (PALL, Life Sciences, Ann Arbor, Michigan), which can let molecules with a molecular weight size of less than 3 kD pass though, while retaining 100% of molecules larger than 10 kD. Briefly, tritium (3H)-labeled hot FL118 (3H-FL118) was mixed with non-labeled cold FL118 at a ratio of 1:10 to make an FL118 concentration at 10 μM in 1× Phosphate buffered saline (PBS, pH7.4) containing 8% DMSO (designated as hot FL118 solution). Then, 5-10 μg of the FLAGR M Purification Kit (Signa)-purified Flag-USP2a and Flag-UbE2T proteins in a volume of 20-50 μL, respectively, was transferred into the sample reservoir of a 1.5 mL Nanosep 3K Centrifugal Device. In parallel, a negative control Nanosep 3K Centrifugal Devices containing 10 μg bovine serum albumin (BSA) in 50 μL volume was also set up. Then, the 3 prepared Nanosep 3K Devices were centrifuged at 10,000 rpm for 10-15 min to eliminate the solution and retain the proteins in the sample reservoir. Next, the proteins in the reservoirs of the 3 Nanosep 3K Devices were resuspended with 100-200 μL hot FL118 solution for FL 118-protein binding at room temperature for 30 min. Then the free FL118 in the solution was removed by a low speed of centrifugation (2000-4000 rpm) of the three Nanosep 3K Devices for 15-30 min until there was no solution in the sample reservoir. Lastly, all three of the sample reservoir devices were transferred into a scintillation vial containing enough scintillation solution to cover the entire sample reservoir devices for tritium (3H) counting on the LS 6500 Scintillation System (Backman Couler™).

Overexpression of USP2 USP2a in prostate cancer LNCaP cells: The USP2 expression plasmid of pCMV6-entry-myc-DDK (Flag)-tagged-USP2 was purchased from OriGene (Cat #RC200273), it contained a CMV promoter for driving high-level of constitutive expression of USP2 gene and a Kan^{r}/Neo^{r} selection marker for bacterial plasmid selection and stable mammalian cell lines establishment. LNCaP cells were maintained in RPMI-1640 medium with 10% (V/V) fetal bovine serum (FBS) and penicillin/streptomycin (100 U/ml) at 37° C. in a 5% CO₂ incubator. For transfection, LNCaP cells were plated in 6 well culture plates with 3 ml of RPMI-1640 medium containing 10% FBS and 1% Pen/Strep. When the cells reached 60-80% confluence, transfection was done following the instruction of Lipofectamine™ 2000 (Invitrogen). For each well, the DNA-Lipofectamine 2000 complexes contained 2.3-2.5 μL of Lipofectamine™ 2000, 1.5 μg of plasmid DNA and 250 μl of Optim-1 medium. After the complexes was gently swirl and incubated at RT for 20-30 min, the cells were transfected by replacing the cell culture medium with the DNA/lipofectamine complex and then the cells were incubated at 37° C. for 2-4 hours in a 5% CO2 incubator. Then, 3 ml of RPMI-1640 medium containing 10% FBS and 1% Pen/Strep was added to each well and the cells were cultured for another 16-24 hours. After that, the cell culture medium was replaced with fresh complete culture medium containing neomycin or G418 for selection of transfected cells for up to 5 days. The empty vector without USP2 (OriGene) was used as control and the transfection condition was same as above. The resulted cells from empty vector (control) and USP2 overexpressed cells were used for experiments.

Preparation of lentiviral particles containing UbE2T shRNA: UbE2T shRNA #2 and #5 selected from a pre-experiment in the pGIPZ lentiviral vectors (with Puromycin for selection and TurboGFP as visual marking of shRNA expressing cells) were first packaged in the HEK293T packaging cells by transfecting cells at 80% confluence as follows: Cells were incubated in a 5% CO₂ incubator for 24 h at 37° C. and then transfected by gently replacing the cell medium in a 100 mm culture dish with 500 μl DNA/Lipofectamine 2000 complex per dish with gentle swirling. Three mL of DMEM with 10% FBS and 1% Pen/Strep were added after a few minutes and then put back to the 5% CO₂ incubator for 16 hours at 37° C. The 500 μl DNA/Lipofectamine 2000 complex was prepared as follows: 250 μl DMEM containing 2.5 μg pGIPZ shRNA, 2.5 μg psPAX2 (or pCMV-dR8.74), 1.0 μg pMD2.G in one tube were mixed with 250 μl DMEM containing 6.5-7 μL Lipofectamine 2000 and kept at room temperature for 20 minutes. The medium in the transfected HEK297T cells in the dish was replaced with new media on the next day, and the dish was put back to the 5% CO₂ incubator for additional 24 h at 37° C. Virus-containing supernatant was harvested from control shRNA transfected dishes and UbE2T shRNA transfected dishes and combined, respectively. The TurboGFP expression was checked before collection of the virus in the supernatant. The transfected 293T cells in the dish were added with another 3 mL media and incubated in the CO₂ incubator at 37° C. overnight. The supernatant collected and combined together. The finally collected virus particle supernatants were then filtered through a 0.45 um cellulose acetate (low protein binding) syringe filter, and the virus stored at 4° C. for use in the experiments (suplus viral particles can be aliquoted in 0.5-1.5 Eppendorf tubes and stored in −80° C. for later use.

Target cell infection with lentiviral particles: For infection of target cells (DU145) with lentiviral stock, cancer cells grown to sub-confluence in 6-well plates were infected with 0.5-1 mL lentiviral stock prepared as above in the presence of 4 μg/mL polybrene (infection stimulator). To increase cell infection rates, the plate was spun at 1800 rpm for 45 minutes at room temperature on a microtiter rotor. The infected cells in the plate were then incubated in a CO₂ incubator at 37° C. for 3-6 h, an additional 1 mL complete media was added and cells were incubated overnight in the CO₂ incubator at 37° C. Cells in individual wells were then diluted five times (one plate to five plates) and incubated for 24 h, followed by selection with puromycin (2 μg/ml) for 3-7 days. The resultant puromycin-selected cells were directly used for the experiments or were further used for obtaining single cell clones by limited dilution if single cell clones were needed.

In vivo function-relevant studies: All in vivo human tumor animal model-relevant studies were performed following the mouse protocol (1192M) approved by the Institutional Animal Care and Use Committee (IACUC) at Roswell Park Comprehensive Cancer Center (Roswell Park). The fresh pancreatic cancer tumor tissue specimens used for generating data related to this invention were obtained from Roswell Park Pathology Network Hospital Clinic under the IRB BDR protocol BDR-111819 in a deidentified manner without directly linking to the patient identity. Thus, the use of these tumor tissue specimens can be classified in in the category of non-human subject. The obtained fresh individual tumor tissues were immediately implanted in the flank area of SCID mice for the establishment of PDX tumors and the surplus fresh tissues were frozen at −80° C. for potential analytical applications. The specimens (frozen or fresh) were initially examined by a certified pathologist. The examined specimens were then coded by the designated investigator(s), and no patient identifiers (Protected Health Information) were/are available to the project PI and team members. Instead, each code number will be the only link for obtaining clinical information from patients with their tumors.

The FL118 maximum tolerated dose (MTD) for the weekly×4 schedule is about 10 mg/kg for mice. For in vivo studies, eight to ten-week-old female and/or male SCID mice (18-22g) were obtained from the Division of Laboratory Animal Resources (DLAR), Roswell Park. Female SCID mice were housed at 5 mice per cage with water and food ad libitum, and male SCID mice were housed at 4 mice per cage with water and food ad libitum. Two types of human tumor models were used: human pancreatic cancer PDX tumor models and human (colorectal and prostate) cancer cell-established tumor models. Specifically, they were the Kras, p53 and APC triple mutated SW620 colorectal cancer cell line-established xenograft tumor as a model system for antitumor efficacy studies of the FL118 powder product manufactured through both the pure ethanol-involved and the glacial acetic acid-involved Spray Dry Dispersion (SDD)-process. Experimental human tumor animal model setup: Tumor establishment in SCID mice from cancer cell lines used 2×10⁶ cancer cells per tumor site (with or without 50% Matrigel were subcutaneously injected in each site at the flank area of SCID mice, respectively for 3-5 mice (dependent on the study need). These human tumors were maintained on SCID mice. tumors maintained on SCID mice were isolated, and a piece of non-necrotic tumor tissue (30-40 mg) was subcutaneously transplanted into the flank area of SCID mice. When human tumors grew to 100-200 mm³ (defined as day 0), mice were randomly divided into the required groups (5 mice per group) for FL118 oral administration with weekly×4 schedules (when applicable). Tumor growth was monitored and documented over time.

Tumor size measurement: Tumor length (L) and width (W) were measured using digital vernier calipers 2-3 times per week until the end of experimental studies. The tumor volume (v) was calculated using the formula: v=0.5 (L× W2). Then the tumor size was divided by the day 0 tumor size as percentage tumor size versus day 0. The mean tumor volume+standard deviation (SD) at each time point was derived from 2-5 mice in each group (in different experiments). The tumor curves were plotted using Microsoft Excel.

Studies with human patient tumor specimens (frozen and fresh): Frozen clinical prostate cancer tissue specimens were obtained from Roswell Pathology network based on the IRB BDR protocol I 35504. The fresh pancreatic cancer tumor tissue specimens were originally obtained from Roswell Park Hospital Clinic under the IRB BDR protocol BDR-111819. The patient tumor tissues were obtained from the pathology network hospital clinic could not be directly linked to the patient identity; and therefore, the use of these tumor tissue specimens is classified in the non-human subject category.

General method used for the hERG Test: FL118's effects on hERG activity experiment was performed on an IonWorks™ HT instrument (Molecular Devices Corporation), which automatically performs electrophysiology measurements in 48 single cells simultaneously in a specialized 384-well plate (PatchPlate™). All cell suspensions, buffers, and test compound solutions were at room temperature during the experiment. Briefly, Chinese hamster ovary (CHO) cells stably transfected with hERG (cell line obtained from Cytomyx, UK) were seeded in each well of a PatchPlate™ positioned over a small hole at the bottom of each well to form an electrical seal. The resistance of each seal was measured via a common ground electrode in the intracellular compartment and individual electrodes placed into each of the upper wells. Electrical access to the cell was then achieved by circulating a perforating agent, amphotericin B, underneath the PatchPlate™. The hERG channels were activated by applying a depolarizing step to +40 mV for 5 s and then clamped at 50 mV for 4 s to elicit the hERG tail current, before returning to 80 mV for 0.3 s. After test article FL118 was pre-diluted into a range of distinct concentrations of each with the same DMSO vehicle control concentration at 0.25%, the IonWorks™ HT instrument automatically added FL118 dilutions from a 96-well microtiter-plate to the upper wells of the PatchPlate™. FL118 was left in contact with the cells for 300 s before recording currents using the same voltage step protocol as in the pre compound scan. Quinidine, an established hERG inhibitor, was included as a positive control, and buffer containing 0.25% DMSO was included as a negative control. Each concentration was tested in 4 replicate wells on the PatchPlate™. For a data point to be used to assess hERG blockade, the cell must maintain a seal resistance of greater than 50 MOhm and a pre compound current of at least 0.1 nA. Additional filters were also applied to pre and post FL118 data to assess cell stability.

Specimen preparation for Pharmacokinetics (PK) studies in tumor bearing mice: FL118 PK studies were performed as follows: Individual SCID mice were first inoculated with human SW620 and FaDu tumors (i.v. route) or human SW620 and HT29 tumors (oral route) at both left and right flank sites for comparison, when tumor size grew to approximate 800-1000 mm³, a single i.v. or oral administration of FL118 was given at a dose of 1.5 mg/kg to six groups of SCID mice (3 mice per group). Tumor tissues and blood samples were collected at a series of time points of 10 min (i.v. only), 30 min (oral only), 1 hour, 4 hours, 12 hours, 24 hours and 48 hours (i.v. only). Each tumor tissue was collected in a tube and immediately frozen with liquid nitrogen; each blood sample was collected in a Li-Heparin LH/1.3 tube (SARSTEDT), and plasma was recovered by centrifugation (1500 rpm×2 minutes). The collected plasma from each blood sample was transferred into a new tube and immediately frozen in liquid nitrogen. Liquid nitrogen-frozen specimens were then transferred to an −80° C. freezer for analysis.

FL118 extraction from the prepared tumor tissue and serum: FL118 in plasma was extracted with acidified methanol. An 800 μL aliquot of ice-cold acidified methanol was added to 200 μL plasma and vortexed for 15 sec. In parallel, FL118 in human xenograft tumor tissue was first homogenized in 1×PBS (W/V=1g tissue/3 ml 1× PBS) and then extracted with acidified methanol. An 800 μL aliquot of ice-cold acidified methanol was added to 200 μL homogenized tissue and then vortexed for 1 min. The samples are then centrifuged at 13,000 rpm for 5 min. and the supernatant was transferred to a clean 13×100 mm glass tube. Samples were dried under vacuum and stored at −20° C. until analysis.

Analysis of FL118 PK: Dried samples were then reconstituted in 200 μL of mobile phase (80% 3% TEA and 20% acetonitrile pH 5.5) and 15 μL were injected. Analysis was carried out using an Acquity UPLC system with Fluorescence detection interfaced with Empower software. Separation was carried out on an Acquity BEH Shield RP18 1.7 μm, 2.1 mm×100 mm column (Waters). The fluorescence detector was set at the following Excitation (Ex) and Emissions (Em) wavelengths: Ex 370 nm Em 510 nm. The calibration standards were prepared by spiking plasma with FL118; the calibration curve range is 5 ng/ml-500 ng/mL. To ensure quality assurance, quality control samples are prepared in plasma at 25 and 250 ng/ml aliquoted and stored at −20° C. The QC's were injected in duplicate at the beginning and end of the assay. Assay has been validated. Validation consisted of running twelve standard curves over the course of 5 days. QC samples were analyzed with each curve. The overall precision (% CV=6.4) and overall accuracy (101%) of the assay calibrators were shown to be excellent. QC precision measured as % CV was equal to 7.5% and overall QC accuracy was 96%.

Laboratory methods used for FL118 formulation and prototype formulated FL118 product production from the identified organic solvents for initial in vivo testing: For these liquid organic solvents that could dissolve HPβCD and reach a concentration of HPβCD ≥10% in the organic solvent to become a pure solution were further used for FL118 formulation to produce the prototype FL118 product. The FL118 prototype product formation in brief are: (1) dissolving 500 mg HPβCD into each organic solvent identified in a volume of 2.5 mL organic solvent to make a solution with a HPβCD concentration less than 20% in the solution; (2) dissolving 50 mg FL118 DS into the organic solvent-HPβCD solution to reach a ratio of FL118: HPβCD to 1:10 (W/W) by vortex to form an organic solvent-HPβCD-FL118 complex; (3) then, the organic solvent-HPβCD-FL118 complex in a solution or s suspending status was processed to remove organic solvent through lyophilization; and (4) finally, the lyophilized dry HPβCD-FL118 complex product was resuspended with saline containing 0-2.5% propylene glycol (PG) and 0-2.5% polyethylene glycol 400 (PEG400) for the studies of their anti-human tumor efficacy and toxicity (mainly animal body weight changes).

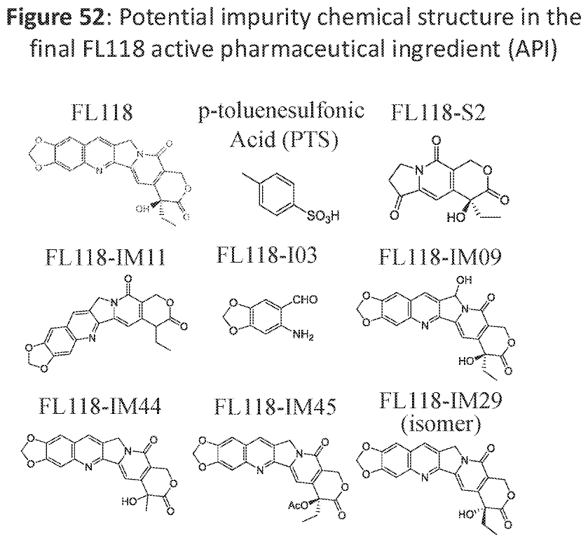

Analysis of FL118 drug substance (DS) stability: The FL118 active pharmaceutical ingredient (API) stability in purity and impurity was determined by using high-performance liquid chromatography (HPLC). Since FL118 is water-insoluble, through a solvent screening process, it was found that DMSO is the most appropriate solvent for dissolution of FL118 API for purity and impurity analyses. Additionally, according to the information provided from the non-GMP (good manufacturing practice) and GMP FL118 API synthesis, the potential impurities in FL118 API may include the known potential impurities of p-toluenesulfonic acid (PTS), FL118-S2, FL118-IM11, FL118-103, FL118-IM09, FL118-IM44, FL118-IM45 and FL118-IM29 (FIG. 52). Therefore, based on the FDA purity and impurity regulatory criteria, an FDA regulation-matched standard resolution solution containing FL118 (0.1 mg/mL), the known impurity PTS, FL118-S2 (0.001 mg/mL), FL118-IM11 (0.001 mg/mL), FL118-103 (0.001 mg/mL), FL118-IM09 (0.001 mg/mL), FL118-IM44 (0.001 mg/mL), FL118-IM45 (0.001 mg/mL), and FL118-IM29 (0.001 mg/mL) was prepared in DMSO as the FL118 API purity and impurity analysis positive control to establish various specific analytic conditions for impurity analyses using HPLC. In parallel, use of the non-GMP and GMP FL118 API substance stock, a solution containing 0.2 mg/mL of FL118 in DMSO as the highest concentration was prepared and in turn diluted into linear solutions with different FL118 API concentrations, and then injected each of the defined concentrations of FL118 solution into HPLC for analyses at the established HPLC analytic condition to find the signal-to-noise (S/N) ratio of FL118 at various low concentrations as well as the linear correlation between concentration and peak area to confirm the final HPLC condition for analysis of FL118 purity and impurities. Since FL118 is very sensitive to alkaline conditions, all analysis conditions avoided basic conditions did not use acidic solution for a neutralization process in order to avoid unreliable analytical results. With the developed and validated HPLC methodological conditions, the FL118 stability in terms of purity and impurity from each of the three batches of FL118 API (two batches of non-GMP FL118 API and one batch of GMP FL118 API, Table 5) was analyzed through the defined HPLC analytic conditions.

Manufacturing of FL118 products by using clinically compatible novel formulation: The clinically compatible formulation and manufacturing of FL118 products is a hydroxypropyl methylcellulose (HPMC)-free FL118 product suspension in saline as the final format, which is the most recent developed formulation. For production of the FL118-HPβCD complex powder product, the organic solvents, glacial acetic acid and ethanol were used in the process to formulate FL118 with the excipient HPβCD first through the microfluidizer-involved process to reduce FL118-HPβCD complex particle size, followed by the Spray Dry Dispersion (SDD) process to get rid of the glacial acetic acid and ethanol to obtain the FL118-HPβCD complex powder product. The FL118 product obtained in this way could be simply resuspended with saline used in the clinic for patients through oral administration. Specifically, different amounts of the HPβCD-FL118 complex powder in a vial, tube or an appropriate bottle can be directly resuspended with a desired volume of saline by gently up-and-down inverting the vial, tube or a bottle contained the FL118 powder product to result in the required concentration of FL118 product suspension before oral administration. Such FL118 suspension products can be used for up to 3 months without clearly detectable decreasing of antitumor efficacy if stored in cold room or refrigerator (2-4° C.). The FL118 product suspension in the container can be simply gently up-and-down inverted for several times every time before oral administration. The Canget-hired CROs provided multiple small batches of 100% ethanol-involved SDD-processed, 100% glacial acetic acid-involved SDD-processed, 50% glacial acetic acid/50% ethanol-involved SDD-processed and 10% glacial acetic acid[[GAA]]/90% ethanol-involved SDD-processed FL118-HPβCD complex powder products. The final lot of the 100% ethanol-involved SDD-processed FL118-HPβCD complex powder product is FR00535-5-191104-01, which was manufactured by WuXi AppTec (Shanghai, China); and the final lot of 10% glacial acetic acid/90ethanol-involved SDD-processed FL118-HPβCD complex powder product is CGT SDC LOT 2020-252-23, which was manufactured by BioDuro (San Diego, CA, USA) in July 2021; both lots of the FL118 product used GMP FL118 API (batch No. C180402127-BF18001). The detailed manufacturing process description were provided in the “Pharmaceutical formulation process” section as well as in some relevant examples.

FL118-HPβCD complex powder product analysis: Three methods were used for the analysis of the FL118-HPβCD complex powder product manufactured using the novel method and process with defined organic solvents and equipment. The methods are described as below.

X-ray powder diffractometer (XRPD)): XRPD was used to determine the FL118 crystal status versus amorphous status. The crystal or amorphous status of each batch of the manufactured FL118 products were determined using XRPD. Specifically, a small amount (e.g., 5 mg) of each batch of FL118 products manufactured through the process described above was spread onto the center of Si-substrate (sample area will be 1 cm in diameter) for XRPD testing. Laboratory XRPD patterns from the samples will be collected at 25° C. The diffraction data were collected over the angular range of 2θ=3°−40° with a step size of 2θ=0.02° and accounting time of 0.12 s/step.

Differential scanning calorimetry (DSC) and modulated DSC (mDSC): DSC and mDSC were used to determine the status of the formulated FL118 product miscibility with HPβCD. The method used is that DSC/mDSC analyses were carried out on a Q2000 differential scanning calorimeter (TA, USA) at a 2° C./min (mDSC) or 10° C./min (DSC) heating rate over temperatures ranging from room temperature to 300° C. in a dynamic nitrogen atmosphere. Each sample from a batch of FL118 products was weighed using a Tzero aluminum sample pan covered by a pinhole lid; an empty pan served as the reference control for testing. Additionally, the glass transition temperature (Tg) of the FL118 product from each batch sample of the FL118 product was determined by using mDSC.

High-performance liquid chromatography (HPLC): HPLC was used to determine the FL118 weight percentage loading in the HPβCD-FL118 product complex by using a batch sample of the manufactured FL118 product. The Waters XSelect CSH C18 column (3.5 μm, 4.6×150 mm) was used for the testing. The mobile phase was a gradient program of 0.5% aqueous trifluoroacetic acid (mobile phase A) and acetonitrile with 0.5% trifluoroacetic acid (mobile phase B), which was pumped at a total flow rate of 1 mL per min. The gradient was as follows: initial conditions were 10% B in A; then a linear gradient of 10% to 60% B in A was realized over 15 min; then the linear gradient of 60% to 90% B in A was realized over 10 min, and held at 90% B in A for 5 min, then returned to initial conditions for 0.01 min and held for 10 min. The column temperature was maintained at 30° C., and the eluent was monitored at a wavelength of 220 nm. The injection volume was 5.0 μL. The diluent was DMSO.

Methods for toxicology and MTD studies in standard numbers of rats using the non-clinically compatible FL118 HPMC thick suspension: The CRO, Covance has performed the studies using a rat protocol approved by the Covance IACUC. The brief experiment method is as follows: Based on the FL118 MTD in human tumor SCID mice, the FL118 dose range was set as 1.65, 3.3, and 6.6 mg/kg weekly for two weeks (2 doses) on days 1 and 8 for each group. Twenty male and 20 female Hsd: Sprague Dawley®SDR rats were received from Envigo RMS, Inc. (Indianapolis, Indiana). Animals were acclimated to the test facility for 7 days prior to initiation. At initiation of dosing, animals were 7 to 8 weeks old, and their body weights ranged from 196 to 216 g for males and 179 to 200 g for females.

Prior to group assignment, animals were excluded from the selection pool/sex to produce minimal variation. Animals were assigned to the study using a computerized procedure designed to achieve body weight balance with respect to group assignment with the following experimental design.

| | No. of | | | Level Dose |
|---|---|---|---|---|
| | Animals | | Dose | Concentration |
| Group^{a} | Male | Female | (mg/kg/dose) | (mg/mL) |
| 1 (Control/vehicle) | 5 | 5 | 0 | 0 |
| 2 (Low) | 5 | 5 | 1.65 | 0.11 |
| 3 (Mid) | 5 | 5 | 3.3 | 0.22 |
| 4 (High) | 5 | 5 | 6.6 | 0.44 |

^{a}Group 1 was administered vehicle control article only.

The formulation of FL118 in this study are FL118 at 0.11, 0.22 and 0.44 mg/mL in 0.44% HPβCD, 2% HPMC and 1% PG in sterile saline (0.85% NaCl). This oral administration suspension without FL118 was the vehicle control. Dose formulations were administered by oral gavage once on Days 1 and 8 of the dosing phases at a dose volume of 15 mL/kg. Doses were based on the most recently recorded scheduled body weight. Animals were checked twice daily (a.m. and p.m.) for mortality, abnormalities, and signs of pain or distress. Cage-side observations were conducted for each animal once daily during the dosing phase, except on days when detailed observations were conducted. Abnormal findings were recorded. Detailed observations were conducted for each animal once during the pre-dose phase, and on Days 1, 4 and 8 (prior to dosing, as applicable) of the dosing phase. Detailed observations were also collected for each animal on days of scheduled sacrifice (all surviving animals). On each day of dosing, cage-side observations were conducted for each animal approximately 1, 4, and 24 hours postdose. Postdose observations were based on the time dosing was completed for each group/sex. Body weights were recorded once during the pre-dose phase, and on Days 1, 4, and 8 (prior to dosing, as applicable) of the dosing phase. The amount of food consumed by each cage of was recorded Days 1 to 4, Days 4 to 8, and Days 8 to 9. Blood samples for hematology, coagulation, and clinical chemistry were collected from fasted toxicity animals via a jugular vein. Blood samples were collected on the day of scheduled sacrifice. Blood samples for hematology and clinical chemistry were also collected via a jugular vein at an unscheduled interval from relvant animals: (1) Group 3 and 4 males, and Group 4 females, on Day 4 of the dosing phase; (2) one animal (Group 3 female) on Day 4 of the dosing phase; and (3) two animals (Group 3 females) on Day 5 of the dosing phase. The anticoagulants were sodium citrate for coagulation tests and potassium EDTA for hematology tests. Samples for clinical chemistry were collected without anticoagulant. A necropsy was conducted on all Group 3 and 4 males on Day 4 of the dosing phase. A necropsy was also conducted on three animals (Group 4 females) at an unscheduled interval on Day 5 of the dosing phase.

On Day 10 of the dosing phase, all surviving animals, having been fasted overnight, were anesthetized via isoflurane inhalation, exsanguinated, and necropsied. Terminal body weights were recorded for animals sacrificed at a scheduled interval. A macroscopic examination of the external features of the carcass; external body orifices; abdominal, thoracic, and cranial cavities; organs; and tissues was performed. Organ weights were recorded at the scheduled sacrifice. Paired organs were weighed together. Bone marrow smears (two slides) were prepared from the femur of each animal.

Various models of calculators, computers, and computer programs were used to analyze data in this study. Values in some tables (e.g., means, standard deviations, or individual values) may differ slightly from those in other tables, from individually calculated data, or from statistical analysis data, because different models round off or truncate numbers differently. However, neither the integrity nor the interpretation of the data was affected by these differences.

Data for each sex were analyzed separately; only data collected on or after the first day of dosing were analyzed statistically. Analysis of variance (ANOVA) and pairwise comparisons were used to analyze absolute body weight, Body weight change and continuous clinical pathology values. Levene's test was done to test for equality of variances between groups. Where Levene's test was significant (P≤0.5), a rank transformation (to stabilize the variances) was applied before ANOVA was conducted (note: Levene's test was not applied to the rank-transformed data). Where Levene's test was not significant (P >0.5), ANOVA was conducted. One-way ANOVA was used (if applicable) to analyze the obtained data types. If the group effect of the ANOVA was significant (P≤0.5), Dunnett's t-test was used for pairwise comparisons between each treated and control group. Group comparisons (Groups 2 through 4 versus Group 1) were evaluated at the 5.0%, two-tailed probability level. If the ANOVA was not significant (P>0.5), no further analyses were conducted. In the current rat studies, the sex/group was omitted for full analyses when the number of data points for a given interval and data type from that sex/group fell below three.

Methods for toxicology and MTD studies in limited numbers of rats using the clinically compatible FL118-HP BCD) complex powder suspension: The test article FL118 was used from the glacial acetic acid/ethanol and microfluidizer-involved SDD-processed FL118-HPβCD complex powder product (LOT 2020-252-23). This FL118 product was resuspended using saline to form a suspension in different appropriate concentrations for oral gavage application. The FL118 suspension was gently inverted 5-10 times prior to each use. The control article was the same solution without FL118 API (i.e., an appropriate concentration of HPβCD in saline). Naïve 6-8-week-old Sprague-Dawley male (167 g-185 g) and female (130 g-148 g) rats with 7-day acclimation were used for toxicology and MTD finding studies. Males and females housed separately in autoclaved polycarbonate/polysulfone microisolator cages (≤2 per cage) with autoclaved contact bedding. Environmental conditions in the animal housing room were maintained at an ambient temperature of 70° F.±3° F., relative humidity of 30%-70%, and a 12:12 light-dark cycle. Animal housing rooms were ventilated with 100% fresh, HEPA filtered air with 10-15 air changes per hour. All procedures were performed in compliance with all state, local, and federal laws and the PHS Policy on the Humane Care and Use of Laboratory Animals. The study was conducted in an AAALAC accredited facility at Roswell Park. All animal work was based on a facility rat protocol approved by the IACUC prior to the initiation of the study. Individual rats were identified via tail marking prior to dosing. Each rat received a unique identification number.

Rats were randomly assigned to 4 treatment groups consisting of 2 males and 2 females, and 1 male and 1 female for the vehicle control group. Each rat received weekly oral gavage of FL118 at various doses or vehicle control on Days 1, 8, 15, 22, 29, 36, and 43. The FL118 dose dilutions were prepared so as to match the dose volume of 5 mL/kg for the corresponding mg/kg dose level based on the rat body weight then, and the treatment groups received the doses with corresponding dose concentration as outlined the Example 63 in the “Examples” section (Table 29).

In accordance with the Roswell Park Laboratory Animal Shared Resources (LASR) SOP, Physical examination was conducted on all rats' pre-study to evaluate general appearance, posture, behavioral status, respiratory system, skin, fur, eyes, nose, urine, feces, locomotion, and body orifices. Cage side clinical observations of experimental rats for mortality and signs of pharmacologic and/or toxicologic effects were conducted once daily in the morning on Days 1-50 of the study. Daily cage side clinical observations included but were not limited to evaluation of the general appearance, posture, behavioral status, respiratory system, skin, fur, eyes, nose, urine, feces, locomotion, and body orifices. The nature, onset, severity, and duration of all gross or visible pharmacologic or toxicologic signs were recorded. Rats were weighed and weights were recorded immediately prior to dosing, and then twice weekly and at termination, using a digital scale (Ohaus CS 2000 balance). In addition to daily clinical observations, all rats were observed at least once daily for morbidity, mortality, signs of pain or distress, and availability of food and water. Any rats in poor health were identified for further monitoring or possible euthanasia. Food consumption was qualitatively assessed daily and recorded. All rats were euthanized with carbon dioxide on Day 50, followed by blood collection via caudal vena cava venipuncture from rats of Group 1 (0 mg/kg FL118 vehicle controls), Group 3 (4.77 mg/kg FL118), Group 4 (3.5 mg/kg FL118), and Group 5 (1.56 mg/kg FL118). Blood samples were placed in pre-labeled serum separator tubes and in K3EDTA plastic tubes, and tightly capped. The tube label contained the LASR study number, rat ID number, nature of the specimen, and the date and time interval of collection. The following tests were conducted.

Clinical hematology: Blood samples were collected into tubes containing K3EDTA. Whole blood samples were stored on wet ice or under refrigeration (4° C.) until they were processed the same day using the IDEXX ProCyte A5088 Hematology Analyzer. The following hematopoietic parameters were evaluated for the whole blood: (1) Red Blood Cell Count (RBC, M/μL), (2) Hemoglobin (HGB, g/dL), (3) Hematocrit (HCT, %), (4) Mean Cell Volume (MCV, fL), (5) Mean Corpuscular Hemoglobin (MCH, pg), (6) Mean Corpuscular Hemoglobin Concentration (MCHC, g/dL), (7) Red Cell Distribution Width (RDW, %), (8) Reticulocytes, absolute (RET, K/μL), (9) Reticulocytes, relative (RET, %), (10) Platelet count (PLT, K/μL), (11) White Blood Cell Count (WBC, K/μL), (12) White Blood Cell Count Differential, absolute (WBCDa, L/μL), and (13) White Blood Cell Count Differential, relative (WBCDr, %).

Clinical chemistry: Blood samples were collected into serum separator tubes. Blood samples were stored at room temperature until they were centrifuged, and serum was removed. Samples were centrifuged at 3000 RPM for 15 minutes at ambient temperature. Serum was removed and the clinical chemistry analyses of the isolated serum were run the same day on the IDEXX Catalyst DS Chemistry Analyzer to analyze (1) albumin/globulin ratio (A/G, no unit), (2) alanine aminotransferase (ALT, U/L), (3) albumin (ALB, g/dL), (4) alkaline phosphatase (ALKP, U/L), (5) amylase (AMYL, U/L), (6) bilirubin, total (TBIL, mg/dL), (7) calcium (CA, mg/dL), (8) chloride (Cl, mmol/L), (9) creatinine (CREA, mg/dL), (10) cholesterol, total (CHOL, mg/dL), (11) gamma glytamyltransferase (GGT, U/L), (12) globulin, calculated (GLOB, g/dL), (13) glucose (GLU, mg/dL), (14) lipase (LIPA, U/L), (15) phosphorus, inorganic (PHOS, mg/dL), (16) potassium (K, mmol/L), (17) sodium (Na, mmol/L), (18) total protein (TP, g/dL), and (19) urea nitrogen (BUN, mg/dL).

Gross necropsy (examination of external surfaces, orifices, cranial, thoracic, and abdominal cavities, carcass and all organs) was performed on all rats at termination. The cranial, thoracic, and abdominal cavities were opened and examined for gross abnormalities or lesions. The following tissues were examined grossly: adrenals, aorta, brain, cecum, colon, duodenum, epididymis, esophagus, eyes, heart, ilium, jejunum, kidneys, liver, lungs, lymph nodes, mammary glands, ovaries (females only), pancreas, prostate (males only), salivary glands, seminal vesicles (males only), skeletal muscle, skin, spleen stomach, testes (males only), thymus, thyroid, trachea, urinary bladder, and uterus (females only).

Method brief outline for toxicology and MTD studies in standard numbers of rats using the clinically compatible FL118-HPβCD complex powder suspension: The Canget-hired CRO WuXi AppTec Hong Kong Toxicology subsidiary performed this study. Twenty male and 20 female rats (Crl: CDR [SD] VAF/Plus®/SPF) were randomly assigned to 4 groups to determine the toxicity of FL118 products (i.e., the glacial acetic acid/ethanol and microfluidizer-involved SDD-processed FL118-HPβCD complex powder product, LOT 2020-252-23) when administered once weekly on Days 1 and 8 orally via oral gavage. Rats at the age of 8 to 9 weeks with the weight of 189.62 to 229.64 g for females and 326.69 to 402.98 g for males. The control group was administered vehicle only. Animals were randomly assigned to groups by Provantis based on body weight. The study design with the treatment groups received the dose and volume is outlined the Example 64 in the “Examples” section (Table 32). Various study content and procedures were similar to and described in detail in the early section of “Methods for toxicology and MTD studies in limited numbers of rats using the clinically compatible FL118-HPβCD complex powder suspension” above.

Method brief outline for toxicology and MTD studies in standard numbers of dogs using the clinically compatible FL118-HPβCD complex powder suspension: The WuXi AppTec Hong Kong Toxicology subsidiary also performed the FL118 product toxicology study with dogs. A total of Twenty-four dogs (Canis familiaris, 12 per sex) were randomly assigned to 4 groups of 3/sex/group and given control article/vehicle [9.4 mg/mL hydroxypropyl-β-cyclodextrin (HPβCD) in 0.85% NaCl (w/v) in Sterile Water, test article FL118 at 2.5-9.9, 3.3→6.6, or 4.4 mg/kg/dose by oral gavage once weekly on Day 1 and Day 8 at the dose volume of 5 mL/kg. Animals were approximately 7 to 8 months of age with body weights ranging from 5.9 kg to 8.0 kg in females and 7.6 kg to 9.3 kg in males at dosing initiation.

Twenty-four beagle dogs were randomly assigned to 4 groups of 3/sex/group to determine the toxicity of FL118 products (i.e., the glacial acetic acid/ethanol and microfluidizer-involved SDD-processed FL118-HPβCD complex powder product, LOT 2020-252-23). Animals were randomly assigned to groups by Provantis based on body weight. The study design with the treatment groups received the dose and volume is outlined the Example 65 in the “Examples” section (Table 39). The FL118 dose level selection shown in Table 39 are based on the following three facts: (1) as shown in the Example 62, by using non-clinically compatible old FL118 formulation products, rats obtained the highest non-severely toxic dose (HNSTD)/MTD is 1.65 mg/kg, while dogs obtained an MTD at 2.2 mg/kg (Ling, et al. J Exp Clin Cancer Res. 2018; 37:240); (2) as shown in the Example 63, the use of the newly developed FL118 product (LOT 2020-252-23) for toxicology studies performed by Roswell Park Animal Center in a limited numbers of rats (4 rats per group: 2 male and 2 female) with weekly ×up to 7 oral administration indicated that at the dose range of 3.81 and 4.77 mg/kg, rats showing very minor clinical observation toxicity without observing adverse effects on hematology values and without observing adverse effects on clinical chemistry values; and (3) as shown in the Example 64, the use of FL118 products (LOT 2020-252-23) for toxicology studies performed by CRO WuXi AppTec Hong Kong Toxicology subsidiary in a FDA requirement-matched standard numbers of rats (10 rats per group: 5 male and 5 female) with weekly×2 oral administration on Days 1 and 8 indicated that when compared with concurrent controls, while no body weight loss was noted in female rats, statistically significant decreased body weight was noted in scheduled terminated males at 3, 3.75→2.5, and 4.69→2 mg/kg/wk, decrease of 18.15%, 17.41% and 47.53% in males on Day 14 and plus two male mice death on days 6 and 12 from the doses of 4.69-+2 mg/kg/wk. Based on the previous three animal experiments' outcomes, for the dog toxicity and MTD study with the new FL118 product (LOT 2020-252-23), first the doses were set at 2.5 mg/kg, 3.3 mg/kg, and 4.4 mg/kg for the new FL118 product (LOT 2020-252-23) for the groups 2, 3 and 4 dogs on day 1. Because there were no observations of any clinical toxicity for the dogs within 7 days after oral administration of the new FL118 products, the doses were increased to 2.5 mg/kg/dose to 9.9 mg/kg/dose for group 2 and increased 3.3 mg/kg/dose to 6.6 mg/kg/dose for group 3 on day 8 oral administration.

The new FL118 powder product (LOT 2020-252-23) was directly resuspended with the required volume of saline by gently up-and-down inverting the container to result in the required concentration of FL118 product suspension for oral administration. After use, the FL118 product suspension was stored in cold room and every time before oral administration, gently up-and-down inverting the container 5-10 time and then take the required volume for oral administration.

Detailed observations were conducted once during pretest for all animals (including spare animals), once prior to dosing on Day 1 for all study animals, and once weekly thereafter during the dosing phase (at 6+0.5 hours postdose). Detailed observations were also conducted once on the day of scheduled necropsies.

Parameters evaluated during the study included morbidity/mortality, clinical observations, body weights, food consumption, clinical pathology (hematology, serum chemistry, coagulation, and urinalysis), gross pathology, organ weights, and histopathological evaluation.

The study schedule was once weekly on day 1 and day 8 orally via gavage for 14 days' observation. The control group were administered control article (vehicle) only without the FL118. Various study content and procedures were similar to the study outline in the early section of “Methods for toxicology and MTD studies in limited numbers of rats using the clinically compatible FL118-HP BCD complex powder suspension”.

Additional statistical analysis: Real-time quantitative (q) RT-PCR and MTT assay results were analyzed using Microsoft Excel and presented as the mean±standard deviation (SD) derived from ≥3 independent assays. The statistical significance of differences was determined by Student's t-test, with a p-value of ≤0.5 considered as significant. * P<0.5; ** P<0.01; *** P<0.001.

Appendix 1: Analytical methods
LiquidChromatography:
Column: Thermo BDS Hypersil C18 30×2.0 mm, 3 um, with guard column
M.P. Buffer: 25 mM ammonium formate buffer, pH 3.5
Aqueous Reservoir (A): 90% water, 10% buffer
Organic Reservoir (B): 90% acetonitrile, 10% buffer
Flow Rate: 350 μL/minute
Gradient Program:

| Time | | |
|---|---|---|
| (min) | % A | % B |
| 0.0 | 100 | 0 |
| 0.5 | 50 | 50 |
| 1.0 | 0 | 100 |
| 1.5 | 0 | 100 |
| 1.6 | 100 | 0 |
| 2.5 | 100 | 0 |

Total Run Time: 2.5 minutes
Autosampler: 10 OL Injection Volume
Autosampler Wash: water/methanol/2-propanol: 1/1/1; with 0.2% formic acid
Mass spectrometer:
Instrument: PE SCIEX API 4000
Interface: Turbo Ionspray
Mode: Multiple reaction monitoring
Method: 2.5-minute duration
Settings:

| Test Article | Q1/Q3 | DP | EP | CE | CXP | IS | TEM | CAD | CUR | GS1 | GS2 |
|---|---|---|---|---|---|---|---|---|---|---|---|
| FL118 | +393.2/349.4 | 110 | 10 | 35 | 11 | 5500 | 500 | 7 | 20 | 20 | 30 |

Experimental procedure for FL118 blood partitioning (Table 46): Studies were carried out in mixed-gender human whole blood (Lot #BRH1038596), obtained from Bioreclamation and collected on sodium heparin. The blood was kept on ice prior to the experiments. Hematocrit (the ratio of the volume of red blood cells (RBC) to the total blood) was measured (n=3) by centrifugation of the whole blood for 5 minutes using micro-hematocrit capillary tubes. Control plasma was obtained from a portion of the whole blood by centrifugation at 1,000g for 10 minutes. The whole blood and control plasma were adjusted to pH 7.4 and then warmed at 37° C. in a water bath for 10 minutes. Aliquots of the whole blood and control plasma were spiked with the test article and the control compound chlorthalidone. The final concentration of test article was 1 μM in the incubation. All samples were then incubated at 37° C. in a shaking water bath. The total organic solvent content in the incubation was ≤1.0%. After incubation for 60 minutes (n=3), the incubated whole blood was removed from the water bath and the plasma was separated by centrifugation at 1,000g for 10 minutes. Aliquots of the control plasma were also removed. All samples were treated with four volumes of ice-cold acetonitrile (ACN) containing internal standard. After the removal of plasma protein by centrifugation at 1,640g (3,000 rpm) for 10 minutes at 4° C., the supernatants were diluted with water and analyzed by LC-MS/MS. Analytical conditions are outlined in Appendix 1.

$${K}_{RBC / P} = ( 1 / H ) * ( {C}_{CP} / {C}_{P} - 1 ) + 1$$

$${K}_{WB / P} = {C}_{CP} / {C}_{P}$$

Note: H=Hematocrit (Human=0.413); CCP=Response ratio in control plasma; Cp=Response ratio in blood plasma.
Chlorthalidone acceptance criteria: KRBC/P ≥5.0

Experimental procedure for FL118 Caco-2 permeability (Table 47): Caco-2 cells (clone C2BBe1) were obtained from American Type Culture Collection (Manassas, VA). Cell monolayers were grown to confluence on collagen-coated, microporous, polycarbonate membranes in 12-well Costar Transwell R plates. Details of the plates and their certification are shown below. The permeability assay buffer was Hanks' balanced salt solution (HBSS) containing 10 mM HEPES and 15 mM glucose at a pH of 7.4. The buffer in the receiver chamber also contained 1% bovine serum albumin. The dosing solution concentration was 1 μM of test article in the assay buffer+/−1 μM valspodar. Cells were first pre-incubated for 30 minutes with HBSS containing+/−1 μM valspodar. Cell monolayers were dosed on the apical side (A-to-B) or basolateral side (B-to-A) and incubated at 37° C. with 5% CO₂ in a humidified incubator. Samples were taken from the donor and receiver chambers at 120 minutes. Each determination was performed in duplicate. The flux of co-dosed lucifer yellow was also measured for each monolayer to ensure no damage was inflicted to the cell monolayers during the flux period. All samples were assayed by LC-MS/MS using electrospray ionization. Analytical conditions are outlined in Appendix 1. The apparent permeability (Papp) and percent recovery were calculated as follows:

$${p}_{app} = ( {dC}_{r} / dt ) \times {V}_{r} / ( A \times {C}_{A} ) \qquad ( 1 )$$

$$Percent Recovery = 100 \times ( ( {V}_{r} \times {C}_{r} ) + ( {V}_{d} \times {C}_{d} ) ) / ( {V}_{d} \times {C}_{N} ) \qquad ( 2 )$$

Where, dCr/dt is the slope of the cumulative concentration in the receiver compartment versus time in μM s⁻¹;Vᵣ is the volume of the receiver compartment in. cm³; Vd is the volume of the donor compartment in cm³; A is the area of the insert (1.13 cm² for 12-well Transwell); CA is the average of the nominal dosing concentration and the measured 120 minute donor concentration in μM; CN is the nominal concentration of the dosing solution in μM; Cᵣ^{Final} is the cumulative receiver concentration in μM at the end of the incubation period; and C_{d}^{Final} is the concentration of the donor in μM at the end of the incubation period.

Efflux ratio (ER) is defined as Papp (B-to-A)/Papp (A-to-B)

Experimental procedure for plasma protein binding by FL118 (Table 48): (1) Studies were carried out in mixed-gender human plasma (Lot #AS1690-6), obtained from Bioreclamation and collected on sodium heparin. A Pierce Rapid Equilibrium Dialysis (RED) device was used for all experiments. Stock solutions of the test articles and control compound were first prepared in DMSO. Aliquots of the DMSO solutions were dosed into 1.0 mL of plasma at a dosing concentration of 1 μM for the test article and 10 μM for the co-dosed control compound, warfarin. Plasma (300 μL), containing test article and control compound, was loaded into two wells of the 96-well dialysis plate. Blank phosphate-buffered saline (PBS) (500 μL) was added to each corresponding receiver chamber. The device was then placed into an enclosed heated rocker that was pre-warmed to 37° C. and allowed to incubate for four hours. After 4 hours of incubation, both sides were sampled. (2) Aliquots (50 μL for donor, 200 μL for receiver) were removed from the chambers and placed into a 96-well plate. Plasma (50 μL) was added to the wells containing the receiver samples, and 200 μL of PBS was added to the wells containing the donor samples. Two volumes of acetonitrile (ACN) were added to each well, and the plate was mixed and then centrifuged at 3,000 rpm for 10 minutes. Aliquots of the supernatant were removed, diluted 1:1 into water, and analyzed by LC-MS/MS. (3) Protein binding values were calculated as follows: % Bound=[(PARR in Donor-PARR in Receiver)/(PARR in Donor)]×100. Of note, PARR=peak area response ratio to internal standard, including applicable dilution factors.

Experimental procedure for FL118 human hepatocyte stability studies (Table 49a, 49b): Mixed-gender human cryopreserved hepatocytes (Lot #1410266) were purchased from XenoTech. The hepatocytes were thawed and prepared according to the vendor's instructions, pooled into Krebs Henseleit buffer (KHB, pH 7.4), and kept on ice prior to the experiments. The hepatocyte suspension was equilibrated in a shaking water bath at 37° C. for 3 minutes, and then the reaction was initiated by spiking the test article into the hepatocyte suspension (1.5×10⁶ cells/mL) at a final test article concentration of 0.5 μM. The final DMSO content in the incubation mixture was ≤0.1%. The reaction mixture was incubated in a shaking water bath at 37° C. Positive controls, testosterone (1 μM) and 7-hydroxycoumarin (7-HC) (100 μM), were performed in parallel to confirm the activity of the hepatocytes. Aliquots of the test article were withdrawn (n=1) at 0, 15, 30, 60, and 120 minutes. Aliquots of testosterone were withdrawn (n=1) at 0, 5, 15, 30, 60, and 120 minutes. Aliquots of 7-HC were withdrawn (n=1) at 0 and 15 minutes. The reaction was immediately terminated by adding three volumes of ice-cold acetonitrile (ACN) containing internal standard. The samples were then mixed and centrifuged to precipitate proteins. An aliquot of the supernatant was then diluted with water. Calibration standards for the analysis of 7-HC metabolites were prepared in matched matrix. Test articles and testosterone samples were analyzed without calibration standards. All samples were analyzed by LC-MS/MS. Analytical conditions are outlined in Appendix 1. The peak area response ratio (PARR) vs. internal standard was compared to the PARR at time 0 to determine the percent remaining at each time point. Half-lives and clearance values were calculated using GraphPad software, fitting to a single-phase exponential decay equation.

Experimental procedure for FL118 human liver microsome stability studies (Table 50): Mixed-gender human liver microsomes (Lot #1210347) were purchased from XenoTech. The reaction mixture, minus NADPH, was prepared as described below. The test article was added into the reaction mixture at a final concentration of 0.5 μM. The control compound, testosterone, was run simultaneously with the test article in a separate reaction. An aliquot of the reaction mixture (without cofactor) was equilibrated in a shaking water bath at 37° C. for 3 minutes. The reaction was initiated by the addition of cofactor, and the mixture was incubated in a shaking water bath at 37° C. Aliquots (100 μL) were withdrawn at 0, 10, 20, 30, and 60 minutes for the test article and 0, 10, 30, and 60 minutes for testosterone. Test article and testosterone samples were immediately combined with 400 μL of ice-cold 50/50 acetonitrile (ACN)/H2O containing 0.1% formic acid and internal standard to terminate the reaction. The samples were then mixed and centrifuged to precipitate proteins. All samples were assayed by LC-MS/MS using electrospray ionization. Analytical conditions are outlined in Appendix 1. The peak area response ratio (PARR) to internal standard was compared to the PARR at time 0 to determine the percent remaining at each time point. Half-lives were calculated using GraphPad software, fitting to a single-phase exponential decay equation.

Reaction composition:

| | | |
|---|---|---|
| Liver Microsomes | 0.5 | mg/mL |
| NADPH (cofactor) | 1 | mM |
| Potassium Phosphate, pH 7.4 | 100 | mM |
| Magnesium Chloride | 5 | mM |
| Test Article (FL118) | 1 | μM |

Experimental procedure for FL118 stability studies in human liver S9 fraction (Table 51): Mixed-gender human liver S9 fraction (Lot #0810304) was purchased from XenoTech. The reaction mixture, minus cofactors, was prepared as described below. The test article was added into the reaction mixture at a final concentration of 0.5 μM. The control compounds, testosterone and 7-hydroxycoumarin (7-HC), were run simultaneously with the test article in a separate reaction. An aliquot of the reaction mixture (without cofactors) was equilibrated in a shaking water bath at 37° C. for 3 minutes. The reaction was initiated by the addition of cofactors (see below), and the mixture was then incubated in a shaking water bath at 37° C. Aliquots (100 μL) were withdrawn at 0, 10, 20, 30, and 60 minutes for the test articles and at 0, 10, 30 and 60 for testosterone and 7-HC. Test article and control samples were immediately combined with 400 μL of ice-cold 50/50 acetonitrile (ACN)/dH2O containing 0.1% formic acid and internal standard to terminate the reaction. The samples were then mixed and centrifuged to precipitate proteins. Supernatants were diluted 5-fold in 50/50 Hanks' balanced salt solution/ACN. All samples were analyzed by LC-MS/MS using electrospray ionization. Analytical conditions are outlined in Appendix 1. The peak area response ratio (PARR) to internal standard was compared to the PARR at time 0 to determine the percent remaining at each time point. Half-lives were calculated using GraphPad software, fitting to a single-phase exponential decay equation.

Reaction composition:

| | | |
|---|---|---|
| Liver S9 | 1.0 | mg/mL |
| NADPH (cofactor) | 1 | mM |
| UDPGA (cofactor) | 1 | mM |
| PAPS (cofactor) | 1 | mM |
| GSH (cofactor) | 1 | mM |
| Potassium Phosphate, pH 7.4 | 100 | mM |
| Magnesium Chloride | 5 | mM |
| Test Article | 1 | μM |

Experimental procedure for FL118 stability studies in human plasma and human whole blood (Table 52): Studies were carried out in mixed-gender human plasma (Lot #AS1690-6) and mixed-gender human whole blood (Lot #BRH1038596), obtained from Bioreclamation and collected on sodium heparin. Both matrices were adjusted to pH 7.4 prior to initiating the experiments. A DMSO stock as first prepared for the test article. Aliquots of the DMSO solution were dosed into 1 mL of matrix, which had been pre-warmed to 37° C., at a final test article concentration of 0.5 μM. The vials were kept in a benchtop Thermomixer for the duration of the experiment. Aliquots (100 μL) were taken at each time point (0, 15, 30, 60, and 120 minutes) and added to 96-well plates which had been pre-filled with 300 μL of acetonitrile (ACN). Samples were stored at 4° C. until the end of the experiment. After the final time point was sampled, the plate was mixed and then centrifuged at 3,000 rpm for 10 minutes. Aliquots of the supernatant were removed, diluted 1:1 into distilled water, and analyzed by LC-MS/MS. Analytical conditions are outlined in Appendix 1. The peak area response ratio to internal standard (PARR) was compared to the PARR at time 0 to determine the percent of test article remaining at each time point. Half-lives were calculated using GraphPad software, fitting to a single-phase exponential decay equation.

Experimental procedure to test whether FL118 is a ABCG2 BCRP (breast cancer resistance protein) substrate (Table 53): Caco-2 cells (clone C2BBe1) were obtained from American Type Culture Collection (Manassas, VA). Cell monolayers were grown to confluence on collagen-coated, microporous, polycarbonate membranes in 12-well Costar® plates. Details of the plates and their certification are shown below. The Transwell permeability assay buffer was Hanks' balanced salt solution (HBSS) containing 10 mM HEPES and 15 mM glucose at a pH of 7.4. The buffer in the receiver chamber also contained 1% bovine serum albumin. The dosing solution concentration was 1 μM of test article in the assay buffer +/−0.5 μM Ko143. Cells were first pre-incubated for 30 minutes with HBSS containing+/−0.5 μM Ko143. Cell monolayers were dosed on the apical side (A-to-B) or basolateral side (B-to-A) and incubated at 37° C. with 5% CO₂ in a humidified incubator. Samples were taken from the donor and receiver chambers at 120 minutes. Each determination was performed in duplicate. The flux of lucifer yellow post-experimentally was also measured for each monolayer to ensure no damage was inflicted to the cell monolayers during the flux period. All samples were assayed by LC-MS/MS using electrospray ionization. Analytical conditions are outlined in Appendix 1. The apparent permeability (Papp) and percent recovery were calculated as follows:

$${p}_{app} = ( {dC}_{r} / dt ) \times {V}_{r} / ( A \times {C}_{A} ) \qquad ( 1 )$$

$$Percent Recovery = 100 \times ( ( {V}_{r} \times {C}_{r}^{final} ) + ( {V}_{d} \times {C}_{d}^{final} ) ) / ( {V}_{d} \times {C}_{N} ) \qquad ( 2 )$$

Where, dCr/dt is the slope of the cumulative concentration in the receiver compartment versus time in μM s⁻¹; Vᵣ is the volume of the receiver compartment in cm³; V_{d} is the volume of the donor compartment in cm³; A is the area of the insert (1.13 cm² for 12-well Transwell); C_{A} is the average of the nominal dosing concentration and the measured 120 minute donor concentration in μM; C_{N} is the nominal concentration of the dosing solution in μM; Cᵣ^{final} is the cumulative receiver concentration in M at the end of the incubation period; and C_{d}^{final} is the concentration of the donor in μM at the end of the incubation period.

Efflux ratio (ER) is defined as Papp (B-to-A)/Papp (A-to-B).

Experimental procedure to test whether FL118 inhibits ABCG2 BCRP to take other substrates (Table 54): BCRP-MDCK cell monolayers were grown to confluence on collagen-coated, microporous, polycarbonate membranes in 12-well Costar Transwell plates. Cell monolayers were grown to confluence on collagen-coated, microporous, polycarbonate membranes in 12-well Costar Transwell plates. Details of the plates and their certification are shown below. The permeability assay buffer was Hanks' balanced salt solution containing 10 mM HEPES and 15 mM glucose at a pH of 7.4. The dosing solution concentration was 10 IM of cladribine in the assay buffer+/−0.5 M Ko143 or 1.0 μM FL118. Cell monolayers were first pre-incubated for 30 minutes with assay buffer+/−0.5 μM Ko143 or 1.0 μM FL118. After 30 minutes the buffer was removed, replaced with fresh dosing solution/assay buffer, and time was recorded as 0. Cell monolayers were dosed on the apical side (A-to-B) or basolateral side (B-to-A) and incubated at 37° C. with 5% CO₂ in a humidified incubator. Samples were taken from the donor and receiver chambers at 120 minutes. Each determination was performed in duplicate. The flux of lucifer yellow post-experimentally was also measured for each monolayer to ensure no damage was inflicted to the cell monolayers during the flux period. All samples were assayed by LC-MS/MS using electrospray ionization. Analytical conditions are outlined in Appendix 1.

The apparent permeability (Papp) and percent recovery were calculated as follows:

$${p}_{app} = ( {dC}_{r} / dt ) \times {V}_{r} / ( A \times {C}_{N} ) \qquad ( 1 )$$

$$Percent Recovery = 100 \times ( ( {V}_{r} \times {C}_{r}^{final} ) + {( {V}_{d} \times {C}_{d} )}^{final} ) / ( {V}_{d} \times {C}_{N} ) \qquad ( 2 )$$

Where, dCr/dt is the slope of the cumulative concentration in the receiver compartment versus time in IM s⁻¹; Vᵣ is the volume of the receiver compartment in cm³; V_{d} is the volume of the donor compartment in cm³; A is the area of the insert (1.13 cm² for 12-well Transwell); CN is the nominal concentration of the dosing solution in μM; Cᵣ^{final} is the cumulative receiver concentration in IM at the end of the incubation period; and C_{d}^{final} is the concentration of the donor in μM at the end of the incubation period.

Efflux ratio (ER) is defined as Papp (B-to-A)/Papp (A-to-B).

Experimental procedure to test whether FL118 inhibits P-gp to take other substrates (Table 55): Caco-2 cells (clone C2BBe1) were obtained from American Type Culture Collection (Manassas, VA). Cell monolayers were grown to confluence on collagen-coated, microporous, polycarbonate membranes in 12-well Costar Transwell R plates. Details of the plates and their certification are shown below. The permeability assay buffer was Hanks' balanced salt solution containing 10 mM HEPES and 15 mM glucose at a pH of 7.4. The dosing solution concentration was 10 IM of digoxin in the assay buffer+/−1.0 μM valspodar or 1.0 μM FL118. Cell monolayers were first pre-incubated for 30 minutes with assay buffer+/−1.0 μM Valspodar or 1.0 μM FL118. After 30 minutes the buffer was removed, replaced with fresh dosing solution/assay buffer, and time was recorded as 0. Cell monolayers were dosed on the apical side (A-to-B) or basolateral side (B-to-A) and incubated at 37° C. with 5% CO₂ in a humidified incubator. Samples were taken from the donor and receiver chambers at 120 minutes. Each determination was performed in duplicate. The flux of lucifer yellow post-experimentally was also measured for each monolayer to ensure no damage was inflicted to the cell monolayers during the flux period. All samples were assayed by LC-MS/MS using electrospray ionization. Analytical conditions are outlined in Appendix 1. The apparent permeability (Papp) and percent recovery were calculated as follows:

$${p}_{app} = ( {dC}_{r} / dt ) \times {V}_{r} / ( A \times {C}_{N} ) \qquad ( 1 )$$

$$Percent Recovery = 100 \times ( ( {V}_{r} \times {C}_{r}^{final} ) + ( {V}_{d} \times {C}_{d}^{{f}^{final}} ) ) / ( {V}_{d} \times {C}_{N} ) \qquad ( 2 )$$

Where, dCr/dt is the slope of the cumulative concentration in the receiver compartment versus time in μM s⁻¹; Vᵣ is the volume of the receiver compartment in cm³; V_{d} is the volume of the donor compartment in cm³; A is the area of the insert (1.13 cm² for 12-well Transwell); CN is the nominal concentration of the dosing solution in μM; Cᵣ^{final} is the cumulative receiver concentration in μM at the end of the incubation period; and C_{d}^{final} is the concentration of the donor in μM at the end of the incubation period.

Efflux ratio (ER) is defined as Papp (B-to-A)/Papp (A-to-B).

Experimental procedure to test whether FL118 inhibits CYP P450 (CYP1A2, CYP2B6, CYP3A) activity (Tables 11, 12): CYP inhibition was evaluated for one test article for CYP 1A2, 2B6, and 3A (with testosterone as the probe substrate) in human liver microsomes (HLM) obtained from XenoTech (Lot #1210347). The test article, at 1 μM, were incubated with pooled microsomes (0.25 mg protein/mL) at 37° C. in the presence of phosphate buffer (100 mM, pH 7.4), MgCl₂ (5 mM), CYP-specific probe substrate (at approximately Km), and NADPH (1 mM). The CYP reaction was initiated by adding a CYP probe substrate and incubating at 37° C. for 10-20 minutes, depending on the individual CYP isoform. The reaction was terminated with the addition of 400 μL ice-cold acetonitrile (ACN). Negative (vehicle) controls were conducted using the incubation medium without a test article. Positive controls were performed in parallel using a known inhibitor of the individual CYP isoform. After the removal of protein by centrifugation at 1,640g (3,000 rpm) for 10 minutes at 4° C., the supernatants were diluted 1:1 with water containing internal standard (stable isotope-labeled CYP probe metabolite). The samples were then transferred to 96-well plates and analyzed by LC-MS/MS (Appendix 1).

Experimental materials to test whether FL118 induces CYP P450 (CYP1A2, CYP2B6, CYP3A) activity (Table 58a, 58b): (1) The test articles (TAs), FL118 and HPβCD-formulated FL118, were provided by the Sponsor. The stock solution of FL118 (1 mM) was prepared in dimethyl sulfoxide (DMSO). The stock solution of HPβCD-formulated FL118 (1 mM) was prepared in saline with 4% DMSO. (2) CYP positive inducers (Table 58a) and Williams' Medium E (WME) were purchased from Sigma-Aldrich (St. Louis, MO, USA). Dulbecco's phosphate-buffered saline (DPBS, pH 7.4) was purchased from Invitrogen (Carlsbad, CA, USA). Hepatocyte culture medium (serum-free) for CYP induction was purchased from XenoTech (catalog #K2300, Lenexa, KS, USA). The CellTiter 96 AQueous ONE Solution Cell Proliferation Assay was obtained from Promega (Madison, WI, USA). All other chemicals and reagents were of analytical grade or higher. (3) Fresh human hepatocytes (Table 58b) seeded at ˜0.15 million cells/well in 48-well plates (˜0.75 million cells/mL) were purchased from Thermo Fisher Scientific (Grand Island, NY, USA) and BioreclamationIVT (Baltimore, MD, USA).

TABLE 58a
Positive Inducers for CYP Induction

| CYP | Positive Inducer |
|---|---|
| CYP1A2 | Omeprazole (OME) |
| CYP2B6 | Phenobarbital (PB) |
| CYP3A4 | Rifampicin (RIF) |

TABLE 58b
Human Hepatocytes for CYP Induction

| Donor | Vendor | Gender | Age | Race | Cat # | Lot # |
|---|---|---|---|---|---|---|
| 1 | Thermo Fisher | Female | 43 | Caucasian | HMFN48 | Hu1806 |
| 2 | BioreclamationIVT | Male | 51 | Caucasian | M91556 | MHUL110615 |
| 3 | BioreclamationIVT | Female | 53 | Caucasian | F91556 | FHUL010816 |

Experimental methods to test whether FL118 induces CYP P450 (CYP1A2, CYP2B6, CYP3A) activity (Tables 59-62): (1) Fresh human hepatocytes were recovered by incubating with hepatocyte culture medium in a 95% air/5% CO₂ incubator at 37° C. for 24 hours prior to the induction experiments. Hepatocytes were then treated with hepatocyte culture medium fortified with the TA at three concentrations (0.01, 0.1, and 1 μM FL118 for donor 1; 0.001, 0.005, and 0.01 μM FL118 for donor 2; 0.025 and 0.05 μM FL118, and 1 μM HPβCD-formulated FL118 for donor 3; based on the cell viability results and the Sponsor's request). Positive controls were treated in parallel with hepatocyte culture medium fortified with the positive inducers omeprazole (OME) at 50 M for CYP1A2, phenobarbital (PB) at 1,000 μM for CYP2B6, or rifampicin (RIF) at 50 μM for CYP3A4. Vehicle controls were treated in parallel with hepatocyte culture medium. The hepatocyte incubation was conducted in a 95% air/5% CO₂ incubator at 37° C. for three days with daily replacement of the hepatocyte culture medium containing TA, positive inducer, or vehicle. (2) After the CYP induction treatment, the cells were used for a cell viability assay. The viability of cells was measured by analyzing the cellular conversion of tetrazolium compound [3-(4,5-dimethylthiazol-2-yl)-5-(3-carboxymethoxyphenyl)-2-(4-sulfophenyl)-2H tetrazolium, inner salt; MTS] into a formazan product by dehydrogenases, which are active only in viable cells. The absorbance of formazan, which is proportional to the number of viable cells, was measured spectrophotometrically using the CellTiter 96 AQueous ONE Solution Cell Proliferation Assay. Briefly, the wells were rinsed with DPBS, and then 200 μL of hepatocyte culture medium and 40 μL of the CellTiter 96 AQueous ONE Solution Cell Proliferation Assay reagent were added to each well, and the cells were incubated for 1 hour at 37° C. in a 95% air/5% CO₂ incubator. The absorbance of formazan in each well was measured at 492 nm using a FLUOStar OPTIMA Microplate Reader (BMG Lab Technologies, Durham, NC, USA). (3) Total RNA was isolated from the treated cells using the RNeasy mini kit (Qiagen, Valencia, CA, USA) and treated with RNase-free DNase (Qiagen) following the manufacturer's protocols. The concentration of RNA was determined using a Qubit Fluorometer with a Qubit RNA HS assay kit (Invitrogen). cDNA was synthesized from up to 1 μg of the total RNA harvested from the cells using a QuantiTect RT kit (Qiagen). Analysis of gene expression by qPCR was performed using the LightCycler 480 System (Roche Diagnostics Corporation, Indianapolis, IN, USA).

Experimental procedure to test whether FL118 inhibit hERG activity (Table 63, 64a, 64b): (1) The experiment was performed on an IonWorks™ HT instrument (Molecular Devices Corporation), which automatically performs electrophysiology measurements in 48 single cells simultaneously in a specialized 384 well plate (PatchPlate™). All cell suspensions, buffers and test compound solutions were at room temperature during the experiment. (2) The cells used were Chinese hamster ovary (CHO) cells stably transfected with hERG (cell line obtained from Cytomyx, UK). A single cell suspension was prepared in extracellular solution (Dulbecco's phosphate buffered saline with calcium and magnesium pH 7.2) and aliquots added automatically to each well of a PatchPlate™. The cells were then positioned over a small hole at the bottom of each well by applying a vacuum beneath the plate to form an electrical seal. The vacuum was applied through a single compartment common to all wells which was filled with intracellular solution (buffered to pH 7.2 with HEPES). The resistance of each seal was measured via a common ground electrode in the intracellular compartment and individual electrodes placed into each of the upper wells. Electrical access to the cell was then achieved by circulating a perforating agent, amphotericin B, underneath the PatchPlate™. (3) The pre compound hERG current was then measured. An electrode was positioned in the extracellular compartment and a holding potential of 80 mV applied for 15 sec. The hERG channels were then activated by applying a depolarizing step to +40 mV for 5 sec and then clamped at 50 mV for 4 sec to elicit the hERG tail current, before returning to 80 mV for 0.3 sec. (4) Compound dilutions were prepared by diluting 10 mM DMSO solutions of the test compound (FL118) by a factor of 5 in DMSO, followed by dilution into extracellular buffer such that the final concentrations tested are 0.008, 0.04, 0.2, 1, 5 and 25 μM (final DMSO concentration 0.25%). (5) The IonWorks™ HT instrument automatically added test compound dilutions to the upper wells of the PatchPlate™ from a 96 well microtitre plate. The test compound was left in contact with the cells for 300 sec before recording currents using the same voltage step protocol as in the pre compound scan. Quinidine, an established hERG inhibitor, was included as a positive control and buffer containing 0.25% DMSO was included as a negative control. (6) Each concentration was tested in 4 replicate wells on the PatchPlate™ (maximum of 24 data points). For a data point to be used to assess hERG blockade, the cell must maintain a seal resistance of greater than 50 MOhm and a pre compound current of at least 0.1 nA. Additional filters were also applied to pre and post compound (FL118) data to assess cell stability. The experimental conditions were summarized in Table 63 below.

TABLE 63
Summary of the experimental conditions for hERG inhibition

| Test | Test | Reference | |
|---|---|---|---|
| Articles * | concentrations | compound | Analytical method |
| FL118 | 0.003 μM-1 μM | quinidine | electrophysiology |
| (bl-4) | | | (patch clamp) |

* Test article (FL118) stored at −20° C. (in dark).

The percentage inhibition was calculated using the following equation:

$$Percent inhibition = ( Post - compound current ) / ( Pre - compound current ) \times 100$$

The percentage inhibition was plotted against concentration for the test compound and, where concentration dependent inhibition was observed, the data were fitted to the following equation and an IC50 value calculated:

$$y = \frac{{y}_{max} - {y}_{min}}{{( 1 + \frac{x}{{x}_{50}} )}^{s}} + {y}_{min}$$

Where, y=percentage inhibition; x=concentration; x50=concentration required to inhibit current by 50% (IC50); and s=slope of the graph.

Experimental methods for FL118 Ames Bacterial Reverse Mutation Assay studies (Ames Test: Tables 65-69): The Ames assay measures the ability of a test chemical or test article extract to induce mutations in strains of Salmonella typhimurium and Escherichia coli bacteria. The Ames Bacterial Reverse Mutation (AB™) used five bacterial tester strains (TA97a, TA98, TA100, TA1535, WP2-uvrA). The test article is FL118; the negative control is the corresponding concentration of DMSO; The positive controls are (a) ICR-191 Acridine for TA97a; (b) 2-nitrofluorence/2-NF for TA98; (c) sodium azide for TA100 and TA1535; and 4-nitroquinoline 1-oxide (4NQO) for WP2-uvrA.

Tester strains were added to flasks containing nutrient broth and incubated at 37+2° C. overnight. The flasks were removed from the overnight incubation and the final concentration was determined (the OD was measured and determined whether growth was suitable for testing). Top agar was melted, and 2.0 mL was dispensed into heated culture tubes. The S9 mix (metabolic activation) was prepared and stored on ice. The test article dose (or vehicle (negative) control or positive control), S9 mix/PBS sham, and appropriate tester strain were added to the tubes containing the melted agar. Each culture tube was vortexed and immediately poured onto the labeled minimal glucose agar plates. All plates were inverted and placed into a 37±1° C. incubator for 48-72 hours. The plates were counted via an automated colony counter or by hand and the background lawn was microscopically evaluated for any signs of toxicity. Genotypic control plates were prepared for each tester strain and incubated for 24-72 hours at 37±1° C.

Experimental methods for FL118 Mouse Lymphoma Assay studies (Tables 70-75): (1) The test system consisted of L5178Y TK+/−clone (3.7.2C) cells, which were obtained from ATCC and grown in suspension at 37±1° C. with 5+1% CO₂. The cells were prepared in Fischer's or RPMI media supplemented with 10% heat-inactivated horse serum, penicillin-streptomycin, Lglutamine, sodium pyruvate and Pluronic F68. In the initial assay, prior to use in the test, the culture was purged of preexisting TK−/− mutations by exposure to THMG treated medium for 1 day, followed by a transfer to THG medium for 1 to 2 days. This was performed 5 to 8 days prior to testing. In the expanded assay a vial of frozen cells was thawed and used within 5 to 8 days of growth. No washing was needed because the cells were purged of preexisting TK−/− mutations by exposure to THMG treated medium for 1 day and then THG medium for 1 day prior to freezing. The cells tested negative for the presence of mycoplasma and were confirmed to have 2 normal copies of chromosome 11 through completion of karyotyping. The L5178Y TK+/− cell line has significant historical use for this type of assay, has proven sensitivity to leachates, and is an established cell line that produces reliable and consistent results. (2) Experiments: The L5178Y TK+/−mouse lymphoma suspension cells were plated in a petri dish with soft agar media exposed to the test article (FL118)/controls for 4 hours (with the presence and absence of metabolic activation S9) and for 24 hours (without S9). Colonies were counted using an automated colony counter or manually. After 4 or 24 hours of exposure, the cells were grown for recovery and expression for approximately 48 hours before plated in TFT cloning media. A second set of plates were prepared without TFT to assess viability of cells following treatment. After 10-14 days, the colonies were counted to determine genotoxicity. Colonies observed on the TFT plate were defined as small or large colonies. Absolute Cloning Frequency (ACE), Relative Total Growth (RTG) and Mutant Frequency (MF) data are obtained from the plate counts and the FL118 test article results were compared to that of the negative control. (3) Of note, the metabolic activation system in this assay consisted of a rat liver homogenate (S-9 microsomal fraction), which was delivered to the test system in the presence of a core mix consisting of NADP and cofactors for NADPH-supported oxidation. The S9 was prepared using the following recipe (per treatment tube); 1 mL of 20 mg/mL S9, 32 mg of NADP, 60 mg of isocitric acid, 40 μL of calcium chloride (CaCl2) and 3.0 mL of cell cultural media. All treatments were performed in duplicate. The treatment tubes were placed in a CO₂ incubator at 37±1° C. with continuous agitation.

Experimental methods for FL118 oral administration pharmacokinetics (PK) studies (Tables 76, 77): (1) The objective of this study was to determine the plasma and tumor FL118 pharmacokinetics after oral administration to severe combined immunodeficiency (SCID) female mice (8 weeks of age). The study was 8 groups, 3 mice per group. FL118 was administered orally to SCID mice as a single dose of 1.5 mg/kg. Each of the SCID mice was first inoculated with both SW620 and HT29 human colon tumors in the left and right flank sites, respectively. Once the tumor size reached approximately 800-1,000 mm³, a single oral administration of FL118 was given at a dose of 1.5 mg/kg. The FL118 was formulated in a DMSO-free saline solution containing FL118 at 0.15 mg/mL, hydroxypropyl-β-cyclodextrin at 0.15%, hydroxypropyl methylcellulose. (HPMC) at 2.0%, and propylene glycol at 1.0%. Mice were numbered and dosed as follows: mice were designated as 1, 2 and 3 at each time point and each received a 1.5 mg/kg dose of FL118 following tumor development. Tumor tissues and blood samples were collected at a series of time points: 0.50, 1.0, 2.0, 4.0, 8.0, 12.0, 24.0 and 48.0 hours after FL118 oral administration. Each tumor tissue was harvested, transferred to a specimen tube, and immediately frozen with liquid nitrogen. Each blood sample was collected in a Li-Heparin LH/1.3 tube (SARSTEDT) and the plasma recovered by centrifugation (1500 rpm×2.00 minutes). The collected plasma was transferred into a new tube and immediately frozen in liquid nitrogen. The frozen specimens were then transferred to a −80° C. freezer for storage until analysis. (2). FL118 concentrations in the serum and tumor samples were determined by Roswell Park BMPK (Bioanalytics, metabolomics, and pharmacokinetics Shared Resource) Facility using the BMPK Method MN1055.v01, a liquid chromatographic assay with fluorescence detection having a lower limit of quantitation (LLOQ) of 1.00 ng/mL. The calibration range of the assay is 1.00-500 ng/mL for FL118 with quality control (QC) samples prepared at concentrations of 3.00, 100, and 375 ng/mL. Lithium heparinized human plasma was used as the matrix for the preparation of calibration and QC samples that were used for the analysis of the study samples. The study samples were analyzed in 2 runs on June 4^{th} and 8^{th}, 2015. The calibration curve was analyzed using a weighted linear regression analysis with a weighting factor of 1/x². Acceptance criteria for the analytical run required all calibration samples used in the regression analysis to have a deviation of no more than 15% from the nominal value except at the LLOQ, which was allowed 20%. The correlation coefficient for the calibration curves were 0.9992 and 0.9998, respectively. Acceptance criteria for the FL118 QC samples required two-thirds of the QC samples in the analytical run to have results within 85-115% of the nominal values and an overall precision of ≤15% (% CV). The concentrations of the study samples in each run were back-calculated using the slope and y-intercept from the calibration curve within that run. The FL118 calibrator and QC accuracy and precision results from the analysis of the study samples are summarized in the following tables.

| | Calibrator Accuracy (%) | | Calibrator Precision (%) | |
|---|---|---|---|---|
| Compound | Mean | Range | Mean | Range |
| FL118 | 98.2 | 85.7-111 | 2.68 | 0.322-8.30 |

| | |
|---|---|
| QC Accuracy (%) | QC Precision (%) |

| Compound | Mean | Range | Mean | Range |
|---|---|---|---|---|
| FL118 | 97.3 | 96.7-97.9 | 5.42 | 5.00-6.16 |

(3) A noncompartmental analysis (NCA) was performed on individual plasma and tumor concentration-time data using a linear trapezoidal, linear interpolation sparse sampling plasma model with uniform weighting. The software program utilized for the analysis was Phoenix 64 WinNonlin (Pharsight Corp., St. Louis, MO, Version 6.3). Data from all reportable time points were used for the calculation of plasma and tumor pharmacokinetic parameters following single oral dose administration. A test for pharmacokinetic outliers was performed by identifying values that fell outside three standard deviations from the mean concentration at each time point. Based on this determination, no values in this study were found to be pharmacokinetic outliers; therefore, the concentration data at all reportable time points were included in the pharmacokinetic parameter calculations.

An example of Protein-FL118 formulation process is described below.

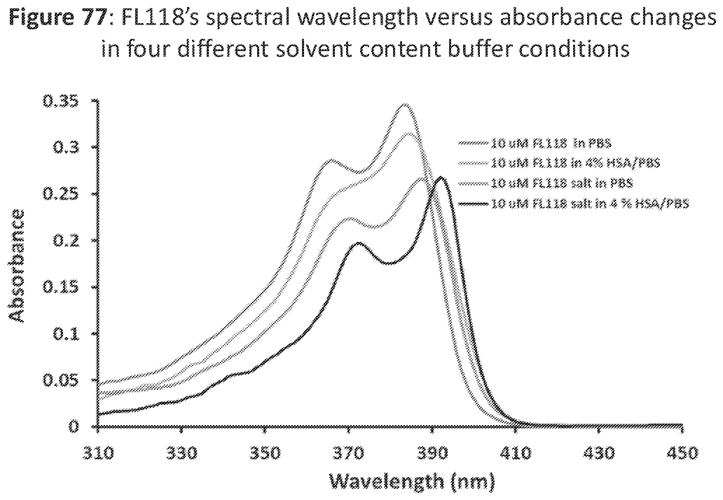

Solution preparation: (1) Preparation of FL118 salt solution—To 13.35 mg of FL118 was added 7300 μL of 50% ethanol/water mixture, followed by adding 200 μL of 0.5 M NaOH solution, the resulting mixture was sonicated for 15 min, a clear solution of FL118 salt with a concentration of 1.78 mg/mL (4538 μM) FL118 salt was formed. (2) Preparation of FL118 in DMSO solution—To 10.24 mg of FL118 was added 12 mL of DMSO and the resulting mixture was sonicated a hot water bath for 15 min, a clear solution with a concentration of 2174 μM of FL118 was formed. 10 μM of either FL118 and FL118 salt solutions in a PBS or a PBS containing 4% HSA (w/v.) or a PBS containing 2% IGG (w/v) were prepared and analyzed by a UV spectrometer, scanning from 310 nm to 450 nm. All UV spectra were shown in FIGS. 77-79

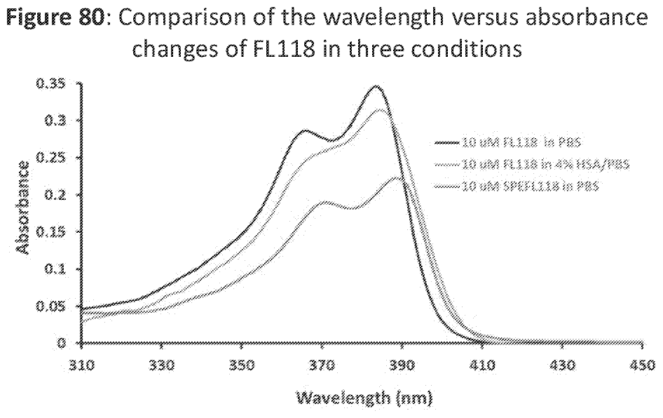

The preparation of SPEFL118, was prepared by using FL118 salt solution. The final SPEFL118 solution is filtered through 0.22 um membrane (PES) and lyophilized into a powder product. 10 μM of FL118 equivalent solutions in PBS or a PBS containing 4% HSA (w/v) were prepared and analyzed by a UV spectrometer, scanning from 310 nm to 450 nm. The UV spectra were shown in FIG. 80.

Experiment methods for testing anticancer drug efficacy and toxicity using human tumor animal models: All in vivo experimental studies were approved by the Institutional Animal Care and Use Committee (IACUC) and performed following the IACUC-approved mouse protocol at Roswell Park Animal Center. Methods in brief: Human SW620 colorectal cancer xenograft tumors or human HT1080 fibrosarcoma tumors were first generated through implanting 2×10⁶ SW620 cancer cells or HT1080 cancer cells at the flank area of severe combined immunodeficiency (SCID) mice. After tumor grew to 800-1,200 mm³, the tumors were isolated and individual experimental mice were subcutaneously implanted with 30-50 mg non-necrotic tumor masses at the flank area of individual mice. Seven to 10 days after tumor transplantation at which the implanted SW620 xenograft tumors or HT1080 xenograft tumors were grown to 100-200 mm³ (defined as day 0), mice were randomly divided into the required groups for treatment via administration of vehicles or drugs in the defined formulation through intravenous routes (i.v.), intraperitoneal route (i.p.) or oral routes (p.o.). The schedule for treatment was weekly for up to 4-times (weekly×4) or bi-weekly for up to 2-times (bi-weekly×2). Tumor length (L) and width (W) were measured using digital vernier calipers 2-3 times per week until the end of experiments. The tumor volume (v) was calculated using the formula: v=0.5 (L×W2). Then the tumor size was divided by the day 0 tumor size as percentage tumor size versus day 0. The tumor curves were made using Microsoft Excel.

### EXAMPLES

The present disclosure is further illustrated by the following examples, which should not be construed as limiting in any way. The following is a description of the examples.

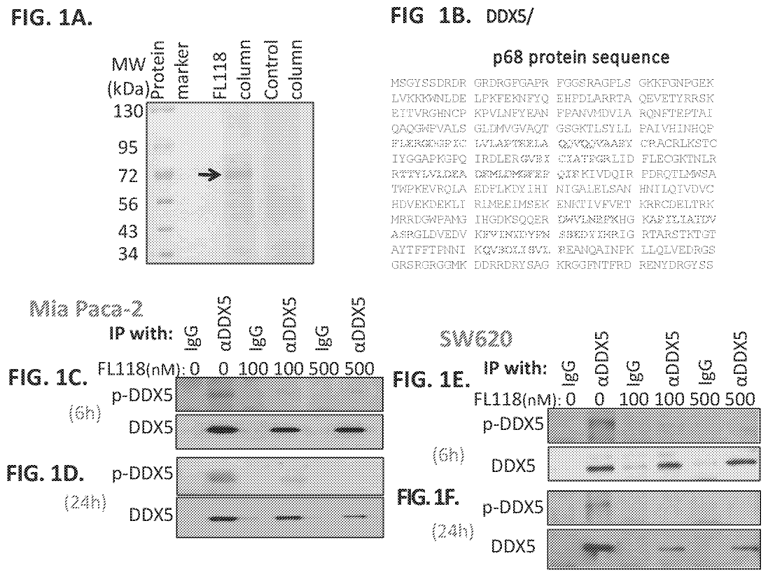

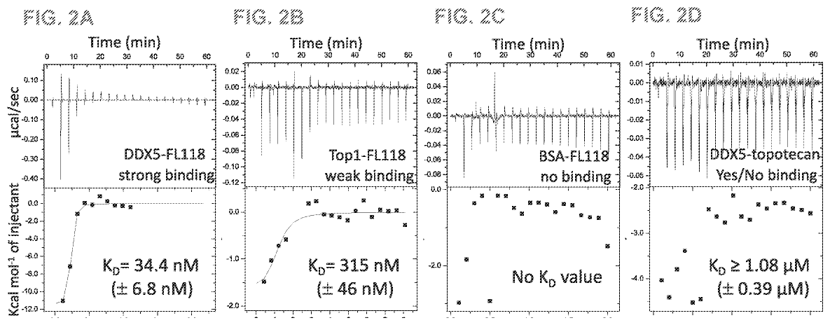

Example 1—FL118 affinity purification identified that the oncogenic protein DDX5 is the physical and functional direct biochemical target for FL118: The FL118 affinity purification and mass spectrometry analysis studies have discovered that FL118 physically binds to the oncogenic protein DDX5 (also called p68 in early time studies) and rapidly induces DDX5 dephosphorylation (i.e., functional inactivation), followed by DDX5 protein degradation (FIG. 1). Isothermal Titration calorimetry (ITC) studies indicated that FL118 binding to DDX5 is about 10-time stronger that FL 118 binding to Top1 (KD=34.4 nM versus KD=315 nM), while topotecan showed a Yes/No binding with a KD ≥1080 nM (FIG. 2). These studies indicated that DDX5 is a bona fide target for FL118.

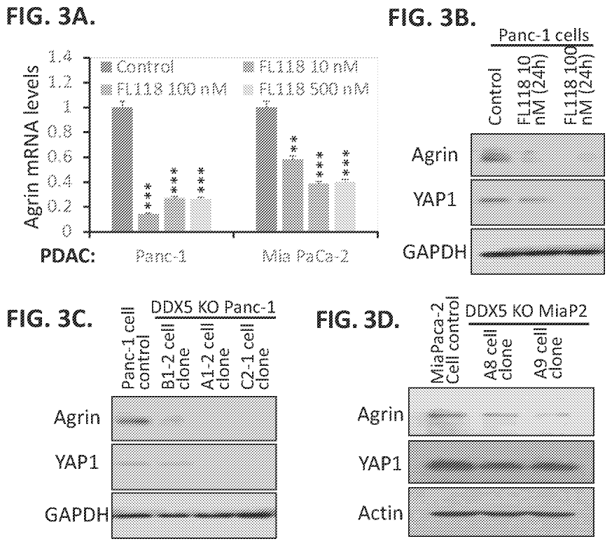

Example 2—The tumor microenvironment (TME) stiff stroma extracellular matrix (ECM) regulator, proteoglycan Agrin, can be inhibited by FL118 and Agrin appears to be a DDX5 downstream target: The fact that FL118 exhibits high efficacy to regress pancreatic cancer PDX tumors suggests that the pancreatic cancer dense/stiff stroma ECM (defined as desmoplasia) is not an issue for FL118, and also it is known that Agrin (secreted from cancer cells) acts as a tissue and ECM stiffness signal to activate the Hippo effector and transcription coactivator YAP1 to exert oncogenic potential (Chakraborty, et al. Cell Rep 2017, 18:2464-79). FL118 treatment of pancreatic cancer cells inhibits both Agrin mRNA (determined by real-time RT-PCR) and protein (determined by Western blots) as well as the Agrin downstream target YAP1 (FIGS. 3A and 3B). Convincingly, Knockout (KO) of DDX5 in pancreatic cancer cells resulted in the downregulation of both Agrin and YAP1 expression (FIGS. 3C and 3D). These studies indicated that Agrin is a downstream target of DDX5 and FL118-induced inhibition of Agrin is through the FL118-DDX5-Agrin signaling pathway.

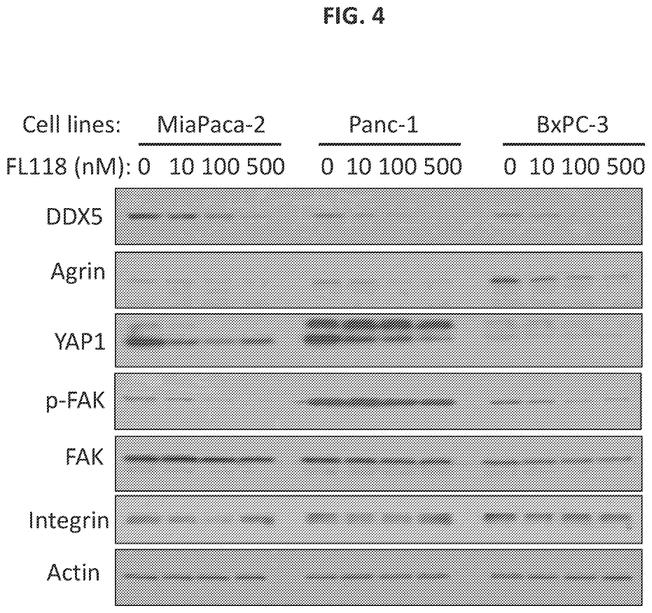

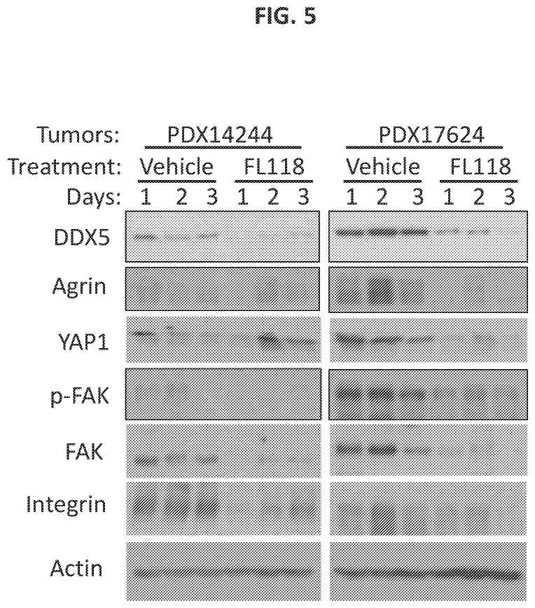

Example 3—FL118 is extensively involved in the inhibition of Agrin downstream signaling pathway: Previous studies demonstrated that YAP1, FAK and Integrin are Agrin downstream targets (Chakraborty, et al. Nat Commun 2015, 6, 6184; Chakraborty, et al. Cell Rep 2017, 18, 2464-2479). In this regard, by using multiple pancreatic cancer cell lines, the expression and/or phosphorylation status of DDX5, Agrin, YAP1, FAK and Integrin were tested. The studies indicated that FL 118 treatment of pancreatic cancer cells inhibits the expression of DDX5, Agrin, YAP1 and Integrin as well as the FAK phosphorylation status (FIG. 4). More excitingly, when pancreatic cancer PDX tumors were isolated from FL118 treated tumor-bearing SCID mice to determine the same parameters and found that these in vivo data is consistant with the in vitro cell-based results but a more profound decrease (FIG. 5).

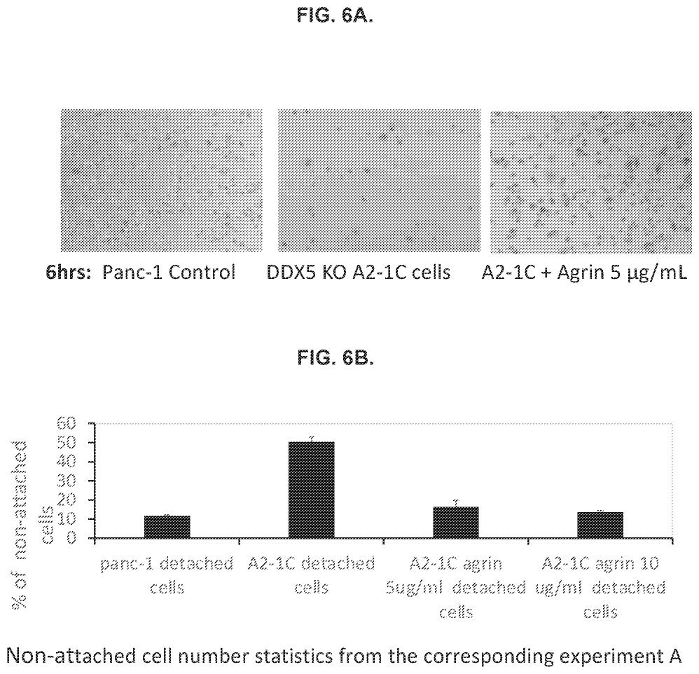

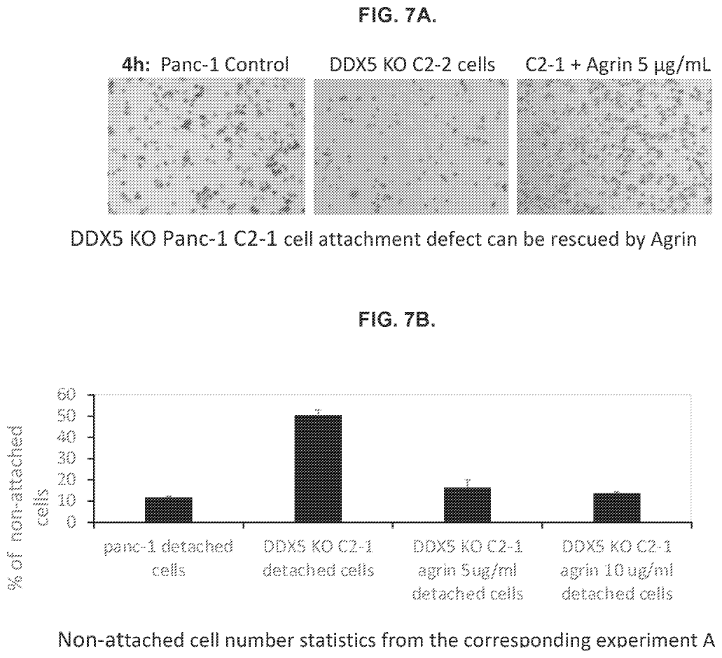

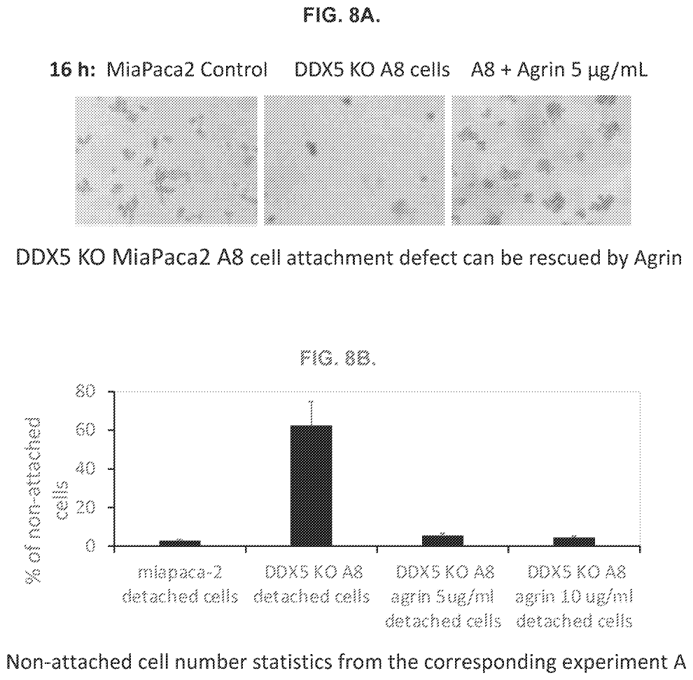

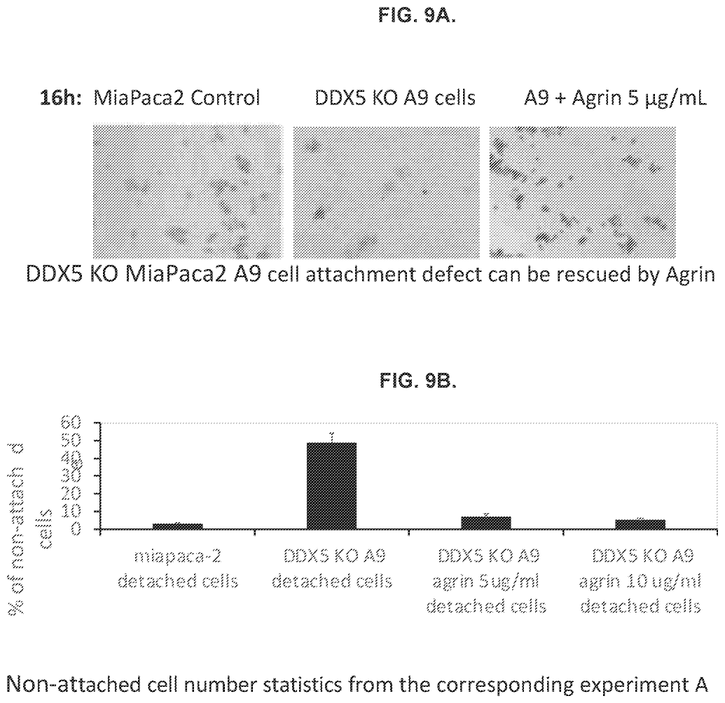

Example 4-Knockout (KO) of DDX5 induces pancreatic cancer cell attachment defect, which can be rescued by adding exogenous Agrin in the cell culture medium: In the cell attachment studies, DDX5 KO pancreatic cancer Panc-1 cells exhibited cell attachment defect in comparison with the wild type panc-1 cells; however, this DDX5 KO-induced cell attachment defect could be restored/rescued by adding the exogenous Agrin in the cell culture medium (FIG. 6, 7). The same observed results were also obtained from DDX5 KO pancreatic cancer Mia Paca-2 cells (FIGS. 8, 9).

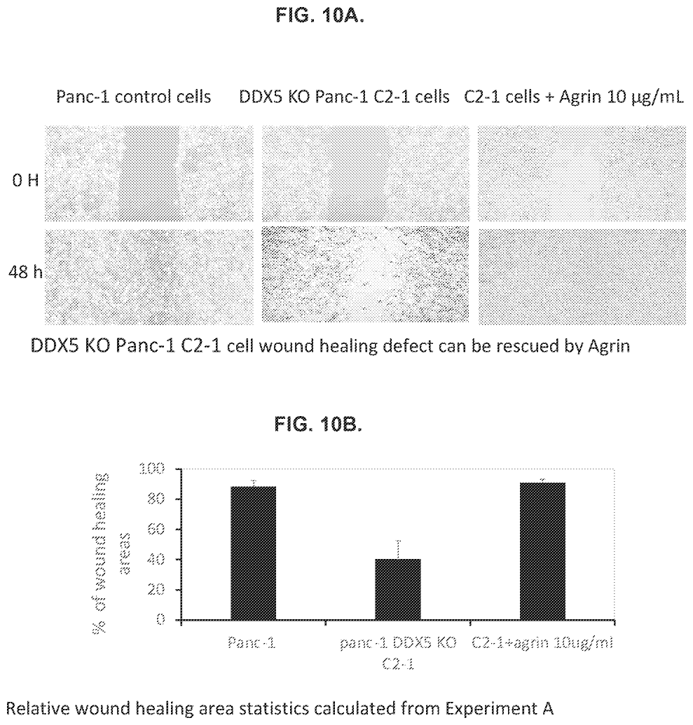

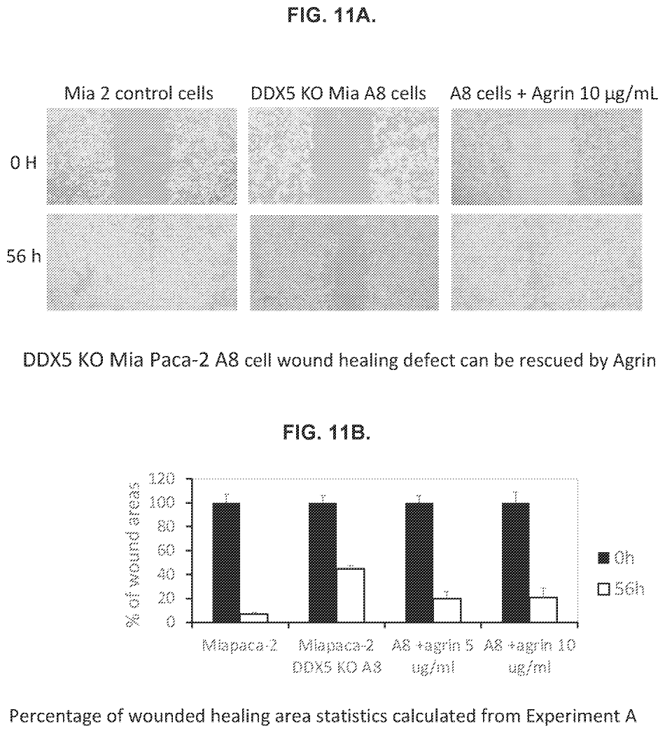

Example 5-Knockout (KO) of DDX5 induces pancreatic cancer cell wound healing defect, which can be rescued by adding exogenous Agrin in the cell culture medium: In the cell wound healing studies, DDX5 KO pancreatic cancer Panc-1 cells exhibited cell migration wound healing defect in comparison with the wild type panc-1 cells; however, the DDX5 KO-induced cell attachment defect could be restored/rescued by adding the exogenous Agrin in the cell culture medium (FIG. 10). The same observed results were also obtained from DDX5 KO pancreatic cancer Mia Paca-2 cells (FIG. 11).

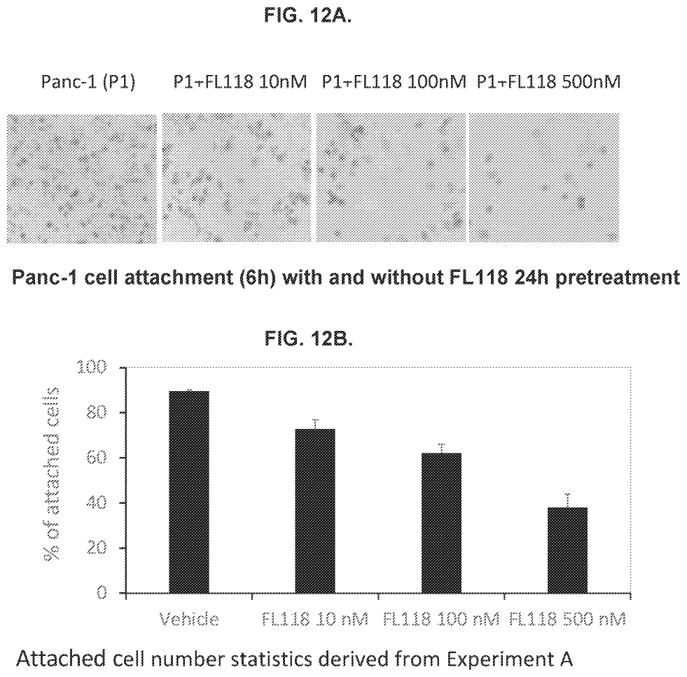

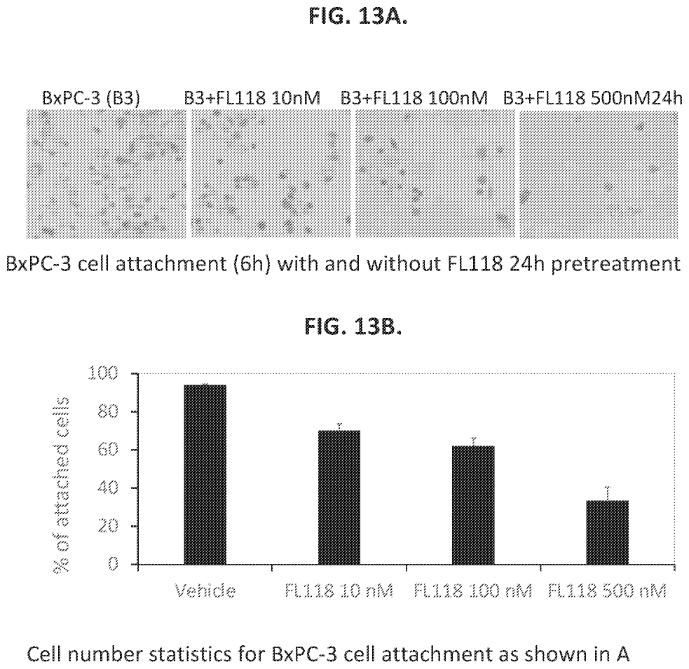

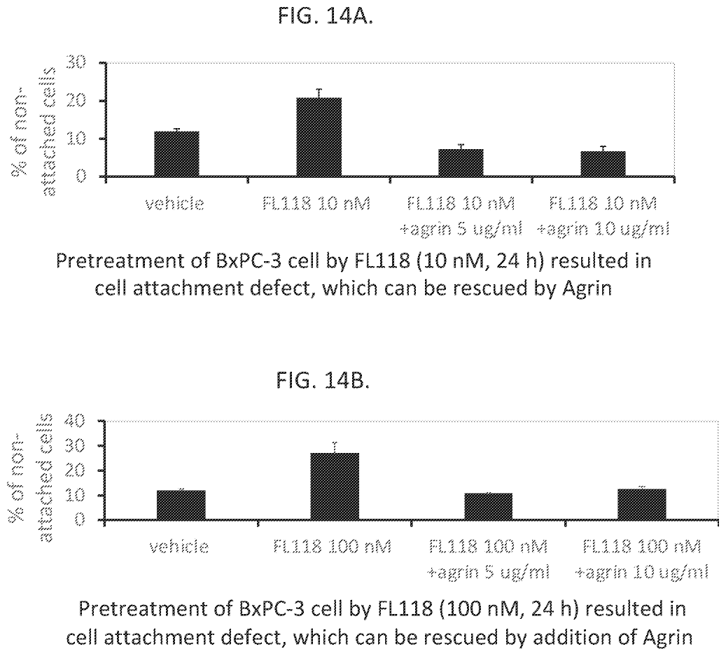

Example 6—FL118 pretreatment of pancreatic cancer cells mimics DDX5 KO and induces cell attachment defect, which can also be rescued by adding exogenous Agrin in the cell culture medium: In the cell attachment studies, wild type pancreatic cancer cells without DDX5 KO but pretreated with and without FL118 for 24 hours were used. Then the alive cells were reseeded for observing cell attachment. Pancreatic cancer Panc-1 cells or BxPC-3 cells pretreated with FL118 exhibited cell attachment defect in comparison with the wild type cells (FIG. 12, 13). However, the cell attachment defect from the FL118-pretreated cells can be rescued by adding the exogenous Agrin in the cell culture medium (FIG. 14).

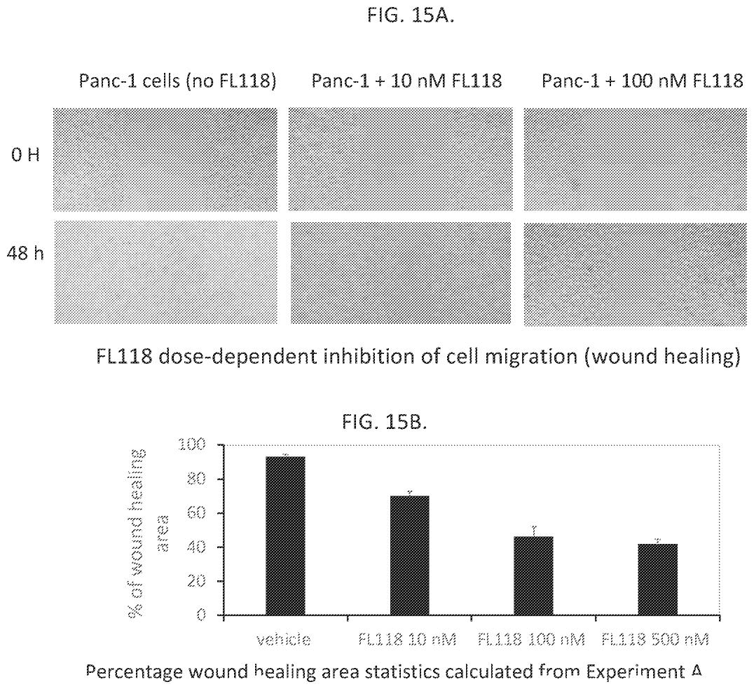

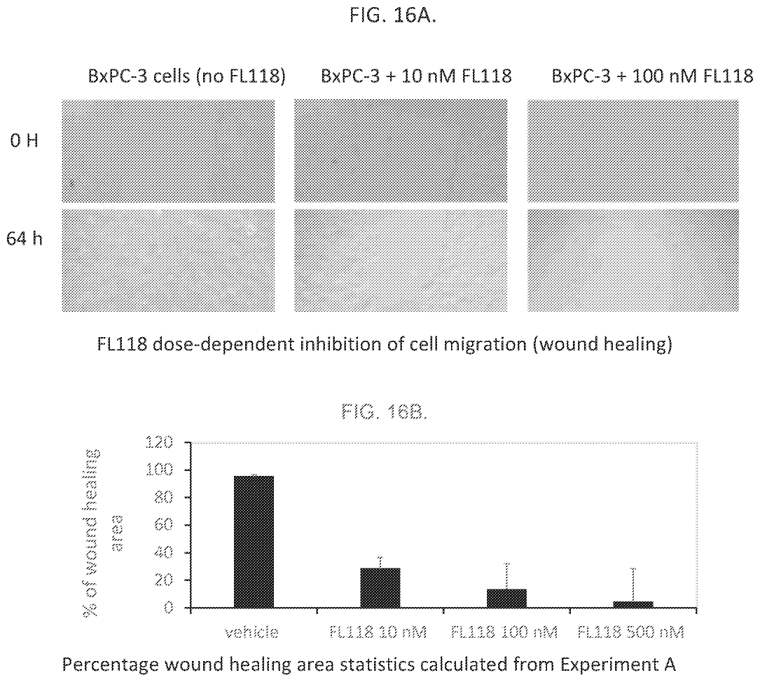

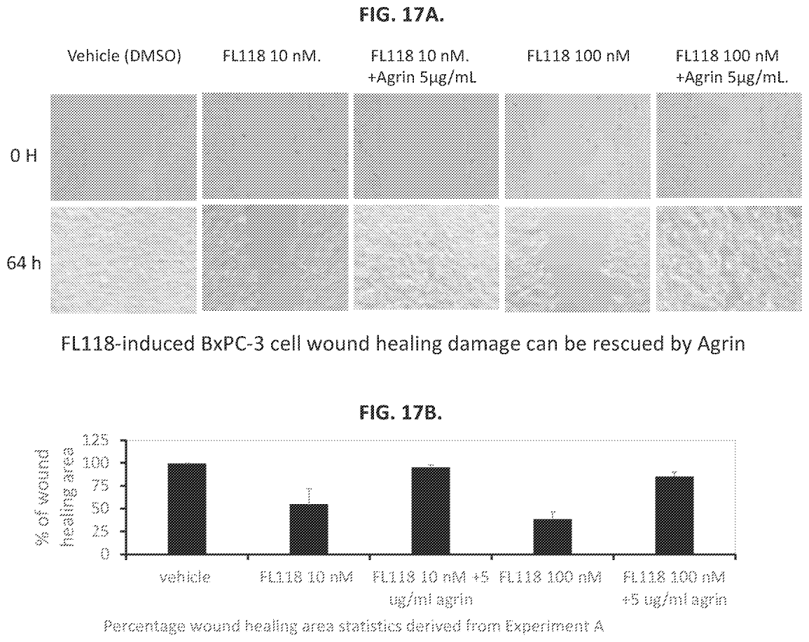

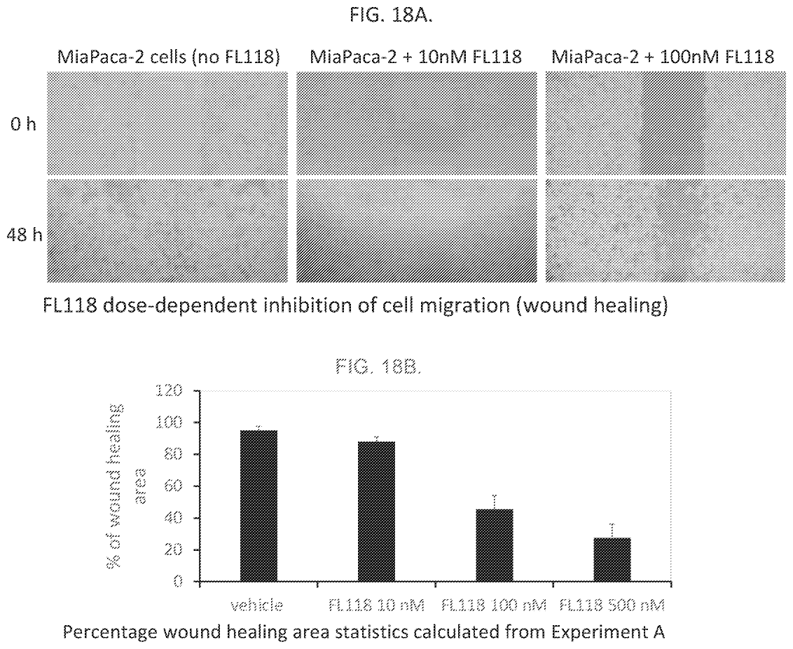

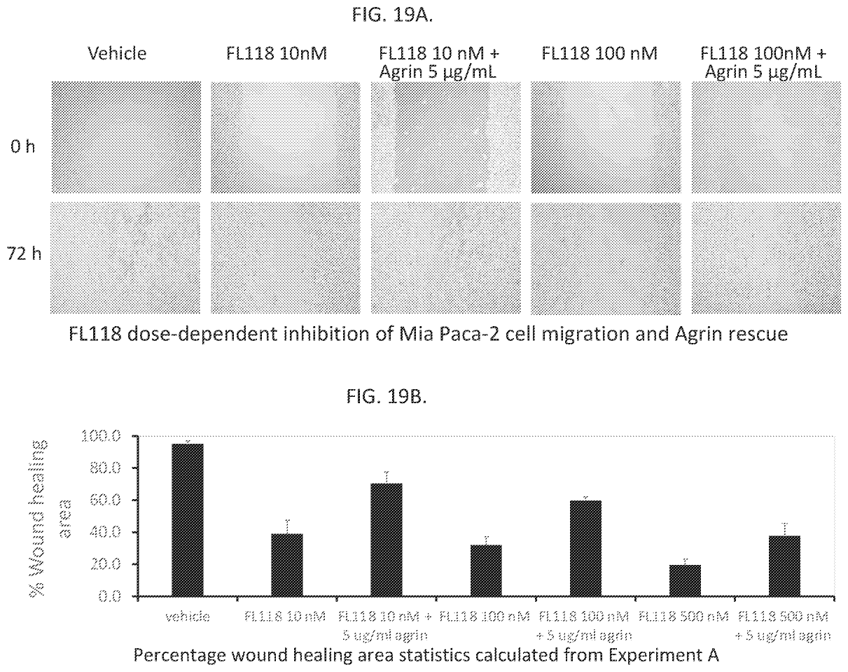

Example 7—FL118 treatment of pancreatic cancer cells mimics DDX5 KO and induces cell wound healing defect, which can be rescued by adding exogenous Agrin in the cell culture medium: In the cell wound healing studies, wild type pancreatic cancer cells without DDX5 KO but with and without FL118 treatment during the cell would healing process were used. In comparison with cells without FL118 treatment, pancreatic cancer cells treated with FL118 exhibited cell migration wound healing defect (FIGS. 15, 16 and 18). However, the FL118 treated cell wound healing defect could be restored/rescued by adding the exogenous Agrin during cell wound healing process in the cell culture medium (FIGS. 17 and 19).

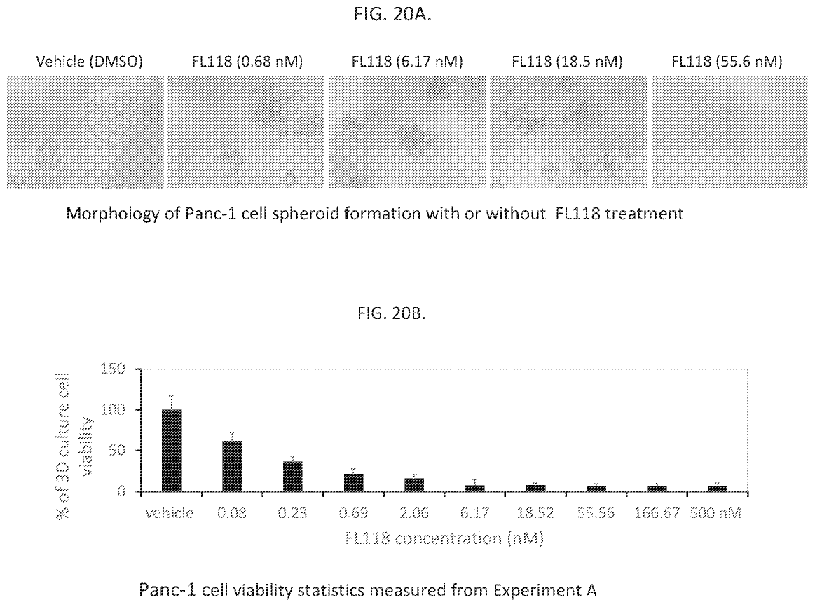

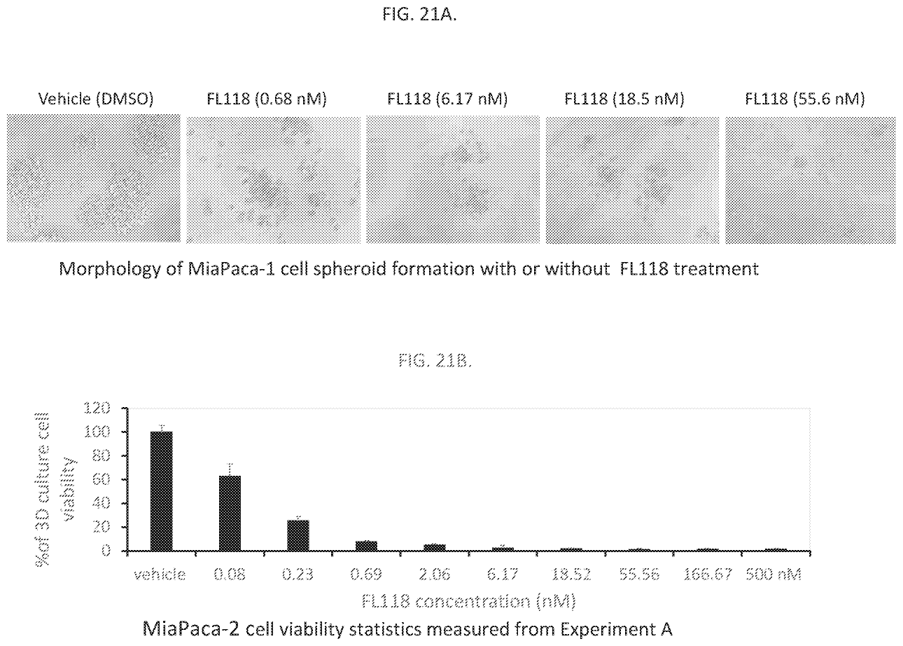

Example 8—FL118 treatment of pancreatic cancer cells induces cell 3D) spheroid formation and cell viability inhibition: When pancreatic cancer cells are seeded on the cell culture-untreated petri dish to block cells attaching to the bottom surface to form cell monolayer. In this way cells will form 3D spheroid. Pancreatic cancer cell spheroid formation is inhibited after FL118 treatment in comparison with cells without FL118 treatment (FIGS. 20A and 21A). Accordingly, the cell viability is inhibited in a FL118 concentration-dependent manner (FIGS. 20B and 21B).

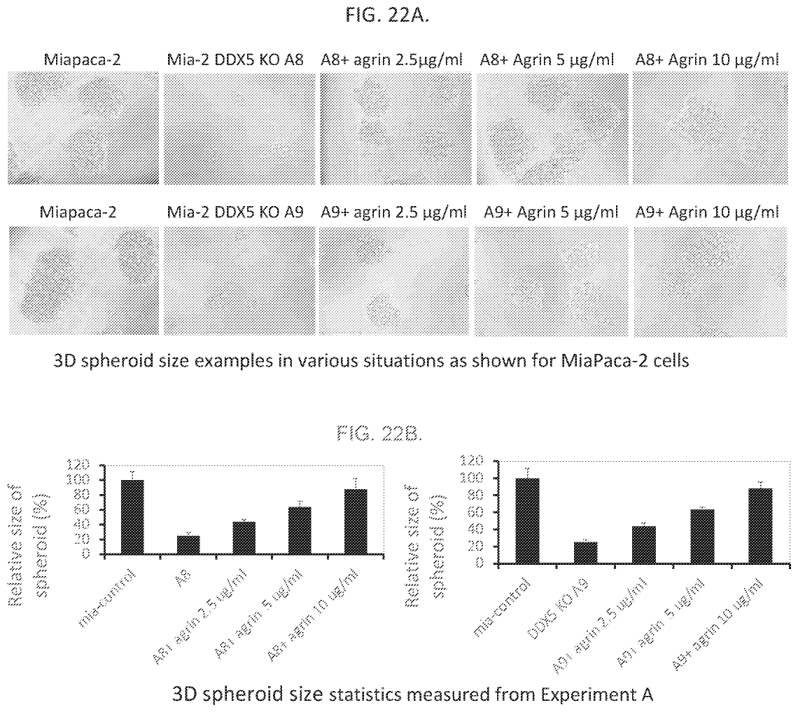

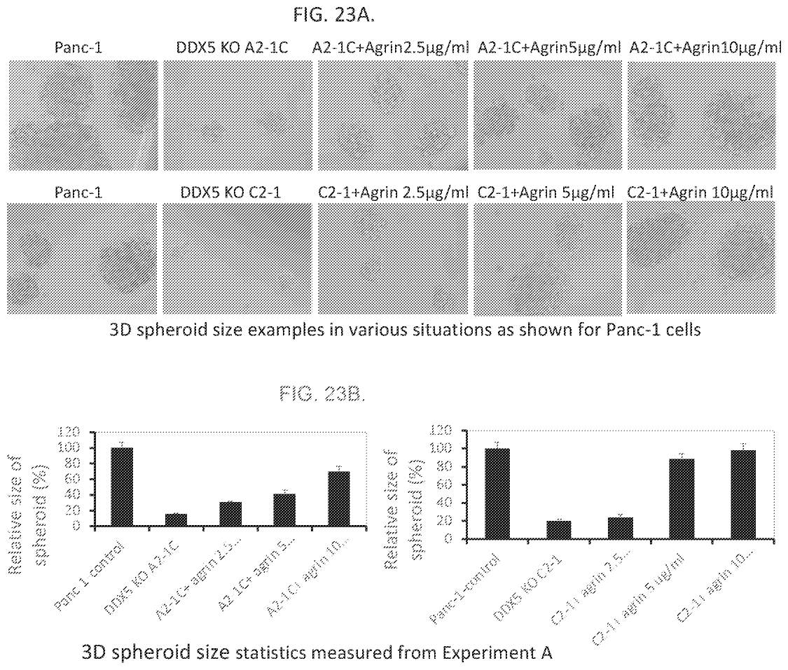

Example 9-Knockout (KO) of DDX5 induces pancreatic cancer cell 3D) spheroid formation defect, which can be rescued by adding exogenous Agrin in the cell culture medium: In the cell 3D spheroid formation studies, DDX5 KO pancreatic cancer Mia Paca-2 cells exhibited cell 3D spheroid formation defect in comparison with the wild type Mia Paca-2 cells; however, the DDX5 KO-induced cell 3D spheroid formation defect could be restored/rescued by adding the exogenous Agrin in the cell culture medium (FIG. 22). The same observed results were also obtained from DDX5 KO pancreatic cancer Panc-1 cells (FIG. 23).

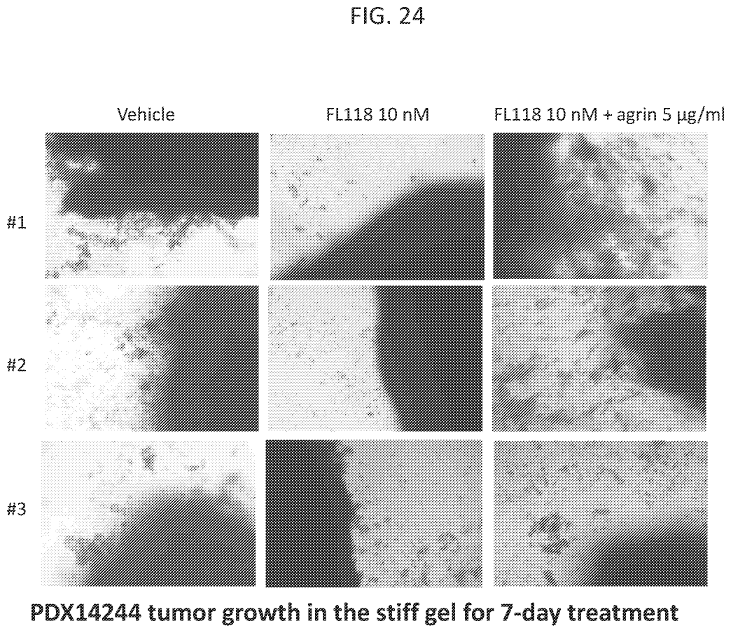

Example 10—FL118 inhibits pancreatic cancer cell and stroma spreading from the ex vivo-cultured pancreatic tumor tissues, which can be restored by adding Agrin in the ex vivo culture medium: By culturing pancreatic cancer PDX tumors (isolated from SCID mice) with and without FL118 treatment for 7 days in the presence or absence of exogenous Agrin, FL118 could effectively inhibit the pancreatic cancer cells and stroma spreading from the tissue edge, which can be restored in the presence of Agrin in the ex vivo culture meditum (FIG. 24)

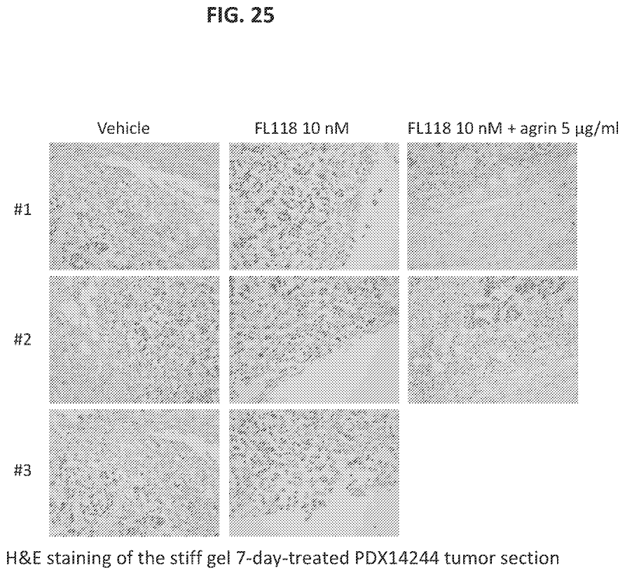

Example 11—The FL118-treated ex vivo pancreatic cancer PDX tumors exhibited significantly loosing of desmoplasia, which can be restored in the presence of exogenous Agrin in the ex vivo culture medium: Next, the PDX tumors ex vivo-cultured with and without FL118 treatment in the presence and absence of Agrin were processed in tissue slide section cutting and H&E staining, FL118 7-day treated tissue slide section exhibited significantly tumor stroma intensity loose in comparison with the non-FL 118-treated tissue slide section; however, the loosing stroma intensity can be restored in the presence of Agrin during ex vivo culturing the PDX tumor tissues (FIG. 25).

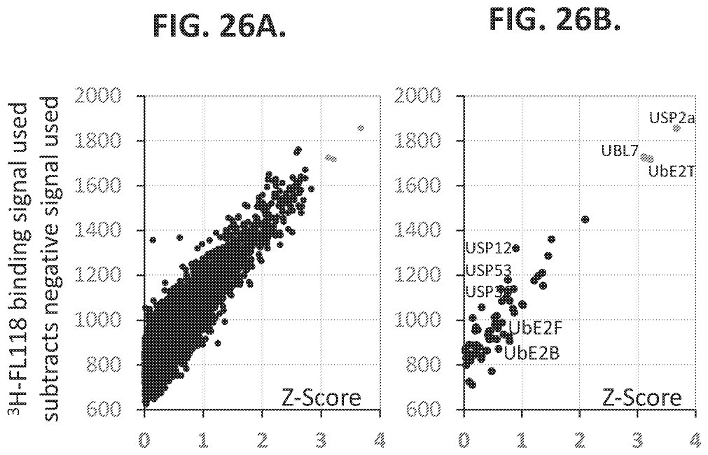

Example 12—FL118 physically binding to USP2a and UbE2T with very high selectivity: Use of the tritium (3H)-labeled FL118 (3H-FL118) to probe protein microarray (ProtoArray) with over 9,400 human proteins discovered that FL118 physically binds to ubiquitin-specific protease 2a (USP2a, an oncogenic deubiquitinase enzyme) and ubiquitin-conjugating enzyme E2T (UbE2T, an oncogenic E2 enzyme) with high Z-score >3 (Table 1, FIG. 26).

TABLE 1
Protein microarray (ProtoArray, Invitrogen) identified
the interaction of FL118 with USP2a and UbE2T using tritium
(³H)-labeled FL118 as probes

| | | | Signal | Neg |
|---|---|---|---|---|
| Database ID | Protein/Assay | Z-Score* | used¹ | signal¹ |
| BC041366.2 | USP2a/FL118 0.5 μM | 3.7 | 1959 | 104 |
| NM_014176.1 | UbE2T/FL118 0.5 μM | 3.2 | 1812 | 94 |

*Z-score is a protein-binding ³H signal subtracted from the mean signal from all the >9,400 human proteins on the ProtoArray (Invitrogen), then divided by the standard deviation (SD) of all proteins. A Z-Score greater than 3.0 indicates a signal greater than 3x SD above the mean signal from all the >9,400 human proteins on the ProtoArray. Negative Z-score is around 1.
¹the number used is ³H counts per min.

In contrast, FL118 did not bind to any of the other 109 ubiquitin-related proteins/enzymes from the >9,400 human proteins on the ProtoArray (FIG. 26B). This includes USP3, USP4, USP5, USP12, USP13, USP15, USP28, USP33, USP36, USP39, USP45, USP47, USP48, USP53, UbE2A, UbE2B, UbE2C, UbE2D1, UbE2D2, UbE2D3, UbE2DNL, UbE2E2, UbE2E3, UbE2F, UbE2G1, UbE2G2, UbE2H, UbE2I, UbE2J2, UbE2L3, UbE2L6, UbE2N, UbE20, UbE2Q1, UbE2R2, UbE2S, UbE2U, UbE2V1, UbE2W, UbE2Z. Alternatively, using a 3K OMEGA Centrifugal Device (PALL, Life Sciences) to separate the free ³H-FL118 from the protein (BSA, USP2a, or UbE2T))-bound ³H-FL118, a 10-20-fold higher binding of ³H-FL118 (³H counts per min) to USP2a and UbE2T were obtained in comparison with the negative control of bovine serum albumin (BSA, Table 2).

TABLE 2
FL118-Protein binding

| Protein on 3K sep | ³H CPM counts in retained parts |
|---|---|
| BSA | 4,230 ± 370 |
| USP2a | 97,436 ± 2960 |
| UbE2T | 52,382 ± 1930 |

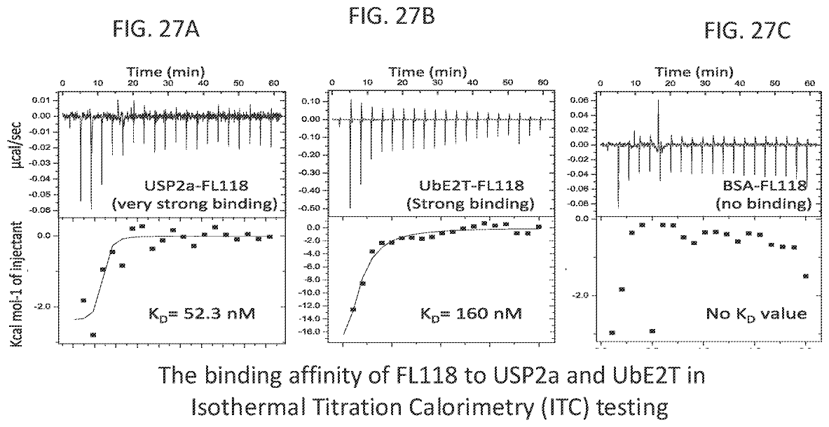

Example 13—FL118 binding to USP2a and UbE2T with high affinity: Next, with the confirmed binding data in hand, the FL118 binding affinity (KD) to USP2a and UbE2T was determined using Isothermal Titration calorimetry (ITC). The ITC results revealed that FL118 exhibits a high binding affinity with USP2a (KD=52.3 nM, FIG. 27A) and UbE2T (KD=160 nM, FIG. 27B) and no binding to BSA (FIG. 27C).

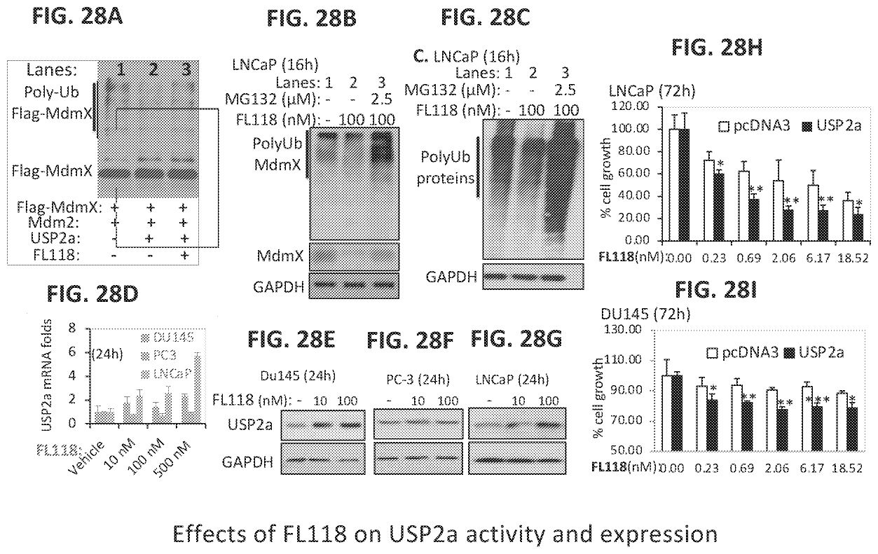

Example 14—FL118 inhibits USP2a deubiquitinase activity without decreasing its mRNA and protein levels: Consistent with the finding that FL118 physically binds to USP2a with high binding affinity (Tables 1 and 2; FIG. 27), FL118 inhibited USP2a deubiquitinase activity in the in vitro reconstitution assay (FIG. 28A). As shown, MdmX is poly-ubiquitinated by Mdm2 (FIG. 28, lane 1), which can be deubiquitinated by USP2a (FIG. 28A, lane 2). However, USP2a deubiquitinase activity is inhibited by FL118 (FIG. 28A, lane 3, polyUb increase). Compared lane 3 with lane 1 (FIG. 28A), FL118 only weakly inhibited USP2a-mediated deUb of MdmX. This may suggest a need for additional cofactor(s) to attain full function. Consistent with this notion, cell-based analysis showed that FL 118 promotes polyUb-mediated MdmX degradation (FIG. 28B, lane 2), and functionality was rescued in the presence of proteasome inhibitor MG132 (FIG. 28B, lane 3). Alternatively, FL118 promotes the overall polyUb-mediated protein degradation induced by FL118-mediated inhibition of USP2a de-ubiquitination (Ub) activity (FIG. 28C, lane 2,), but in the presence of MG132, FL118 enhances total polyUb proteins by abrogating USP2a deUb activity (FIG. 28C, lane 3).

Example 15-USP2a expression increase is not a treatment resistant but sensitive factor for FL118 to exhibit high anti-PCa cell growth efficacy: Intriguingly, FL118-mediated inhibition of USP2a activity is associated with USP2a increases in PCa cells (FIG. 28DEFG). However, the studies demonstrated that USP2a overexpression enhances FL 118-mediated inhibition of PCa cell growth (FIG. 28HI), which suggests that USP2a increases are not an issue but an advantage for FL118 to exhibit higher efficacy, which may in part resulted from FL118 directly inhibiting USP2a deubiquitinase enzyme activity. The results shown in FIG. 28HI suggest that stable overexpression of USP2a in PCa cells makes cells more dependent on USP2a for survival (i.e., PCa cell addiction to USP2a for survival and thus, more sensitive to FL118 treatment).

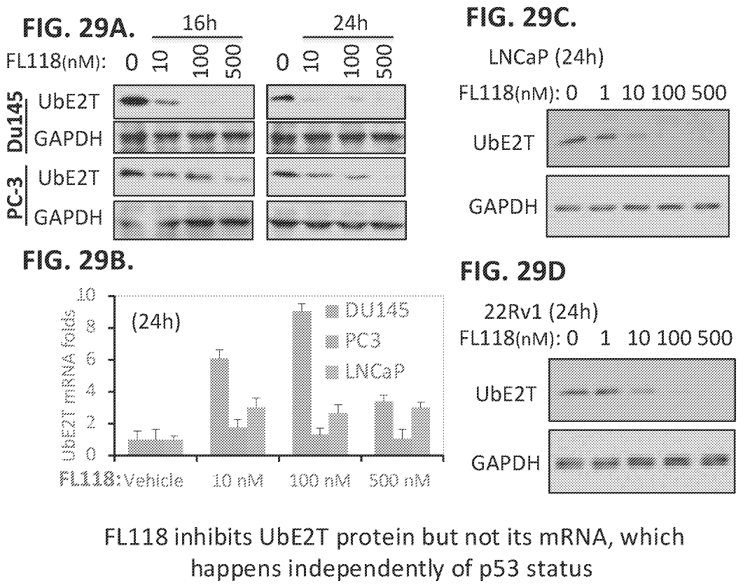

Example 16—FL118 destabilizes UbE2T protein without decreasing its mRNA: Consistent with the discovery that FL118 physically binds to UbE2T with high affinity (Tables 1 and 2; FIG. 27), FL118 inhibits UbE2T protein in a dose- and time-dependent manner (FIG. 29ACD) without inhibiting its mRNA levels (FIG. 29B) in PCa cells with mutant p53 (DU145), null p53 (PC-3), or wild type p53 (LNCaP, 22Rv1). This indicates that FL118 acts as a molecular glue degrader and directly binds to and destabilizes UbE2T in a p53-independent manner.

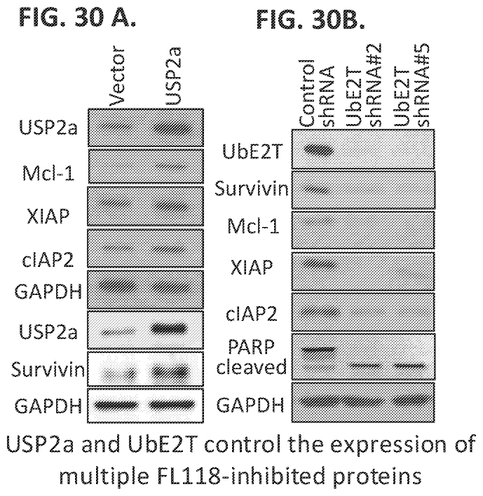

Example 17-Genetic overexpression of USP2a upregulates the expression of survivin, Mcl-1, XIAP, and clAP2: Next, the relationship of USP2a expression with the expression of survivin, Mcl-1, XIAP, and cIAP2 was studied. The studies indicated that forced expression of USP2a in LNCaP cells (relatively low native USP2a expression) upregulates the expression of survivin, Mcl-1, XIAP, and cIAP2 in comparison with the vector-expressed LNCaP cell control (FIG. 30A). This observation indicates that survivin, Mcl-1, XIAP, and cIAP2 are USP2a downstream targets controlled by USP2a.

Example 18-Silencing of UbE2T expression using UbE2T shRNA downregulates the expression of survivin, Mcl-1, XIAP, and clAP2 in parallel with PCa cell apoptosis (PARP cleavage): The relationship of UbE2T expression with the expression of survivin, Mcl-1, XIAP, and cIAP2 was studied. For this study, UbE2T was silenced in DU145 cells (high native UbE2T expression) using UbE2T shRNA and generated control cells using control shRNA. The silencing of UbE2T resulted in the downregulation of survivin, Mcl-1, XIAP, and cIAP2 in parallel with cell apoptosis induction as indicated by PARP (poly (ADP-ribose) polymerase) cleavage (FIG. 30B).

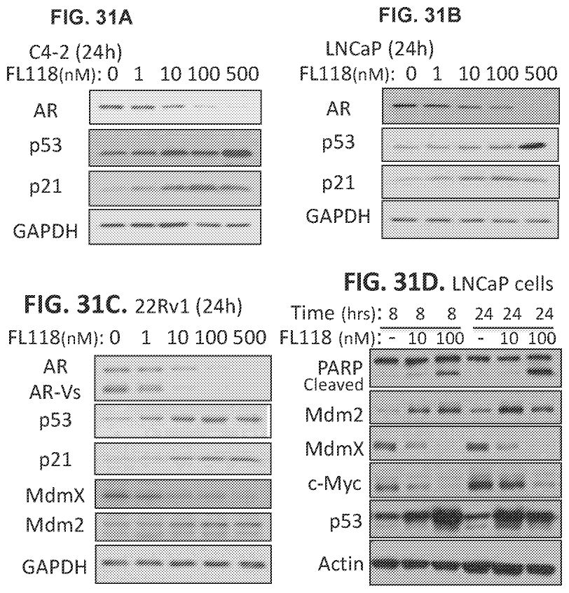

Example 19-Inhibition of USP2a activity (FIG. 28ABC) and UbE2T expression (FIG. 29ACD) by FL118 is associated with the activation of p53 and Mdm2, but inhibition of androgen receptor (AR), AR variants (AR-Vs), c-Myc, and MdmX in parallel with PCa apoptosis: Previous studies demonstrated that USP2a decreases p53 by stabilizing Mdm2 and MdmX/4 (Stevenson, et al. Embo J 2007, 26:976-86; Allende-Vega, et al. Oncogene 2010, 29:432-41). Additionally, Mdm2 ubiquitinates and degrades AR in LNCap46 (Gaughan, et al. Nucleic Acids Res 2005, 33:13-26); USP2a increases c-Myc expression in PCa (Benassi, et al. Cancer Discov 2012, 2:236-47; Nelson, et al. Cancer Discov 2012, 2:206-7); and c-Myc promotes AR gene transcription and enhances AR and AR-Vs protein stability, while the inhibition of c-Myc sensitizes enzalutamide-resistant cells by decreasing AR-Vs expression (Bai, et al. Oncogene 2019, 38:4977-89). Consistent with these previous finding, FL118 induces the expression of p53 (p21 increase is a hallmark of p53 activation) and Mdm2 but inhibits AR, AR-Vs, MdmX, and c-Myc in p53 wild type PCa cells (FIG. 31). It is important to note that the inhibition of both AR and AR-Vs by FL118, as shown in FIG. 31C, is a highly innovative feature of FL118. This addresses a challenge that is unmet by the current AR ligand-binding domain (LBD)-targeted FDA-approved drugs (e.g., enzalutamide) as well as those under development (Ge, et al. Curr Cancer Drug Targets 2018, 18:652-67). Such drugs are unable to degrade AR-Vs because AR-Vs lack the LBD region. Thus, by inhibiting AR and AR-Vs (FIG. 31), FL118 can overcome AR and AR-Vs-associated drug resistance.

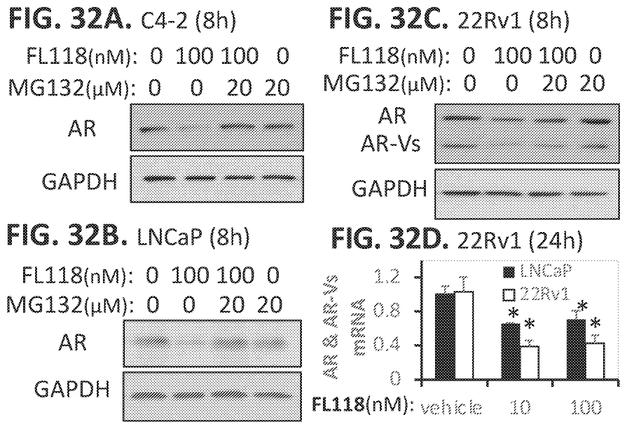

Example 20—FL118-mediated inhibition of AR and AR-V's can be at least partially rescued in the presence of proteasome inhibitor MG132 in wild-type p53 PCa cells: It was demonstrated that Mdm2 ubiquitinates AR and induces AR degradation in PCa cells (Gaughan, et al. Nucleic Acids Res 2005, 33:13-26); and other studies indicated that AR Ub and degradation can also occur through the Akt-Mdm2 E3 ligase-AR pathway in PCa cells88. 89 (Lin, et al. EMBO J 2002, 21:4037-48; Deep, et al. Oncogene 2008, 27:3986-98). The inhibition of AR (and AR-Vs, if any) by FL118 in prostate cancer C4-2, LNCaP, and 22Rv1 cells, as shown in FIG. 31, can be at least partially restored/rescued in the presence of MG132 (FIG. 32ABC). Real-time RT-PCR analyses of AR and AR-Vs mRNA expression using primers from the AR DNA binding domain (DBD) region indicated that FL 118 can also decrease AR mRNA levels in LNCaP and 22Rv1 cells (FIG. 32D), thus suggesting that AR inhibition by FL118 uses both the Ub-mediated proteasome degradation pathway and the transcriptional regulation pathway at differing degrees depending on cell types. As shown in the recent publication (Ling, et al. Clin Transll Med 2022, 12: e881), this likely involves DDX5's function as a transcription co-activator for AR and AR-Vs transcriptional activation, and this can be inhibited by FL118.

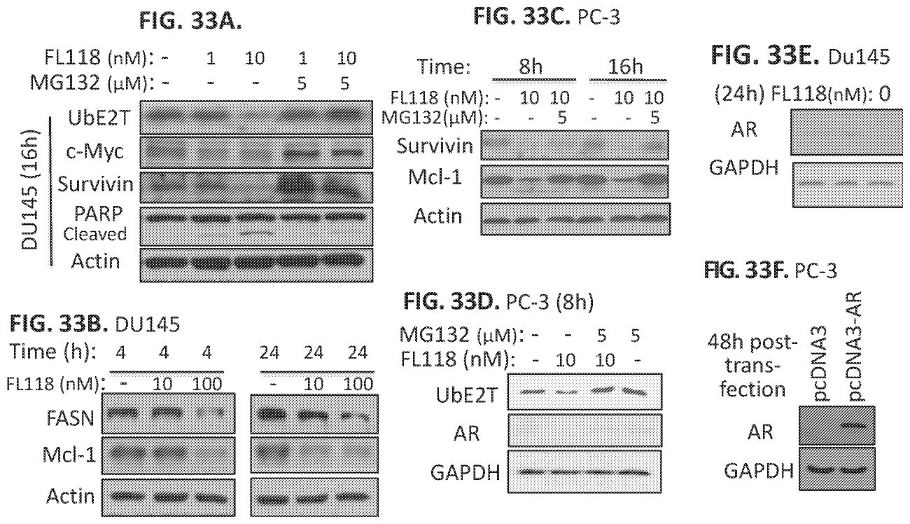

Example 21-Inhibition of UbE2T, survivin, Mcl-1, c-Myc, FASN, and AR by FL118 in p53 mutant null PCa cells can also be at least partially restored rescued by MG132: Mechanistically, the studies using DU145 and/or PC-3 PCa cells revealed that the FL118-mediated inhibition of UbE2T, c-Myc, survivin, Mcl-1, and AR can be reversed by the ubiquitin-proteasome inhibitor MG132 (FIG. 33). This suggests the involvement of the ubiquitination (Ub)-mediated proteasome degradation pathway. Furthermore, consistent with the previously reported finding that DU145 and PC-3 cells express ARs (Alimirah, et al. FEBS Lett 2006, 580:2294-300), PC-3 and DU145 express low-levels of ARs, which can be inhibited by FL118 (FIG. 33DE). Therefore, inhibition of ARs by FL118 can be realized in PCa cells with wild type (FIG. 31ABC), null (FIG. 33D), and mutant p53 (FIG. 33E).

The extensive studies presented above (Tables 1, 2; FIGS. 26-33) have revealed that through the inhibition of USP2a deubiquitinase activity and UbE2T expression by FL118, FL118 can indirectly control a panel of USP2a and UbE2T downstream protein targets including survivin, Mcl-1, c-Myc, FASN, AR, AR-Vs (if existing), Mdm2, and MdmX in p53-mutant,-null, and-wild-type PCa cells. This strongly suggests that USP2a and UbE2T are master regulators and thus, targeting USP2a and UbE2T by FL118 should be effective for achieving high anti-PCa tumor efficacy against CRPC and metastatic CRPC (mCRPC) lethal PCa. This expectation appears to be the case (see the example blow).

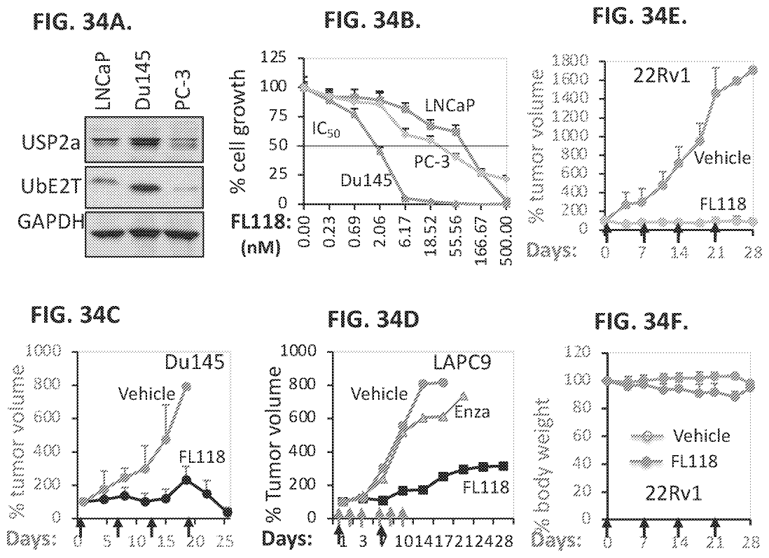

Example 22—FL118 possesses high efficacy against human (RPC and mCRPC tumor in animal models: Consistent with FL118's unique ability to control gene expression through the master regulators, USP2a and UbE2T, among the 3 most used PCa cell lines (LNCaP, DU145, PC-3), DU145 highly expresses both USP2a and UbE2T (FIG. 34A), and accordingly DU145 cells exhibited higher sensitivity to FL118-induced cell growth inhibition than the other two PCa cells (FIG. 34B). Consistently, DU145 cells can rapidly form xenograft tumors in SCID mice without the need to use Matrigel, and the DU145 tumor can be well inhibited by a single cycle of FL118 treatment (arrowed, FIG. 34C). Next, using Dr. Dean Tang Lab-established LAPC9 CRPC tumor model (Li, et al. Nat Commun 2018, 9:3600), the antitumor efficacy of FL118 was compared with enzalutamide (Enza). These studies indicated that such CRPC tumors are sensitive to FL118 but not sensitive to Enza, even with only a 2-time oral administration of FL118 (black arrows) versus 6-time intraperitoneal administration of Enza (green arrows, FIG. 34D). Additionally, the 22Rv1 CRPC tumor, which exhibited both AR and AR-Vs expression, is also sensitive to FL118 at the above-indicated dose, which completely inhibited 22Rv1 tumor growth (FIG. 34E). Importantly, FL118 in the used therapeutic dose exhibited no toxicity, as judged by a panel of clinical observations including mouse behavior, movement, fur shining status, diarrhea, and body weight. The mouse body weight change (a critical toxicity parameter) is presented in FIG. 34F. Consistent with all of the in vitro and in vivo observations presented above, FL118 exhibits selective toxicity against the AR^{-/lo} LAPC9-androgen-independent (AI) vs. AR⁺ LAPC9-androgen-dependent (AD) cells in organoid assay studies.

The study examples presented above have addressed (1) the mechanistic versatile novelty of FL118 in regard to its capacity to bind to and inhibit the master oncogenic regulators (USP2a and UbE2T), and thus, indirectly control a panel of US2a/UbE2T downstream protein targets involved in cancer initiation, development, metastasis, relapse, and drug resistance. These USP2a/UbE2T downstream targets include, but may not be limited to, AR, AR-Vs, survivin, c-Myc, Mcl-1, XIAP, fatty acid synthase (FASN), Mdm2, and MdmX; and (2) the potential high anti-PCa efficacy of FL118 in lethal PCa. Some targets such as FASN, appear to play an important role as a good target in cancer treatment (Sardesai, et al. Clin Cancer Res 2021, 27:5810-7; Wang, et al. Cancer Lett 2021, 509:1-12), although it may not be well known by others. However, one potential concern as to whether the finding of USP2a and UbE2T acting as oncogenic master regulators for FL118 to target will have clinical relevance in lethal PCa (CRPC, mCRPC). This concern is reasonable and important and was well addressed in clinically relevant studies using the PCa patient-derived tumor specimen data. An example is presented below.

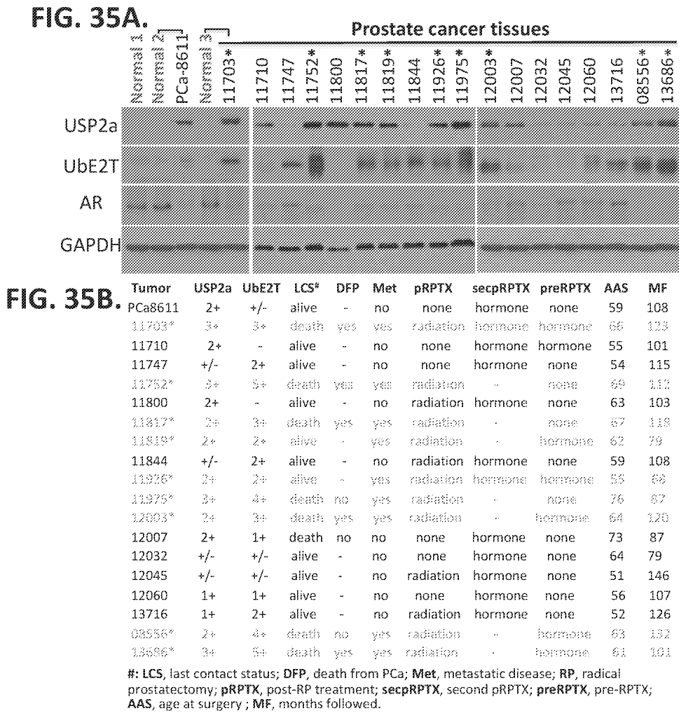

Example 23-Overexpression of USP2a and UbE2T is associated with advanced lethal PCa: Consistent with the high anti-PCa efficacy of FL118 in the in vivo human CRPC tumor models (FIG. 34), the idea to use USP2 and UbE2T as therapeutic targets for lethal CRPC and mCRPC treatment is clinically supported in the literature as well as the studies using clinical PCa tumor specimens. USP2a is overexpressed in ˜50% of PCa but negative in prostate epithelial cells (Graner, et al. Cancer cell 2004, 5:253-61; Priolo, et al. Cancer research 2006, 66:8625-32); and UbE2T expression is elevated in PCa, especially those with distant metastasis (Wen, et al. Oncotarget 2015, 6:25226-39). Consistently, data derived from the clinically normal prostate tissues and PCa tissues indicated that most PCa tissues express either USP2a and/or UbE2T proteins, while normal prostate tissues show no USP2a and UbE2T expression (FIG. 35A). Significantly, the 9 PCa tissues collected from metastatic sites or primary sites with metastatic disease highly expressed both USP2a and UbE2T (FIG. 35A, marked with “*”). Using a Wilcoxon-Mann-Whitney test, the clinical information from 19 PCa tumor tissue-related patients (FIG. 35B) showed expression of USP2a (P=0.003) and UbE2T (P=0.0006) in metastatic PCa tissues was significantly higher than that in non-metastatic PCa tissues (FIG. 35B), thus suggesting that USP2a and UbE2T are potential prognostic markers and therapeutic targets for treating CRPC and mCRPC. The Human Protein Atlas data further support this idea, and showed that PCa tissues express a moderate to high level of USP2 (no UbE2T protein data were available), which is further supported by a publication from Dr. Dean Tang's Lab, which revealed that there is a 2.3-fold USP2 increase in CRPC tumors, as presented in their “supplementary data 5” in the publication (Li, et al. Nat Commun 2018, 9:3600). Considering the extensive Studies presented early, FL118 appears to be a novel drug candidate for treating CRPC and mCRPC lethal PCa.

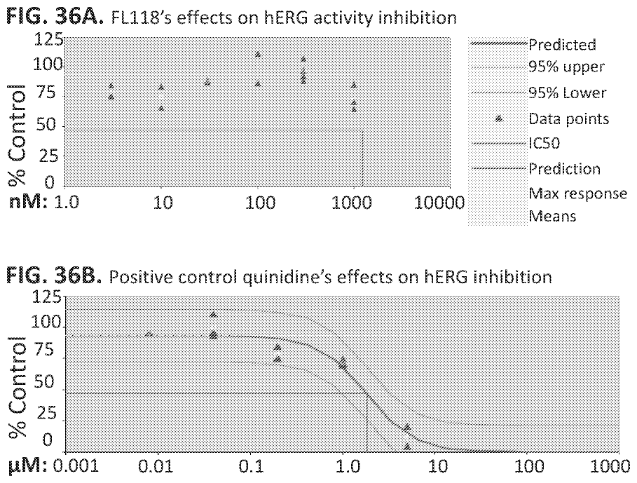

Example 24—FL118 exhibited no potential cardiac toxicity: The hERG potassium channel is a voltage-gated ion channel found in the heart. It is essential for cardiac re-polarization, yet many pharmacological agents can inhibit the hERG current and give rise to potentially fatal cardiac toxicity in animals and humans. To determine the potential cardiac toxicity of FL118, Applicants performed a “hERG Test” by partnering with an FDA-certified CRO (Cyprotex US LLC). The “hERG Test” studies indicated that FL118 exhibited no inhibitory effects on hERG activity over the FL118 concentration range tested (3-1000 nM) (FIG. 36A, of note, 1000 nM of FL118 is the highest concentration that can be reached without FL118 precipitation using in vitro testing), while the positive control cardiotoxic drug, quinidine, showed high inhibitory effects on hERG activity (FIG. 36B).

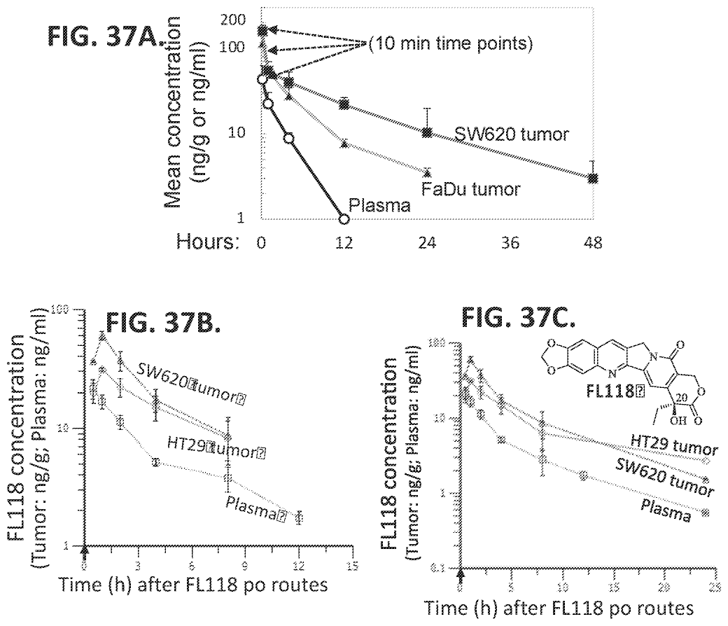

Example 25—FL118 possesses highly favorable pharmacokinetics (PK) profiles: Either intravenous (i.v.) (FIG. 37A) or oral (p.o.) (FIG. 37BC) administration of FL118 resulted in the accumulation of FL118 in human tumors, while FL118 is rapidly cleared in blood stream (plasma) (FIG. 37). Consistently, either i.v. (Table 3) or p.o. (Table 4) administration of FL118 exhibited favorable PK parameters. For example, the AUC is higher in human tumor than in mouse plasma (Tables 3, 4). As shown in Table 3 (i.v. route with a single dose at 1.5 mg/kg), ratios of AUC, for FaDu tumor vs AUC, for plasma and AUC, for W620 tumors vs AUC, for plasma are 4.31 and 8.63, respectively, indicative of a multifold increased accumulation of FL118 in tumors over plasma.

TABLE 3
Pharmacokinetics (PK) parameters of FL118 in human tumor and mouse plasma
following single-dose intravenous (i.v.) administration of 1.5 mg/kg FL118.

| | T½ | Tmax | Cmax | AUC | AUCo | AUC % | Vz (g/kg) | Cl (g/hr/kg) |
|---|---|---|---|---|---|---|---|---|
| Matrix | (hr) | (hr) | (ng/g, ml) | (hr*ng/g) | (hr*ng/g) | Extrap (%) | (ml/kg) | (ml/hr/kg) |
| FaDu | 6.852 | 0.167 | 115 | 413 | 448 | 7.74 | 33052 | 3343 |
| SW620 | 12.75 | 0.167 | 158 | 842 | 897 | 6.17 | 30742 | 1671 |
| Plasma | 1.788 | 0.167 | 43 | 82 | 104 | 21.7 | 36849 | 14287 |

| | |
|---|---|
| Ratio of AUC_{∞} for FaDu/AUC_{∞} for Plasma | 448/104 = 4.31 |
| Ratio of AUC_{∞} for SW620/AUC_{∞} for Plasma | 897/104 = 8.63 |

TABLE 4
Pharmacokinetics (PK) parameters of FL118 in human tumor and mouse plasma
following single-dose oral (p.o.) administration of 1.5 mg/kg FL118.

| Matrix | Tₘₐₓ | t_{1/2} | Cₘₐₓ | SE Cₘₐₓ | AUCₐₗₗ | AUC_{∞} | AUC 0-8 | V_{D} | Cl |
|---|---|---|---|---|---|---|---|---|---|
| | (hr) | (hr) | (ng/ml) | (ng/ml) | (hr*ng/ml) | (hr*ng/ml) | (hr*ng/ml) | (mL/kg) | (mL/hr/kg) |
| Plasma | 0.50 | 5.12 | 21.4 | 3.88 | 74.1 | 88.0 | 63.0 | 126,000 | 17,000 |
| | Tₘₐₓ | t_{1/2} | Cₘₐₓ | SE Cₘₐₓ | AUCₐₗₗ | AUC_{∞} | AUC 0-8 | V_{D} | Cl |
| Matrix | (hr) | (hr) | (ng/g) | (ng/g) | (hr*ng/g) | (hr*ng/g) | (hr*ng/g) | (g/kg) | (g/hr/kg) |
| HT29 | 1.00 | 4.24 | 31.2 | 0.822 | 128 | 178 | 128 | 51,700 | 8,440 |
| Tumor | | | | | | | | | |
| SW620 | 1.00 | 2.99 | 60.5 | 4.74 | 188 | 224 | 188 | 28,900 | 6,710 |
| Tumor | | | | | | | | | |

| | |
|---|---|
| Ratio of AUC_{∞} for HT29/AUC_{∞} for Plasma | 178/88 = 2.02 |
| Ratio of AUC_{∞} for SW620/AUC_{∞} for Plasma | 224/88 = 2.55 |
| Oral Bioavailability for FL118 (p.o. or i.v.) | 88/104 = 84.6% |

It is also true for the p.o. route, as shown in Table 4. Furthermore, the oral bioavailability of FL118 can be calculated to be >84% (Table 4), which is impressive. Due to the great oral bioavailability, the p.o. route has been chosen for human clinical trials, which is an enormous advantage over other therapeutic agents for treating pancreatic cancer.

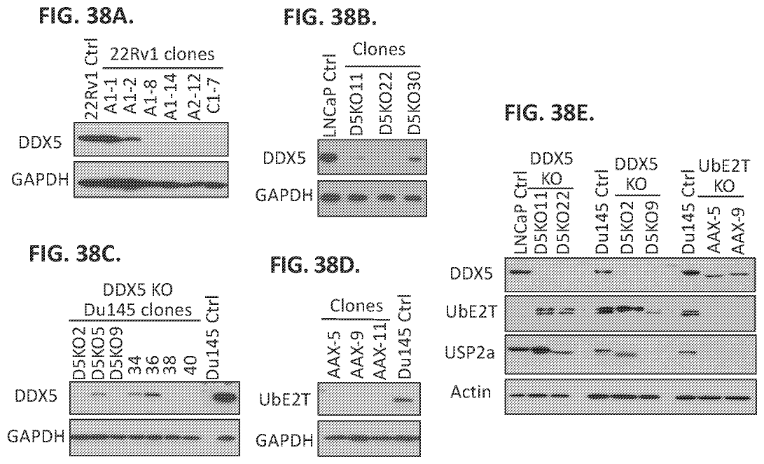

Example 26-Knockout (KO) of DDX5 or KO of UbE2T in prostate cancer cells demonstrated that DDX5, USP2a and UbE2T could be mutually regulated and possibly existed in the same protein complex, at least during certain functional situation: Next, vector-free Crispr-Cas9 technology was used to knock out DDX5, USP2 and UbE2T in prostate cancer cells. Interestingly, although USP2 KO failed in any prostate cancer cell lines, DDX5 KO was possible in multiple prostate cancer cell lines (22Rv1, LNCaP, Du145; FIG. 38ABC) and UbE2T KO in Du145 (FIG. 38D). One possibility for failure to obtain USP2 KO cells is that the alive cells with USP2 KO are too rare to be found by using limited cell dilution, suggesting the importance of USP2a in cell survival. Nonetheless, to find out the expression relationship of the three FL118 targets (DDX5, USP2a and UbE2T), the expression of USP2a and UbE2T in the DDX5 KO cells as well as the expression of DDX5 and USP2a in the UbE2T KO cells was studied. DDX5 KO LNCaP cells increased the expression of UbE2T, while increasing or decreasing the expression of USP2a (FIG. 38E, left panel). In contrast, DDX5 KO DU145 cells decreased the expression of UbE2T and USP2a (FIG. 38E, meddle panel). Intriguingly, UbE2T KO DU145 cells showed the significant decrease DDX5 expression, while abrogating the expression of USP2a (FIG. 38E, right panel).

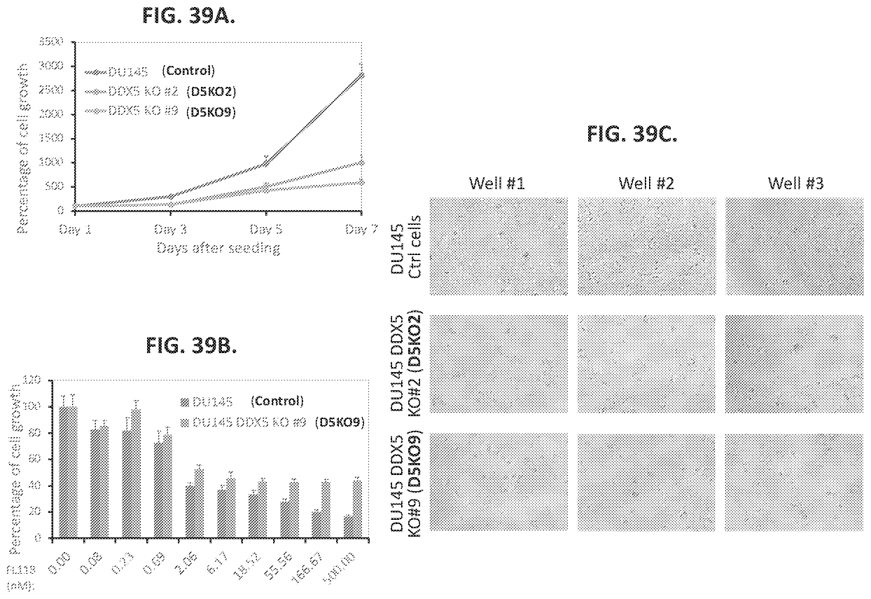

Example 27-KO of DDX5 in PCa DU145 cells delays cell growth, increases cell resistance to FL118-induced cell growth inhibition and change cell morphology: To determine whether PCa cells without DDX5 can cause cell growth slowing down, parental control DU145 cells and DDX5 KO DU145 cells (two cell clones: D5KO2 and D5KO9) were growing in complete cell culture medium for 7 days and cell growth was determined using MTT assay on days 1, 3, 5 and 7. The results indicated that DDX5 KO DU145 cells significantly delay cell growth (FIG. 39A). Furthermore, DDX5 KO DU145 cells exhibited less sensitive to FL118-mediated cell growth inhibition in comparison with the parental control DU145 cells. DDX5 KO DU145 cells in parallel with the control parental DU145 cells were treated with a series of FL 118 concentrations for 72h; cell growth/viability inhibition by FL118 was then determined using MTT assay. The results indicated that DDX5 KO DU145 cells significantly increased resistance to FL118 treatment (FIG. 39B). Furthermore, DDX5 KO DU145 cells exhibited cell morphology changes in comparison with the parental control DU145 cells (FIG. 39C).

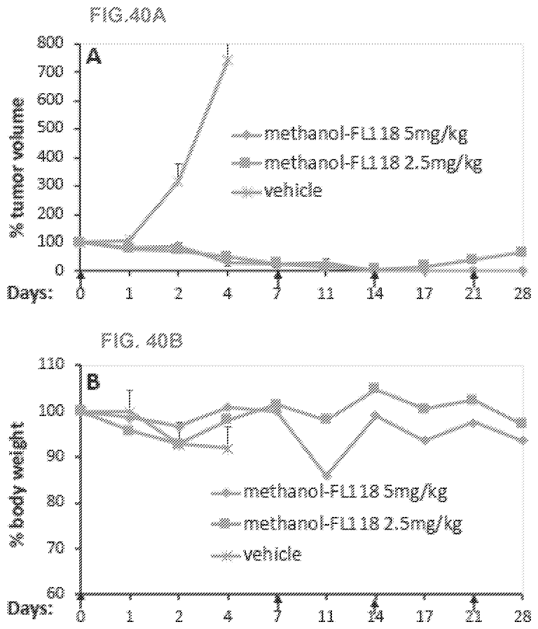

Example 28—The scope of the identified solvents that are used to dissolve a type of CDs (e.g. HPβCD): In this disclosure, three different categories of organic solvents are identified: (1) methanol or ethanol; (2) formic acid (FA), glacial acetic acidacetic acid (GAA), zinc acetate (ZA) or glyoxal (ethanedial) and (3) ethylene glycol (EG), propylene glycol (PG), formamide (FAD), (N,N,N′,N′)-tetramethyl-ethylenediamine (TEMED), ethanolamid (EA) or 2-mercaptoethanol (MercE) alone or in different ratios of combination of the defined certain two or three solvents are used to dissolve a type of CDs (e.g. HPβCD) for FL118 formulation. However, most of the FDA listed organic solvents including the following organic solvents alone or in any ratio of combination of two, three or more are not good for dissolving any types of cyclodextrins (CDs) including HPβCD for FL118 formulation. These non-suitable solvents include, but are not be limited to, methyl formate, ethyl formate, isobutyl acetate, methyl acetate, ethyl acetate, butyl acetate, acetic anhydride, acetone, anisole, acetonitrile, benzene, 1-butanol, 2-butanol, tert-butyl methyl ether, carbon tetrachloride, benzyl chloride, benzyl benzoate, chlorobenzene, chloroform, cumene, cyclohexane, 1,2-dibromoethene, 1,1-dichloroethene, 1,2-dichloroethene, dichloromethane, 1, 2-dimethoxyethane 1,4-dioxane, 2-ethoxyethanol, 1,2-dimethoxyethanol, diethyl ether, heptane, hexane, isoamyl alcohol, amyl alcohol, 2-hexanone, cyclohexylmethane, methylethyl ketone, methylisobutyl ketone, 2-methyl-1-propanol, 2-Methyl-2-propanol, pentane, propyl alcohol, isopropyl alcohol, propyl acetate, toluene, xylene, benzaldehyde, benzaldehyde dimethyl acetal, tetralin, (1,1,2)-trichloroethene, tetrahydrofuran, pyridine, sulfolane, glycerine, diethyl pyrocarbonate, and dimethyl sulfate. Examples from each unique working organic solvents are presented below through Example 29 to Example 40 through testing demonstration of antitumor activity and toxicity profile with each of the data presented in from FIG. 40 to FIG. 51 below.

Example 29-Methanol-HP BCD-formulated FL118 antitumor activity and toxicity (body weight changes) profiles: The antitumor activity and toxicity of FL118 formulated through the methanol-HPβCD muster solution were tested in the human APC/p53/Kras-triple-mutated SW620 colorectal cancer xenograft tumor animal models. The data is shown in FIG. 40 (tumor curves in A and body weight change in B). As shown, while vehicle-treated mice with tumors grew to the maximal tumor size allowed and the tumor mice had to be euthanized on day 4, FL118-treated mice exhibited tumor regression with potential tumor elimination, although FL118 dosing level should be controlled based on the animal body weight changes.

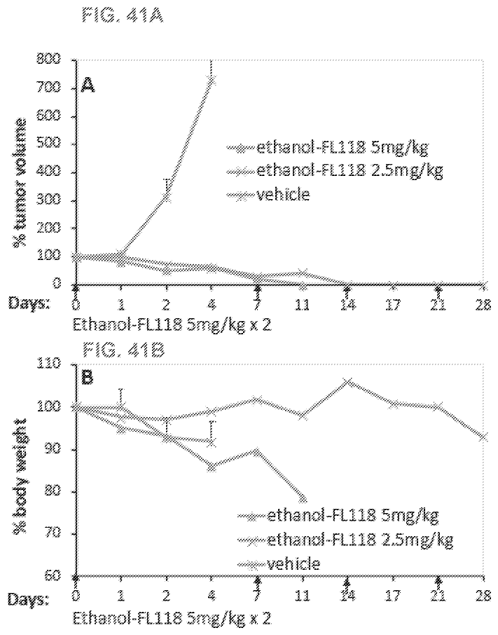

Example 30-Ethanol-HP BCD-formulated FL118 antitumor activity and toxicity (body weight changes) profiles: The antitumor activity and toxicity of FL118 formulated through the ethanol-HPβCD muster solution were tested in the human APC/p53/Kras-triple-mutated SW620 colorectal cancer xenograft tumor animal models. The data is shown in FIG. 41 (tumor curves in A and body weight change in B). As shown, while vehicle-treated mice with tumors grew to the maximal tumor size allowed and the tumor mice had to be euthanized on day 4, FL118-treated mice exhibited tumor regression with tumor elimination. However, FL118 dosing level should be controlled based on the animal body weight changes.

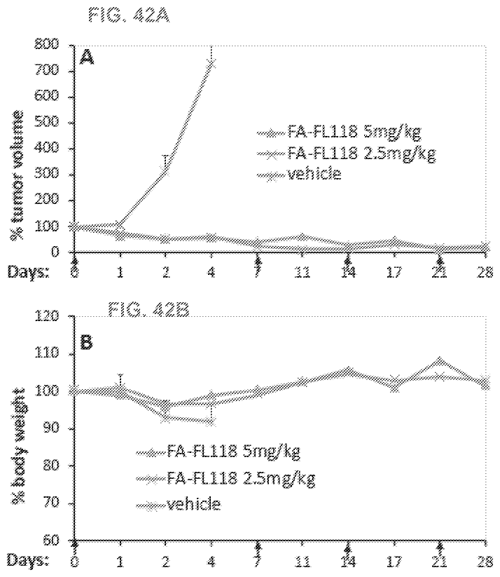

Example 31-Formic acid (FA)-HPβCD-formulated FL118 antitumor activity and toxicity (body weight changes) profiles: The antitumor activity and toxicity of FL118 formulated through the FA-HPβCD muster solution were tested in the human SW620 colorectal cancer xenograft tumor animal models. The data is shown in FIG. 42 (tumor curves in A and body weight change in B). As shown, while vehicle-treated mice with tumors grew to the maximal tumor size allowed and the tumor mice had to be euthanized on day 4, FL118-treated mice exhibited tumor regression with potential tumor elimination. Importantly, at the FL118 dosing levels used, there is no body weight loss. Therefore, high dosing levels could be used when required.

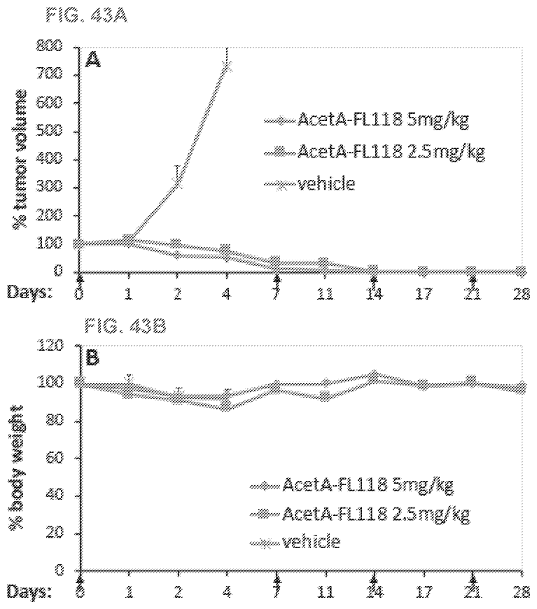

Example 32-Glacial Acetic acid HPβCD-formulated FL118 antitumor activity and toxicity (body weight changes) profiles: The antitumor activity and toxicity of FL118 formulated through the glacial acetic acid-HPβCD muster solution were tested in the human SW620 colorectal cancer xenograft tumor animal models. The data is shown in FIG. 43 (tumor curves in A and body weight change in B). As shown, while vehicle-treated mice with tumors grew to the maximal tumor size allowed and the tumor mice had to be euthanized on day 4, FL118-treated mice exhibited tumor regression with tumor elimination, Importantly, at the FL118 dosing levels used, there is no body weight loss. Therefore, high dosing levels could be used when required.

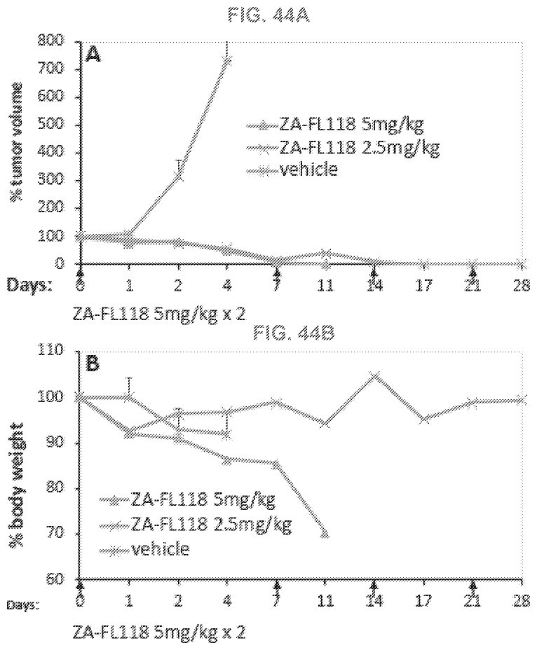

Example 33-Zinc acetate (ZA)-HP BCD-formulated FL118 antitumor activity and toxicity (body weight changes) profiles: The antitumor activity and toxicity of FL118 formulated through the ZA-HPβCD muster solution were tested in the human SW620 colorectal cancer xenograft tumor animal models. The data is shown in FIG. 44 (tumor curves in A and body weight change in B). As shown, while vehicle-treated mice with tumors grew to the maximal tumor size allowed and the tumor mice had to be euthanized on day 4, FL118-treated mice exhibited tumor regression with tumor elimination. However, FL118 dosing levels should be controlled based on the animal body weight changes.

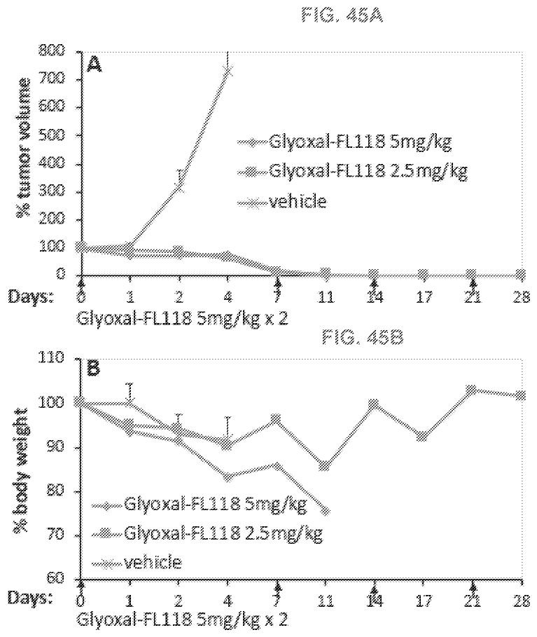

Example 34-Glyoxal-HP BCD-formulated FL118 antitumor activity and toxicity (body weight changes) profiles: The antitumor activity and toxicity of FL118 formulated through the glyoxal-HPβCD muster solution were tested in the human SW620 colorectal cancer xenograft tumor animal models. The data is shown in FIG. 45 (tumor curves in A and body weight change in B). As shown, while vehicle-treated mice with tumors grew to the maximal tumor size allowed and the tumor mice had to be euthanized on day 4, FL118-treated mice exhibited tumor regression with tumor elimination. However, FL118 dosing levels should be controlled based on the animal body weight changes.

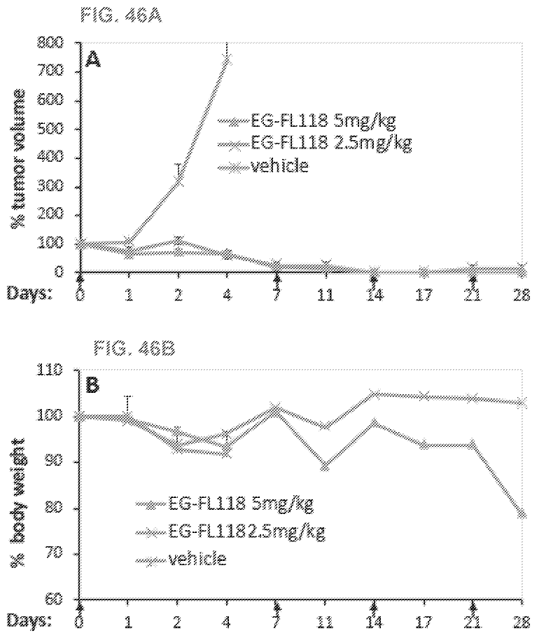

Example 35-Ethylene glycol (EG)-HP BCD-formulated FL118 antitumor activity and toxicity (body weight changes) profiles: The antitumor activity and toxicity of FL118 formulated through the EG-HPβCD muster solution were tested in the human SW620 colorectal cancer xenograft tumor animal models. The data is shown in FIG. 46 (tumor curves in A and body weight change in B). As shown, while vehicle-treated mice with tumors grew to the maximal tumor size allowed and the tumor mice had to be euthanized on day 4, FL118-treated mice exhibited tumor regression with potential tumor elimination, although FL118 dosing levels should be controlled based on the animal body weight changes.

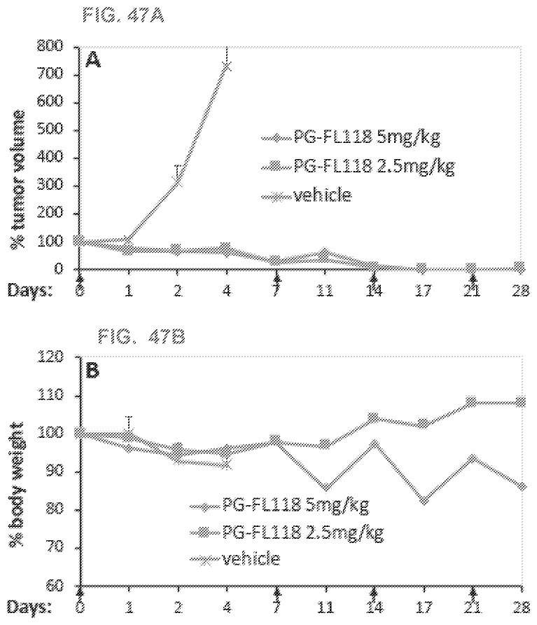

Example 36-Propylene glycol (PG)-HP BCD-formulated FL118 antitumor activity and toxicity (body weight changes) profiles: The antitumor activity and toxicity of FL118 formulated through the PG-HPβCD muster solution were tested in the human SW620 colorectal cancer xenograft tumor animal models. The data is shown in FIG. 47 (tumor curves in A and body weight change in B). As shown, while vehicle-treated mice with tumors grew to the maximal tumor size allowed and the tumor mice had to be euthanized on day 4, FL118-treated mice exhibited tumor regression with potential tumor elimination, although FL118 dosing levels should be controlled based on the animal body weight changes.

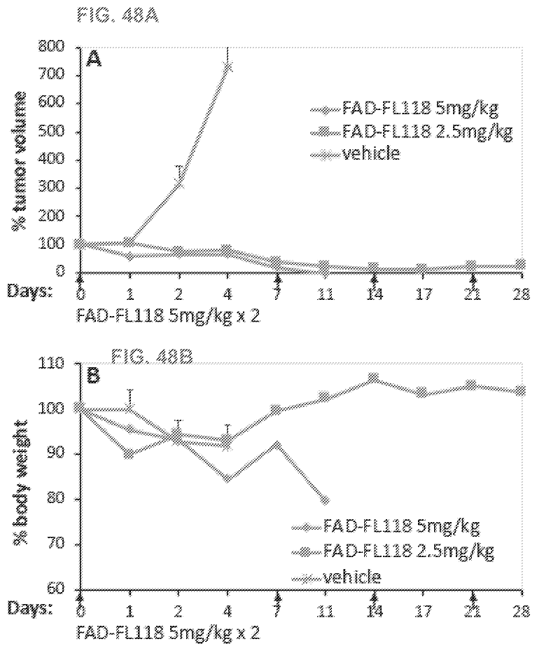

Example 37-Formamide (FAD)-HP BCD-formulated FL118 antitumor activity and toxicity (body weight changes) profiles: The antitumor activity and toxicity of FL118 formulated through the FAD-HPβCD muster solution were tested in the human SW620 colorectal cancer xenograft tumor animal models. The data is shown in FIG. 48 (tumor curves in A and body weight change in B). As shown, while vehicle-treated mice with tumors grew to the maximal tumor size allowed and the tumor mice had to be euthanized on day 4, FL118-treated mice exhibited tumor regression with potential tumor elimination. However, FL118 dosing levels should be controlled based on the animal body weight changes.

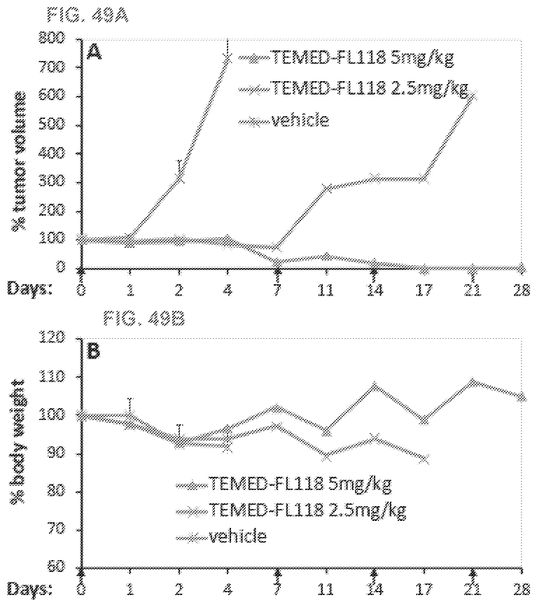

Example 38-N,N,N′,N′-tetramethyl ethylene diamine (TEMED)-HP BCD-formulated FL118 antitumor activity and toxicity (body weight changes) profiles: The antitumor activity and toxicity of FL118 formulated through the TEMED-HPβCD muster solution were tested in the human SW620 colorectal cancer xenograft tumor animal models. The data is shown in FIG. 49 (tumor curves in A and body weight change in B). As shown, while vehicle-treated mice with tumors grew to the maximal tumor size allowed and the tumor mice had to be euthanized on day 4, FL118-treated mice exhibited tumor regression with potential tumor elimination at the high dosing level. However, FL118 at the low dosing level only exhibited limited antitumor activity. Interestingly high dosing FL118 did not exhibit toxicity.

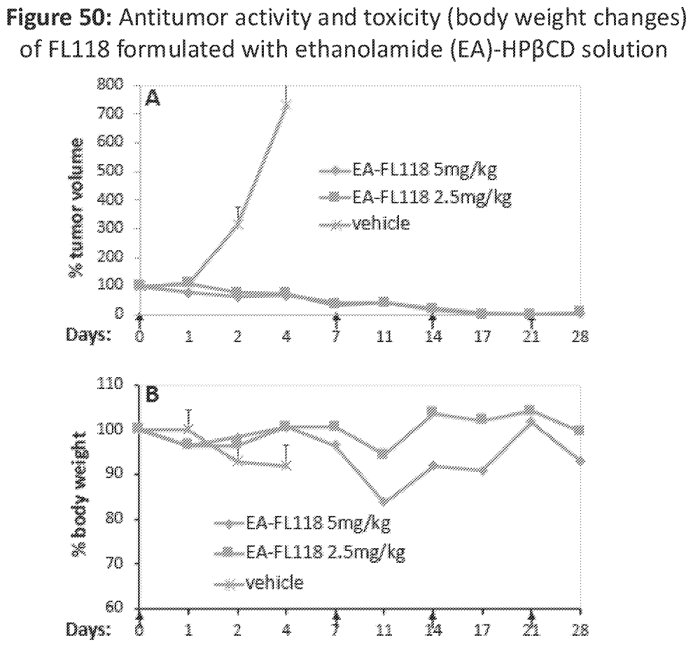

Example 39-Ethanolamide (EA)-HP BCD-formulated FL118 antitumor activity and toxicity (body weight changes) profiles: The antitumor activity and toxicity of FL118 formulated through the EA-HPβCD muster solution were tested in the human SW620 colorectal cancer xenograft tumor animal models. The data is shown in FIG. 50 (tumor curves in A and body weight change in B). As shown, while vehicle-treated mice with tumors grew to the maximal tumor size allowed and the tumor mice had to be euthanized on day 4, FL118-treated mice exhibited tumor regression with tumor elimination, although FL118 dosing levels should be controlled based on the animal body weight changes.

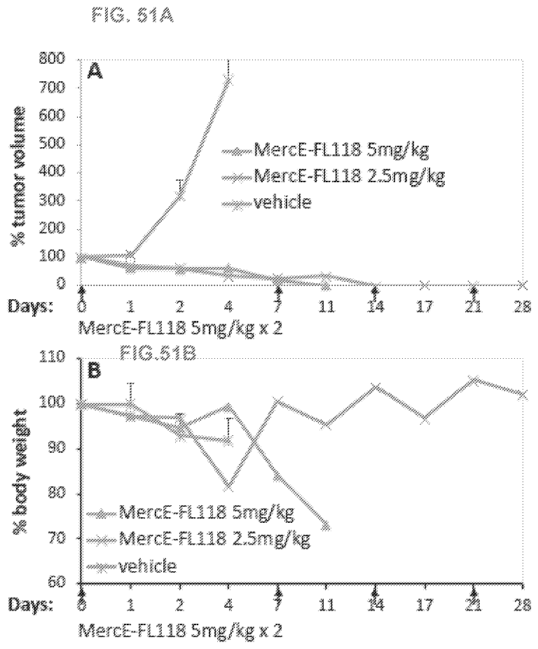

Example 40-Mercaptoethanol (MercE)-HP BCD-formulated FL118 antitumor activity and toxicity (body weight changes) profiles: The antitumor activity and toxicity of FL118 formulated through the MercE-HPβCD muster solution were tested in the human SW620 colorectal cancer xenograft tumor animal models. The data is shown in FIG. 51 (tumor curves in A and body weight change in B). As shown, while vehicle-treated mice with tumors grew to the maximal tumor size allowed and the tumor mice had to be euthanized on day 4, FL118-treated mice exhibited tumor regression with tumor elimination. However, FL 118 dosing levels should be controlled based on the animal body weight changes.

Example 41—FL118 drug substance (DS) manufacturing and chemical stability study plans: Through contract services, CRO WuXi AppTec (Wuhan) and STA Pharmaceutical Co. Ltd (a subsidiary of WuXuAppTec) provided two batches of non-GMP and one batch of GMP FL118 active pharmaceutical ingredient (API) substances and performed FL118 API stabilities in the planed conditions (Table 5). Detailed FL118 DS stability studies are presented in the following examples.

TABLE 5
The FL118 chemical stability test plan outline.

| FL118 API batch | | | Manufacturing | | | |
|---|---|---|---|---|---|---|
| number | Batch size | | date | Study type | Start date | Production location |
| P12211-005- | 238.5 | g | 12 Oct. 2018 | Stress testing | 7 Mar. 2019 | WuXi AppTec |
| P1(non-GMP) | | | | | | (Wuhan) Co. Ltd. |
| P12211-005- | 238.5 | g | 12 Oct. 2018 | Accelerated test | 7 Mar. 2019 | WuXi AppTec |
| P1(non-GMP) | | | | and long-term | | (Wuhan) Co. Ltd. |
| | | | | stability tests | | |
| P12211-006- | 256.5 | g | 13 Oct. 2018 | Accelerated test | 7 Mar. 2019 | WuXi AppTec |
| P1(non-GMP) | | | | and long-term | | (Wuhan) Co. Ltd. |
| | | | | stability tests | | |
| Cl80402127- | 810 | g | 30 Dec. 2018 | Accelerated test | 7 Mar. 2019 | STA Pharmaceutical |
| BF18001 (GMP) | | | | and long-term | | (Shanghai) Co., Ltd |
| | | | | stability tests | | (a WuXi AppTec |
| | | | | | | Subsidiary) |

Example 42—FL118 DS exhibited high chemical stability at 60° C. for up to 30 days: One non-GMP batch (P12211-005-P1) of FL118 DS chemical stability under high temperature at 60° C. for 5, 10 and 30 days (Stress Test), FL118 showed high stability (Table 6). Specifically, the FL118 purity on day 0 (98.8%), day 5 (98.6%), day 10 (98.7%) and day 98.5%) are within the testing assay variation range (Table 6), while slightly increased total impurities on day 10 and day 30 (i.e., day 0, 0.6%; day 5, 0.6%; day 10, 0.38%; and day 0, 0.31%), but the total impurities are far below the allowed level (≤2.0%).

TABLE 6
FL118 chemical stability results from the stress test at 60° C. for up to 30 days.

| | | Batch number: P12211-005-P1 | | High temperature: 60° C. | |
|---|---|---|---|---|---|
| Items | Specification | 0 day | 5 days | 10 days | 30 days |
| Appear- | Off-white to light | Top layer: Light yellow | Top layer: Light yellow | Top layer: Light yellow | Top layer: Light yellow |
| ance | brown solid | solid; middle and bottom | solid; middle and bottom | solid; middle and bottom | solid; middle and bottom |
| | | layer: Off-white solid | layer: Off-white solid | layer: Off-white solid | layer: Off-white solid |
| Related | PTS ≤ 0.20% | Not detected | Not detected | Not detected | Not detected |
| sub- | FL118-S2 ≤ 0.20% | 0.06% | 0.06% | 0.07% | 0.07% |
| stances | FL118-IM11 ≤ 0.20% | Not detected | Not detected | Not detected | Not detected |
| | FL118-I03 ≤ 0.20% | Not detected | Not detected | Not detected | Not detected |
| | FL118-IM09 ≤ 0.20% | Not detected | 0.02% (<LOQ) | 0.08% | 0.05% |
| | FL118-IM44 ≤ 0.20% | Not detected | Not detected | Not detected | Not detected |
| | FL118-IM45 ≤ 0.20% | Not detected | Not detected | Not detected | Not detected |
| | Unknown impurity ≤ | RRT = 1.05:0.03%(<LOQ) | RRT = 0.56:0.03%(<LOQ) | RRT = 0.47:0.03%(<LOQ) | RRT = 0.78:0.05% |
| | 0.15% | | RRT = 0.66:0.04%(<LOQ) | RRT = 0.88:0.07% | RRT = 0.96:0.06% |
| | | | RRT = 0.92:0.03%(<LOQ) | RRT = 0.92:0.12% | RRT = 1.03:0.08% |
| | | | RRT = 1.03:0.03%(<LOQ) | RRT = 1.03:0.05% | RRT = 1.05:0.02%(<LOQ) |
| | | | RRT = 1.09:0.02%(<LOQ) | | RRT = 1.08:0.04%(<LOQ) |
| | | | RRT = 1.16:0.03%(<LOQ) | | |
| | Total impurities ≤ | 0.06% | 0.06% | 0.38% | 0.31% |
| | 2.0% | | | | |
| Isomer | FL118-IM29 ≤ 0.15% | <0.10% | <0.10% | <0.10% | <0.10% |
| Assay | 98.0%~102.0% | 98.8% | 98.6% | 98.7% | 98.5% |
| Water | ≤0.5% | 0.09% | 0.07% | 0.15% | 0.09% |

Example 43—FL118 DS exhibited high chemical stability at 25° C. with high relative humility (RH) for up to 30 days: In the Stress Test under room temperature (25° C.) with high relative humility (RH92.5%) for 5, 10 and 30 days, FL118 also exhibited high stability (Table 7). Specifically, the FL118 purity on day 0 (98.8%), day 5 (98.7%), day 10 (98.7%) and day 98.7%) are within the testing assay variation range (Table 7), while slightly increased total impurities on day 10 and day 30 (i.e., day 0, 0.6%; day 5, 0.5%; day 10, 0.12%; and day 0, 0.19%), but the total impurities are far below the allowed level (≤2.0%).

TABLE 7
FL118 chemical stability results from the stress test at 25° C. with high relative humility for up to 30 days.

| | | Batch number: P12211-005-P1 | | High humidity: 25° C./92.5% RH | |
|---|---|---|---|---|---|
| Items | Specification | 0 day | 5 days | 10 days | 30 days |
| Appear- | Off-white to light | Off-white solid | Off-white solid | Off white solid | Off-white solid |
| ance | brown solid | | | | |
| Related | PTS ≤ 0.20% | Not detected | Not detected | Not detected | Not detected |
| sub- | FL118-S2 ≤ 0.20% | 0.06% | 0.05% | 0.06% | 0.08% |
| stances | FL118-IM11 ≤ 0.20% | Not detected | Not detected | Not detected | Not detected |
| | FL118-I03 ≤ 0.20% | Not detected | Not detected | Not detected | Not detected |
| | FL118-IM09 ≤ 0.20% | Not detected | Not detected | Not detected | Not detected |
| | FL118-IM44 ≤ 0.20% | Not detected | Not detected | Not detected | Not detected |
| | FL118-IM45 ≤ 0.20% | Not detected | Not detected | Not detected | 0.02%(<LOQ) |
| | Unknown impurity ≤ | RRT = 1.05:0.03%(<LOQ) | RRT = 0.21:0.02%(<LOQ) | RRT = 0.47:0.04%(<LOQ) | RRT = 0.96:0.05% |
| | 0.15% | | RRT = 0.97:0.03%(<LOQ) | RRT = 0.92:0.06% | RRT = 1.03:0.06% |
| | | | RRT = 1.03:0.04%(<LOQ) | RRT = 1.03:0.04%(<LOQ) | RRT = 1.05:0.02%(<LOQ) |
| | | | | | RRT = 1.08:0.03%(<LOQ) |
| | Total impurities ≤ | 0.06% | 0.05% | 0.12% | 0.19% |
| | 2.0% | | | | |
| Isomer | FL118-IM29 ≤ 0.15% | <0.10% | <0.10% | <0.10% | <0.10% |
| Assay | 98.0%~102.0% | 98.8% | 98.7% | 98.7% | 98.7% |
| Water | ≤0.5% | 0.09% | 0.11% | 0.16% | 0.10% |
| Hygro- | ≤0.5% | N/A | 0.07% | 0.20% | 0.38% |
| scope | | | | | |

Example 44—FL118 DS exhibited high chemical stability from the 10-day light stress test at 25° C./60% RH: In the light Stress Test at 25° C. with normal relative humility (RH60%) for 5 and 10 days, FL118 also exhibited high stability (Table 8). Specifically, the FL118 purity on day 0 (98.8%), day 5 (98.5%) and day 10 (98.8%) are within the testing assay variation range (Table 8), while slightly increased total impurities on day 5 and day 10 (i.e., day 0, 0.6%; day 5, 0.21% and day 10, 0.26%), but the total impurities are far below the allowed level (≤2.0%).

TABLE 8
FL118 chemical stability results from the 10-day light stress test at 25° C./60% RH.

| | | Batch number: P12211-005-P1 Light | | |
|---|---|---|---|---|
| | | | 5 days | |
| Items | Specification | 0 day | Sample | Reference |
| Appearance | Off-white to light | Off-white solid | Top layer. Light yellow | Off-white solid |
| | brown solid | | solid; middle and bottom | |
| | | | layer: Off-white solid | |
| Related | PTS ≤0.20% | Not detected | Not detected | Not detected |
| substances | FL118-S2 ≤0.20% | 0.06% | 0.06% | 0.06% |
| | FL118-IM11 ≤0.20% | Not detected | Not detected | Not detected |
| | FL118-103 ≤0.20% | Not detected | Not detected | Not detected |
| | FL118-IM09 ≤0.20% | Not detected | Not detected | Not detected |
| | FL.118-IM44 ≤0.20% | Not detected | Not detected | Not detected |
| | FL118-IM45 ≤0.20% | Not detected | Not detected | Not detected |
| | Unknown | RRT = 1.05:0.03%(<LOQ) | RRT = 0.20:0.08% | RRT = 0.92:0.02%(<LOQ) |
| | impurity ≤0.15% | | RRT = 0.39:0.07% | RRT = 0.97:0.03%(<LOQ) |
| | | | RRT = 0.97:0.03%(<LOQ) | RRT = 1.03:0.04%(<LOQ) |
| | | | RRT = 1.03:0.04%(<LOQ) | |
| | | | RRT = 1.09:0.02%(<LOQ) | |
| | Total | 0.06% | 0.21% | 0.06% |
| | impurities ≤2.0% | | | |
| Isomer | FL118-IM29 ≤0.15% | <0.10% | <0.10% | <0.10% |
| Assay | 98.0%~102.0% | 98.8% | 98.5% | 98.3% |
| Water | ≤0.5% | 0.09% | 0.07% | 0.08% |

| | | Batch number: P12211-005-P1 Light | |
|---|---|---|---|
| | | 10 days | |
| Items | Specification | Sample | Reference |
| Appearance | Off-white to light | Top layer. Light yellow | Off-white solid |
| | brown solid | solid; middle and bottom | |
| | | layer: Off-white solid | |
| Related | PTS ≤0.20% | Not detected | Not detected |
| substances | FL118-S2 ≤0.20% | 0.06% | 0.07% |
| | FL118-IM11 ≤0.20% | Not detected | Not detected |
| | FL118-103 ≤0.20% | Not detected | Not detected |
| | FL118-IM09 ≤0.20% | Not detected | 0.06% |
| | FL.118-IM44 ≤0.20% | Not detected | Not detected |
| | FL118-IM45 ≤0.20% | Not detected | Not detected |
| | Unknown | RRT = 0.92:0.08% | RRT = 0.92:0.08% |
| | impurity ≤0.15% | RRT = 1.03:0.05% | RRT = 1.03:0.05% |
| | Total | 0.25% | 0.26% |
| | impurities ≤2.0% | | |
| Isomer | FL118-IM29 ≤0.15% | <0.10% | <0.10% |
| Assay | 98.0%~102.0% | 98.9% | 98.8% |
| Water | ≤0.5% | 0.14% | 0.13% |

Example 45—FL118 DS exhibited high chemical stability from the accelerated test condition (40° C./75% RH) for up to 6 months: All the three batches (P12211-005-P1, P12211-006-P1 and C180402127-BF18001) of FL118 chemical stability under the accelerated condition (40° C./75% RH). In this condition, the FL118 stability result for the non-GMP batch of P12211-005-P1 was summarized in Table 9.

TABLE 9
FL118 chemical stability results from the accelerated test condition
(40° C./75% RH) for the batch of P12211-005-P1 for up to 6 months.

| | | Batch NO .: P12211-005-P1 Accelerated condition: 40° C./75% RH | | |
|---|---|---|---|---|
| Item | Specification | 0 day | 1 month | 2 months |
| Appearance | Off-white to light | Off-white solid | Off-white solid | Off-white solid |
| | brown solid | | | |
| Related | PTS ≤0.20% | Not detected | Not detected | Not detected |
| substances | FL118-S2 ≤0.20% | 0.06% | 0.07% | 0.06% |
| | FL118-IM11 ≤0.20% | Not detected | Not detected | Not detected |
| | FL118-103 ≤0.20% | Not detected | Not detected | Not detected |
| | FL118-IM09 ≤0.20% | Not detected | Not detected | 0.02% (<LOQ) |
| | FL118-IM44 ≤0.20% | Not detected | Not detected | Not detected |
| | FL118-IM45 ≤0.20% | Not detected | 0.02% (<LOQ) | 0.02% (<LOQ) |
| | Unknown | RRT = 1.05:0.03%(<LOQ) | RRT = 0.92:0.02%(<LOQ) | RRT = 0.96:0.04%(<LOQ) |
| | impurity ≤0.15% | | RRT = 0.96:0.04%(<LOQ) | RRT = 0.97:0.06% |
| | | | RRT = 1.03:0.06% | RRT = 1.03:0.06% |
| | | | RRT = 1.05:0.02%(<LOQ) | RRT = 1.05:0.03%(<LOQ) |
| | | | RRT = 1.08:0.03%(<LOQ) | RRT = 1.09:0.03%(<LOQ) |
| | Total | 0.06% | 0.13% | 0.18% |
| | impurities ≤2.0% | | | |
| Isomer | FL118-IM29 ≤0.15% | <0.10% | <0.10% | <0.10% |
| Assay | 98.0%~102.0% | 98.8% | 98.8% | 98.8% |
| Water | ≤0.5% | 0.09% | 0.07% | 0.11% |

| | | Batch NO .: P12211-005-P1 Accelerated condition: 40° C./75% RH | |
|---|---|---|---|
| Item | Specification | 3 months | 6 months |
| Appearance | Off-white to light | Off-white solid | Off-white solid |
| | brown solid | | |
| Related | PTS ≤0.20% | Not detected | Not detected |
| substances | FL118-S2 ≤0.20% | 0.06% | 0.06% |
| | FL118-IM11 ≤0.20% | Not detected | Not detected |
| | FL118-103 ≤0.20% | Not detected | Not detected |
| | FL118-IM09 ≤0.20% | Not detected | Not detected |
| | FL118-IM44 ≤0.20% | Not detected | Not detected |
| | FL118-IM45 ≤0.20% | Not detected | Not detected |
| | Unknown | RRT = 0.97:0.06% | RRT = 0.35:0.10% |
| | impurity ≤0.15% | RRT = 1.03:0.08% | RRT = 0.96:0.07% |
| | | RRT = 1.04:0.04%(<LOQ) | RRT = 1.03:0.07% |
| | | | RRT = 1.05:0.03%(<LOQ) |
| | Total | 0.20% | 0.30% |
| | impurities ≤2.0% | | |
| Isomer | FL118-IM29 ≤0.15% | <0.10% | <0.10% |
| Assay | 98.0%~102.0% | 98.6% | 98.4% |
| Water | ≤0.5% | 0.13% | 0.27% |

The FL118 stability result for the non-GMP batch of P12211-006-PI in this condition was summarized in Table 10.

TABLE 10
FL118 chemical stability results from the accelerated test condition
(40° C./75% RH) for the batch of P12211-006-P1 for up to 6 months.

| | | Batch NO.: P12211-006-P1 Accelerated condition: 40° C./75% RH | | |
|---|---|---|---|---|
| Item | Specification | 0 day | 1 month | 2 months |
| Appearance | Off-white to light | Off-white solid | Off-white solid | Off-white solid |
| | brown solid | | | |
| Related | PTS ≤2.0% | Not detected | Not detected | Not detected |
| substances | FL118-S2 ≤2.0% | 0.05% | 0.06% | 0.05% |
| | FL118-IM11 ≤2.0% | Not detected | Not detected | Not detected |
| | FL118-103 ≤2.0% | Not detected | Not detected | Not detected |
| | FL118-IM09 ≤2.0% | Not detected | Not detected | Not detected |
| | FL118-IM44 ≤2.0% | Not detected | Not detected | Not detected |
| | FL.118-IM45 ≤2.0% | Not detected | 0.02%(<LOQ) | Not detected |
| | Unknown | RRT = 1.05:0.03%(<LOQ) | RRT = 0.96:0.05% | RRT = 0.96:0.06% |
| | impurity ≤0.15% | | RRT = 1.03:0.07% | RRT = 1.03:0.07% |
| | | | RRT = 1.05:0.03%(<LOQ) | RRT = 1.05:0.03% (<LOQ) |
| | | | RRT = 1.09:0.03%(<LOQ) | |
| | Total impurities ≤2.0% | 0.05% | 0.18% | 0.18% |
| Isomer | FL118-IM29 ≤0.15% | <0.10% | <0.10% | <0.10% |
| Assay | 98.0%~102.0% | 99.0% | 98.9% | 99.0% |
| Water | ≤0.5% | 0.06% | 0.06% | 0.12% |

| | | Batch NO.: P12211-006-P1 Accelerated condition: 40° C./75% RH | |
|---|---|---|---|
| Item | Specification | 3 months | 6 months |
| Appearance | Off-white to light | Off-white solid | Off white solid |
| | brown solid | | |
| Related | PTS ≤2.0% | 0.03%(<LOQ) | Not detected |
| substances | FL118-S2 ≤2.0% | 0.05% | 0.05% |
| | FL118-IM11 ≤2.0% | Not detected | Not detected |
| | FL118-103 ≤2.0% | Not detected | Not detected |
| | FL118-IM09 ≤2.0% | Not detected | Not detected |
| | FL118-IM44 ≤2.0% | Not detected | Not detected |
| | FL.118-IM45 ≤2.0% | Not detected | Not detected |
| | Unknown | RRT = 0.97:0.06% | RRT = 0.96:0.09% |
| | impurity ≤0.15% | RRT = 1.03:0.08% | RRT = 1.03:0.079% |
| | | RRT = 1.04:0.02%(<LOQ) | RRT = 1.05:0.03%(<LOQ) |
| | | RRT = 1.05:0.03%(<LOQ) | |
| | Total impurities ≤2.0% | 0.19% | 0.21% |
| Isomer | FL118-IM29 ≤0.15% | <0.10% | <0.10% |
| Assay | 98.0%~102.0% | 98.6% | 99.8% |
| Water | ≤0.5% | 0.10% | 0.24% |

The FL118 stability result for the GMP batch of C180402127-BF 18001 was summarized in Table 11.

TABLE 11
FL118 chemical stability results from the accelerated test condition (40°
C./75% RH) for the batch of C180402127-BF18001-P1 for up to 6 months.

| | | Batch NO.: C180402127-BF18001-P1 Accelerated condition: 40° C./75% RHI | | |
|---|---|---|---|---|
| Item | Specification | 0 day | 1 month | 2 months |
| Appearance | Off-white to light | Off-white solid | Off-white solid | Off-white solid |
| | brown solid | | | |
| Related | PTS ≤2.0% | Not detected | Not detected | Not detected |
| substances | FL118-S2 ≤2.0% | 0.08% | 0.10% | 0.08% |
| | FL118-IM11 ≤2.0% | Not detected | Not detected | Not detected |
| | FL118-103 ≤2.0% | Not detected | Not detected | Not detected |
| | FL118-IM09 ≤2.0% | Not detected | 0.03%(<LOQ) | 0.03%(<LOQ) |
| | FL118-IM44 ≤2.0% | Not detected | Not detected | Not detected |
| | FL118-IM45 ≤2.0% | Not detected | Not detected | Not detected |
| | Unknown | RRT = 1.05:0.04%(<LOQ) | RRT = 0.92:0.03%(<LOQ) | RRT = 0.96:0.06% |
| | impurity ≤0.15% | | RRT = 0.96:0.05% | RRT = 1.03:0.07% |
| | | | RRT = 1.03:0.07% | RRT = 1.05:0.04%(<LOQ) |
| | | | RRT = 1.05:0.04%(<LOQ) | RRT = 1.08:0.02%(<LOQ) |
| | | | RRT = 1.08:0.02%(<LOQ) | |
| | Total | 0.08% | 0.22% | 0.21% |
| | impurities ≤2.0% | | | |
| Isomer | FL118-IM29 ≤0.15% | <0.10% | <0.10% | <0.10% |
| Assay | 98.0%~102.0% | 98.8% | 99.2% | 98.6% |
| Water | ≤0.5% | 0.03% | 0.07% | 0.12% |

| | | Batch NO.: C180402127-BF18001-P1 Accelerated condition: 40° C./75% RHI | |
|---|---|---|---|
| Item | Specification | 3 months | 6 months |
| Appearance | Off-white to light | Off-white solid | Off-white solid |
| | brown solid | | |
| Related | PTS ≤2.0% | 0.03% | Not detected |
| substances | FL118-S2 ≤2.0% | 0.08% | 0.08% |
| | FL118-IM11 ≤2.0% | Not detected | Not detected |
| | FL118-103 ≤2.0% | Not detected | Not detected |
| | FL118-IM09 ≤2.0% | Not detected | 0.03%(<LOQ) |
| | FL118-IM44 ≤2.0% | Not detected | Not detected |
| | FL118-IM45 ≤2.0% | Not detected | Not detected |
| | Unknown | RRT = 0.97:0.05% | RRT = 0.35:0.11% |
| | impurity ≤0.15% | RRT = 1.03:0.07% | RRT = 0.96:0.08% |
| | | RRT = 1.04:0.05% | RRT = 1.03:0.08% |
| | | | RRT = 1.05:0.04%(<LOQ) |
| | Total | 0.25% | 0.35% |
| | impurities ≤2.0% | | |
| Isomer | FL118-IM29 ≤0.15% | <0.10% | <0.10% |
| Assay | 98.0%~102.0% | 99.4% | 98.4% |
| Water | ≤0.5% | 0.10% | 0.25% |

As shown in Tables 9-11, all the three batches of FL118 API are highly stable in the 75% RH at 40° C. For example, as shown in Table 10 for the second non-GMP batch of FL118 (P12211-006-P1), the FL118 purity on day 0 (99.0%), 1 month (98.9%), 2 months (99.0%), 3 months (98.6%) and 6 months (99.8%) under this accelerated condition is within the testing assay variation range, while slightly increased total impurities on 1 month, 2 months, 3 months and 6 months (i.e., day 0, 0.5%; 1 month, 0.18%; 2 months, 0.18%; 3 months, 0.19% and 6 months, 0.21%), but the total impurities are far below the allowed level (≤2.0%).

Example 46—FL118 DS exhibited high chemical stability from the long-term condition (25° C./60% RH) for up to 24 months: All the three batches (P12211-005-P1, P12211-006-P1 and C180402127-BF18001) of FL118 chemical stability for 24-month long-term conditions at an ambient of 25° C./60% RH. In this condition, the FL118 chemical stability result for the non-GMP batch of P12211-005-P1 was summarized in Table 12.

TABLE 12
FL118 chemical stability results from the long-term condition (25°
C./60% RH) for the batch of P12211-005-P1 for up to 24 months.

| | | Batch NO.: P12211-005-P1 Long term condition: 25° C./60% RH | | | |
|---|---|---|---|---|---|
| Item | Specification | 0 day | 3 months | 6 months | 9 months |
| Appearance | Off-white to light | Off-white solid | Off-white solid | Off white solid | Off-white solid |
| | brown solid | | | | |
| Related | PTS ≤2.0% | Not detected | Not detected | Not detected | Not detected |
| substances | FL118-S2 ≤2.0% | 0.06% | 0.06% | 0.07% | 0.06% |
| | FL118-IM11 ≤2.0% | Not detected | Not detected | Not detected | Not detected |
| | FL118-103 ≤2.0% | Not detected | Not detected | Not detected | Not detected |
| | FL118-IM09 ≤2.0% | Not detected | Not detected | Not detected | 0.02%(<LOQ) |
| | FL118-IM44 ≤2.0% | Not detected | Not detected | Not detected | Not detected |
| | FL118-IM45 ≤2.0% | Not detected | Not detected | 0.02%(<LOQ) | 0.02%(<LOQ) |
| | Unknown | RRT = 1.05:0.03% | RRT = 0.97:0.07% | RRT = 0.35:0.10% | RRT = 0.97:0.04% |
| | impmity ≤0.15% | (<LOQ) | RRT = 1.03:0.06% | RRT = 0.96:0.08% | (<LOQ) |
| | | | RRT = 1.05:0.03% | RRT = 1.03:0.07% | RRT = 1.04:0.03% |
| | | | (<LOQ) | RRT = 1.05:0.03% | (<LOQ) |
| | | | | (<LOQ) | |
| | | | | RRT = 1.08:0.03% | RRT = 1.05:0.03% |
| | | | | (<LOQ) | (<LOQ) |
| | | | | RRT = 1.09:0.02% | RRT = 1.09:0.02% |
| | | | | (<LOQ) | (<LOQ) |
| | Total | 0.06% | 0.19% | 0.32% | 0.06% |
| | impurities ≤2.0% | | | | |
| Isomer | FL118-IM29 ≤0.15% | <0.10% | <0.10% | <0.10% | <0.10% |
| Assay | 98.0%~102.0% | 98.8% | 98.4% | 98.6% | 99.0% |
| Water | ≤0.5% | 0.09% | 0.12% | 0.26% | 0.12% |

| | | Batch NO.: P12211-005-P1 Long term condition: 25° C./60% RH | | |
|---|---|---|---|---|
| Item | Specification | 12 months | 18 months | 24 months |
| Appearance | Off-white to light | Off-white solid | Off-white solid | Off-white solid |
| | brown solid | | | |
| Related | PTS ≤2.0% | Not detected | Not detected | Not detected |
| substances | FL118-S2 ≤2.0% | 0.06% | 0.06% | 0.06% |
| | FL118-IM11 ≤2.0% | Not detected | Not detected | Not detected |
| | FL118-103 ≤2.0% | Not detected | Not detected | Not detected |
| | FL118-IM09 ≤2.0% | Not detected. | 0.02%(<LOQ) | 0.02%(<LOQ) |
| | FL118-IM44 ≤2.0% | Not detected | Not detected | Not detected |
| | FL118-IM45 ≤2.0% | Not detected | 0.02%(<LOQ) | Not detected |
| | Unknown | RRT = 0.96:0.03%(<LOO) | RRT = 0.97:0.02%(<LOQ) | RRT = 0.78:0.02%(<LOQ) |
| | impmity ≤0.15% | RRT = 1.03:0.02%(<LOO) | RRT = 1.03:0.03%(<LOQ) | RRT = 0.96:0.03%(<LOQ) |
| | | RRT = 1.09:0.02%(<LOQ) | RRT = 1.05:0.03%(<LOQ) | RRT = 1.03:0.03%(<LOQ) |
| | | | RRT = 1.09:0.02%(<LOQ) | RRT = 1.05:0.02%(<LOQ) |
| | | | | RRT = 1.09:0.02%(<LOQ) |
| | Total | 0.06% | 0.06% | 0.06% |
| | impurities ≤2.0% | | | |
| Isomer | FL118-IM29 ≤0.15% | <0.10% | <0.10% | <0.10% |
| Assay | 98.0%~102.0% | 99.1% | 98.5% | 98.5% |
| Water | ≤0.5% | 0.05% | 0.07% | 0.11% |

The FL118 stability result for the non-GMP batch of P12211-006-P1 in this condition was summarized in Table 13.

TABLE 13
FL118 chemical stability results from the long-term condition (25° C./60% RH) for the batch of P12211-006-P1 for up to 24 months.

| | | Batch NO.: P12211-006-P1 Long-term condition: 25° C./60% RH | | | |
|---|---|---|---|---|---|
| Item | Specification | 0 day | 3 months | 6 months | 9 months |
| Appearance | Off-white to light | Off-white solid | Off-white solid | Off white solid | Off-white solid |
| | brown solid | | | | |
| Related | PTS ≤2.0% | Not detected | Not detected | Not detected | Not detected |
| substances | FL118-S2 ≤2.0% | 0.05% | 0.05% | 0.05% | 0.05% |
| | FL118-IM11 ≤2.0% | Not detected | Not detected | Not detected | Not detected |
| | FL118-103 ≤2.0% | Not detected | Not detected | Not detected | Not detected |
| | FL118-IM09 ≤2.0% | Not detected | Not detected | Not detected | Not detected |
| | FL118-IM44 ≤2.0% | Not detected | Not detected | Not detected | Not detected |
| | FL118-IM45 ≤2.0% | Not detected | Not detected | Not detected | Not detected |
| | Unknown | RRT = 1.05:0.03% (<LOQ) | RRT = 0.97:0.05% | RRT = 0.35:0.11% | RRT = 0.97:0.04% (LOQ) |
| | impurity ≤0.15% | | RRT = 1.03:0.08% | RRT = 0.96:0.07% | RRT = 1.03:0.02% (LOQ) |
| | | | RRT = 1.05:0.05% | RRT = 1.03:0.06% | RRT = 1.05:0.03% (LOQ) |
| | | | | RRT = 1.08:0.03% (LOQ) | RRT = 1.09:0.02% (LOQ) |
| | Total | 0.05% | 0.23% | 0.29% | 0.05% |
| | impurities ≤2.0% | | | | |
| Isomer | FL118-IM29 ≤0.15% | <0.10% | <0.10% | <0.10% | <0.10% |
| Assay | 98.0%~102.0% | 99.0% | 99.5% | 98.8% | 99.4% |
| Water | <0.5% | 0.06% | 0.09% | 0.24% | 0.11% |
| XRPD | Refer to the | Refer to the | Refer to the | Refer to the | N/A |
| | result tested | spectrum | result tested | result tested | |
| | at 0 day | of reference | at 0 day | at 0 day | |

| | | Batch NO.: P12211-006-P1 Long-term condition: 25° C./60% RH | | |
|---|---|---|---|---|
| Item | Specification | 12 months | 18 months | 24 months |
| Appearance | Off-white to light | Off-white solid | Off-white solid | Off-white solid |
| | brown solid | | | |
| Related | PTS ≤2.0% | Not detected | Not detected | Not detected |
| substances | FL118-S2 ≤2.0% | 0.05% | 0.05% | <LOQ |
| | FL118-IM11 ≤2.0% | Not detected | Not detected | Not detected |
| | FL118-103 ≤2.0% | Not detected | Not detected | Not detected |
| | FL118-IM09 ≤2.0% | 0.02%(<LOQ) | Not detected | 0.02%(<LOQ) |
| | FL118-IM44 ≤2.0% | Not detected | Not detected | Not detected |
| | FL118-IM45 ≤2.0% | Not detected | Not detected | Not detected |
| | Unknown | RRT = 0.97:0.06% | RRT = 0.97:0.03% (LOQ) | RRT = 0.96:0.03%(LOQ) |
| | impurity ≤0.15% | RRT = 1.03:0.04% (LOQ) | RRT = 1.03:0.03% (LOQ) | RRT = 1.03:0.03%(LOQ) |
| | | RRT = 1.05:0.03% (LOQ) | RRT = 1.05:0.03% (LOQ) | RRT = 1.05:0.03%(LOQ) |
| | | RRT = 1.07:0.02% (LOQ) | RRT = 1.09:0.02% (LOQ) | |
| | | RRT = 1.09:0.03% (LOQ) | | |
| | Total | 0.11% | 0.05% | <LOQ |
| | impurities ≤2.0% | | | |
| Isomer | FL118-IM29 ≤0.15% | <0.10% | <0.10% | <0.10% |
| Assay | 98.0%~102.0% | 98.8% | 98.8% | 99.1% |
| Water | <0.5% | 0.04% | 0.06% | 0.08% |
| XRPD | Refer to the | Refer to the | N/A | Refer to the |
| | result tested | result tested | | resit tested |
| | at 0 day | at 0 day | | at 0 day |

0 day

The FL118 stability result for the GMP batch of C180402127-BF 18001 was summarized in Table 14.

TABLE 14
FL118 chemical stability results from the long-term condition (25°
C./60% RH) for the batch of C180402127-BF18001-P1 for up to 24 months.

| | | Batch NO.: C180402127-BF18001-P1 Longterm condition: 25° C./60% RH | | | |
|---|---|---|---|---|---|
| Iter | Specification | 0 day | 3 months | 6 months | 9 months |
| Appearance | Off-white to light | Off-white solid | Off-white solid | Off-white solid | Off-white solid |
| | brown solid | | | | |
| Related | PTS ≤2.0% | Not detected | Not detected | Not detected | Not detected |
| substances | FL118-S2 ≤2.0% | 0.08% | 0.08% | 0.09% | 0.08% |
| | FL118-IM11 ≤2.0% | Not detected | Not detected | Not detected | Not detected |
| | FL118-103 ≤2.0% | Not detected | Not detected | Not detected | Not detected |
| | FL118-IM09 ≤2.0% | Not detected | Not detected | 0.03%(<LOQ) | 0.03%(<LOQ) |
| | FL118-IM44 ≤2.0% | Not detected | Not detected | Not detected | Not detected |
| | FL118-IM45 ≤2.0% | Not detected | Not detected | Not detected | Not detected |
| | Unknown | RRT = 1.05:0.04% | RRT = 0.97:0.04% | RRT = 0.35:0.10% | RRT = 0.97:0.06% |
| | impurity ≤0.15% | (<LOQ) | (<LOQ) | RRT = 0.96:0.07% | RRT = 1.03:0.03% |
| | | | RRT = 1.03:0.07% | RRT = 1.03:0.06% | (<LOQ) |
| | | | RRT = 1.04:0.06% | RRT = 1.05:0.04% | RRT = 1.05:0.04% |
| | | | | (<LOQ) | (<LOQ) |
| | | | | RRT = 1.08:0.03% | |
| | | | | (<LOQ) | |
| | Total | 0.08% | 0.21% | 0.32% | 0.14% |
| | impurities ≤2.0% | | | | |
| Isomer | FL118-IM29 ≤0.15% | <0.10% | <0.10% | <0.10% | <0.10% |
| Assay | 98.0%~102.0% | 98.8% | 99.0% | 98.8% | 99.0% |
| Water | <0.5% | 0.03% | 0.10% | 0.26% | 0.10% |

| | | Batch NO.: C180402127-BF18001-P1 Longterm condition: 25° C./60% RH | | |
|---|---|---|---|---|
| Iter | Specification | 12 months | 18 months | 24 months |
| Appearance | Off-white to light | Off-white solid | Off-white solid | Off-white solid |
| | brown solid | | | |
| Related | PTS ≤2.0% | Not detected | Not detected | Not detected |
| substances | FL118-S2 ≤2.0% | 0.07% | 0.07% | 0.07% |
| | FL118-IM11 ≤2.0% | Not detected | Not detected | Not detected. |
| | FL118-103 ≤2.0% | Not detected | Not detected | Not detected |
| | FL118-IM09 ≤2.0% | 0.03%(<LOQ) | 0.02%(<LOQ) | 0.02%(<LOQ) |
| | FL118-IM44 ≤2.0% | Not detected | Not detected | Not detected |
| | FL118-IM45 ≤2.0% | Not detected | Not detected | Not detected |
| | Unknown | RRT = 0.97:0.07% | RRT = 0.97:0.04% (<LOQ) | RRT = 0.96:0.03% (<LOQ) |
| | impurity ≤0.15% | RRT = 1.03:0.05% | RRT = 1.03:0.03% (<LOQ) | RRT = 1.03:0.05% |
| | | RRT = 1.05:0.03% (<LOQ) | RRT = 1.05:0.04% (<LOQ) | RRT = 1.05:0.05% |
| | | RRT = 1.09:0.03% (<LOQ) | RRT = 1.09:0.02% (<LOQ) | RRT = 1.09:0.02% (<LOQ) |
| | Total | 0.19% | 0.07% | 0.17% |
| | impurities ≤2.0% | | | |
| Isomer | FL118-IM29 ≤0.15% | <0.10% | <0.10% | <0.10% |
| Assay | 98.0%~102.0% | 98.7% | 98.5% | 98.7% |
| Water | <0.5% | 0.05% | 0.06% | 0.08% |

As shown in Tables 12-14, all the three batches of FL118 API are highly stable under the up to 24-month long-term condition of 60% RH at 25° C. For example, as shown in Table 14 for the GMP batch of FL118 (C180402127-BF18001), the FL118 purity on day 0 (98.8%), 3 months (99.0%), 6 months (98.8%), 9 months (99.0%), 12 months (98.7%), 18 months (98.5%) and 24 months (98.7%) under this long-term condition is highly stable within the testing assay variation range, while slightly increased total impurities on 3 months, 6 months, 9 months, 12 months and 24 months (i.e., day 0, 0.8%; 3 month, 0.21%; 6 months, 0.32%; 9 months, 0.14%, 12 months, 0.19%, and 24 months, 0.17%). However, impurity on 18 months (0.7%) is even less than the impurity on day 0 (0.8%). Thus, the slight increase of impurity is basically within the test system variation. Nevertheless, the total impurities are far below the allowed level (≤2.0%). Furthermore, the GMP FL118 DS batch C180402127-BF18001 purity and impurity retest in January 2023 for the 48-month stability (GMP FL118 manufacturing date: Dec. 30, 2018) was still well passing the purity and impurity requirement (Certificate of Analysis/COA issued on Jan. 12, 2023 by the CRO is available up on request).

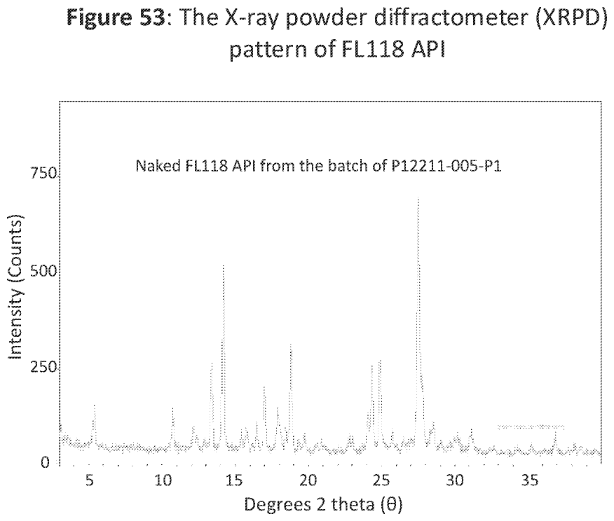

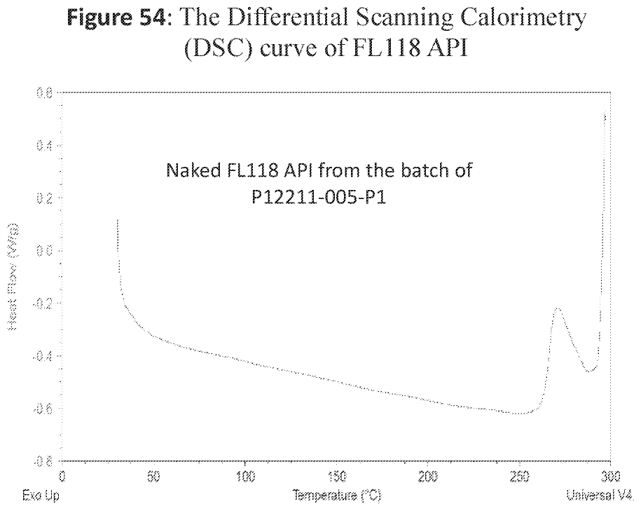

Example 47-Physical characterization of FL118 Active Pharmaceutical Ingredient (API): Physical characterization of FL118 API crystal versus amorphous statuses was performed using X-ray powder diffractometer (XRPD) and Differential Scanning calorimetry (DSC) technologies. As shown in FIG. 53, FL118 API (from the non-GMP FL118 batch of P12211-005-P1) displayed crystalline diffraction peak, which indicated that FL118 is a crystal. However, the DSC curves shown in FIG. 54 did not have a melting point peak, indicating that FL118 is in a weak crystal status.

Example 48-HPβCD-FL118 API complex powder at the 300 mg API level using the Spray Drying processes: One example for the FL118 formulation was as follows: (1) Weighed 10 g HPβCD into 50 mL volumetric flask containing a magnetic stir bar, added 40 mL 100% ethanol. Placed the volumetric flask on a Hotplate Stirrer and was magnetically stirring at room temperature under 1000 rpm for 30 min. Then added ethanol to a final volume of 50 mL to make a ethanol-HPβCD solution. (2) Transferred 15 mL of the prepared 20% HPβCD solution (containing 3g HPβCD) into three 40 mL glass bottles containing 300 mg FL118 API (from the non-GMP FL118 batch of P12211-005-P1 to produce HPβCD-FL118 complex batch of FR00535-01-190708) and magnetically stirred under 1000 rpm in the following 3 conditions at room temperature to prepare master suspensions: Condition 1: Stirring for 1 hour. Condition 2: Stirring for 6 hours. Condition 3: Stirring for 24 hours to obtain ethanol-HPβCD-FL118 complex suspension that contains 300 mg FL118 at a concentration of 20 mg/mL in each of the three 40 mL glass bottles. (3) A spray-dryer (Model: Buchi B290) was employed to prepare HPβCD-FL118 complex and the stock suspensions were stirred during spray drying. Detailed process parameters for spray drying are listed in Table 15. The obtained HPβCD-FL118 complex powders were dried under vacuum at 30° C. overnight, and the yield of the three products were 83.3%, 79.70% and 81.81% respectively.

TABLE 15
Spray drying parameters for HPβCD-FL118 complex preparation.
Parameter settings

| Instrument | Buchi B290 | | |
|---|---|---|---|
| Solvent | EtOH | | |
| Concentration (mg/mL) | 20 mg/mL of API | | |
| Nozzle orifice size (mm) | 0.7 | | |
| Batch No | Condition 1: | Condition 2: | Condition 3: |
| | FR00535-01-190708-01 | FR00535-01-190708-02 | FR00535-01-190708-3 |
| | Stirring for 1 h | Stirring for 6 h | Stirring for 24 h |
| Set inlet Temp. (° C.) | 85 | 85 | 85 |
| Actual air Temp. (° C.) | 85 | 85 | 83 |
| Chamber out Temp. (° C.) | 57 | 59 | 57 |
| Q-flow. (Bar) | 0.3 | | |
| Pump (%) | 40 | | |
| Aspirator. (%) | 100 | | |
| Product amount (g) | 2.74 | 2.63 | 2.70 |
| Yield (%) | 83.3 | 79.70 | 81.81 |

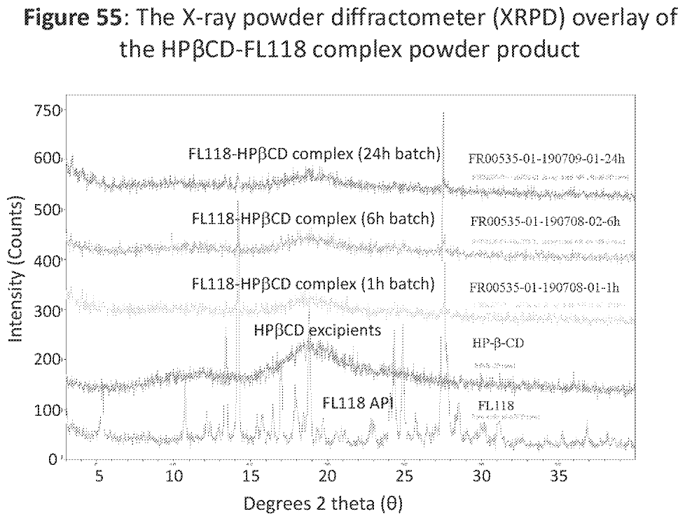

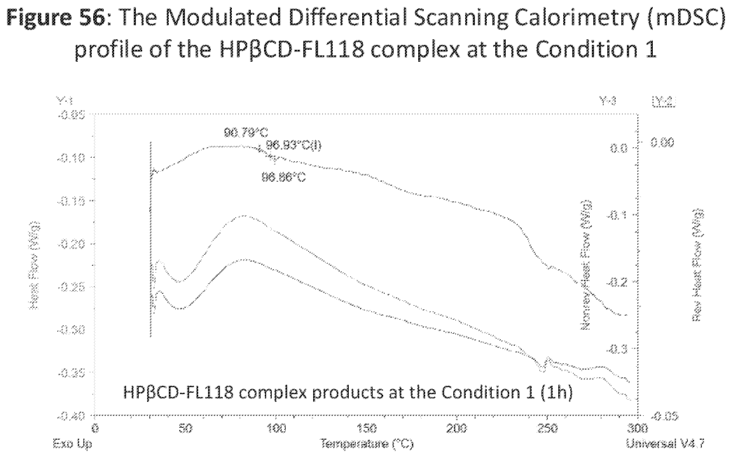

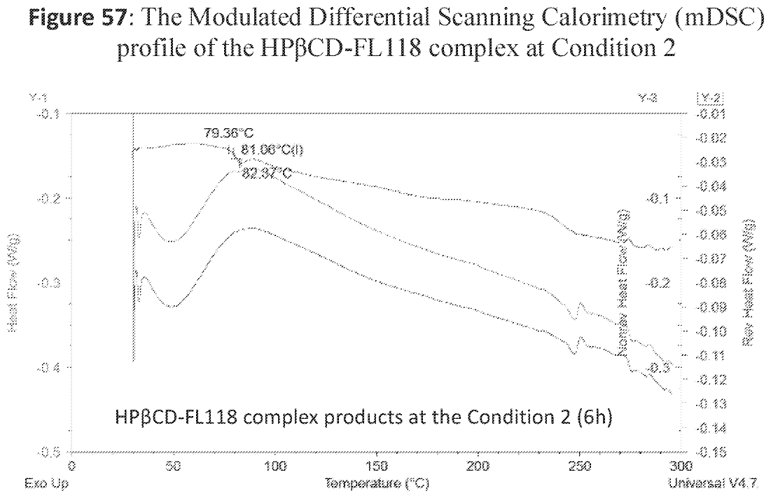

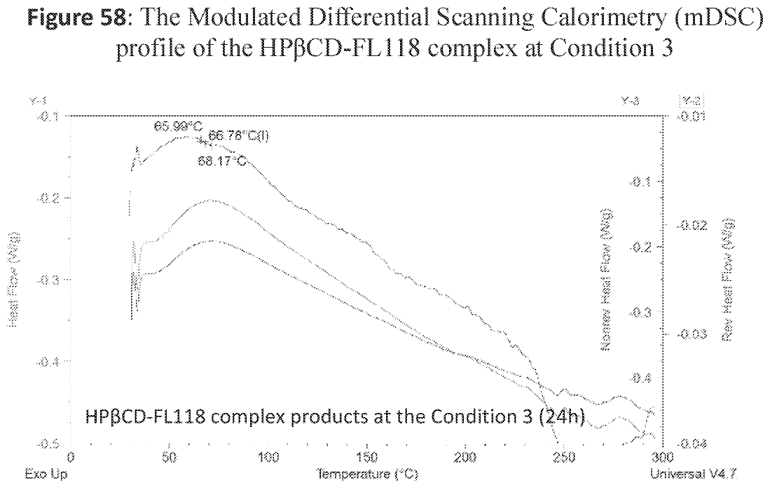

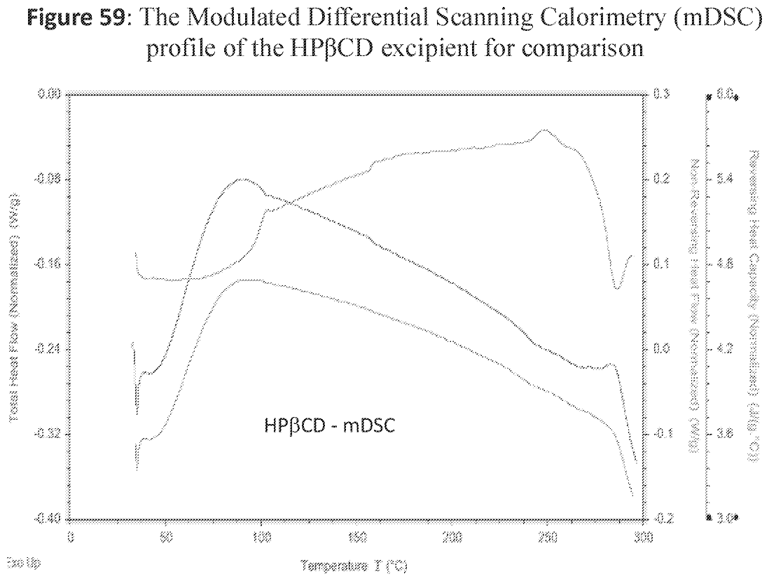

Example 49-Characterization of the HPβCD-FL118 API complex powder at the 300 mg level for 3 conditions (FL118 from the non-GMP FL118 batch of P12211-005-P1 to produce HPβCD-FL118 complex batch of FR00535-01-190708): The HPβCD-FL118 complex powder was characterized using X-ray powder diffractometer (XRPD), Modulated Differential Scanning calorimetry (mDSC) and High-Performance Liquid Chromatography (HPLC). The results are summarized in Table 16. Based on XRPD results shown in FIG. 55, the HPβCD-FL118 complex powder products were all amorphous. Only one glass transition temperature (Tg) was observed in mDSC results for the HPβCD-FL118 complex products (FIG. 56: Condition 1, FIG. 57: Condition 2 and FIG. 58: Condition 3), indicating the good miscibility of FL118 API with HPβCD. The glass transition temperature of the three HPβCD-FL118 complex products was 96.93° C., 81.6° C. and 66.78° C. respectively (Table 16). The mDSC profile of the HPβCD excipient itself is shown in FIG. 59 for comparison.

TABLE 16
Characterization of HPβCD-FL118 complex prepared through spray drying.

| Test item | | FL118 | Stirring for 1 h | Stirring for 6 h | Stirring for 24 h |
|---|---|---|---|---|---|
| Batch No. | | P12211-005- | FR00535-01- | FR00535-01- | FR00535-01- |
| | | P1 | 190708-01 | 190708-02 | 190708-3 |
| Appearance | | Off-white | Off-white | Off-white | Off-white |
| | | powder | powder | powder | powder |
| XRPD | | Weak crystal | Amorphous | Amorphous | Amorphous |
| Melting point by DSC(° C.) | | No melting | N/A | | |
| | | point | | | |
| Tg by mDSC (° C.) | | N/A | 96.93 | 81.6 | 66.78 |
| Water content (%) | | 0.7 | 4.13 | 3.75 | 4.8 |
| Residual solvent | | 0.01 | 0.01 | 0.9 | 0.01 |
| (EtOH, %) | | | | | |
| HPLC test | Drug load (%) | N/A | 7.96 | 8.32 | 8.86 |
| | Purity (%) | 99.84 | 100.0 | 100.0 | 100.0 |

Example 50-HPβCD-FL118 complex powder at the 2 g API scale-up level using the Spray Drying processes: The HPβCD-FL118 complex preparation process was: (1) Twenty grams of HPβCD was put into a 200 mL glass bottle containing a magnetic stir bar and then 70 mL 100% ethanol was added. Then, the glass bottle was stirred on a Hotplate Stirrer at 30° C. under 1000 rpm overnight to make sure the HPβCD dissolved in the ethanol evenly to become a solution. (2) Then two grams GMP FL118 powder (batch No. C180402127-BF18001) was put into the ethanol-HPβCD solution and magnetically stirred under 1000 rpm at room temperature for 24 hours to make an ethanol-HPβCD-FL118 complex suspension at concentration of FL118 at 20 mg/mL. (3) Then, the spray-dryer (Model: Buchi B290) was employed to prepare HPβCD-FL118 complex powder from the stock suspension, which was stirred during spray drying. Detailed process parameters for spray drying are listed in Table 17. The spray-drying process-obtained HPβCD-FL118 complex powder was dried under vacuum at 30° C. overnight, and the yield of the products was 90.0% (Table 17). This batch was defied as FR00535-02-190904-01.

TABLE 17
Spray drying parameters for HPβCD-FL118 complex
preparation at the 2-g scale
Parameter settings

| Instrument | Buchi B290 |
|---|---|
| Solvent | EtOH |
| Concentration (mg/mL) | 20 mg/mL of API |
| Nozzle orifice size (mm) | 0.7 |
| Batch No | FR00535-02-190904-01 |
| | Stirring for 24 h |
| Set inlet Temp. (° C.) | 85 |
| Actual air Temp. (° C.) | 88 |
| Chamber out Temp. (° C.) | 61 |
| Q-flow. (Bar) | 0.3 |
| Pump (%) | 40 |
| Aspirator. (%) | 100 |
| Product amount (g) | 20.0 |
| Yield (%) | 90.0 |

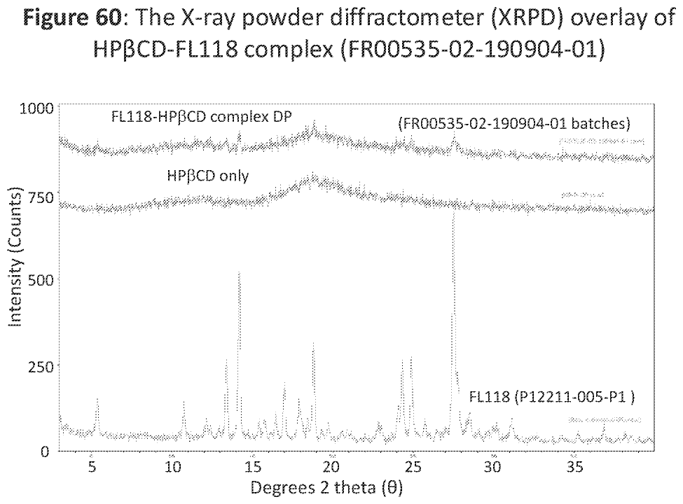

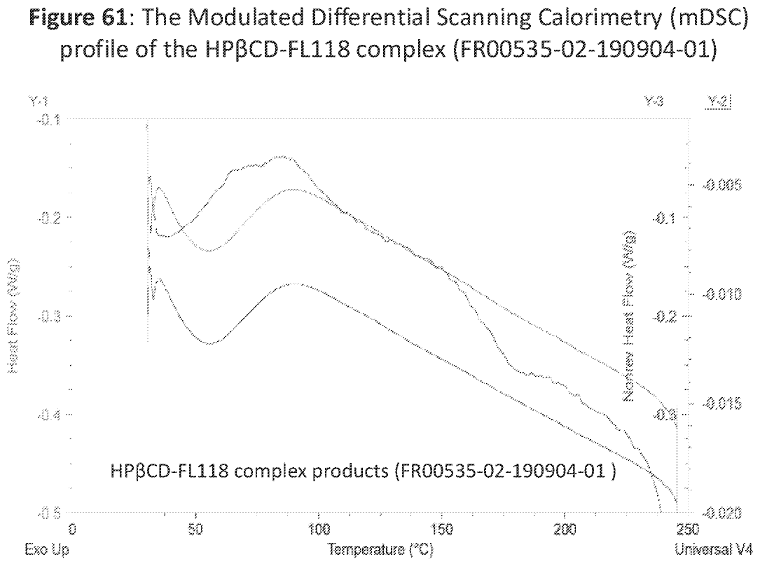

Example 51-Characterization of the HPβCD-FL118 complex powder of the two-gram scale level: The HPβCD-FL118 complex powder was characterized using X-ray powder diffractometer (XRPD), Modulated Differential Scanning calorimetry (mDSC) and High Performance Liquid Chromatography (HPLC). The results are summarized in Table 18. Based on XRPD results, similar to the 300 mg FL118 scale situation for the HPβCD-FL118 formulation, the HPβCD-FL118 complex products (Batch No.: FR00535-02-190904-01) were amorphous (FIG. 60). No glass transition temperature (Tg) of HPβCD-FL118 complex products (Batch No.: FR00535-02-190904-01) was observed in mDSC results (FIG. 61).

TABLE 18
Characterization of HPβCD-FL118
complex prepared via spray drying.

| | | | HPβCD-FL118 complex |
|---|---|---|---|
| Test item | | FL118 | (Stirring for 24 h) |
| Batch No. | | P12211-005-P1 | FR00535-02-190904-01 |
| Appearance | | Off-white powder | Off-white powder |
| XRPD | | Weak crystal | Amorphous |
| Melting point by DSC(° C.) | | No melting point | N/A |
| Tg by mDSC (° C.) | | N/A | No obvious glass |
| | | | transition temperature |
| Water content (%) | | 0.7 | 6.548 |
| Residual solvent | | 0.01 | 0.41 |
| (EtOH, %) | | | |
| HPLC test | Drug load (%) | N/A | 9.3 |
| | Purity (%) | 99.84 | 99.72 |

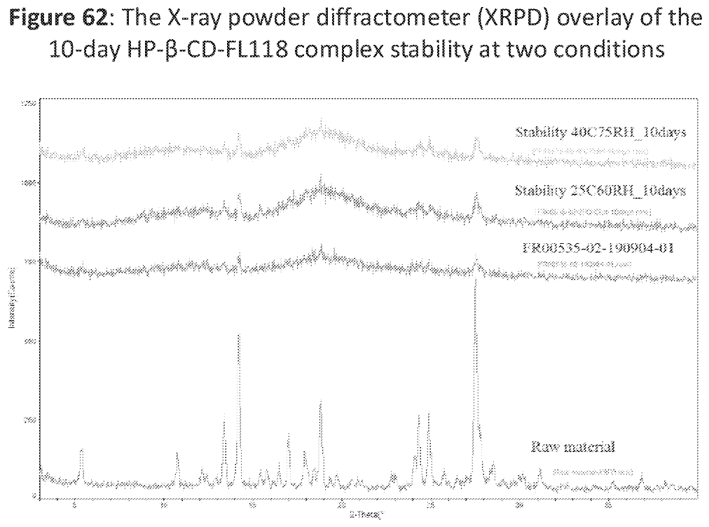

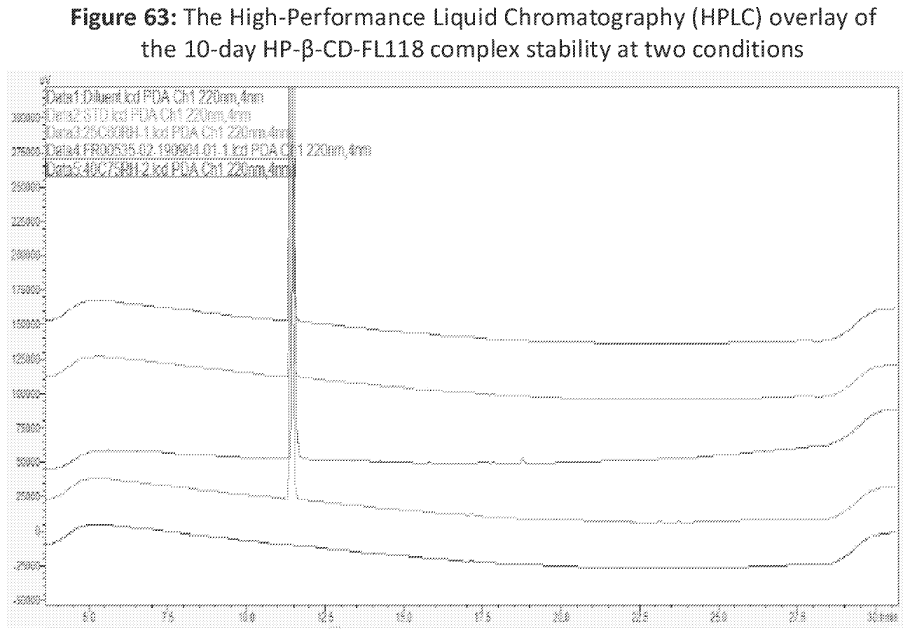

Example 52-HPβCD-FL118 API complex stability test and characterization: The HPβCD-FL118 complex samples (FR00535-02-190904-01) were set up for a 10-day stability study as shown in Table 19. About 20 mg of the HPβCD-FL118 complex (FR00535-02-190904-01) was weighed into 40 mL vials. Then the samples were covered by aluminum foil with pinholes and stored in a stability chamber at 25° C./60% RH (open) and 40° C./75% RH (open) for 10 days. The HPβCD-FL118 complex stability test results are summarized in Table 20 and Table 21. The analytical results from X-ray powder diffractometer (XRPD) and High-Performance Liquid Chromatography (HPLC) are presented in FIG. 62 and FIG. 63, respectively.

TABLE 19
HPβCD-FL118 complex 10-day stability study design

| Conditions | Initial | 10 days |
|---|---|---|
| 25° C./60% RH | XRPD analysis | XRPD analysis |
| | (Appearance and purity) | (Appearance and purity) |
| 40° C./75% RH | | XRPD analysis |
| | | (Appearance and purity) |

TABLE 20
HP-β-CD-FL118 complex 10-day stability test results

| | Initial | | 10 days | |
|---|---|---|---|---|
| Conditions | Appearance | Purity | Appearance | Purity |
| 25° C./60% RH | Off-white | 99.77 | Off-white | 99.79 |
| | powder | | powder | |
| 40° C./75% RH | | | Off-white | 99.80 |
| | | | powder | |

TABLE 21
Relative Retention Time (RRT) results of
10-day stability study at two conditions

| Relative | Area (%) | | | |
|---|---|---|---|---|
| Retention Time | | | 10 days | |
| (RRT) | STD | Initial | 25° C./60% RH | 40° C./75% RH |
| 1.00 | 99.91 | 99.77 | 99.79 | 99.80 |
| 1.17 | 0.9 | 0.8 | 0.7 | 0.7 |
| 1.38 | — | 0.15 | 0.14 | 0.13 |

Example 53-HPβCD-FL118 API complex dissolution test: The HPβCD-FL118 complex samples (batch: FR00535-02-190904-01) 220 mg was weighed into a 40 mL glass bottle containing a magnetic stir bar, added 20 mL 37° C. pH 1.2 buffer and pH 6.8 buffer into the vial, respectively; the vials were stirred at 100 rpm at 37±0.5° C. for 2 h. The sample solution of 300 μL was collected at each time point of 5, 10, 15, 30, 45, 60, 90, 120 min. The collected samples were analyzed for the concentrations of the supernatants by HPLC. The results are summarized in Table

22.

TABLE 22
Results of HPβCD-FL118 complex (Batch No.: FR00535-
02-190904-01) dissolutiontest in two mediums

| | Concentration (μg/mL) | | | | | | | |
|---|---|---|---|---|---|---|---|---|
| Time | pH 1.2 | | | | pH 6.8 | | | |
| (min) | API | HPβCD-FL118 complex | | | API | HPβCD-FL118 complex | | |
| 5 | 0.43 | 3.12 | 4.12 | 2.74 | 0.96 | 7.4 | 5.9 | 4.85 |
| 10 | 1.55 | 2.27 | 4.60 | 3.29 | 0.19 | 5.47 | 2.40 | 5.15 |
| 15 | 0.47 | 2.81 | 3.4 | 4.86 | 0.31 | 2.28 | 7.9 | 4.78 |
| 30 | 0.64 | 2.97 | 3.19 | 2.73 | 1.11 | 3.99 | 3.38 | 10.10 |
| 45 | 0.51 | 3.83 | 3.56 | 4.51 | 0.31 | 2.93 | 4.39 | 4.9 |
| 60 | 1.43 | 2.88 | 5.45 | 3.51 | 0.90 | 1.66 | 2.68 | 4.92 |
| 90 | 1.36 | 5.28 | 4.46 | 5.22 | 0.97 | 5.45 | 4.78 | 10.00 |
| 120 | 4.47 | 4.95 | 3.34 | 2.83 | 1.43 | 4.62 | 5.23 | 3.72 |

Example 54-Alternative process development of HPβCD-FL118 complex preparation by spray drying: The process was as follows. (1) A total of 10 grams of HPβCD was put into a 50 mL glass flask with a magnetic stir bar inside, and 40 mL 100% ethanol was added. The flask was then stirred at room temperature under 1000 rpm for 30 min, followed by adding 100% ethanol to a final volume of 50 mL to make a 20% HPβCD-ethanol solution by continuously magnetically stirring overnight. (2) Next day, 15 mL of the prepared 20% HPβCD-ethanol solution were transferred into each of three 40 mL glass bottles containing 300 mg GMP FL118 powder (batch No. C180402127-BF18001) and homogenized by Dispersion homogenizer (Model: T10 BASIC, IKA) at 5grade (every 10 min homogenizer followed by 5 min break) for different conditions: Condition 1: Homogenization for 40 min (total mix time 30 min). Condition 2: Homogenization for 85 min (total mix time 60 min). Condition 3: Homogenization for 175 min (total mix time 120 min). This generated three ethanol-HPβCD-FL118 suspensions with a concentration of FL118 at 20 mg/mL. (3) A Spray-Dryer (Model: Buchi B290) was subsequently employed to prepare HPβCD-FL118 complex powder. During the spray drying process, the ethanol-HPβCD-FL118 suspension stocks were stirred. Detailed process parameters for spray drying are summarized in Table 23. The obtained HPβCD-FL118 complex powders were dried under vacuum at 30° C. overnight, and the yield of the three products was 76.7%, 81.3% and 82.1% respectively (Table 23). This FL118 product batch was defined as FR00535-4-191022-01.

TABLE 23
A modified process for HPβCD-FL118 complex preparation
Parameter settings

| Instrument | | Buchi B290 | | |
|---|---|---|---|---|
| Solvent | | Ethanol (or methanol) | | |
| Concentration (mg/mL) | | 20 mg/mL of API | | |
| Nozzle orifice size (mm) | | 0.7 | | |
| Batch No | | FR00535-4-191022-01 | FR00535-4-191022-01 | FR00535-4-191022-01 |
| | | homogenizer for 30 min | homogenizer for 60 min | homogenizer for 120 min |
| | | (Condition 1) | (Condition 2) | (Condition 3) |
| Set inlet Temp. (° C.) | | 85 | 85 | 85 |
| Actual air Temp. (° C.) | | 85 | 85 | 83 |
| Chamber out Temp. (° C.) | | 57 | 59 | 56 |
| Q-flow. (Bar) | | 0.3 | | |
| Pump (%) | | 40 | | |
| Aspirator. (%) | | 100 | | |
| Product amount (g) | | 2.38 | 2.52 | 2.71 |
| Yield (%) | | 76.7 | 81.3 | 82.1 |
| HPLC | Theoretical | 9.70 | 9.70 | 9.12 |
| test | Drug load (%) | | | |
| | Real | 9.80 | 9.88 | 9.26 |
| | Drug load (%) | | | |
| | Purity (%) | 99.64 | 99.49 | 99.12 |

Example 55-Scale-up preparation of HPβCD-FL118 complex at the 10-gram level of FL118 API by spray drying: Based on the early small scale and the optimal 30-min homogenization, the process was then scaled up to 10g FL110 in this 30-min homogenization condition. The process is as follows. (1) A total of 100 grams of HPβCD was put into a 1000 mL glass bottle with a magnetic stir bar inside, and 400 mL 100% ethanol was added. The bottle was then stirred at 50° C. under 1000 rpm for 1h on a Hotplate Stirrer to make sure the HPβCD being completely dissolved in the ethanol evenly to become a complete solution. Then add additional ethanol to reach a total volume of 500 mL to make a 20% HPβCD ethanol solution. (2) Then, 10 grams of GMP FL118 powder (batch No. C180402127-BF18001) were put into the HPβCD-ethanol solution in the 1000 ml bottle, followed by homogenization using a Dispersion homogenizer (Model: T18, IKA) at 15000 rpm (every 5 min homogenizer followed by 5 min break) for 55 min (total mix time 30 min). The generated ethanol-HPβCD-FL118 suspension had a concentration of FL118 at 20 mg/mL. (3) While the HPβCD-FL118 complex suspension was stirred, the HPβCD-FL118 complex suspension was processed for spray drying using A spray-dryer equipment (Model: Buchi B290) to produce the HPβCD-FL118 complex powder product. Detailed process parameters for spray drying are presented in Table 24. The obtained HPβCD-FL118 complex powders were dried under vacuum at 30° C. overnight, and the yield of the products was 90.8% (Table 24). This FL 118 product was defined as a batch number of FR00535-5-191104-01.

TABLE 24
Spray drying parameters for HPβCD-FL118
complex preparation at 10 g scale
Parameter settings

| Instrument | Buchi B290 |
|---|---|
| Solvent | EtOH |
| Concentration (mg/mL) | 20 mg/mL of API (FL118: |
| | C180402127-BF18001) |
| Nozzle orifice size (mm) | 0.7 |
| Batch No | FR00535-5-191104-01 |
| | (Homogenizer for 0.5 h) |
| Set inlet Temp. (° C.) | 85 |
| Actual air Temp. (° C.) | 85 |
| Chamber out Temp. (° C.) | 58 |
| Q-flow. (Bar) | 0.41 |
| Pump (%) | 40 |
| Aspirator. (%) | 100 |
| Product amount (g) | 99.97 |
| Yield (%) | 90.8 |

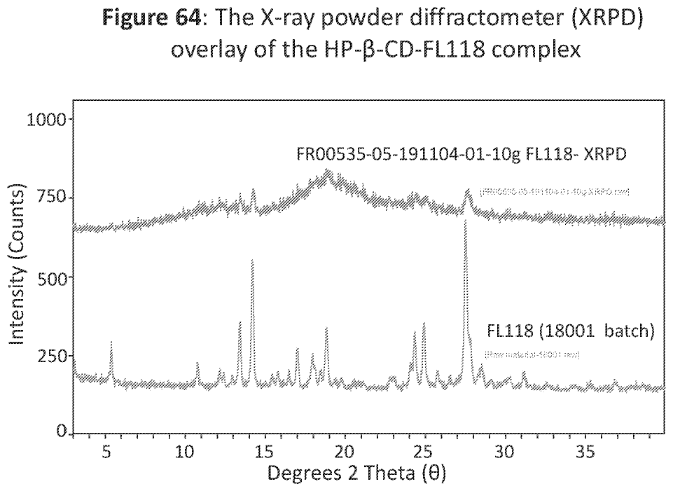

Example 56-Characterization of the HPβCD-FL118 complex at the 10-gram level of FL118 by X-ray powder diffractometer (XRPD): The obtained new batch HPβCD-FL118 complex powders (batch No.: FR00535-5-191104-01) were further characterized by XRPD. Results are summarized in Table 25. Based on XRPD results, the HPβCD-FL118 complex products were largely amorphous. The result from the XRPD analysis is shown in FIG. 64.

TABLE 25
Characterization for the scale-up HPβCD-FL118 complex
prepared via spray drying at the 10 g level of the FL118 API

| | | |
|---|---|---|
| | | Homogenizer |
| Test item | FL118 | for 0.5 h |

| Batch No. | | | batch No.: | FR00535-5- |
|---|---|---|---|---|
| | | | C180402127- | 191104-01 |
| | | | BF18001 | |
| Appearance | | | Off-white | Off-white |
| | | | powder | powder |
| XRPD | | | Weak crystal | Amorphous |
| | HPLC | Drug load (%) | N/A | 9.47 |
| | test | Purity (%) | 99.70 | 99.75 |

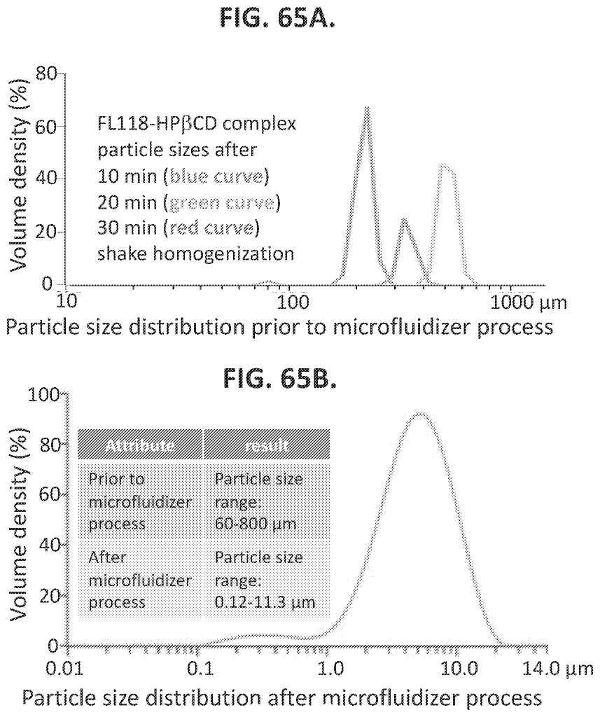

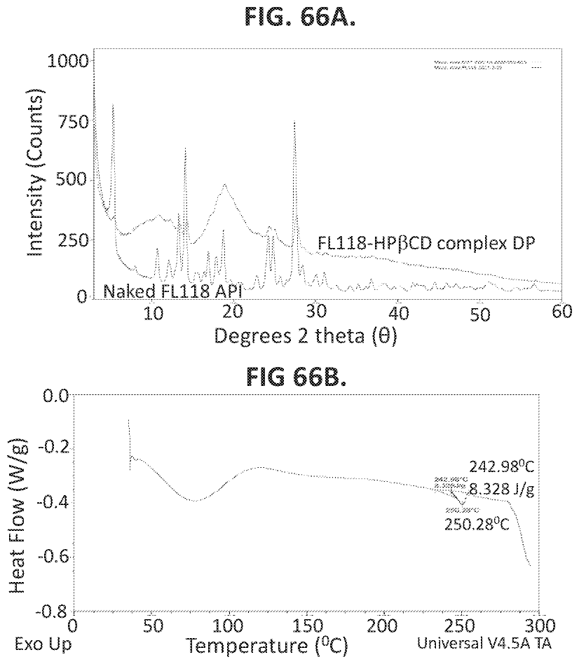

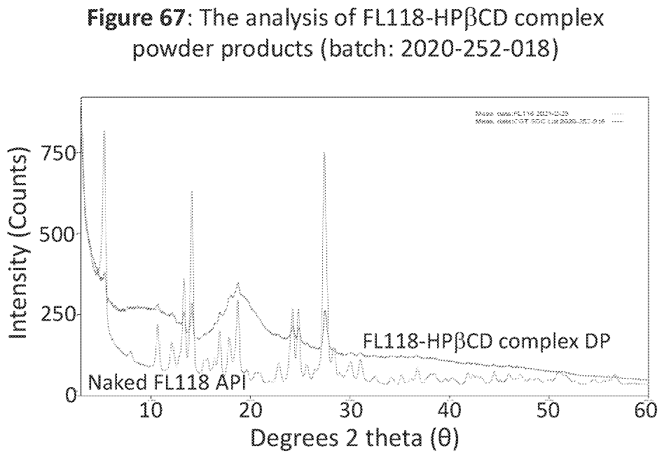

Example 57-Based on all these different formulation conditions and processes, glacial acetic acid is a better alternative organic solvent to be used for FL118 formulation with HPβCD. Through trial and error, 10% glacial acetic acid with 90% ethanol organic mixed solvent appears to be the optimal ratio for the formulation of FL118 API with HPβCD. In order to reduce FL118-HPβCD complex particle size to around 10 μm, the microfluidizer-process step was added and achieved the reduced FL118-HPβCD complex particle sizes from the range of 60-800 um to the particle sizes into the range of 0.1-11.3 μm (FIG. 65). Such fine FL118-HPβCD complex suspension in glacial acetic acid-ethanol solvent was then processed through Spray Dry Dispersion (SDD) to obtain FL118-HPβCD complex powder products (batch: 2020-252-23). XRPD analysis indicated that the product is in a co-amorphous status in parallel with micro-cocrystal status (FIG. 66A). Consistently, mDSC analysis indicated that there is a weak melting point (indicated by a weal glass transition temperature) at the approximate 250° C. (FIG. 66B), suggesting a portion of the FL118-HPβCD complex product is in a co-crystal status. Significantly, this phenomenon is quite consistency in different batches of glacial acetic acid and microfluidizer process-involved and SDD-processed FL118 DP as shown in a different batch (2020-252-18) (FIG. 67), which is important for further scaling up to make clinical batches in a GLP manufacturing condition. No observation of cocrystal status was found during pure ethanol-involved formulation of FL118 without the use of microfluidizer-mediated reduction of HPβCD-FL118 complex particles, and importantly such amorphous HPβCD-FL118 complex particles with partial cocrystal status exhibits higher antitumor efficacy with reduced toxicity to animals.

Example 58-Development of a clinically compatible approach for clinical batches of FL118 product manufacturing to match FL118 clinical trials: In preclinical in vivo human tumor animal model studies, a 2% hydroxypropyl methylcellulose (HPMC) in saline containing a range of 1-2.5% propylene glycol (PG) and 1-2.5% polyethylene glycol 400 or 300 (PEG400 or 300) was used to resuspend FL118 products for oral administration, which is not clinically compatible and thus could not be used in the next step FL118 clinical trials. So, (1) a pure ethanol-involved formulation of FL118 was further developed (2) glacial acetic acid-involved formulation of FL118 as disclosed in this patent together with the application of the state-of-the art microfluidizer-involved processing Spray-Dry Dispersion (M-SDD) technology to get rid of ethanol and/or glacial acetic acid from the fine size (FIG. 65B) of FL118-HPβCD complex suspension. Importantly, the resultant FL118-HPβCD complex powder product through such process can be simply resuspended with clinically used saline before animal or human use. Therefore, such FL118 product is clinically compatible with convenient use.

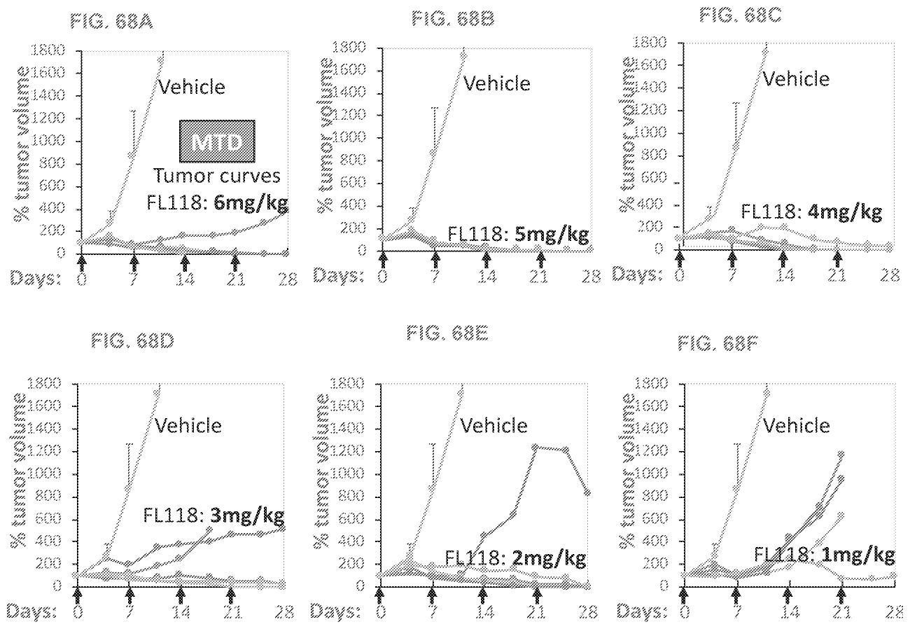

Example 59—The pure ethanol-involved SDD-processed FL118 products in the clinically compatible suspension exhibited great antitumor efficacy: Using FL118-HPβCD complex powder product manufactured by the hired CRO though the ethanol-involved SDD process, various aqueous solutions including clinical saline, saline with 1-2.5% PG, saline with 1-2.5% PG plus 1-2.5% PEG400, 2% milk and others were used to resuspend the FL118 product for oral administration. Simply resuspending the FL118 powder product in saline for oral administration is as good as these resuspended in other aqueous solution in terms of antitumor activity and toxicity. In comparison with the previous the preclinically compatible FL118-HPβCD suspension, the ethanol-processed formulation of FL118-HPβCD suspension extends FL118 ability to eliminate tumors at much lower dosing levels than the MTD level of 6 mg/kg (FIG. 68).

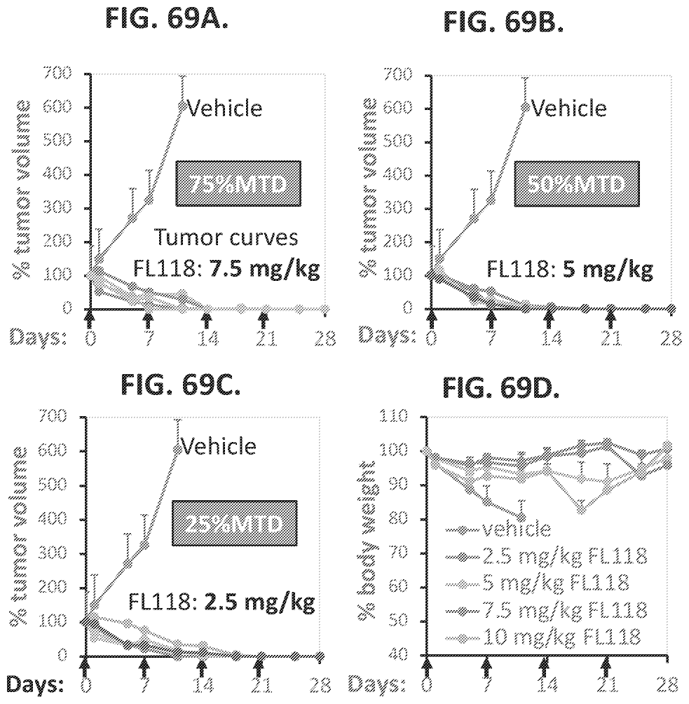

Example 60-Glacial acetic acid and microfluidizer-involved and SDD)-processed FL118 products possess even superior antitumor efficacy to the pure ethanol-involved SDD-processed FL118 products: Good FL118 oral bioavailability as disclosed early in this invention (Table 4, FIG. 37) implies that FL118 is resistant to stomach HCl acids. Attempting to further improve the FL118 efficacy-toxicity ratio, FDA-recommended acid solvents and their corresponding salts were screened to test the formulation of FL118 with HPβCD. Glacial acetic acid (GAA) was identified to be the best one through a series of in vivo human tumor animal model testing. Trying the FL118-HPβCD formulation using 100% glacial acetic acid, 50% glacial acetic acid with 50% ethonol and 10% glacial acetic acid with 90%, respectively, the following process-manufactured FL118-HPβCD complex powder product as exhibited superior antitumor efficacy with low toxicity: First dissolving HPβCD in the 10% glacial acetic acid/90% ethanol to make a master solution; and then formulating FL118 into the HPβCD loop structure in the master solution, then using microfluidizer to reduce FL118-HPβCD complex particle size prior to using Spray-Dry Technology to get rid of the organic solvent and obtain FL118-HPβCD complex powder products. A typical in vivo testing study is presented here using the saline resuspended FL118 products for oral administration. The glacial acetic acid involvement-manufactured FL118 products exhibited a superior capacity to eliminate APC/p53/Kras-triple mutated human SW620 colorectal cancer (CRC) tumors at both the maximum tolerated dose (MTD) and doses under MTD levels. In comparison with the ethanol-processed formulation of FL118-HPβCD suspension (FIG. 68), the glacial acetic acid-processed formulation of FL118-HPβCD suspension increased the FL118 MTD from 6 mg/kg to 7.5-10 mg/kg and extended the consistency of FL118 treatment-mediated elimination of human tumors under the MTD dose levels (FIG. 69ABC), while showing no unacceptable clinical toxicity including mouse body weight loss. Only a transient body weight decrease at the 10 mg/kg dose level was detected, but data were still within 20% (FIG. 69D).

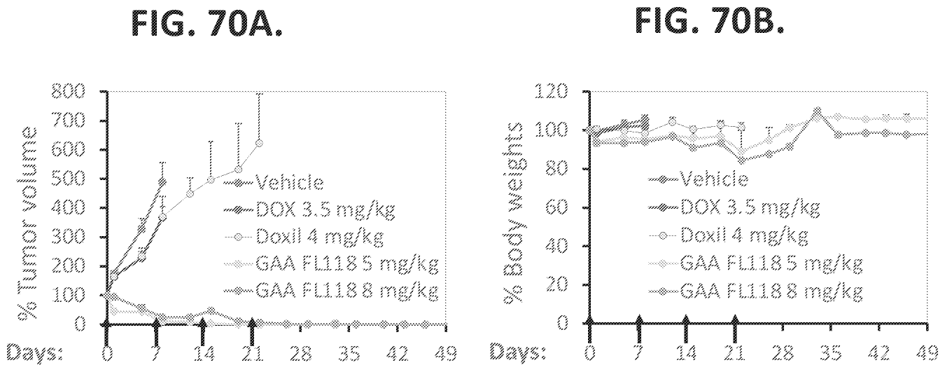

Example 61—Recent studies have demonstrated that DDX5 plays critical roles in multiple rare cancers inducing neuroblastoma, osteosarcoma and rthabdomyosarcoma and FL118 effectively regressed human soft tissue sarcoma (STS) tumors with favorable toxicity profile in animal models (Li F et al., J Exp Clin Cancer Res 2023, 42:213). Further studies demonstrated that FL118 also effectively eliminated human osteosarcoma xenograft tumors in animal models (FIG. 70). These results were not seen with doxorubicin (DOX) and Doxil (FIG. 70). These exciting results further reveal the great potential for FL118 as a demonstrated triple-target inhibitor to simultaneously inhibit DDX5, USP2a and UbE2T as demonstrated in this invention everywhere.

Example 62—Toxicology and MTD studies with 10 rats per group (5 males and 5 males) using the non-clinically compatible FL118 product (performed by Covance): Sprague Dawley SDR rats had high toxicity sensitivity to the preclinically compatible FL118 product formulated in 0.44% HPβCD, 2% HPMC and 1% PG in saline. Specifically, among the mouse FL118 MTD-calculated 3 dosing levels (1.65 mg/kg, 3.3 mg/kg and 6.6 mg/kg, weekly), oral administration of 3.3 or 6.6 mg/kg/day FL118 was not tolerated and resulted in the moribund condition/death of the experimental rats on Day 4 or 5 with significant body weight loss following the first dose (Table 26). Only the rats administered at 1.65 mg/kg weekly survived to their scheduled sacrifice. Brown haircoat in the perineal area, thin, hunched, and mucoid feces were noted on Days 4 and 5 but no longer observed by Day 6 at the dose of 1.65 mg/kg weekly. A transient body weight loss of 9% and 4% were observed in males and females, respectively, from Days 1 through 4 (Table 26), which was generally similar to controls from Days 4 to 8. Lower food consumption was also observed from Days 1 through 4, which was resolved in females and partly resolved in males from Days 4 to 9 at 1.65 mg/kg/dose.

TABLE 26
Summary of Mean Body Weights by Group

| | Sex | | | | | | | |
|---|---|---|---|---|---|---|---|---|
| | Male | | | | Female | | | |
| Dose (mg/kg/dose) | 0 | 1.65 | 3.3 | 6.6 | 0 | 1.65 | 3.3 | 6.6 |
| Day 1 (g) | 208 | 207 | 207 | 206 | 189 | 188 | 192 | 189 |
| Day 4 (g) | 221 | 189* | 183* | 181* | 192* | 180* | 168* | 169* |
| BW Change (g) | 14 | −18* | −25* | −25* | 2 | −7* | −26* | −21* |
| % BW Change (Day 4) | 7% | −9% | −12% | −12% | 1% | −4% | −14% | −11% |
| Day 8 (g) | 239 | 202 | NA | NA | 193 | 181 | NA | NA |
| BW Change (g) | 18 | 13 | NA | NA | 4 | 1 | NA | NA |
| % BW Change (Day 8) | 12% | 15% | NA | NA | 2% | 1% | NA | NA |

− = Decrease; BW = Body Weight; NA = Not applicable due to rats in moribund condition/death.
*= Statistically significant (P ≤0.5).

Several FL118-related clinical pathology effects at the dose of 1.65 mg/kg were observed, although it was much weaker than the two high dose levels (3.3 mg/kg and 6.6 mg/kg). Hematology findings including mildly lower red cell mass (males only); markedly lower reticulocyte count; markedly lower platelet count in a few individual animals; moderately lower counts in white blood cell, lymphocyte, monocyte, eosinophil, basophil, and LUC in males and eosinophil and LUC in females were observed (Table 27).

TABLE 27
Effects of FL118 versus vehicle on rat' hematological parameters on day 10.

| | RBC* | HGB | HCT | MCV | MCH | MCHC | RDW | RET | PLT | WBC |
|---|---|---|---|---|---|---|---|---|---|---|
| | (M/μL) | (g/dL) | (%) | (fL) | (pg) | (g/dL) | (%) | (K/μL) | (K/μL) | (K/μL) |
| Male rats | | | | | | | | | | |
| Vehicle (mean, N = 3): | 8.31 ± 0.3 | 16.0 ± 0.4 | 47.9 ± 0.2 | 57.7 ± 2.0 | 19.3 ± 0.2 | 33.4 ± 1.0 | 12.3 ± 0.2 | 285 ± 52.1 | 994 ± 147 | 8.9 ± 0.8 |
| FL118# (mean, N = 4): | 6.91 ± 1.1 | 13.5 ± 2.1 | 40.1 ± 6.8 | 58.0 ± 1.1 | 19.5 ± 0.3 | 33.7 ± 0.8 | 11.8 ± 0.2 | 26.4 ± 19.6 | 361 ± 334 | 4.0 ± 0.7 |
| Female rats | | | | | | | | | | |
| Vehicle (mean, N = 5): | 8.11 ± 0.4 | 15.3 ± 0.6 | 44.2 ± 1.9 | 54.5 ± 0.8 | 18.9 ± 0.4 | 34.6 ± 0.4 | 11.0 ± 0.2 | 158 ± 36.5 | 868 ± 165 | 4.5 ± 1.0 |
| FL118# (mean, N = 4): | 7.6 ± 1.1 | 14.4 ± 1.3 | 41.6 ± 6.4 | 54.8 ± 1.6 | 18.9 ± 0.7 | 34.5 ± 0.3 | 10.9 ± 0.2 | 15.6 ± 10.0 | 672 ± 7783 | 352 ± 1.0 |

| | NEUT | LYM | MONO | EOS | BASO | LUC^{a} | PT | APTT | FIB |
|---|---|---|---|---|---|---|---|---|---|
| | (K/μL) | (K/μL) | (K/μL) | (K/μL) | (K/μL) | (K/μL) | (sec) | (sec) | (mg/dL) |
| Male rats | | | | | | | | | |
| Vehicle (mean, N = 3): | 0.91 ± 0.2 | 37.6 ± 0.5 | 0.18 ± 0.13 | 0.06 ± 0.03 | 0.09 ± 0.03 | 0.06 ± 0.01 | 9.6 ± 0.9 | 17.9 ± 1.8 | 241 ± 10.6 |
| FL118# (mean, N = 4): | 1.13 ± 0.6 | 2.81 ± 0.5 | 0.04 ± 0.03 | 0.03 ± 0.03 | 0.02 ± 0.01 | 0.01 ± 0.01 | 8.7 ± 0.2 | 14.7 ± 2.1 | 241 ± 10.6 |
| Female rats | | | | | | | | | |
| Vehicle (mean, N = 5): | 0.71 ± 0.2 | 3.6 ± 1.0 | 0.07 ± 0.02 | 0.10 ± 0.04 | 0.03 ± 0.01 | 0.02 ± 0.01 | 8.5 ± 0.2 | 17.4 ± 4.2 | 225 ± 20.0 |
| FL118# (mean, N = 4): | 1.13 ± 1.2 | 2.28 ± 1.1 | 0.06 ± 0.07 | 0.02 ± 0.00 | 0.02 ± 0.01 | 0.01 ± 0.01 | 8.3 ± 0.15 | 15.5 ± 1.9 | 221 ± 10.1 |

*RBC, red blood cell; HGB, hemoglobin; HCT, hematocrit; MCV, RBC mean cell volume; MCH, mean corpuscular/cell hemoglobin; MCHC, mean corpuscular/cell hemoglobin concentration; RDW, red cell distribution; RET, reticulocyte; PLT, platelet; WBC, white blood cell; NEUT, neutrophil; LYM, lymphocyte; MONO, monocyte; EOS, eosinophil; BASO, basophil; LUC, large unstained cells; PT, prothrombin time; APTT, activated partial thromboplastin time: FIB, Fibrinogen. M, million; K, 1000/thousand.
#FL118 data from the highest non-severely toxic dose; (HNSTD) at 1.65 mg/kg weekly (2 doses at days 1, 8) - a dose slightly higher than MTD

Other clinical pathology findings (may be a secondary due to reduced food consumption) included mildly lower total protein, albumin, and globulin concentrations; mildly higher albumin: globulin ratio; moderately lower triglyceride concentration (males only); mildly lower calcium concentration; and mildly lower aspartate aminotransferase (females only), alanine aminotransferase (males only), and alkaline phosphatase activities (Table 28). However, no FL118-related findings were observed in coagulation test results.

TABLE 28
Effects of FL118 versus vehicle on rats' serum biochemical parameters on day 10.

| | GLU* | UN | CREA | TP | ALB | GLOB | A:G | CHOL | TRIG | TBIL |
|---|---|---|---|---|---|---|---|---|---|---|
| | (mg/L) | (mg/dL) | (mg/dL) | (g/dL) | (g/dL) | (g/dL) | Ratio | (mg/dL) | (mg/dL) | (mg/dL) |
| Male rats | | | | | | | | | | |
| Vehicle | 80 ± 9.7 | 15 ± 2.3 | 0.3 ± 0.05 | 6.1 ± 0.15 | 4.1±0.1 | 2.0 ± 0.13 | 2.1 ± 0.13 | 153 ± 19.8 | 75 ± 20.6 | 0.1 ± 0.00 |
| (mean, N = 5): | | | | | | | | | | |
| FL118# | 146 ± 67.1 | 18 ± 4.4 | 0.3 ± 0.05 | 5.5 ± 0.6 | 3.9 ± 0.4 | 1.7 ± 0.2 | 2.4 ± 0.15 | 121 ± 26.0 | 32 ± 5.7 | 0.1 ± 0.00 |
| (mean, N = 5): | | | | | | | | | | |
| Female rats | | | | | | | | | | |
| Vehicle | 85 ± 11.1 | 19 ± 1.6 | 0.3 ± 0.05 | 6.3 ± 0.3 | 4.2 ± 0.24 | 2.1 ± 0.13 | 2.0 ± 0.12 | 108 ± 14.8 | 46 ± 6.9 | 0.1 ± 0.00 |
| (mean, N = 5): | | | | | | | | | | |
| FL118# | 114 ± 17 | 22 ± 13.2 | 0.3 ± 0.04 | 5.6 ± 0.66 | 3.9 ± 0.41 | 1.7 ± 0.3 | 2.3 ± 0.25 | 112 ± 36.5 | 50 ± 12.9 | 0.1 ± 0.04 |
| (mean, N = 5): | | | | | | | | | | |

| | AST | ALT | ALP | GGT | CK | Ca | PHOS | Na | K | Cl |
|---|---|---|---|---|---|---|---|---|---|---|
| | (U/L) | (U/L) | (U/L) | (U/L) | (U/L) | (mg/dL) | (mg/dL) | (mmol/L) | (mmol/L) | (mmol/L) |
| Male rats | | | | | | | | | | |
| Vehicle | 102±15 | 66 ± 7.6 | 164 ± 14.1 | <3 ± 0.0 | 1234±1290 | 10.5±0.12 | 8.5 ± 0.36 | 143 ± 0.8 | 5.4 ± 0.52 | 100 ± 2.0 |
| (mean, N = 5): | | | | | | | | | | |
| FL118# | 90 ± 4.5 | 47 ± 7.5 | 123 ± 27.3 | <3 ± 0.0 | 1045 ± 463 | 9.9 ± 0.48 | 8.2 ± 0.45 | 143 ± 2.0 | 5.3 ± 0.61 | 101 ± 1.5 |
| (mean, N = 5): | | | | | | | | | | |
| Female rats | | | | | | | | | | |
| Vehicle | 94 ± 7.9 | 47 ± 7.6 | 82 ± 11.0 | <3 ± 0.0 | 901 ± 296.6 | 10.3 ± 0.1 | 6.8 ± 1.32 | 144 ± 1.2 | 5.3 ± 0.09 | 102 ± 1.3 |
| (mean, N = 5): | | | | | | | | | | |
| FLI18# | 80 ± 9.9 | 39 ± 4.9 | 56 ± 11.3 | <3 ± 0.0 | 669 ± 290.5 | 9.9 ± 0.34 | 6.8 ± 0.56 | 144 ± 0.7 | 5.0 ± 0.38 | 103 ± 0.5 |
| (mean, N = 5): | | | | | | | | | | |

*GLU, glucose; UN/BUN, (blood) urea nitrogen; CREA, creatinine; TP, total protein; ALB, albumin; GLOB, globulin; A:G, albumin:globulin; CHOL, cholesterol; TRIG, triglyceride; TBIL, total bilirubin; AST, aspartate aminotransferase; ALT, alanine transaminase/aminotransferase; ALP/ALKP, alkalinephosphatase; GGT, gamma glutamyl transferase; CK, creatine kinase; Ca, calcium; PHOS, phosphorus; Na, sodium; K, potassium; Cl, chloride.
#FL118 data from the highest non-severely toxic dose; (HNSTD) at 1.65 mg/kg weekly (2 doses at days 1, 8) - a dose slightly higher than MTD

In conclusion, oral gavage administration of the non-clinically compatible FL118 product (formulated in 0.44% HPβCD, 2% HPMC and 1% PG in saline) at dose levels of 1.65, 3.3, or 6.6 mg/kg/dose once weekly for two doses one day 1 and day 8 was tolerated at the 1.65 mg/kg/dose in male and female rats. However, this dose level resulted in pathology changes. Thus, the highest non-severely toxic dose (HNSTD) is 1.65 mg/kg/dose, which is a dosing level slightly higher than MTD.

Example 63-Toxicology and MTD studies with a limited member of rats (2 males and 2 males per group) using clinically compatible FL118 product (LOT 2020-252-23): The toxicology/MTD studies in rats using the non-clinically compatible FL118 product suspension got 1.65 mg/kg/dose as the FL118 HNSTD (a dose slightly higher then MTD). Therefore, for testing the clinically compatible FL118 product suspension, initially 4-dose levels of 0.8, 1.0, 1.25 and 1.56 mg/kg was used on dosing days 1, 8, 15 and 22 for the Groups 2-5, respectively, as outlined in Table 29. Because no rats at these doses exhibited obvious toxicity signals, three increased doses of 1.95, 2.4 and 3.4 mg/kg were given on dosing days 29 and 36 for the Groups 2-4, respectively (Table 29). In order to observe a clearer toxicity, further two increased doses of 3.81 and 4.77 mg/kg were given on dosing day 43 for the Groups 2 and 3 (Table 29), respectively.

TABLE 29
FL118 oral treatment groups for toxicology testing with a limited number of rats

| | | Rat | | Number | |
|---|---|---|---|---|---|
| Treatment | FL118 Dosage (mg/kg) versus using | Identification Codes | | of Rats | |
| Group | dose concentration (mg/mL) | Male | Female | Male | Female |

Dosing days 1, 8, 15, and 22 (dose volume: 5 mL/kg)

| | | | | | |
|---|---|---|---|---|---|
| Group 1 | (0 mg/kg) vs. using (0 mg/mL) | Rat 1 | Rat 10 | 1 | 1 |
| Group 2 | (0.8 mg/kg) vs. using (0.16 mg/mL) | Rats 2, 3 | Rats 11, 12 | 2 | 2 |
| Group 3 | (1.0 mg/kg) vs. using (0.2 mg/mL) | Rats 4, 5 | Rats 13, 14 | 2 | 2 |
| Group 4 | (1.25 mg/kg) vs. using (0.25 mg/mL) | Rats 6, 7 | Rats 15, 16 | 2 | 2 |
| Group 5 | (1.56 mg/kg) vs. using (0.31 mg/mL) | Rats 8, 9 | Rats 17, 18 | 2 | 2 |

Dosing days 29 and 36 (dose volume: 5 mL/kg)

| | | | | | |
|---|---|---|---|---|---|
| Group 1 | (0 mg/kg) vs. using (0 mg/mL) | Rat 1 | Rat 10 | 1 | 1 |
| Group 2 | (1.95 mg/kg) vs. using (0.39 mg/mL) | Rats 2, 3 | Rats 11, 12 | 2 | 2 |
| Group 3 | (2.4 mg/kg) vs. using (0.48 mg/mL) | Rats 4, 5 | Rats 13, 14 | 2 | 2 |
| Group 4 | (3.05 mg/kg) vs. using (0.61 mg/mL) | Rats 6, 7 | Rats 15, 16 | 2 | 2 |
| Group 5 | (1.56 mg/kg) vs. using (0.31 mg mL) | Rats 8, 9 | Rats 17, 18 | 2 | 2 |

Dosing day 43 (dose volume: 5 mL/kg)

| | | | | | |
|---|---|---|---|---|---|
| Group 1 | (0 mg/kg) vs. using (0 mg/mL) | Rat 1 | Rat 10 | 1 | 1 |
| Group 2 | (3.81 mg/kg) vs. using (0.76 mg/mL) | Rats 2, 3 | Rats 11, 12 | 2 | 2 |
| Group 3 | (4.77 mg/kg) vs. uSng (0.95 mg/mL) | Rats 4, 5 | Rats 13, 14 | 2 | 2 |
| Group 4 | (3.05 mg/kg) vs. using (0.61 mg/mL) | Rats 6, 7 | Rats 15, 16 | 2 | 2 |
| Group 5 | (1.56 mg/kg) vs. using (0.31 mg/mL) | Rats 8, 9 | Rats 17, 18 | 2 | 2 |

Dose administration by orogastric gavage (i.e., orally) was successful at all time points, and all rats received their target dose volume. No mortality occurred during the study. All rats survived to the completion of the study and were euthanized 7 days after final dosing on Day 50.

There were no serious adverse clinical signs seen in female rats in any of the Treatment Groups. Select male rats in the Treatment Groups receiving the 3 highest doses of FL118 (Group 4, 7th dose [3.5 mg/kg], Group 2, 7th dose [3.81 mg/kg], and Group 3, 7th dose [4.77 mg/kg]) experienced mild adverse clinical signs for 4 to 7 days after dosing. Clinical signs included hunched posture and ruffled fur, seen in all these 3 groups; porphyrin staining around the eyes and on fur on the muzzle, seen in Groups 4 (3.5 mg/kg) and 3 (4.77 mg/kg); and diarrhea and squinting of the eyes, seen in Group 3 (4.77 mg/kg). Adverse clinical signs were dose dependent, as they were seen only in the 3 highest dose groups and were most severe in the highest dose group, and were also sex dependent, as they were seen only in males. In all instances the seen clinical signs were mild and did not require treatment or intervention.

Orogastric gavage administration of FL118 had adverse effects on body weights in male rats receiving 2.44, 3.5, and 3.81 mg/kg. For all the female rats in all the treatment groups the weight loss was mild and did not exceed 10% of their total body weight. Select male rats receiving 2.44 mg/kg FL118, 3.5 mg/kg FL118, and 3.81 mg/kg FL118 had weight loss that exceeded 10% of their pre-dose baseline body weight following administration. Male rat #4 in Group 3 received 2.44 mg/kg FL118 on Day 36 (dose 6) and experienced >10% weight loss on Days 39-41. Male rats #6 and #7 in Group 4 received 3.5 mg/kg FL118 on Day 29 (dose 5), Day 36 (dose 6), and Day 43 (dose 7) and experienced >10% body weight loss on Days 33-34 (rat #7), Days 32-34 (rat #6), Day 32 (rat #7), Days 47-49 (rat #7) and Day 48 (rat #6). Male rat #3 in Group 2 received 3.81 mg/kg FL118 on Day 43 (dose 7) and experienced >10% weight loss on Days 46-49. Weight loss following administration of FL118 was dose related, occurring in 2nd, 3rd, and 4th, highest dose groups, and sex-related, occurring only in males.

Clinical hematology: Select rats from Groups treated with FL118 dosing levels of 1.56, 3.5 and 4.77 mg/kg had various hematology values and some of them slightly outside the normal reportable range (Table 30). However, this was not related to administration of FL118, as it occurred in the vehicle-treated control group as well. In short, administration of FL118 did not have adverse effects on hematology values (Table 30).

TABLE 30
Effects of FL118 versus vehicle on rats' hematological parameters.

| | RBC* | HGB | HCT | MCV | MCH | MCHC | RDW-SD | RET |
|---|---|---|---|---|---|---|---|---|
| | (M/μL) | (g/dL) | (%) | (fL) | (pg) | (g/dL) | (fL) | (K/μL) |
| Normal range: | 6.44-8.7 | 12.5-16.2 | 40.1-52.3 | 57.1-65.2 | 18.1-20.0 | 30.0-32.2 | 24.9-34.3 | 187-689 |
| Vehicle: | 8.4-9.4 | 15.4-16.1 | 48.2-51.7 | 55.2-57.4 | 17.2-18.3 | 31.2-32.0 | 28.1-28.3 | 319.5-327.6 |

FL118 (at different dosing levels)

| | | | | | | | | |
|---|---|---|---|---|---|---|---|---|
| 1.56 mg/kg: | 7.67-8.73 | 14.2-16 | 44.5-50.5 | 56.1-58.6 | 17.8-18.6 | 31.2-32.1 | 30-33.7 | 401-524 |
| 3.05 mg/kg: | 6.15-8.04 | 11.8-14.8 | 34.6-46.5 | 55.6-57.8 | 18.4-19.2 | 31.8-34.1 | 29.3-35.3 | 208-625 |
| 4.77 mg/kg: | 6.72-8.77 | 12-16.1 | 36-50.3 | 53.6-57.4 | 17.9-18.6 | 32-33.3 | 26.2-33.4 | 21-460 |

| | PLT | PDW | MPV | WBC | NEUT | LYM | MONO | EOS | BASO |
|---|---|---|---|---|---|---|---|---|---|
| | (K/μL) | (fL) | (fL) | (K/μL) | (K/μL) | (K/μL) | (K/μL) | (K/μL) | (K/μL) |
| Normal range: | 506-1143 | 7.5-9.9 | 7.8-10 | 5.75-17 | 0.21-1.1 | 5.07-15 | 0-1.51 | 0-0.16 | 0-0.03 |
| Vehicle: | 911-914 | 9.7-10 | 8.1-8.5 | 7.3-15.8 | 0.68-1.3 | 6.3-13.4 | 0.23-1.0 | 0.05-0.16 | 0.02-0.03 |

FL118 (at different dosing levels)

| | | | | | | | | | |
|---|---|---|---|---|---|---|---|---|---|
| 1.56 mg/kg: | 864-1070 | 9.2-10 | 7.9-8.1 | 6.5-10.7 | 0.38-1.37 | 5.56-8.7 | 0.19-0.58 | 0.04-0.07 | 0.01-0.02 |
| 3.05 mg/kg: | 665-1259 | 9.4-9.9 | 7.6-7.8 | 6.0-7.9 | 0.92-1.42 | 4.83-6.03 | 0.09-0.43 | 0.02-0.1 | 0.01-0.04 |
| 4.77 mg/kg: | 316-1442 | 8.8-9.7 | 7.3-9.3 | 5.8-11.0 | 0.08-0.48 | 5.31-7.9 | 0.08-3.41 | 0.03-0.08 | 0-0.02 |

*RBC, red blood cell; HGB, hemoglobin; HCT, hematocrit; MCV, RBC mean cell volume; MCH, mean corpuscular/cell hemoglobin; MCHC, mean corpuscular/cell hemoglobin concentration; RDW-SD, red cell distribution width-standard deviation; RET, reticulocyte; PLT, platelet; PDW, platelet distribution width; MPV, mean platelet volume; WBC, white blood cell; NEUT, neutrophil; LYM, lymphocyte; MONO, monocyte; EOS, eosinophil; BASO, basophil. M, million; K, 1000/thousand.

Clinical chemistry: All evaluated rats from Groups treated with vehicle and FL118 at 1.56, 3.5 and 4.77 mg/kg had elevated levels of serum glucose due to not having been fasted prior to blood collection. Select rats from Groups treated with FL118 dosing levels of 1.56, 3.5 and 4.77 mg/kg had various clinical chemistry values, and some of them slightly outside the normal reportable range (Table 31). However, this was not related to administration of FL118, as it also occurred in the vehicle-treated control group as well. In short, administration of FL118 did not have adverse effects on clinical chemistry values (Table 31).

TABLE 31
Effects of FL118 versus vehicle on rats' serum biochemical parameters

| | GLU^{#} | BUN | CREA | PHOS | Ca | TP | ALB | ALT |
|---|---|---|---|---|---|---|---|---|
| | (mg/dL) | (mg/dL) | (mg/dL) | (mg/dL) | (mg/dL) | (g/dL) | (g/dL) | (U/L) |
| Normal range: | 50-135 | 9-21 | 0.1-0.6 | 5.8-11.2 | 5.3-11.6 | 5.3-6.9 | 3.8-4.8 | 20-61 |
| Vehicle: | 353-439 | 17-18 | 0.4-0.5 | 10-11.9 | 12.1-12.2 | 6.5-6.6 | 3.4-4.8 | 93-109 |

FL118 (at different dosing levels)

| | | | | | | | | |
|---|---|---|---|---|---|---|---|---|
| 1.56 mg/kg: | 206-296 | 15-20 | 0.1-0.5 | 10.8-13.2 | 11.2-11.6 | 6.2-6.7 | 3.4-4.4 | 63-134 |
| 3.05 mg/kg: | 282-438 | 15-21 | 0.1-0.5 | 4.1-10.5 | 11.3-11.9 | 5.4-6.3 | 3.6-4.3 | 85-111 |
| 4.77 mg/kg: | 229-433 | 16-23 | 0.1-0.5 | 8.5-12.9 | 11-12.1 | 5.5-6.0 | 3.0-4.0 | 60-145 |

| | ALP | TBIL | CHOL | AMYL | K | Na | Cl |
|---|---|---|---|---|---|---|---|
| | (U/L) | (mg/dL) | (mg/dL) | (U/L) | (mmol/L) | (mmol/L) | (mmol/L) |
| Normal range: | 16-302 | 0.1-0.7 | 20-92 | 326-2246 | 5.0-11 | 130-160 | 100-110 |
| Vehicle: | 199-337 | 0.1-0.4 | 99-117 | 1609-2263 | ≥10 | 146-153 | 102-106 |

FL118 (at different dosing levels)

| | | | | | | | |
|---|---|---|---|---|---|---|---|
| 1.56 mg/kg: | 111-239 | 0.2-0.8 | 89-111 | 1547-2358. | 9.4->10 | 143-154 | 102-107 |
| 3.05 mg/kg: | 37-168 | 0.3-0.7 | 38-87 | 1614-2162. | 9.8->10 | 147-154 | 104-108 |
| 4.77 mg/kg: | 27-159 | 0.3-0.8 | 63-99 | 1518-2449. | 9.4->10 | 143-152 | 103-109 |

^{#}GLU, glucose; BUN/UN, (blood) urea nitrogen; CREA, creatinine; PHOS, phosphorus; Ca, calcium; TP, total protein; ALB, albumin; ALT, alanine transaminase/aminotransferase; ALP/ALKP, alkalinephosphatase; TBIL, total bilirubin; CHOL, cholesterol; AMYL, amylase; K, potassium; Na, sodium; Cl, chloride.

Additionally, no obvious FL118-related gross pathology macroscopic findings (gross lesions) were noted for all doses used at the end of the study doses of Group 1 (vehicle control), Group 2 (3.81 mg/kg FL118), Group 3 (4.77 mg/kg FL118), Group 4 (3.5 mg/kg FL118) and Group 5 (1.56 mg/kg FL118). A conservative MTD for oral administration is ≥2.44 mg/kg for male rats (likely at ˜3 mg/kg) and ≤4.77 mg/kg for female rats.

Example 64-Toxicology and MTD studies with the FDA requirement-matched standard numbers of rats (5 males and 5 males per group) using clinically compatible FL118 product (LOT 2020-252-23): The Canget-hired CRO WuXi AppTec Hong Kong Toxicology subsidiary performed the study. Twenty female and 20 male rats (Crl: CD® [SD] VAF/Plus®/SPF) were randomly assigned to 4 groups. Then vehicle (Group 1) and FL118 (the glacial acetic acid/ethanol and microfluidizer-involved SDD-processed FL118-HPβCD complex powder product, LOT 2020-252-23) was orally dosed on Days 1 and 8 via oral gavage as defined in Table 32. Based on the toxicity/MTD testing outcome derived from limited numbers of rats presented in the Example 62 above, the starting dose levels in the three FL118 test groups 2, 3 and 4 were 3 mg/kg, 3.75 mg/kg and 4.69 mg/kg on Day 1 were used (Table 32). However, based on the rat body weight and food consumption situation, for the FL118 oral treatment on Day 8 the FL118 dose levels 3 mg/kg, 2.5 mg/kg and 2 mg/kg in the three test groups 2, 3 and 4 were adjusted as shown in Table 32.

TABLE 32
FL118 oral treatment groups for toxicology testing with the standard numbers of rats.

| | | FL118 Doses ^{a} | | | | | Main study | |
|---|---|---|---|---|---|---|---|---|
| Group | Group | Dose | | Volume | | Conc. | numbers of animals | |
| Numbers | Designation | (mg/kg/wk) | | (mL/kg) | | (mg/mL) | M | F |
| 1 | Control/HPβCD ^{b} | 0 | | 5 | | 0 | 1001-1005 | 1501-1505 |
| 2 | Dose 1(FL118) | 3 | (Day 1) | 5 | (Day 1) | 0.6 | 2001-2005 | 2501-2505 |
| | | 3 | (Day 8) | 5 | (Day 8) | | | |
| 3 | Dose 2 ^{c} (FL118) | 3.75 | (Day 1) | 5 | (Day 1) | 0.75 | 3001-3005 | 3501-3505 |
| | | 2.5 | (Day 8) | 3.33 | (Day 8) | | | |
| 4 | Dose 3 ^{c} (FL118) | 4.69 | (Day 1) | 5 | (Day 1) | 0.94 | 4001-4005 | 4501-4505 |
| | | 2 | (Day 8) | 2.13 | (Day 8) | | | |

Note:
In this report, “dose level” and “dosage” are used interchangeably.
^{a} Doses represent active ingredient.
^{b} The control formulation was 9.4 mg/mL HPβCD in vehicle. For control group, the dose of HPβCD was 47 mg/kg/wk.
^{c} Due to body weights and food consumption decreased were noted in the first week, the dose of group 3 and 4 was reduced on Day 8.
Conc. = Concentration M = Male F = Female TK = Toxicokinetics.

In the defined FL118 treatment profile described above (Table 32), in the group 3 mice treated with the dose of 4.69 mg/kg/wk (given on day 1) and 2 mg/kg/wk (given on day 8), two male mice died on day 6 and day 12, respectively, and 1 male mouse was euthanized due to clinical observations. Before being found dead, activity decreased, prostration, cold to touch, thin and abnormal stool were noted in these animals. All other male and female mice survived to scheduled terminations. No test article-related changes were noted on coagulation and urinalysis at scheduled terminations.

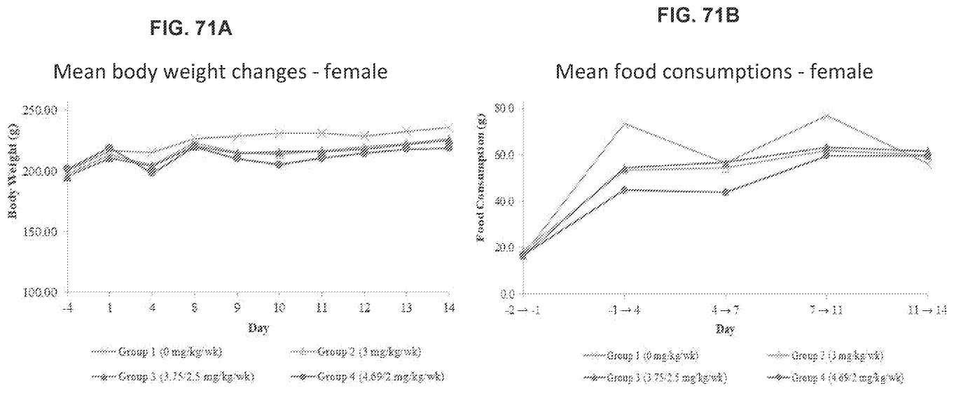

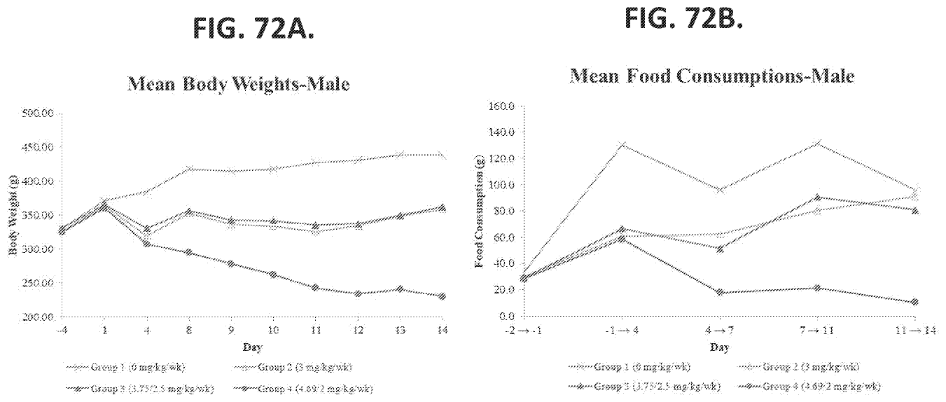

When compared with concurrent controls, while no significant female rat body weight loss and food consumption decrease were found (FIG. 71), statistically significant decreased body weight was noted in scheduled, terminated males at 3, 3.75→2.5, and 4.69→2 mg/kg/wk, decrease of 18.15%, 17.41% and 47.53% in males on Day 14 (FIG. 72A). These changes correlated with lower food consumption with body weight loss (FIG. 72B).

FL118 related hematologic changes were limited to decreased leukocyte counts (WBC) and lymphocytes (LYMP) on Day 15. Specifically, decreased WBC was noted in males at 3, 3.75→2.5 and 4.69→2 mg/kg/wk and in females at 4.69→2 mg/kg/wk on Day 15. Decreased LYMP was noted in both sexes at 4.69→2 mg/kg/wk on Day 15 (Table 33). Additional hematology parameters were shown in detail for male rats in Table 34 and for female rats in Table 35.

TABLE 33
FL118-related changes in hematology on Day 15 (percent
difference in mean values from concurrent controls)

| | Dose (mg/kg/wk) | | | | | | | |
|---|---|---|---|---|---|---|---|---|
| | 0 | | 3 | | 3.75→ 2.5 | | 4.69→ 2 | |
| Sex | Male | Female | Male | Female | Male | Female | Male | Female |
| No. of animals | 5 | 5 | 5 | 5 | 5 | 5 | 2 | 5 |
| WBC (10³/μL) | 10.74 | 7.31 | −16.11% | — | −11.98% | — | −52.17% | −33.69% |
| #LYMP (10³/μL) | 9.16 | 6.47 | — | — | — | — | −57.17% | −37.76% |

— No noteworthy change
Data are shown as group means for the control group and percent difference in mean values vs. concurrent control means for test article groups.

TABLE 34
Effects of FL118 (Group 2, 3, 4) versus vehicle (group 1) on male rats' hematological parameters (WuXi AppTec)
Day 15: Relative to the start date (Day 1)

| | | WBC | RBC | HGB | HCT | MCV | MCH | MCHC | RDW | #RET | % RET | #NEUT | % NEUT |
|---|---|---|---|---|---|---|---|---|---|---|---|---|---|
| | | (10\hat{ }3/μL) | (10\hat{ }6/μL) | (g/dL) | (%) | (fL) | (pg) | (g/dL) | (%) | (10\hat{ }9/L) | (%) | (10\hat{ }3/μL) | (%) |
| Sex: Male | | [a] | [a] | [a] | [a] | [a] | [a] | [a] | [a1] | [a2] | [a2] | [a] | [a] |
| Group 1, | Mean | 10.74 | 7.63 | 15.1 | 45.8 | 60.0 | 19.8 | 33.1 | 11.7 | 220.3 | 2.89 | 1.09 | 10.4 |
| 0 mg/kg/wk | SD | 3.78 | 0.22 | 0.5 | 2.1 | 1.6 | 0.4 | 1.0 | 0.2 | 24.1 | 0.27 | 0.35 | 1.9 |
| | N | 5 | 5 | 5 | 5 | 5 | 5 | 5 | 5 | 5 | 5 | 5 | 5 |
| Group 2, | Mean | 9.01 | 7.22 | [symbol] | 42.1* | 58.3 | 19.2 | 32.8 | 13.5** | 337.2 | 4.68 | 0.72 | 7.9 |
| 3 mg/kg/wk | SD | 2.89 | 0.37 | 0.7 | 2.2 | 1.4 | 0.9 | 1.0 | 1.4 | 115.8 | 1.66 | 0.29 | 1.2 |
| | N | 5 | 5 | 5 | 5 | 5 | 5 | 5 | 5 | 5 | 5 | 5 | 5 |
| | % Diff | −16.11 | [symbol] | −8.9 | −8.0 | −2.7 | −3.4 | −0.8 | 15.7 | 53.1 | 62.09 | −34.07 | −24.1 |
| Group 3, | Mean | 9.43 | [symbol] | 13.8** | 42.3* | 58.2 | 19.0 | 32.6 | 14.1*** | 342.9 | 4.74 | 1.04 | 11.2 |
| 3.75/2.5 | SD | 0.98 | 0.29 | 0.3 | 1.3 | 3.4 | 0.9 | 0.9 | 1.4 | 209.2 | 2.97 | 0.18 | 2.4 |
| mg/kg/wk | N | 5 | 5 | 5 | 5 | 5 | 5 | 5 | 5 | 5 | 5 | 5 | 5 |
| | % Diff | −11.98 | [symbol] | −8.6 | −7.5 | −2.9 | −4.1 | −1.3 | 20.5 | 55.7 | 64.10 | −4.40 | 7.9 |
| Group 4, | Mean | [symbol] | [symbol] | [symbol] | [symbol] | [symbol] | [symbol] | [symbol] | [symbol] | [symbol] | [symbol] | [symbol] | [symbol] |
| 4.69/2 | SD | 1.96 | 0.59 | 1.3 | 4.5 | 1.4 | 0.1 | [symbol] | 0.6 | — | — | 0.28 | 12.9 |
| mg/kg/wk | N | 2 | 2 | 2 | 2 | 2 | 2 | 2 | 2 | 1 | 1 | 2 | 2 |
| | % Diff | −52.17 | 8.98 | 7.1 | 1.7 | −6.8 | −1.7 | 5.4 | 4.3 | −87.2 | −87.53 | −19.87 | 88.7 |

[a] - Anova & Dunnett:
*= p ≤ 0.05;
**= p ≤ 0.01; n—Inappropriate for statistics
[a1] - Kruskal-Wallis & Dunnett on Ranks:
**= p ≤ 0.01;
***=p ≤ 0.001; n—Inappropriate for statistics
WBC, white blood cell; RBC, red blood cell; HGB, hemoglobin; HCT, hematocrit; MCV, mean corpuscular volume; MCH, mean corpuscular
hemoglobin; MCHC, mean corpuscular hemoglobin concentration; RDW, RBC distribution width; #RET, reticulocytes, absolute; % RET,
reticulocytes, percent; #NEUT, neutrophils, absolute; % NEUT, neutrophils, percent.

| | | #LYMP | % LYMP | #MONO | % MONO | #EOS | % EOS | #BASO | % BASO | PLT | MPV |
|---|---|---|---|---|---|---|---|---|---|---|---|
| | | (10\hat{ }3/μL) | (%) | (10\hat{ }3/μL) | (%) | (10\hat{ }3/μL) | (%) | (10\hat{ }3/μL) | (%) | (10\hat{ }3/μL) | (fL) |
| Sex: Male | | [a] | [a] | [a] | [a] | [a] | [a] | [a] | [a] | [a] | [a] |
| Group 1, | Mean | 9.16 | 85.0 | 0.29 | 2.7 | 0.11 | 1.1 | 0.02 | 0.2 | 1024 | 6.8 |
| 0 mg/kg/wk | SD | 3.38 | 2.2 | 0.08 | 0.3 | 0.64 | 0.6 | 0.02 | 0.1 | 94 | 0.2 |
| | N | 5 | 5 | 5 | 5 | 5 | 5 | 5 | 5 | 5 | 5 |
| Group 2, | Mean | 7.94 | 88.2 | 0.21 | 2.5 | 0.06 | 0.7 | 0.02 | 0.2 | 1172 | 6.6 |
| 3 mg/kg/wk | SD | 2.36 | 1.1 | 0.09 | 1.3 | 0.03 | 0.4 | 0.01 | 0.0 | 106 | 0.3 |
| | N | 5 | 5 | 5 | 5 | 5 | 5 | 5 | 5 | 5 | 5 |
| | % Diff | −13.38 | 3.7 | −25.17 | −6.6 | −43.40 | −35.2 | −25.00 | −8.3 | 14 | −2.7 |
| Group 3, | Mean | 8.02 | 84.7 | 0.23 | 2.4 | 0.07 | 0.8 | 0.02 | 0.2 | 1129 | 6.5 |
| 3.75/2.5 | SD | 1.04 | 3.0 | 0.08 | 0.7 | 0.06 | 0.7 | 0.01 | 0.1 | 190 | 0.3 |
| mg/kg/wk | N | 5 | 5 | 5 | 5 | 5 | 5 | 5 | 5 | 5 | 5 |
| | % Diff | −12.51 | −0.4 | −18.88 | −10.3 | −30.19 | −22.2 | [symbol] | [symbol] | 10 | [symbol] |
| Group 4, | Mean | [symbol] | [symbol] | [symbol] | [symbol] | [symbol] | [symbol] | [symbol] | [symbol] | [symbol] | [symbol] |
| 4.69/2 | SD | 1.89 | 8.2 | 0.30 | 4.1 | 0.01 | 0.1 | 0.00 | 0.1 | 58 | 0.3 |
| mg/kg/wk | N | 2 | 2 | 2 | 2 | 2 | 2 | 2 | 2 | 2 | 2 |
| | % Diff | −57.17 | −11.9 | −0.35 | 76.5 | −95.28 | −95.4 | −58.33 | −37.5 | −11 | −5.3 |

[a2]—Anova & Dunnett: n—Inappropriate for statistics.
#LYMP, lymphocytes, absolute; % LYMP, lymphocytes, percent; #MONO, monocytes, absolute; % MONO, monocytes, percent; #EOS, eosinophils, absolute; % EOS, eosinophils, percent; #BASO, basophils, absolute; % BASO, basophils, percent; PLT, platelet count; MPV, mean platelet volume. [symbol]
indicates data missing or illegible when filed

TABLE 35
Effects of FL118 (Group 2, 3, 4) versus vehicle (group 1) on female rats' hematological parameters (WuXi)
Day 15: Relative to the start date (Day 1)

| | | WBC | RBC | HGB | HCT | MCV | MCH | MCHC | RDW | #RET | % RET | #NEUT | % NEUT |
|---|---|---|---|---|---|---|---|---|---|---|---|---|---|
| | | (10\hat{ }3/μL) | (10\hat{ }6/μL) | (g/dL) | (%) | (fL) | (pg) | (g/dL) | (%) | (10\hat{ }9/μL) | (%) | (10\hat{ }3/μL) | (%) |
| Sex: Female | | [a] | [a] | [a1] | [a1] | [a] | [a] | [a] | [a1] | [a] | [a] | [a] | [a] |
| Group 1, | Mean | 7.31 | 7.39 | 14.0 | 41.4 | 56.1 | 19.0 | 33.8 | 10.9 | 180.9 | 7.44 | 0.56 | 8.0 |
| 0 mg/kg/wk | SD | 0.82 | 0.26 | 0.4 | 0.7 | 1.3 | 0.3 | 0.6 | 0.5 | 55.0 | 0.69 | 0.31 | 5.2 |
| | N | 5 | 5 | 5 | 5 | 5 | 5 | 5 | 5 | 5 | 5 | 5 | 5 |
| Group 2, | Mean | 6.67 | 7.25 | 13.5 | 40.4 | 55.8 | 18.6 | 33.4 | 12.5* | 308.1 | 4.27 | 0.57 | 9.1 |
| 3 mg/kg/wk | SD | 2.79 | 0.20 | 0.4 | 1.3 | 1.4 | 0.2 | 0.7 | 0.6 | 115.8 | 1.66 | 0.17 | 2.2 |
| | N | 5 | 5 | 5 | 5 | 5 | 5 | 5 | 5 | 5 | 5 | 5 | 5 |
| | % Diff | −8.73 | −1.92 | −3.8 | −2.5 | −0.6 | −1.8 | −1.2 | 14.9 | 70.3 | 74.92 | 1.42 | 14.0 |
| Group 3, | Mean | 7.03 | 6.88 | 13.2 | 19.8 | 58.0 | 19.3 | 33.3 | 12.9* | 340.1 | 5.05 | 0.87 | 12.1 |
| 3.75/2.5 | SD | 1.27 | 0.52 | 0.5 | 2.3 | 1.2 | 0.7 | 0.6 | 1.6 | 162.0 | 7.62 | 0.48 | 5.6 |
| mg/kg/wk | N | 5 | 5 | 5 | 5 | 5 | 5 | 5 | 5 | 5 | 5 | 5 | 5 |
| | % Diff | −3.78 | −6.90 | −5.7 | −3.9 | 3.3 | 1.8 | −1.6 | 19.2 | 88.0 | 106.89 | 53.55 | 51.0 |
| Group 4, | Mean | 4.85 | 6.94 | 13.6 | 40.0 | 57.8 | 19.6 | 33.8 | 13.7* | 311.2 | 4.79 | 0.62 | 12.7 |
| 4.69/2 | SD | 1.33 | 0.67 | 1.2 | 2.9 | 1.6 | 0.4 | 0.5 | 3.4 | 265.6 | 4.42 | 0.21 | 2.5 |
| mg/kg/wk | N | 5 | 5 | 5 | 5 | 5 | 5 | 5 | 5 | 5 | 5 | 5 | 5 |
| | % Diff | −33.69 | −6.04 | −3.4 | −3.4 | 3.0 | 3.2 | 0.1 | 26.3 | 72.0 | 96.48 | 9.22 | 58.8 |

[a] - Anova & Dunnett
[a1] - Kruskal-Wallis & Dunnett on Ranks
*= p ≤ 0.05
WBC, white blood cell; RBC, red blood cell; HGB, hemoglobin; HCT, hematocrit, MCV, mean corpuscular volume; MCH, mean corpuscular
hemoglobin; MCHC, mean corpuscular hemoglobin concentration; RDW, RBC distribution width; #RET, reticulocytes, absolute; % RET,
reticulocytes, percent; #NEUT, neutrophils, absolute; % NEUT, neutrophils, percent.

| | | #LYMP | % LYMP | #MONO | % MONO | #EOS | % EOS | #BASO | % BASO | PLT | MPV |
|---|---|---|---|---|---|---|---|---|---|---|---|
| | | (10\hat{ }3/μL) | (%) | (10\hat{ }3/μL) | (%) | (10\hat{ }3/μL) | (%) | (10\hat{ }3/μL) | (%) | (10\hat{ }3/μL) | (fL) |
| Sex: Female | | [a] | [a] | [a] | [a] | [a] | [a] | [a] | [a] | [a] | [a] |
| Group 1, | Mean | 6.47 | 88.3 | 0.15 | 2.0 | 0.07 | 1.0 | 0.01 | 0.1 | [symbol] | 6.7 |
| 0 mg/kg/wk | SD | 0.94 | 5.3 | 0.04 | 0.4 | 0.02 | 0.2 | 0.00 | 0.0 | 141 | 0.2 |
| | N | 5 | 5 | 5 | 5 | 5 | 5 | 5 | 5 | 5 | 5 |
| Group 2, | Mean | 5.86 | 87.1 | 0.13 | 1.9 | 0.06 | 1.1 | 0.01 | 0.2 | 1310 | 68 |
| 3 mg/kg/wk | SD | 2.56 | 2.5 | 0.06 | 0.1 | 0.02 | 0.3 | 0.01 | 0.1 | 181 | 0.2 |
| | N | 5 | 5 | 5 | 5 | 5 | 5 | 5 | 5 | 5 | 5 |
| | % Diff | −9.52 | −1.3 | −9.59 | −1.0 | −11.76 | 12.5 | −16.67 | 33.3 | 19 | 1.5 |
| Group 3, | Mean | 5.87 | 83.5 | 0.17 | 2.4 | 0.07 | 1.0 | 0.01 | 0.2 | 1295 | 6.8 |
| 3.75/2.5 | SD | 1.09 | 4.8 | 0.07 | 0.9 | 0.01 | 0.3 | 0.01 | 0.0 | 253 | 0.2 |
| mg/kg/wk | N | 5 | 5 | 5 | 5 | 5 | 5 | 5 | 5 | 5 | 5 |
| | % Diff | −9.36 | −5.5 | 16.44 | 22.4 | 0.00 | 4.2 | −16.67 | 50.0 | 18 | 2.7 |
| Group 4, | Mean | 4.03 | 82.7 | 0.13 | 2.9 | 0.05 | 1.0 | 0.01 | 0.1 | 1278 | 6.7 |
| 4.69/2 | SD | 1.16 | 3.2 | 0.06 | 1.7 | 0.02 | 0.4 | 0.01 | 0.1 | 136 | 0.5 |
| mg/kg/wk | N | 5 | 5 | 5 | 5 | 5 | 5 | 5 | 5 | 5 | 5 |
| | % Diff | −37.76 | −6.3 | −12.33 | 46.9 | −32.35 | 4.2 | −50.00 | 16.7 | 16 | 0.3 |

[a] - Anova & Dunnett
#LYMP, lymphocytes, absolute; % LYMP, lymphocytes, percent; #MONO, monocytes, absolute; % MONO, monocytes, percent; #EOS, eosinophils, absolute; % EOS, eosinophils, percent; #BASO, basophils, absolute; % BASO, basophils, percent; PLT, platelet count, MPV, mean platelet volume. [symbol]
indicates data missing or illegible when filed

FL118-related serum chemistry changes were limited to increased alanine aminotransferase (ALT) and decreased glucose (sGLU). Specifically, increased ALT was noted in males at 3, 3.75→2.5 and 4.69→2 mg/kg/wk and in females at 4.69→2 mg/kg/wk and decreased sGLU was noted in both sexes at 4.69→2 mg/kg/wk on Day 15 (Table 36). Additional serum chemistry parameters were shown in detail for male rats in Table 37 and for female rats in Table 38.

TABLE 36
FL118-related changes in serum chemistry on Day 15 (percent
difference in mean values from concurrent controls)

| | Dose (mg/kg/wk) | | | | | | | |
|---|---|---|---|---|---|---|---|---|
| | 0 | | 3 | | 3.75→2.5 | | 4.69→2 | |
| Sex | Male | Female | Male | Female | Male | Female | Male | Female |
| No. of animals | 5 | 5 | 5 | 5 | 5 | 5 | 2 | 5 |
| ALT (U/L) | 35 | 34 | 19% | — | 34% | — | 275% | 122% |
| sGLU(mmol/L) | 10.88 | 10.24 | — | — | — | — | −59.96% | −19.47% |

— No noteworthy change
Data are shown as group means for the control group and percent difference in mean values vs. concurrent control means for test article groups.

TABLE 37
Effects of FL118 (Group 2, 3, 4) versus vehicle (Group 1) on male rats' serum chemical parameters (WuXi AppTec)
Day 15: Relative to the start date (Day 1)

| | | ALT | AST | TP | ALB | BIL-T | ALP | | sGLU | UREA | CRE | [symbol] | P |
|---|---|---|---|---|---|---|---|---|---|---|---|---|---|
| | | (U/L) | (U/L) | (g/L) | (g/L) | ( [symbol] /L) | (U/L) | GGT | (mmol/L) | (mmol/L) | (μmol/L) | (mmol/L) | (mmol/L) |
| Sex: Male | | [a] | [a] | [a] | [a] | [a] | [a] | (U/L) | [a] | [a] | [a] | [a] | [a] |
| Group 1, | Mean | 35 | 136 | 58.0 | 34.4 | 1.71 | 162 | — | 10.88 | 6.57 | 20 | 2.40 | 2.49 |
| 0 mg/kg/wk | SD | 6 | 31 | 2.4 | 1.2 | 0.17 | 45 | — | 2.95 | 0.42 | 3 | 0.08 | 0.16 |
| | N | 5 | 5 | 5 | 5 | 5 | 5 | 0 | 5 | 5 | 5 | 5 | 5 |
| Group 2, | Mean | 42 | 100 | 55.5 | 33.3 | 2.14 | 146 | — | 10.72 | 6.25 | 19 | 2.44 | 2.39 |
| 3 mg/kg/wk | SD | 6 | 19 | 29 | 1.5 | 0.40 | 38 | — | 1.64 | 0.84 | 2 | 0.06 | 0.15 |
| | N | 5 | 5 | 5 | 5 | 5 | 5 | 0 | 5 | 5 | 5 | 5 | 5 |
| | % Diff | 19 | −27 | −4.3 | −3.2 | 25.44 | −9 | — | −1.42 | −4.56 | −2 | 1.66 | −4.02 |
| Group 3, | Mean | 47 | 104 | 56.1 | 34.1 | 1.83 | 113 | — | 10.18 | 5.60 | 19 | 2.41 | 2.38 |
| 3.75/2.5 | SD | 9 | 20 | 3.1 | 1.8 | 0.26 | 33 | — | 1.14 | 0.63 | 2 | 0.10 | 0.34 |
| mg/kg/wk | N | 5 | 5 | 5 | 5 | 5 | 5 | 0 | 5 | 5 | 5 | 5 | 5 |
| | % Diff | 34 | −24 | −3.2 | −1.0 | 8.21 | −30 | — | −6.38 | −13.45 | −3 | 0.08 | −4.34 |
| Group 4, | Mean | [symbol] | [symbol] | [symbol] | [symbol] | [symbol] | [symbol] | — | [symbol] | [symbol] | [symbol] | [symbol] | [symbol] |
| 4.69/2 | SD | 134 | 311 | 0.0 | 2.7 | 4.86 | 5 | — | 1.42 | 10.61 | 3 | 0.02 | 0.72 |
| mg/kg/wk | N | 2 | 2 | 2 | 2 | 2 | 2 | 0 | 2 | 2 | 2 | 2 | 2 |
| | % Diff | 27.5 | 1.54 | −33.1 | −28.9 | 187.51 | −45 | — | −50.96 | 71.64 | −3 | −15.35 | −13.59 |

[a] - Anova & Dunnett: n—Innapropriate for statistics
ALT, alanine aminotransferase ; AST, aspartate aminotransferase; TP, total protein; ALB, albumin; BIL-T, total bilirubin (diasys); ALP,
alkaline phosphatase; GGT, gamma-glutamyltransferase; sGLU, glucose; UREA, urea; CRE, creatinine; Ca, calcium; P, inorganic phosphorus.

| | | TCHO | TG | K | Na | Cl | GLB | | CK |
|---|---|---|---|---|---|---|---|---|---|
| | | (mmol/L) | (mmol/L) | (mmol/L) | (mmol/L) | (mmol/L) | (g/L) | A/G | (U/L) |
| Sex: Male | | [a] | [a] | [a1] | [a] | [a1] | [a] | [a] | [a] |
| Group 1, | Mean | 1.28 | 0.30 | 5.0 | 142 | 103 | 23.5 | 1.47 | 528 |
| 0 mg/kg/wk | SD | 0.08 | 0.07 | 0.4 | 1 | 3 | 1.8 | 0.12 | 239 |
| | N | 5 | 5 | 5 | 5 | 5 | 5 | 5 | 5 |
| Group 2, | Mean | 2.18*** | 0.36 | 4.9 | 143 | 103 | 22.1 | 1.51 | 260* |
| 3 mg/kg/wk | SD | 0.35 | 0.18 | 0.2 | 1 | 1 | 1.6 | 0.08 | 88 |
| | N | 5 | 5 | 5 | 5 | 5 | 5 | 5 | 5 |
| | % Diff | 69.94 | 18.54 | −2.0 | 1 | 1 | −6.0 | 2.75 | −51 |
| Group 3, | Mean | 1.73* | 0.33 | 5.0 | 143 | 105 | 22.0 | 1.56 | 296 |
| 3.75/2.5 | SD | 0.29 | 0.19 | 0.2 | 2 | 1 | 1.8 | 0.12 | 117 |
| mg/kg/wk | N | 5 | 5 | 5 | 5 | 5 | 5 | 5 | 5 |
| | % Diff | 34.42 | 10.60 | 0.4 | 0 | 2 | −6.5 | 5.96 | −44 |
| Group 4, | Mean | [symbol] | [symbol] | [symbol] | [symbol] | [symbol] | [symbol] | [symbol] | [symbol] |
| 4.69/2 | SD | 0.52 | 0.51 | 0.5 | 5 | 6 | 2.7 | 0.52 | 257 |
| mg/kg/wk | N | 2 | 2 | 2 | 2 | 2 | 2 | 2 | 2 |
| | % Diff | 9.81 | 52.32 | 2.6 | −1 | −3 | −39.2 | 19.83 | 13 |

[a] - Anova & Dunnett:
*= p ≤ 0.05;
***=p ≤ 0.001; n—Innapropriate for statistics
[a1] - Kruskal-Wallis & Dunnett on Ranks; n—Innapropriate for statistics
TCHO, total cholesterol; TG, triglyceride; K, potassium; Na, sodium; Cl, chloride; GLB, globulin; A/G, A/G ratio; CK, creatine kinase. [symbol]
indicates data missing or illegible when filed

TABLE 38
Effects of FL118 (Group 2, 3, 4) versus vehicle (Group 1) on female rats' serum chemical parameters (WuXi AppTec)
Day 15: Relative to the start date (Day 1)

| | | ALT | AST | TP | ALB | BIL-T | ALP | | sGLU | UREA | CRE | Ca | P |
|---|---|---|---|---|---|---|---|---|---|---|---|---|---|
| | | (U/L) | (U/L) | (g/L) | (g/L) | (μmol/L) | (U/L) | GGT | (mmol/L) | (mmol/L) | (μmol/L) | (mmol/L) | (mmol/L) |
| Sex: Female | | [a] | [a1] | [a2] | [a2] | [a1] | [a2] | (U/L) | [a2] | [a2] | [a2] | [a2] | [a2] |
| Group 1, | Mean | 34 | 108 | [symbol] | 37.6 | 1.73 | 105 | — | 10.24 | 8.08 | 24 | 2.46 | 2.09 |
| 0 mg/kg/wk | SD | 5 | 28 | 4.7 | 2.8 | 0.47 | 36 | — | 1.58 | 1.43 | 2 | 0.10 | 0.20 |
| | N | 5 | 5 | 5 | 5 | 5 | 5 | 0 | 5 | 5 | 5 | 5 | 5 |
| Group 2, | Mean | 32 | 137 | 58.4 | 35.7 | 1.96 | 101 | — | 8.47 | 7.24 | 24 | 2.41 | 2.16 |
| 3 mg/kg/wk | SD | 6 | 33 | 3.3 | 1.8 | 0.28 | 29 | — | 1.79 | 2.81 | 3 | 0.09 | 0.24 |
| | N | 5 | 5 | 5 | 5 | 5 | 5 | 0 | 5 | 5 | 5 | 5 | 5 |
| | % Diff | −5 | 27 | −4.7 | −4.9 | 13.44 | −3 | — | −17.32 | −10.39 | 3 | −2.12 | 3.35 |
| Group 3, | Mean | 33 | 89 | [symbol] | 37.5 | 1.59 | 83 | — | 9.43 | 6.29 | 24 | 2.42 | 2.20 |
| 3.75/2.5 | SD | 8 | 13 | 1.7 | 0.8 | 0.19 | 24 | — | 1.16 | 1.35 | 3 | 0.03 | 0.30 |
| mg/kg/wk | N | 5 | 5 | 5 | 5 | 5 | 5 | 0 | 5 | 5 | 5 | 5 | 5 |
| | % Diff | −4 | −17 | −1.1 | −0.2 | −7.76 | −21 | — | [symbol] | −22.22 | 0 | −1.38 | [symbol] |
| Group 4, | Mean | 75 | 222 | 58.5 | 36.6 | 2.66 | 88 | — | 8.25 | 9.54 | 24 | 2.37 | 1.92 |
| 4.69/2 | SD | 96 | 268 | 5.1 | 1.7 | 1.91 | 25 | — | 3.16 | 4.47 | 4 | 0.10 | 0.23 |
| mg/kg/wk | N | 5 | 5 | 5 | 5 | 5 | 5 | 0 | 5 | 5 | 5 | 5 | 5 |
| | % Diff | 122 | 106 | −4.4 | −2.7 | 54.23 | −16 | — | −19.47 | 18.06 | 2 | −3.66 | −8.41 |

[a] - Kruskal-Wallis & Dunnett on Ranks
[a1] - Anova & Dunnett(Log)
[a2] - Anova & Dunnett
ALT, alanine aminotransferase; AST, aspartate aminotransferase; TP, total protein; ALB, albumin; BIL-T, total bilirubin (diasys); ALP,
alkaline phosphatase; GGT, gamma-glutamyltransferase; sGLU, glucose; UREA, urea; CRE, creatinine; Ca, calcium; P, inorganic phosphorus.

| | | TCHO | TG | K | Na | Cl | GLB | | CK |
|---|---|---|---|---|---|---|---|---|---|
| | | (mmol/L) | (mmol/L) | (mmol/L) | (mmol/L) | (mmol/L) | (g/L) | A/G | (U/L) |
| Sex: Female | | [a] | [a] | [a] | [a] | [a] | [a] | [a] | [a] |
| Group 1, | Mean | 1.51 | 0.26 | 4.5 | 141 | 104 | 23.7 | 1.59 | 402 |
| 0 mg/kg/wk | SD | 0.41 | 0.04 | 0.4 | 1 | 1 | 2.0 | 0.05 | 261 |
| | N | 5 | 5 | 5 | 5 | 5 | 5 | 5 | 5 |
| Group 2, | Mean | 1.54 | 0.24 | 4.5 | 142 | 104 | 22.7 | 1.58 | 537 |
| 3 mg/kg/wk | SD | 0.14 | 0.04 | 0.3 | 1 | 1 | 1.9 | 0.09 | 227 |
| | N | 5 | 5 | 5 | 5 | 5 | 5 | 5 | 5 |
| | % Diff | 1.99 | −6.15 | 0.0 | 0 | 1 | −4.2 | −0.53 | 34 |
| Group 3, | Mean | 1.70 | 0.26 | 4.3 | 142 | 105 | 23.0 | 1.64 | 247 |
| 3.75/2.5 | SD | 0.24 | 0.05 | 0.3 | 2 | 2 | 1.5 | 0.11 | 83 |
| mg/kg/wk | N | 5 | 5 | 5 | 5 | 5 | 5 | 5 | 5 |
| | % Diff | 12.47 | −1.54 | −4.8 | 1 | 1 | −2.7 | 2.84 | −39 |
| Group 4, | Mean | 1.37 | 0.29 | 4.6 | 142 | 106 | 22.0 | 1.70 | 427 |
| 4.69/2 | SD | 0.52 | 0.11 | 0.4 | 4 | 5 | 3.6 | 0.25 | 155 |
| mg/kg/wk | N | 5 | 5 | 5 | 5 | 5 | 5 | 5 | 5 |
| | % Diff | −9.02 | 10.00 | 1.8 | 1 | 2 | −7.2 | 6.75 | 6 |

[a] - Anova & Dunnett
TCHO, total cholesterol; TG, triglyceride; K, potassium; Na, sodium; Cl, chloride; GLB, globulin; A/G, A/G ratio; CK, creatine kinase. [symbol]
indicates data missing or illegible when filed

In conclusion, administration of FL118 to rats once weekly for Sprague Dawley rats by oral gavage at dosages of 3, 3.75→2.5, or 4.69→2 mg/kg/wk resulted in mortality at 4.69→2 mg/kg/wk, and changes in clinical signs, body weight, food consumption, and clinical pathology at all treatment groups. The conservative no observed adverse effect level (NOAEL) for this study was considered to be 2 mg/kg/wk for males and 3 mg/kg/wk for females.

Example 65-Toxicology and MTD studies with the FDA requirement-matched standard numbers of dogs (3 males and 3 males per group) using clinically compatible FL118 product (LOT 2020-252-23): The WuXi AppTec Hong Kong Toxicology subsidiary also performed the toxicology and MTD studies in beagle dogs using clinically compatible new FL118 product (i.e., the glacial acetic acid/ethanol and microfluidizer-involved SDD-processed FL118-HPβCD complex powder product, LOT 2020-252-23) with the schedule of once weekly for 2 doses on day 1 and day 8 for 14-days observation. Twelve male and 12 female beagle dogs (Canis familiaris) were randomly assigned to 4 groups (3/sex/group) to determine the new FL118 products' toxicology and MTD. Then the vehicle for group 1 and the FL118 product (LOT 2020-252-23) for groups 2, 3 and 4 were orally dosed on Days 1 and 8 via oral gavage as defined in Table 39. Based on the toxicity/MTD testing outcome derived from limited numbers of rats presented in the Example 62 above, the starting dose levels in the three FL118 test groups 2, 3 and 4 were 2.5 mg/kg, 3.3 mg/kg and 4.4 mg/kg on Day 1 were used (Table 39). However, there were no dog body weight loss and other clinical toxicity being observed before the second-time FL118 oral treatment on day 8. Therefore, the FL118 dose levels were adjusted for the FL118 oral treatment on Day 8 to 9.9 mg/kg/dose for group 2, 6.6 mg/kg/dose for group 3 and still 4.4 mg/kg/dose for group 4 as shown in Table 39.

TABLE 39
FL118 oral treatment groups for toxicology testing with
the FDA requirement-matched standard numbers of dogs.

| | | FL118 Doses ^{a} | | | Dosing | | |
|---|---|---|---|---|---|---|---|
| Group | Group | Dose ^{c} | Volume | Conc. | date | Numbering of Animals | |
| Numbers | Designation | (mg/kg/dose) | (mL/kg) | (mg/mL) | (Day) | M | F |
| 1 | Control/HPβCD ^{b} | 0 | 5 | 0 | 1, 8 | 1001-1003 | 1501-1503 |
| 2 | FL118 Dose 1 | 2.5 | 5 | 0.5 | 1 | 2001-2003 | 2501-2503 |
| | | 9.9 | 5 | 1.98 | 8 | | |
| 3 | FL118 Dose 2 | 3.3 | 5 | 0.66 | 1 | 3001-3003 | 3501-3503 |
| | | 6.6 | 5 | 1.32 | 8 | | |
| 4 | FL118 Dose 3 | 4.4 | 5 | 0.88 | 1, 8 | 4001-4003 | 4501-4503 |

Note:
In this protocol, “dose level” and “dosage” are used interchangeably.
^{a} Doses represent active ingredient.
^{b} The control formulation was 8.8 mg/mL HPβCD in vehicle. For control group, the dose of HPβCD was 44 mg/kg/dose (which is equivalent to the amount of HPBCD in the FL118 High Dose group 4)
^{c} Dosing on Day 1 and Day 8. The dose levels of Group 2 and 3 was increased on Day 8 for fully exploring the toxicity of test article as there was no adverse changes in clinical signs and bodyweight during Day 1-7
Conc. = Concentration M = Male F = Female.

A total of twenty-four dogs (12/sex) were randomly assigned to 4 groups of 3/sex/group and given with oral administration of control article (vehicle/group 1:9.4 mg/mL HPβCD in sterile saline), FL118 at 2.5→9.9 (group 2), 3.3→6.6 (group 3), or 4.4 mg/kg/dose (group 4) by oral gavage once weekly on Day 1 and Day 8 at the dose volume of 5 mL/kg (Table 39). Animals were approximately 7 to 8 months of age with body weights ranging from 5.9 to 8.0 kg in females and 7.6 to 9.3 kg in males at dosing initiation.

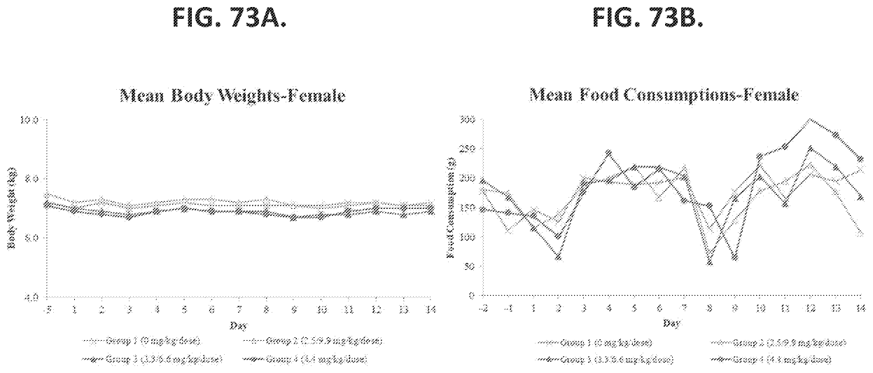

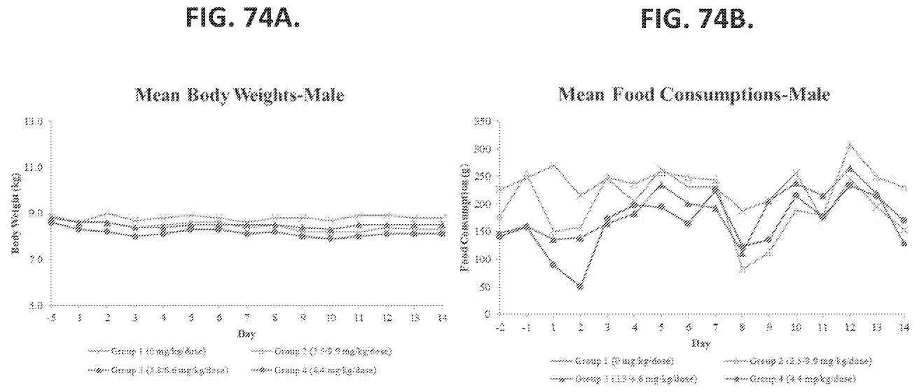

After once weekly oral administration of FL118 test article at 2.5→9.9, 3.3→6.6, or 4.4 mg/kg/dose to male and female dogs for 14 days, all animals survived to the scheduled termination. No FL118-related changes in body weights (FIGS. 73A, 74A), food consumption (FIGS. 73B, 74B), coagulation, urinalysis, gross pathology, organ weights, and histopathological evaluation were noted during the course of study. FL118-related clinical signs limited to abnormal stool noted in males and/or females at all treatment groups.

When compared with concurrent controls, significant decreases in group mean leukocyte (WBC) counts (max decrease of 14.33%) in male, reticulocyte (RET) count absolute (max decrease of 33.4%) in male, RET count percentage (max decrease of 30.65%) in male or neutrophils counts (#NEUT, max decrease of 18.67%) in male were noted at the end of the dosing phase on Day 15 at all treatment groups (Table 40).

TABLE 40
FL118 treatment-related hematology changes (M: male; F: female)

| | Doses (mg/kg/dose) | | | | | | | |
|---|---|---|---|---|---|---|---|---|
| | 0 | | 2.5→9.9 | | 3.3→6.6 | | 4.4 | |
| | No. of Animals | | | | | | | |
| Day 15 | M: 3 | F: 3 | M: 3 | F: 3 | M: 3 | F: 3 | M: 3 | F: 3 |
| WBC (10³/uL) | 11.89 | 8.90 | −14.33% | — | −9.95% | — | — | — |
| RET (10⁹/uL) | 66.3 | 37.5 | −33.4% | — | −17.5% | — | — | — |
| % RET (%) | 1.08 | 0.66 | −30.65% | — | −15.17% | — | — | — |
| #NEUT | 7.50 | 4.87 | −18.67% | — | −16.81% | — | — | — |

— No test article-related changes

The remaining variations in hematological parameters including those that were statistical significance, were of the type seen in untreated dogs in this laboratory, within historical reference range and were unrelated to test article treatment. Detailed hematology parameters were shown for male dogs in Table 41 and for female dogs in Table 42.

TABLE 41-1
Effects of FL118 (Group 2, 3, 4) versus vehicle (group 1) on male dogs' hematological parameters (WuXi AppTec)
Day −4 and Day 15: Relative to the start date (Day 1)

| | | WBC | WBC | RBC | RBC | HGB | HGB | HCT | HCT | MCV | MCV | MCH | MCH |
|---|---|---|---|---|---|---|---|---|---|---|---|---|---|
| | | (10\hat{ }3/μL) | (10\hat{ }3/μL) | (10\hat{ }6/μL) | (10\hat{ }6/μL) | (g/dL) | (g/dL) | (%) | (%) | (fL) | (fL) | (pg) | (pg) |
| | | [a] | [a] | [a1] | [a1] | [a] | [a1] | [a] | [a1] | [a] | [a] | [a] | [a] |
| Sex: Male | | −4 | 15 | −4 | 15 | −4 | 15 | −4 | 15 | −4 | 15 | −4 | 15 |
| Group 1, | Mean | 11.29 | 11.89 | 6.12 | 6.14 | 14.7 | 15.0 | 44.2 | 44.2 | 72.2 | 71.9 | 24.0 | 24.5 |
| 0 mg/kg/wk | SD | 2.41 | 3.05 | 0.19 | 0.04 | 0.5 | 0.3 | 1.9 | 1.1 | 1.0 | 1.0 | 0.1 | 0.2 |
| | N | 3 | 3 | 3 | 3 | 3 | 3 | 3 | 3 | 3 | 3 | 3 | 3 |
| Group 2, | Mean | 11.01 | 10.19 | 6.44 | 5.85 | 15.3 | 13.9 | 45.5 | 41.2 | 70.7 | 70.7 | 23.8 | [symbol] |
| 2.5/9.9 | SD | 0.43 | 0.95 | 0.14 | 0.60 | 0.5 | 1.7 | 1.5 | 5.4 | 2.3 | 2.3 | 0.7 | 0.8 |
| mg/kg/wk | N | 3 | 3 | 3 | 3 | 3 | 3 | 3 | 3 | 3 | 3 | 3 | 3 |
| | % Diff | −2.54 | −14.33 | [symbol] | −4.72 | 4.5 | −7.3 | 2.9 | −6.8 | −2.1 | −2.1 | −0.7 | −2.7 |
| Group 3, | Mean | 9.99 | 10.71 | 6.63 | 6.03 | 15.6 | 14.4 | 46.5 | 42.4 | 70.2 | 70.2 | 23.6 | 23.8 |
| 3.3/6.8 | SD | 3.02 | 1.55 | 0.64 | 0.12 | 0.9 | 0.4 | [symbol] | 1.1 | 2.9 | 2.9 | 1.3 | 1.1 |
| mg/kg/wk | N | 3 | 3 | 3 | 3 | 3 | 3 | 2 | 3 | 2 | 3 | 3 | 3 |
| | % Diff | −11.57 | −9.95 | 8.33 | [symbol] | 6.4 | −4.4 | 5.0 | −4.1 | −2.8 | −2.8 | −1.4 | −3.0 |
| Group 4, | Mean | 9.50 | 11.54 | 6.45 | 6.11 | 15.1 | 14.3 | 44.7 | 42.2 | 69.3 | 69.3 | 23.4 | 23.5 |
| 4.4 mg/kg/wk | SD | 0.90 | 1.78 | 0.30 | 0.07 | 0.8 | 0.1 | 1.7 | 0.6 | 0.5 | 0.5 | 0.5 | 0.2 |
| | N | 3 | 3 | 3 | 3 | 3 | 3 | 3 | 3 | 3 | 3 | 3 | 3 |
| | % Diff | −15.91 | −2.92 | 5.39 | −0.43 | 3.0 | −4.9 | 1.1 | −4.6 | −4.0 | −4.0 | −2.4 | −4.3 |

[a] - Anova & Dunnett;
[a1] - Kruskal-Wallis & Dunnett on Ranks.
WBC, white blood cell; RBC, red blood cell; HGB, hemoglobin; HCT, hematocrit; MCV, mean corpuscular volume; MCH, mean corpuscular
hemoglobin.

| | | MCHC | MCHC | RDW | RDW | #RET | #RET | % RET | % RET | #NEUT | #NEUT | % NEUT | % NEUT |
|---|---|---|---|---|---|---|---|---|---|---|---|---|---|
| | | (g/dL) | (g/dL) | (%) | (%) | (10\hat{ }9/L) | (10\hat{ }9/L) | (%) | (%) | (10\hat{ }3/μL) | (10\hat{ }3/μL) | (%) | (%) |
| | | [a] | [a1] | [a1] | [a1] | [a1] | [a1] | [a1] | [a1] | [a1] | [a1] | [a1] | [a1] |
| Sex: Male | | −4 | 15 | −4 | 15 | −4 | 15 | −4 | 15 | −4 | 15 | −4 | 15 |
| Group 1, | Mean | 33.2 | 34.1 | 12.4 | 12.2 | 57.3 | 66.3 | 0.94 | 1.08 | 7.07 | 7.50 | 62.7 | 63.3 |
| 0 mg/kg/wk | SD | 0.5 | 0.4 | 0.6 | 0.4 | 6.7 | 28.7 | 0.14 | 0.46 | 1.37 | 1.09 | 1.6 | 2.2 |
| | N | 3 | 3 | 3 | 3 | 3 | 3 | 3 | 3 | 3 | 3 | 3 | 3 |
| Group 2, | Mean | 33.6 | 33.9 | 12.5 | 12.3 | 74.4 | 44.2 | 1.15 | 0.75 | 6.71 | 6.10 | 60.9 | 59.7 |
| 2.5/9.9 | SD | 0.2 | 0.3 | 0.7 | 0.3 | 33.9 | 12.2 | 0.52 | 0.17 | 0.44 | 0.86 | 1.6 | 3.3 |
| mg/kg/wk | N | 3 | 3 | 3 | 3 | 3 | 3 | 3 | 3 | 3 | 3 | 3 | 3 |
| | % Diff | 1.3 | −0.6 | 0.8 | 0.8 | 30.0 | −33.4 | 22.70 | −30.65 | −5.05 | −18.67 | −2.9 | −5.6 |
| Group 3, | Mean | 33.6 | 33.9 | 12.1 | 12.0 | 50.1 | 54.7 | 0.75 | 0.91 | 6.15 | 6.24 | 59.2 | 57.5 |
| 3.3/6.8 | SD | 0.4 | 0.3 | 0.1 | 0.3 | 9.0 | 24.8 | 0.14 | 0.43 | 3.04 | 1.74 | 11.9 | 8.0 |
| mg/kg/wk | N | 3 | 3 | 3 | 3 | 3 | 3 | 3 | 3 | 3 | 3 | 3 | 3 |
| | % Diff | 1.3 | −0.6 | −2.2 | −1.9 | −12.5 | −17.5 | −19.15 | −15.17 | −13.02 | −16.81 | −5.6 | −9.2 |
| Group 4, | Mean | 33.8 | 34.0 | 12.8 | 12.4 | 88.2 | 67.0 | 1.36 | 1.10 | 5.77 | 7.93 | 60.2 | 67.4 |
| 4.4 mg/kg/wk | SD | 9.9 | 0.1 | 0.4 | 0.4 | 35.4 | 21.0 | 0.54 | 0.34 | 1.49 | 2.77 | 10.5 | 13.2 |
| | N | 3 | 3 | 3 | 3 | 3 | 3 | 3 | 3 | 3 | 3 | 3 | 3 |
| | % Diff | 1.7 | −0.1 | 3.5 | 1.9 | 54.1 | 1.1 | 45.04 | 1.86 | −18.30 | 5.78 | −4.1 | 6.4 |

[a] - Kruskal-Wallis & Dunnett on Ranks;
[a1] - Anova & Dunnett.
MCHC, mean corpuscular hemoglobin concentration; RDW, RBC distribution width; #RET, reticulocytes, absolute; % RET, reticulocytes, percent, #NEUT, neutrophils, absolute; % NEUT, neutrophils, percent. [symbol]
indicates data missing or illegible when filed

TABLE 41-2
Effects of FL118 (Group 2, 3, 4) versus vehicle (group 1) on male dogs' hematological parameters (WuXi AppTec)
Day −4 and Day 15: Relative to the start date (Day 1) (Cont'd)

| | | #LYMP | #LYMP | % | % | #MONO | #MONO | % | % | #EOS | #EOS | | |
|---|---|---|---|---|---|---|---|---|---|---|---|---|---|
| | | (10\hat{ }3/ | (10\hat{ }3/ | LYMP | LYMP | (10\hat{ }3/ | (10\hat{ }3/ | MONO | MONO | (10\hat{ }3/ | (10\hat{ }3/ | % EOS | % EOS |
| | | μL) | μL) | (%) | (%) | μL) | μL) | (%) | (%) | μL) | μL) | (%) | (%) |
| | | [a] | [a1] | [a1] | [a1] | [a1] | [a1] | [a1] | [a1] | [a1] | [a1] | [a1] | [a1] |
| Sex: Male | | −4 | 15 | −4 | 15 | −4 | 15 | −4 | 15 | −4 | 15 | −4 | 15 |
| Group 1, | Mean | 5.16 | 3.24 | 27.8 | 27.2 | 0.77 | 0.83 | 6.9 | 6.7 | 0.21 | 0.21 | 1.9 | 1.9 |
| 0 mg/kg/wk | SD | 0.86 | 0.67 | 1.6 | 1.2 | 0.18 | 0.44 | 1.5 | 2.8 | 0.04 | 0.09 | 0.4 | 1.1 |
| | N | 3 | 3 | 3 | 3 | 3 | 3 | 3 | 3 | 3 | 3 | 3 | 3 |
| Group 2, | Mean | 3.18 | 3.03 | 28.9 | 29.8 | 0.94 | 0.81 | 8.6 | 8.0 | 0.10 | 0.18 | 0.9 | 1.8 |
| 2.5/9.9 | SD | 0.06 | 0.27 | 0.5 | 2.5 | 0.11 | 0.05 | 1.3 | 1.1 | 0.03 | 0.08 | 0.2 | 0.9 |
| mg/kg/wk | N | 3 | 3 | 3 | 3 | 3 | 3 | 3 | 3 | 3 | 3 | 3 | 3 |
| | % Diff | 0.42 | −6.58 | 3.7 | 9.7 | 22.61 | −2.42 | 24.8 | 18.9 | −53.23 | −14.29 | −52.6 | −1.5 |
| Group 3, | Mean | 2.98 | 3.41 | 51.7 | 32.4 | 0.61 | 0.70 | 6.5 | 6.8 | 0.15 | 0.23 | 1.6 | 2.2 |
| 3.3/6.6 | SD | 0.05 | 0.37 | 9.5 | 5.6 | 0.04 | [symbol] | 1.6 | 2.7 | 0.09 | 0.14 | 1.2 | 1.3 |
| mg/kg/wk | N | 3 | 3 | 3 | 3 | 3 | 3 | 3 | 3 | 3 | 3 | 3 | 3 |
| | % Diff | −5.69 | 5.24 | 14.0 | 19.1 | −20.43 | −15.32 | −5.8 | 1.5 | −25.81 | 9.52 | −14.0 | 14.0 |
| Group 4, | Mean | 2.82 | 2.73 | 30.4 | 24.8 | 0.67 | 0.64 | 7.0 | 5.6 | 0.16 | 0.16 | 1.8 | 1.5 |
| 4.4 | SD | 0.72 | 0.99 | 10.8 | 12.1 | 0.22 | 0.12 | 1.8 | 0.5 | 0.06 | 0.08 | 0.8 | 0.9 |
| mg/kg/wk | N | 3 | 3 | 3 | 3 | 3 | 3 | 3 | 3 | 3 | 5 | 3 | 3 |
| | % Diff | −10.85 | −15.93 | 9.3 | −8.7 | −12.61 | −22.18 | 1.5 | −16.9 | −20.97 | −22.22 | −7.0 | −21.1 |

[a] - Kruskal-Wallis & Dunnett on Ranks;
[a1] - Anova & Dunnett
#LYMP, lymphocytes, absolute; % LYMP, lymphocytes, percent; #MONO, monocytes, absolute; % MONO, monocytes, percent; #EOS, eosinophils,
absolute; % EOS, eosinophils, percent.

| | | #BASO | #BASO | % BASO | % BASO | PLT | PLT | MPV | MPV |
|---|---|---|---|---|---|---|---|---|---|
| | | (10\hat{ }3/μL) | (10\hat{ }3/μL) | (%) | (%) | (10\hat{ }3/μL) | (10\hat{ }3/μL) | (fL) | (fL) |
| | | [a] | [a] | [a] | [a] | [a] | [a] | [a] | [a] |
| Sex: Male | | −4 | 15 | −4 | 15 | −4 | 15 | −4 | 15 |
| Group 1, | Mean | 0.03 | 0.05 | 0.3 | 0.4 | 392 | 400 | 13.5 | 13.7 |
| 0 mg/kg/wk | SD | 0.03 | 0.02 | 0.2 | 0.1 | 100 | 59 | 1.2 | 1.6 |
| | N | 3 | 3 | 3 | 3 | 3 | 3 | 3 | 3 |
| Group 2, | Mean | 0.04 | 0.03 | 0.3 | 0.3 | 396 | 349 | 12.7 | 12.1 |
| 2.5/9.9 | SD | 0.03 | 0.02 | 0.3 | 0.2 | 26 | 8 | 0.9 | 0.3 |
| mg/kg/wk | N | 3 | 3 | 3 | 3 | 3 | 3 | 3 | 3 |
| | % Diff | 10.00 | −40.00 | 25.0 | −27.3 | 1 | −13 | −6.2 | −4.6 |
| Group 3, | Mean | 0.05 | 0.05 | 0.5 | 0.4 | 345 | 314 | 14.1 | 13.7 |
| 3.3/6.6 | SD | 0.01 | 0.02 | 0.1 | 0.2 | 47 | 41 | 0.7 | 1.2 |
| mg/kg/wk | N | 3 | 3 | 3 | 3 | 3 | 3 | 3 | 3 |
| | % Diff | 40.00 | 0.00 | 87.5 | 18.2 | −12 | −21 | 4.7 | −0.5 |
| Group 4, | Mean | 0.03 | 0.04 | 0.4 | 0.4 | 371 | 331 | 14.8 | 13.9 |
| 4.4 mg/kg/wk | SD | 0.02 | 0.02 | 0.2 | 0.3 | 57 | 33 | 2.4 | 1.5 |
| | N | 3 | 3 | 3 | 3 | 3 | 3 | 3 | 3 |
| | % Diff | 0.00 | −13.33 | 37.5 | 9.1 | −5 | −17 | 6.4 | 1.0 |

[a1] - Anova & Dunnett.
#BASO, basophils, absolute; % BASO, basophils, percent; PLT, platelet count; MPV, mean platelet volume. [symbol]
indicates data missing or illegible when filed

TABLE 42-1
Effects of FL118 (Group 2, 3, 4) versus vehicle (group 1) on female dogs'
hematological parameters (WuXi AppTec)
Day −4 and Day 15: Relative to the start date (Day 1)

| | | WBC | WBC | RBC | RBC | HGB | HGB |
|---|---|---|---|---|---|---|---|
| | | (10 [symbol] /μL) | (10 [symbol] /μL) | (10 [symbol] /μL) | (10 [symbol] /μL) | (g/dL) | (g/dL) |
| | | [a] | [a1] | [a] | [a] | [a] | [a] |
| Sex: Female | | −4 | 15 | −4 | 15 | −4 | 15 |
| Group 1, | Mean | 9.66 | 8.90 | 6.02 | 5.67 | 14.1 | 13.6 |
| 5 | SD | 0. [symbol] 4 | 0.41 | 0.46 | 0.37 | 0.8 | 0.9 |
| mg/kg/dose | N | 3 | 3 | 3 | 3 | 3 | 3 |
| Group 2, | Mean | 12.50 | 11.44 | 6.28 | 5.81 | 15.0 | 14.1 |
| 2.5/9.9 | SD | 3.10 | [symbol] .23 | 0.18 | 0.09 | 0.6 | 0.7 |
| mg/kg/dose | N | 3 | 3 | 3 | 3 | 3 | 3 |
| | % Diff | 24.26 | 28.49 | 4. [symbol] 2 | 2.47 | 6.6 | 3.7 |
| Group 3, | Mean | 8.32 | 8.27 | 6.61 | 5.85 | 15.4 | 13.7 |
| 3.3/6.6 | SD | 0.83 | 0.84 | 0.32 | 0.54 | 0.6 | 0.7 |
| mg/kg/dose | N | 3 | 3 | 3 | 3 | 3 | 3 |
| | % Diff | −13.91 | −2.15 | 9.74 | 3.24 | 9.5 | 0.7 |
| Group 4, | Mean | 11.93 | 10.02 | 6.47 | 6.29 | 15.1 | 14.8 |
| 4/4 | SD | 2.87 | 1.41 | 0. [symbol] 0 | 0.46 | 0.5 | 0.7 |
| mg/kg/dose | N | 3 | 3 | 3 | 3 | 3 | 3 |
| | % Diff | 23.46 | 12.58 | 7. [symbol] 6 | 11.06 | 7.3 | 9.5 |

[a] - Anova & Dunnett; [a1] - Kruskal-Wallis & Dunnett on Ranks.
WBC, white blood cell; RBC, red blood cell; HGB, hemoglobin; HCT, hematocrit, MCV,
mean corpuscular volume; MCH, mean corpuscular hemoglobin.

| | | HCT | HCT | MCV | MCV | MCH | MCH |
|---|---|---|---|---|---|---|---|
| | | (%) | (%) | (fL) | (fL) | (pg) | (pg) |
| | | [a] | [a] | [a] | [a] | [a] | [a] |
| Sex: Female | | −4 | 15 | −4 | 15 | −4 | 15 |
| Group 1, | Mean | 42.6 | 40.1 | 70.7 | 70.8 | 23.4 | 24.0 |
| 5 | SD | 3.4 | 3.1 | 2.8 | 2.0 | 1.1 | 0.5 |
| mg/kg/dose | N | 3 | 3 | 3 | 3 | 3 | 3 |
| Group 2, | Mean | 45. [symbol] | 41.1 | 71.7 | 70.8 | 23.8 | 24.2 |
| 2.5/9.9 | SD | 1.6 | 1.9 | 3.3 | 3.8 | 1.2 | 1.3 |
| mg/kg/dose | N | 3 | 3 | 3 | 3 | 3 | 3 |
| | % Diff | 5. [symbol] | 2.7 | 1.5 | 0.0 | 2.0 | 2.1 |
| Group 3, | Mean | 45.4 | 40.5 | 68.8 | 69.4 | 23.3 | 23.4 |
| 3.3/6.6 | SD | 1.4 | 2.7 | 2.8 | 2.5 | 1.1 | 1.2 |
| mg/kg/dose | N | 3 | 3 | 3 | 3 | 3 | 3 |
| | % Diff | 6.7 | 1.2 | −2.7 | −1.9 | −0.3 | −2.5 |
| Group 4, | Mean | 44.9 | 43.8 | 69. [symbol] | 69.6 | 23.4 | 23.6 |
| 4/4 | SD | 1.2 | 2.3 | 1.6 | 1.7 | 0.6 | 0.9 |
| mg/kg/dose | N | 3 | 3 | 3 | 3 | 3 | 3 |
| | % Diff | 5.4 | 9.2 | −1.8 | −1.6 | 0.1 | −1.8 |

[a] - Anova & Dunnett; [a1] - Kruskal-Wallis & Dunnett on Ranks.
WBC, white blood cell; RBC, red blood cell; HGB, hemoglobin; HCT, hematocrit, MCV,
mean corpuscular volume; MCH, mean corpuscular hemoglobin.

| | | MCHC | MCHC | RDW | RDW | #RET | #RET |
|---|---|---|---|---|---|---|---|
| | | (g/dL) | (g/dL) | (%) | (%) | (10\hat{ }9/L) | (10\hat{ }9/L) |
| | | [a] | [a1] | [a1] | [a] | [a] | [a2] |
| Sex: Female | | −4 | 15 | −4 | 15 | −4 | 15 |
| Group 1, | Mean | 33.1 | [symbol] 3.9 | 13.1 | 12.7 | 47.1 | 37.5 |
| 0 | SD | 0.9 | 0.4 | 1.7 | 1.4 | 28.4 | 7.3 |
| mg/kg/dose | N | 3 | 3 | 3 | 3 | 3 | 3 |
| Group 2, | Mean | 35.2 | 34.3 | 11.6 | 11.6 | [symbol] | 45.6 |
| 2.5/9.9 | SD | 0.2 | 0.3 | 0.2 | 0.3 | [symbol] | 32. [symbol] |
| mg/kg/dose | N | 3 | 3 | 3 | 3 | 3 | 3 |
| | % Diff | 0.4 | 1.1 | −11.5 | − [symbol] .7 | 2 [symbol] .7 | 21.7 |
| Group 3, | Mean | 33.9 | 5 [symbol] .7 | 12.5 | 12.0 | 32.3 | 38. [symbol] |
| 3.3/6.6 | SD | 0.3 | 1.3 | 2.3 | 1.7 | 11.5 | 2.5 |
| mg/kg/dose | N | 3 | 3 | 3 | 3 | 3 | 3 |
| | % Diff | 2.4 | −0.5 | −4.6 | −5. [symbol] | −31.4 | 1.8 |
| Group 4, | Mean | 35.7 | 33.9 | 13.1 | 12.5 | 45. [symbol] | 60.5 |
| 4/4 | SD | 0.5 | 0.5 | 0.7 | 0.5 | 12.3 | 10. [symbol] |
| mg/kg/dose | N | 3 | 3 | 3 | 3 | 3 | 3 |
| | % Diff | 1.8 | −0.1 | − [symbol] .9 | −2.9 | −2.5 | 61.4 |

| | | % RET | % RET | #NEUT | #NEUT | % NEUT | % NEUT |
|---|---|---|---|---|---|---|---|
| | | (%) | (%) | (10 [symbol] /μL) | (10 [symbol] /μL) | (%) | (%) |
| | | [a] | [a2] | [a2] | [a] | [a] | [a] |
| Sex: Female | | −4 | 15 | −4 | 15 | −4 | 15 |
| Group 1, | Mean | 0.77 | 0.66 | 5.45 | 4.87 | 56.4 | 54.7 |
| 0 | SD | 0.41 | 0.10 | 0.33 | 0.20 | 2. [symbol] | 0.9 |
| mg/kg/dose | N | 3 | 3 | 3 | 3 | 3 | 3 |
| Group 2, | Mean | 0.89 | 0.78 | 8.34 | 7.76 | 70. [symbol] ** | [symbol] 8.2* |
| 2.5/9.9 | SD | [symbol] .58 | 0. [symbol] 4 | 1.86 | 2. [symbol] | 3.9 | 3. [symbol] |
| mg/kg/dose | N | 3 | 3 | 3 | 3 | 3 | 3 |
| | % Diff | 16.02 | 1 [symbol] | 5 [symbol] .09 | 59.45 | 24.1 | 24.7 |
| Group 3, | Mean | 0.49 | [symbol] .65 | 4.86 | 5.08 | 58.8 | 61.4 |
| 3.3/6.6 | SD | 0.16 | [symbol] .05 | 0.19 | 0.85 | 4.5 | 5.7 |
| mg/kg/dose | N | 3 | 3 | 3 | 3 | 3 | 3 |
| | % Diff | −36.80 | −0.51 | −10.76 | 4.45 | 4.3 | 12. [symbol] |
| Group 4, | Mean | 0.71 | 0. [symbol] | 7.93 | [symbol] .44 | [symbol] 6.5* | 63.9 |
| 4/4 | SD | 0.16 | 0.20 | 2.19 | 1. [symbol] | 3.5 | 4.8 |
| mg/kg/dose | N | 3 | 3 | 3 | 3 | 3 | 3 |
| | % Diff | − [symbol] .23 | 47.72 | 45.57 | 32.40 | 17.0 | 17.0 |

[a] - Anova & Dunnett: * = p ≤ 0.05; ** = p ≤ 0.01; [a1] - Kruskal-Wallis & Dunnett on Ranks; [a2] - Anova & Dunnett (Log).
MCHC, mean corpuscular hemoglobin concentration; RDW, RBC distribution width; #RET, reticulocytes, absolute; % RET, reticulocytes, percent; #NEUT, neutrophils, absolute; % NEUT, neutrophils, percent. [symbol]
indicates data missing or illegible when filed

TABLE 42-2
Effects of FL118 (Group 2, 3, 4) versus vehicle (group 1) on female dogs' hematological parameters (WuXi AppTec)
Day −4 and Day 15: Relative to the start date (Day 1) (Cont'd)

| | | #LYMP | #LYMP | % LYMP | % LYMP | #MONO | #MONO |
|---|---|---|---|---|---|---|---|
| | | (10 [symbol] /μL) | (10 [symbol] /μL) | (%) | (%) | (10 [symbol] /μL) | (10 [symbol] /μL) |
| | | [a] | [a] | [a] | [a] | [a] | [a] |
| Sex: Female | | −4 | 15 | −4 | 15 | −4 | 15 |
| Group 1, | Mean | 3.16 | 3.15 | 32.7 | 35.3 | 0.79 | 0.60 |
| 0 | SD | 0.21 | 0.24 | 2.7 | 1.2 | 0.13 | 0.10 |
| mg/kg/dose | N | 3 | 3 | 3 | 3 | 3 | 3 |
| Group 2, | Mean | 2.65 | 2.70 | 21.7* | 23.2* | [symbol] | 0. [symbol] 7 |
| 2.5/9.9 | SD | 0.90 | [symbol] | 3.5 | 3.7 | 0.30 | 0.22 |
| mg/kg/dose | N | 3 | 3 | 3 | 3 | 3 | 3 |
| | % Diff | −16.24 | −14.30 | −33.6 | −34.2 | −0.85 | 11.60 |
| Group 3, | Mean | 2.62 | 2.44 | 31.2 | 29.7 | 0.61 | 0.49 |
| 3.3/6.6 | SD | 0.77 | 0.47 | 6.2 | 6.1 | 0.17 | 0.12 |
| mg/kg/dose | N | 3 | 3 | 3 | 3 | 3 | 3 |
| | % Diff | −17.19 | −22.35 | −5.0 | −13.8 | −22.03 | −18.78 |
| Group 4, | Mean | 2.85 | 2.75 | 24.4 | 27.8 | 0.84 | 0.55 |
| 4/4 | SD | 0. [symbol] 2 | 0.33 | 3.3 | 4.4 | 0.25 | 0.09 |
| mg/kg/dose | N | 3 | 3 | 3 | 3 | 3 | 3 |
| | % Diff | −9.70 | −12.61 | −25.5 | −21.4 | 6.36 | − [symbol] .84 |

[a] - Anova & Dunnett: * = ≤0.05
#LYMP, lymphocytes, absolute; % LYMP, lymphocytes, percent; #MONO, monocytes, absolute; % MONO, monocytes,
percent; #EOS, eosinophils, absolute; % EOS, eosinophils, percent.

| | | % MONO | % MONO | #EOS | #EOS | % EOS | % EOS |
|---|---|---|---|---|---|---|---|
| | | (%) | (%) | (10 [symbol] /μL) | (10 [symbol] /μL) | (%) | (%) |
| | | [a] | [a] | [a] | [a] | [a] | [a] |
| Sex: Female | | −4 | 15 | −4 | 15 | −4 | 15 |
| Group 1, | Mean | [symbol] | 6.7 | 0.22 | 0.21 | 2.3 | 2.4 |
| 0 | SD | [symbol] | 0.9 | 0.05 | 0.09 | 0.5 | 1.2 |
| mg/kg/dose | N | 3 | 3 | 3 | 3 | 3 | 3 |
| Group 2, | Mean | 6.4 | 6.0 | 0.19 | 0.24 | 1.4 | 1.9 |
| 2.5/9.9 | SD | 1.0 | 1.6 | 0.15 | 0.20 | 0.9 | 1.4 |
| mg/kg/dose | N | 3 | 3 | 3 | 3 | 3 | 3 |
| | % Diff | −21.4 | − [symbol] 0.4 | −13.85 | 14.52 | −36.4 | −18.3 |
| Group 3, | Mean | 7.4 | 5.9 | 0.19 | 0.19 | 2.4 | 2.3 |
| 3.3/6.6 | SD | 1.8 | 1.0 | 0.09 | 0.09 | 1.3 | 1.2 |
| mg/kg/dose | N | 3 | 3 | 3 | 3 | 3 | 3 |
| | % Diff | −9.1 | −12.4 | −13.85 | −9.68 | 7.6 | −2.8 |
| Group 4, | Mean | 6.9 | 5.5 | 0.26 | 0.22 | 2.3 | 2.2 |
| 4/4 | SD | 0.6 | 0.6 | 0.11 | 0.03 | 1.2 | 0.7 |
| mg/kg/dose | N | 3 | 3 | 3 | 3 | 3 | 3 |
| | % Diff | −14.4 | −18.3 | 18.46 | 6.45 | 4.5 | −5.6 |

[a] - Anova & Dunnett: * = ≤0.05
#LYMP, lymphocytes, absolute; % LYMP, lymphocytes, percent; #MONO, monocytes, absolute; % MONO, monocytes,
percent; #EOS, eosinophils, absolute; % EOS, eosinophils, percent.

| | | #BASO | #BASO | % BASO | % BASO | PLT | PLT | MPV | MPV |
|---|---|---|---|---|---|---|---|---|---|
| | | (10 [symbol] /μL) | (10 [symbol] /μL) | (%) | (%) | (10 [symbol] /μL) | (10 [symbol] /μL) | (fL) | (fL) |
| | | [a] | [a] | [a] | [a] | [a] | [a] | [a] | [a1] |
| Sex: Female | | −4 | 15 | −4 | 15 | −4 | 15 | −4 | 15 |
| Group 1, | Mean | 0.03 | 0.04 | 0.3 | 0.5 | 4 [symbol] | 412 | 12.7 | 13.1 |
| 0 | SD | 0.01 | 0.00 | 0.1 | 0.0 | 24 | 24 | 1. [symbol] | 1.2 |
| mg/kg/dose | N | 3 | 3 | 3 | 3 | 3 | 3 | 3 | 3 |
| Group 2, | Mean | 0.02 | 0.03 | 0.2 | 0.3 | 296 | 275 | 13.2 | 13.9 |
| 2.5/9.9 | SD | 0.01 | 0.02 | 0.1 | 0.1 | 63 | 48 | 2.4 | 2.7 |
| mg/kg/dose | N | 3 | 3 | 3 | 3 | 3 | 3 | 3 | 3 |
| | % Diff | −12.50 | −16.6 [symbol] | −25.0 | −40.0 | −29 | −33 | 3.9 | 5. [symbol] |
| Group 3, | Mean | 0.02 | 0.03 | 0.2 | 0.4 | 317 | 282 | 10.9 | 12.6 |
| 3.3/6.6 | SD | 0.01 | 0.01 | 0.1 | 0.2 | [symbol] | [symbol] | 0.8 | 0.4 |
| mg/kg/dose | N | 3 | 3 | 3 | 3 | 3 | 3 | 3 | 3 |
| | % Diff | −37.50 | −25.00 | −25.0 | −26.7 | −24 | − [symbol] 1 | −14.7 | −3.8 |
| Group 4, | Mean | 0.03 | 0.03 | 0.2 | 0.3 | [symbol] 74 | 3 [symbol] 3 | 11.1 | 12.3 |
| 4/4 | SD | 0.01 | 0.01 | 0.0 | 0.1 | 92 | 9 [symbol] | 1.7 | 1.4 |
| mg/kg/dose | N | 3 | 3 | 3 | 3 | 3 | 3 | 3 | 3 |
| | % Diff | 0.00 | − [symbol] | −25.0 | −46.7 | −10 | −12 | −11. [symbol] | −6.3 |

[a] - Anova & Dunnett; [a1] - Kruskal-Wallis & Dunnett on Ranks.
#BASO, basophils, absolute; % BASO, basophils, percent; PLT, platelet count, MPV, mean platelet volume. [symbol]
indicates data missing or illegible when filed

FL118-related increased Total Bilirubin (BIL-T, max increase of 47.34% in males and 16.14% in females, respectively) and decreased Triglyceride (TG, max decrease of 22.56% in males and 37.84% in females, respectively) were noted in males and females at all treatment groups when compared with control group (Table 43).

TABLE 43
FL118 treatment-related serum chemistry changes.

| | Doses (mg/kg/dose) | | | | | | | |
|---|---|---|---|---|---|---|---|---|
| | 0 | | 2.5 . . . , 9.9 | | 3.3 . . . , 6.6 | | 4.4 | |
| | No. of Animals | | | | | | | |
| Day 15 | M: 3 | F: 3 | M: 3 | F: 3 | M: 3 | F: 3 | M: 3 | F: 3 |
| BIL-T | 1.13 | 1.49 | 47.34% | 16.14% | 28.70% | 11.88% | 31.07% | 7.85% |
| TG | 0.44 | 0.49 | −22.56% | −37.84%** | −10.53% | −28.38%* | −15.79% | −27.70%* |

*p < 0.05;
**p < 0.01

The remaining variations in serum biochemical parameters were of the type seen in untreated dogs at this laboratory, lacked dose response, within historical reference, were comparative with controls and considered to be unrelated to the FL118 test article treatment. Detailed serum chemistry parameters were shown for male dogs in Table 44 and for female rats in Table 45.

TABLE 44-1
Effects of FL118 (Group 2, 3, 4) versus vehicle (Group 1) on male dogs'
serum chemical parameters (WuXi AppTec)
Day −4 and Day 15: Relative to the start date (Day 1)

| | | ALT | ALT | AST | AST | TP | TP |
|---|---|---|---|---|---|---|---|
| | | ( [symbol] /L) | ( [symbol] /L) | ( [symbol] /L) | ( [symbol] /L) | (g/L) | (g/L) |
| | | [a] | [a] | [a1] | [a] | [a] | [a] |
| Sex: Male | | −4 | 15 | −4 | 15 | −4 | 15 |
| Group 1, | Mean | 25 | 28 | 33 | 29 | 62.7 | 6 [symbol] .9 |
| 0 | SD | 3 | 4 | 2 | 2 | 3.3 | 3.0 |
| mg/kg/dose | N | 3 | 3 | 3 | 3 | 3 | 3 |
| Group 2, | Mean | 27 | 26 | 27 | 29 | 60.7 | 58.7 |
| 2.5/9.9 | SD | 6 | [symbol] | 6 | 4 | 1.5 | 4.5 |
| mg/kg/dose | N | 3 | 3 | 2 | 3 | 3 | 3 |
| | % Diff | 11 | − [symbol] | 14 | −3 | − [symbol] .1 | −3 [symbol] |
| Group 3, | Mean | 37 | 31 | 32 | 37 | 63.8 | [symbol] 9.6 |
| 3.3/6.6 | SD | 4 | 4 | 3 | 2 | 1. [symbol] | 2.0 |
| mg/kg/dose | N | 3 | 3 | 3 | 3 | 3 | 3 |
| | % Diff | 8 | 11 | 2 | −9 | 1.8 | −8.5 |
| Group 4, | Mean | 25 | 29 | 32 | [symbol] | [symbol] | 58. [symbol] |
| 4/4 | SD | 2 | 1 | 1 | 4 | 0. [symbol] | [symbol] .3 |
| mg/kg/dose | N | 3 | 3 | 3 | 3 | 3 | 3 |
| | % Diff | 3 | 4 | −3 | −31 | − [symbol] .7 | −11. [symbol] |

[a] - Anova & Dunnett: * = p ≤ 0.05; [a1] - Anova & Dunnett (Log).
ALT, alanine aminotransferase; AST, aspartate aminotransferase; TP, total protein; ALB,
albumin; BIL-T, total bilirubin (diasys); ALP, alkaline phosphatase.

| | | ALB | ALB | BIL-T | BIL-T | ALP | ALP |
|---|---|---|---|---|---|---|---|
| | | (g/L) | (g/L) | ( [symbol] /L) | ( [symbol] /L) | ( [symbol] /L) | ( [symbol] /L) |
| | | [a] | [a] | [a] | [a] | [a] | [a] |
| Sex: Male | | −4 | 15 | −4 | 15 | −4 | 15 |
| Group 1, | Mean | 30.7 | [symbol] | 1. [symbol] | 1.1 [symbol] | 149 | 152 |
| 0 | SD | 1.7 | [symbol] .4 | 0.06 | 0.17 | 29 | 4 [symbol] |
| mg/kg/dose | N | 3 | 3 | 3 | 3 | 3 | 3 |
| Group 2, | Mean | 30.9 | 28.9 | [symbol] | 1. [symbol] | 1. [symbol] | 11 [symbol] |
| 2.5/9.9 | SD | [symbol] | 2. [symbol] | 0.21 | [symbol] | 48 | [symbol] |
| mg/kg/dose | N | 3 | 3 | 3 | 3 | 3 | [symbol] |
| | % Diff | [symbol] | 1. [symbol] | 12. [symbol] 0 | 47.34 | [symbol] | −22 |
| Group 3, | Mean | [symbol] | 29. [symbol] | 1. [symbol] | 1.4 [symbol] | 142 | 122 |
| 3.3/6.6 | SD | [symbol] .5 | 0.5 | 0.18 | 0.16 | [symbol] | [symbol] |
| mg/kg/dose | N | 3 | 3 | 3 | 3 | 3 | 3 |
| | % Diff | [symbol] | 2. [symbol] | [symbol] 2.84 | 28. [symbol] | −5 | − [symbol] |
| Group 4, | Mean | 11.0 | [symbol] | 1.67 | 1.48 | 141 | 1 [symbol] |
| 4/4 | SD | 0.9 | 1.3 | 0.21 | 0. [symbol] | [symbol] | [symbol] |
| mg/kg/dose | N | 3 | 3 | 3 | 3 | 3 | 3 |
| | % Diff | [symbol] | [symbol] | [symbol] | 31.07 | −5 | −31 |

[a] - Anova & Dunnett: * = p ≤ 0.05; [a1] - Anova & Dunnett (Log).
ALT, alanine aminotransferase; AST, aspartate aminotransferase; TP, total protein; ALB,
albumin; BIL-T, total bilirubin (diasys); ALP, alkaline phosphatase.

| | | GGT | GGT | sGLU | sGLU | UREA | UREA |
|---|---|---|---|---|---|---|---|
| | | (U/L) | (U/L) | ( [symbol] /L) | ( [symbol] /L) | ( [symbol] /L) | ( [symbol] /L) |
| | | [a] | [a] | [a] | [a] | [a] | [a] |
| Sex: Male | | −4 | 15 | −4 | 15 | −4 | 15 |
| Group 1, | Mean | 3 | 3 | 4.99 | 4.7 [symbol] | [symbol] .84 | [symbol] |
| 0 | SD | 1 | 1 | 0.4 [symbol] | 0. [symbol] 5 | 0. [symbol] 6 | 1.01 |
| mg/kg/dose | N | 3 | 3 | 3 | 3 | 3 | 3 |
| Group 2, | Mean | 3 | 3 | 4.68 | 5.14 | 4. [symbol] 8 | 3.47 |
| 2.5/9.9 | SD | 1 | 1 | 0.5 [symbol] | 0. [symbol] | [symbol] .81 | 0.70 |
| mg/kg/dose | N | 3 | [symbol] | [symbol] | 3 | 3 | 3 |
| | % Diff | −3 [symbol] | [symbol] | −6.21 | 7.4 [symbol] | 6.34 | − [symbol] |
| Group 3, | Mean | [symbol] | 3 | 4.98 | [symbol] | 3.85 | [symbol] |
| 3.3/6.6 | SD | 1 | 1 | 0. [symbol] | 0. [symbol] | 0. [symbol] | 0.21 |
| mg/kg/dose | N | 3 | 3 | 3 | 3 | 3 | 3 |
| | % Diff | 3 [symbol] | [symbol] | −0.27 | [symbol] | [symbol] | − [symbol] |
| Group 4, | Mean | [symbol] | 3 | 4.11 | 4.47 | 4.25 | [symbol] |
| 4/4 | SD | 1 | 1 | 0.48 | 0.45 | 0.30 | 0.4 [symbol] |
| mg/kg/dose | N | 3 | 3 | 3 | 3 | 3 | 3 |
| | % Diff | 3 [symbol] | [symbol] | −1 [symbol] .75 | −6.55 | 10. [symbol] | 2.96 |

| | | CRE | CRE | Ca | Ca | P | P |
|---|---|---|---|---|---|---|---|
| | | ( [symbol] /L) | ( [symbol] /L) | ( [symbol] /L) | ( [symbol] /L) | ( [symbol] /L) | ( [symbol] /L) |
| | | [a] | [a] | [a1] | [a] | [a] | [a] |
| Sex: Male | | −4 | 15 | −4 | 15 | −4 | 15 |
| Group 1, | Mean | 4 [symbol] | 4 [symbol] | 2.55 | 2.68 | [symbol] .65 | [symbol] |
| 0 | SD | [symbol] | [symbol] | 0.12 | 0.15 | 0.12 | 0. [symbol] 6 |
| mg/kg/dose | N | 3 | [symbol] | 3 | 3 | 3 | 3 |
| Group 2, | Mean | 5 [symbol] | [symbol] | [symbol] | 2.52 | 1.49 | 1.48 |
| 2.5/9.9 | SD | [symbol] | [symbol] | 0.08 | 0.10 | 0.08 | 0.03 |
| mg/kg/dose | N | 3 | [symbol] | [symbol] | 3 | 3 | 3 |
| | % Diff | 1 [symbol] | [symbol] | −1.04 | − [symbol] | −10.08 | −1 [symbol] .27 |
| Group 3, | Mean | [symbol] | [symbol] | 2.5 [symbol] | 2.52 | [symbol] | 1. [symbol] |
| 3.3/6.6 | SD | [symbol] | [symbol] | [symbol] | [symbol] | 0.11 | 0.04 |
| mg/kg/dose | N | [symbol] | [symbol] | [symbol] | 3 | 3 | 3 |
| | % Diff | [symbol] | [symbol] | 0. [symbol] | −5.9 [symbol] | − [symbol] .07 | − [symbol] |
| Group 4, | Mean | [symbol] | [symbol] | 7.5 [symbol] | [symbol] .59 | 1.52 | 1.63 |
| 4/4 | SD | [symbol] | [symbol] | 0.02 | 0.11 | [symbol] | 0.24 |
| mg/kg/dose | N | [symbol] | [symbol] | 3 | 3 | 3 | 3 |
| | % Diff | 8 | [symbol] | 0. [symbol] | − [symbol] | − [symbol] | − [symbol] |

[a] - Anova & Dunnett; [a1] - Kruskal-Wallis & Dunnett on Ranks.
GGT, gamma-glutamyltransferase; sGLU, glucose; UREA, urea; CRE, creatinine; Ca, calcium; P, inorganic phosphorus. [symbol]
indicates data missing or illegible when filed

TABLE 44-2
Effects of FL118 (Group 2, 3, 4) versus vehicle (Group 1) on male dogs'
serum chemical parameters (WuXi AppTec)
Day −4 and Day 15: Relative to the start date (Day 1)

| | | TCHO | TCHO | TG | TG | K | K |
|---|---|---|---|---|---|---|---|
| | | (mmol/L) | (mmol/L) | (mmol/L) | (mmol/L) | (mmol/L) | (mmol/L) |
| | | [a] | [a] | [a] | [a] | [a] | [a1] |
| Sex: Male | | −4 | 15 | −4 | 15 | −4 | 15 |
| Group 1, | Mean | 4.96 | [symbol] .51 | 0.44 | 0.44 | 4.8 | 4.9 |
| 0 | SD | 0.47 | 1.04 | 0.09 | 0.09 | 0.2 | 0.1 |
| mg/kg/dose | N | 3 | 3 | 3 | 3 | 3 | 3 |
| Group 2, | Mean | 5.35 | 5.21 | 0. [symbol] 9 | 0.34 | 4.7 | 4.1 |
| 2.5/9.9 | SD | 0. [symbol] | 1.38 | 0.02 | 0.05 | 0.1 | 0.3 |
| mg/kg/dose | N | 3 | 3 | 3 | 3 | 3 | 3 |
| | % Diff | 8.00 | −5.44 | −10.61 | −22.56 | −2.1 | −15.8 |
| Group 3, | Mean | [symbol] .79 | 5.3 [symbol] | 0.48 | 0.40 | 4.5 | 4.5 |
| 3.3/6.6 | SD | 0.44 | 0.48 | 0.06 | 0.04 | 0.1 | 0. [symbol] |
| mg/kg/dose | N | 3 | 3 | 3 | 3 | 3 | 3 |
| | % Diff | 16. [symbol] | −2.96 | 8. [symbol] | −10.53 | −6.2 | −6.8 |
| Group 4, | Mean | 4.85 | 4.77 | 0.40 | 0.3 [symbol] | 4.7 | 4.4 |
| 4/4 | SD | 0. [symbol] 4 | 0. [symbol] 4 | 0.10 | 0.05 | 0.1 | 0.1 |
| mg/kg/dose | N | 3 | 3 | 3 | 3 | 3 | 3 |
| | % Diff | −2.15 | − [symbol] .42 | −8.33 | −15.79 | −2.8 | −10.3 |

[a] - Anova & Dunnett: * = p ≤ 0.05; *** = p ≤ 0.001; [a1] - Kruskal-Wallis & Dunnett on Ranks.
TCHO, total cholesterol; TG, triglyceride; K, potassium; Na, sodium; Cl, chloride; GLB, globulin.

| | | Na | Na | Cl | Cl | GLB | GLB |
|---|---|---|---|---|---|---|---|
| | | (mmol/L) | (mmol/L) | (mmol/L) | (mmol/L) | (g/L) | (g/L) |
| | | [a1] | [a1] | [a] | [a] | [a] | [a] |
| Sex: Male | | −4 | 15 | −4 | 15 | −4 | 15 |
| Group 1, | Mean | 146 | 146 | 109 | 108 | 32.0 | 37.3 |
| 0 | SD | 0 | 1 | 2 | 3 | 0.9 | 4.8 |
| mg/kg/dose | N | 3 | 3 | 3 | 3 | 3 | 3 |
| Group 2, | Mean | 147 | 147 | 111 | 111 | 29.8* | [symbol] |
| 2.5/9.9 | SD | 2 | 2 | 2 | 1 | 1.0 | 2.1 |
| mg/kg/dose | N | 3 | 3 | 3 | 3 | 3 | 3 |
| | % Diff | 0 | 1 | 2 | 3 | − [symbol] .0 | − [symbol] |
| Group 3, | Mean | 146 | 147 | 108 | 110 | 31.3 | 30.5 |
| 3.3/6.6 | SD | 0 | 0 | 0 | 1 | 0.8 | 2.3 |
| mg/kg/dose | N | 3 | 3 | 3 | 3 | 3 | 3 |
| | % Diff | 0 | 1 | −1 | 2 | −2.2 | −18.3 |
| Group 4, | Mean | 146 | 148 | 110 | 111 | 2 [symbol] .0*** | 28.3* |
| 4/4 | SD | 1 | 1 | 1 | 2 | 0. [symbol] | 1.0 |
| mg/kg/dose | N | 3 | 3 | 3 | 3 | 3 | 3 |
| | % Diff | 0 | 2 | 1 | [symbol] | −12.4 | −24.1 |

[a] - Anova & Dunnett: * = p ≤ 0.05; *** = p ≤ 0.001; [a1] - Kruskal-Wallis & Dunnett on Ranks.
TCHO, total cholesterol; TG, triglyceride; K, potassium; Na, sodium; Cl, chloride; GLB, globulin.

| | | | | CK | CK |
|---|---|---|---|---|---|
| | | A/G | A/G | (U/L) | (U/L) |
| | | [a] | [a1] | [a] | [a] |
| Sex: Male | | −4 | 15 | −4 | 15 |
| Group 1, | Mean | 0.96 | 0.78 | 305 | 312 |
| 0 | SD | 0.07 | 0.16 | 57 | 113 |
| mg/kg/dose | N | 3 | 3 | 3 | 3 |
| Group 2, | Mean | 1.04 | 0.97 | 453 | 331 |
| 2.5/9.9 | SD | 0.03 | 0.02 | 201 | 110 |
| mg/kg/dose | N | 3 | 3 | 3 | 3 |
| | % Diff | [symbol] .36 | 24.61 | 4 [symbol] | 6 |
| Group 3, | Mean | 1.04 | 0.96 | 261 | 211 |
| 3.3/6.6 | SD | 0.01 | 0.09 | 84 | 41 |
| mg/kg/dose | N | 3 | 3 | 3 | 3 |
| | % Diff | [symbol] .13 | 23.26 | −15 | − [symbol] |
| Group 4, | Mean | 1.11* | 1.07 | [symbol] 50 | 281 |
| 4/4 | SD | 0.04 | 0.02 | 7 [symbol] | 14 [symbol] |
| mg/kg/dose | N | 3 | 3 | 3 | 3 |
| | % Diff | 15.41 | [symbol] 7.18 | 15 | −10 |

[a] - Anova & Dunnett: * = p ≤ 0.05; [a1] - Kruskal-Wallis & Dunnett on Ranks.
A/G, A/G ratio; CK, creatine kinase. [symbol]
indicates data missing or illegible when filed

TABLE 45-1
Effects of FL118 (Group 2, 3, 4) versus vehicle (Group 1) on female dogs'
serum chemical parameters (WuXi AppTec)
Day −4 and Day 15: Relative to the start date (Day 1)

| | | ALT | ALT | AST | AST | TP | TP |
|---|---|---|---|---|---|---|---|
| | | (U/L) | (U/L) | (U/L) | (U/L) | (g/L) | (g/L) |
| | | [a] | [a1] | [a2] | [a2] | [a2] | [a2] |
| Sex: Female | | −4 | 15 | −4 | 15 | −4 | 15 |
| Group 1, | Mean | 27 | 29 | 34 | 31 | 54.6 | 55.5 |
| 0 | SD | 2 | 4 | 12 | 6 | 1.5 | 1.2 |
| mg/kg/dose | N | 3 | 3 | 3 | 3 | 3 | 3 |
| Group 2, | Mean | 27 | 28 | 32 | 26 | 60.6 | 56.4 |
| 2.5/9.9 | SD | 6 | 7 | 7 | [symbol] | 6.7 | 5. [symbol] |
| mg/kg/dose | N | 3 | 3 | 3 | 3 | 3 | 3 |
| | % Diff | 1 | −5 | −7 | −15 | 10.9 | 1.6 |
| Group 3, | Mean | 25 | [symbol] 0 | 32 | 26 | 56.4 | 54.7 |
| 3.3/6.6 | SD | 1 | 4 | 8 | [symbol] | 4.8 | 2.8 |
| mg/kg/dose | N | 3 | 3 | 3 | 3 | 3 | 3 |
| | % Diff | −5 | 3 | −8 | − [symbol] 6 | [symbol] .2 | −1.4 |
| Group 4, | Mean | 25 | 36 | 31 | 29 | 57.4 | 57.5 |
| 4/4 | SD | 3 | 16 | 6 | 4 | 4.9 | 6.0 |
| mg/kg/dose | N | 3 | 3 | 3 | 3 | 3 | 3 |
| | % Diff | −8 | 24 | −11 | −7 | [symbol] .1 | 3.5 |

[a] - Kruskal-Wallis & Dunnett on Ranks; [a1] - Anova & Dunnett (Log); [a2] - Anova & Dunnett.
ALT, alanine aminotransferase; AST, aspartate aminotransferase; TP, total protein; ALB, albumin; BIL-T,
total bilirubin (diasys); ALP, alkaline phosphatase.

| | | ALB | ALB | BIL-T | BIL-T | ALP | ALP |
|---|---|---|---|---|---|---|---|
| | | (g/L) | (g/L) | (μmol/L) | (μmol/L) | (U/L) | (U/L) |
| | | [a2] | [a2] | [a2] | [a2] | [a2] | [a2] |
| Sex: Female | | −4 | 15 | −4 | 15 | −4 | 15 |
| Group 1, | Mean | 29.5 | 29.3 | 1.32 | 1.49 | 158 | 136 |
| 0 | SD | 1.1 | 1.9 | 0.32 | 0.13 | 33 | 14 |
| mg/kg/dose | N | 3 | 3 | 3 | 3 | 3 | 3 |
| Group 2, | Mean | [symbol] 0.5 | 27.7 | 1.84 | 1.73 | 121 | 94 |
| 2.5/9.9 | SD | 4.4 | 4.3 | 0.5 [symbol] | 0.12 | 2 [symbol] | [symbol] |
| mg/kg/dose | N | 3 | 3 | 3 | 3 | 3 | 3 |
| | % Diff | 3.2 | −5.4 | 3 [symbol] | 16.14 | −23 | −31 |
| Group 3, | Mean | 30.5 | 29.2 | 1.85 | 1.66 | 1 [symbol] | 136 |
| 3.3/6.6 | SD | 2. [symbol] | 1.9 | 0.50 | 0.40 | 49 | 47 |
| mg/kg/dose | N | 3 | 3 | 3 | 3 | 3 | 3 |
| | % Diff | 3.3 | −0.3 | 39.90 | 11.88 | 6 | 0 |
| Group 4, | Mean | 3 [symbol] .1 | 2 [symbol] .8 | 1.6 [symbol] | 1.60 | 129 | 110 |
| 4/4 | SD | 2.3 | 3.1 | 0.3 [symbol] | 0.09 | 57 | 47 |
| mg/kg/dose | N | 3 | 3 | 3 | 3 | 3 | 3 |
| | % Diff | 1.8 | −1.6 | 23.23 | 7.8 [symbol] | −19 | −19 |

[a] - Kruskal-Wallis & Dunnett on Ranks; [a1] - Anova & Dunnett (Log); [a2] - Anova & Dunnett.
ALT, alanine aminotransferase; AST, aspartate aminotransferase; TP, total protein; ALB, albumin; BIL-T,
total bilirubin (diasys); ALP, alkaline phosphatase.

| | | GGT | GGT | sGLU | sGLU | UREA | UREA |
|---|---|---|---|---|---|---|---|
| | | (U/L) | (U/L) | (mmol/L) | (mmol/L) | (mmol/L) | (mmol/L) |
| | | [a] | [a1] | [a1] | [a1] | [a] | [a1] |
| Sex: Female | | −4 | 15 | −4 | 15 | −4 | 15 |
| Group 1, | Mean | 3 | 3 | 4. [symbol] | 4.27 | 4.1 [symbol] | 4.14 |
| 0 | SD | 0 | [symbol] | 0.22 | 0.35 | 0.07 | 0.50 |
| mg/kg/dose | N | 3 | 3 | 3 | 3 | 3 | 3 |
| Group 2, | Mean | 3 | 3 | 5.02 | 4.71 | 4.56 | 3.82 |
| 2.5/9.9 | SD | 0 | 1 | 0. [symbol] 5 | 0.22 | [symbol] | 0.25 |
| mg/kg/dose | N | 3 | 3 | 3 | 3 | 3 | 3 |
| | % Diff | 0 | −20 | 6. [symbol] | 10.22 | 5.56 | − [symbol] |
| Group 3, | Mean | 3 | 3 | 5.12 | 5.01 | 4.7 [symbol] | 3.73 |
| 3.3/6.6 | SD | 1 | [symbol] | 0.5 [symbol] | 0.49 | 0. [symbol] 3 | [symbol] .07 |
| mg/kg/dose | N | 3 | 3 | 3 | 3 | 3 | 3 |
| | % Diff | 11 | −20 | [symbol] .78 | 17.16 | 13.71 | −9. [symbol] 3 |
| Group 4, | Mean | 3 | 3 | 4. [symbol] | 4.59 | 4.17 | 5. [symbol] 8 |
| 4/4 | SD | 0 | 1 | 0.37 | 0.13 | 0. [symbol] | [symbol] .23 |
| mg/kg/dose | N | 3 | 3 | 3 | 3 | 3 | 3 |
| | % Diff | 0 | 0 | −0.07 | 7.41 | [symbol] .97 | −3.71 |

| | | CRE | CRE | Ca | Ca | P | P |
|---|---|---|---|---|---|---|---|
| | | (μmol/L) | (μmol/L) | (mmol/L) | (mmol/L) | (mmol/L) | (mmol/L) |
| | | [a2] | [a2] | [a1] | [a] | [a1] | [a] |
| Sex: Female | | −4 | 15 | −4 | 15 | −4 | 15 |
| Group 1, | Mean | 47 | 48 | 2.55 | 2.5 [symbol] | 1.72 | 1.65 |
| 0 | SD | 5 | 4 | 0.06 | 0.0 [symbol] | 0.1 [symbol] | 0.30 |
| mg/kg/dose | N | 3 | 3 | 3 | 3 | 3 | 3 |
| Group 2, | Mean | 48 | 5 [symbol] | 2.56 | 2. [symbol] | 1. [symbol] | 1.51 |
| 2.5/9.9 | SD | 7 | [symbol] | 0.16 | 0.1 [symbol] | 0.06 | 0.09 |
| mg/kg/dose | N | 3 | [symbol] | 3 | 3 | 3 | 3 |
| | % Diff | 3 | [symbol] | 0.52 | −1.32 | −1 [symbol] .4 [symbol] | −8.50 |
| Group 3, | Mean | 45 | 44 | 2.49 | 2.49 | 1.58 | 1.53 |
| 3.3/6.6 | SD | 5 | 5 | 0.07 | 0.04 | 0.20 | 0.10 |
| mg/kg/dose | N | 3 | 3 | 3 | 3 | 3 | 3 |
| | % Diff | −4 | −8 | −2.23 | − [symbol] .71 | −7.77 | −7.29 |
| Group 4, | Mean | 52 | 54 | 2. [symbol] 9 | 2.53 | 1.61 | 1.4 [symbol] |
| 4/4 | SD | 12 | 12 | 0.08 | 0.08 | 0.15 | 0.01 |
| mg/kg/dose | N | 3 | 3 | 3 | 3 | 3 | 3 |
| | % Diff | 11 | 12 | −2.36 | 0.0 [symbol] | −6.41 | −11.74 |

[a] - Kruskal-Wallis & Dunnett on Ranks; [a1] - Anova & Dunnett; [a2] - Anova & Dunnett (Log).
GGT, gamma-glutamyltransferase; sGLU, glucose; UREA, urea; CRE, creatinine; Ca, calcium; P, inorganic phosphorus. [symbol]
indicates data missing or illegible when filed

TABLE 45-2
Effects of FL118 (Group 2, 3, 4) versus vehicle (Group 1) on female dogs' serum chemical parameters (WuXi AppTec)
Day −4 and Day 15: Relative to the start date (Day 1)

| | | TCHO | TCHO | TG | TG | K | K |
|---|---|---|---|---|---|---|---|
| | | (mmol/L) | (mmol/L) | (mmol/L) | (mmol/L) | (mmol/L) | (mmol/L) |
| | | [a] | [a] | [a1] | [a] | [a] | [a] |
| Sex: Female | | −4 | 15 | −4 | 15 | −4 | 15 |
| Group 1, | Mean | 3.93 | 3.78 | 0.50 | 0.49 | 4.7 | 4.4 |
| 0 | SD | 0.31 | 0.24 | 0.12 | 0.04 | 0.2 | 0.1 |
| mg/kg/dose | N | 3 | 3 | 3 | 3 | 3 | 3 |
| Group 2, | Mean | [symbol] .85 | [symbol] .51 | 0.47 | 0.31** | 4.4 | 4.2 |
| 2.5/9.9 | SD | 1.01 | 1.03 | 0.05 | 0.04 | 0.4 | 0.3 |
| mg/kg/dose | N | 3 | 3 | 3 | 3 | 3 | 3 |
| | % Diff | −1.87 | −6.97 | −7.28 | −37. [symbol] 4 | −5.7 | −3.8 |
| Group 3, | Mean | 4. [symbol] 5 | 3.96 | 0.31 | 0.35* | 4.4 | 4.2 |
| 3.3/6.6 | SD | 0.71 | 0.48 | 0.07 | 0.08 | 0.2 | 0.2 |
| mg/kg/dose | N | 3 | 3 | 3 | 3 | 3 | 3 |
| | % Diff | 3.23 | 4.85 | −3 [symbol] .41 | −2 [symbol] .38 | − [symbol] .7 | −4.5 |
| Group 4, | Mean | 3.66 | 3.52 | 0.45 | 0.36* | 4.3 | [symbol] .0 |
| 4/4 | SD | 0.42 | 0.40 | 0.24 | 0.0 [symbol] | 0.2 | 0.2 |
| mg/kg/dose | N | 3 | 3 | 3 | 3 | 3 | 3 |
| | % Diff | −6.71 | −6.71 | −11.26 | −27.70 | −7. [symbol] | −8.3 |

[a] - Anova & Dunnett: * = p ≤ 0.05; ** = p ≤ 0.01; [a1] - Anova & Dunnett (Log); [a2] - Kruskal-Wallis & Dunnett on Ranks.
TCHO, total cholesterol; TG, triglyceride; K, potassium; Na, sodium; Cl, chloride; GLB, globulin.

| | | Na | Na | Cl | Cl | GLB | GLB |
|---|---|---|---|---|---|---|---|
| | | (mmol/L) | (mmol/L) | (mmol/L) | (mmol/L) | (g/L) | (g/L) |
| | | [a] | [a] | [a2] | [a] | [a] | [a] |
| Sex: Female | | −4 | 15 | −4 | 15 | −4 | 15 |
| Group 1, | Mean | 146 | 147 | 110 | 111 | 25.1 | 26.2 |
| 0 | SD | 1 | 1 | 2 | 1 | [symbol] .8 | 1.3 |
| mg/kg/dose | N | 3 | 3 | 3 | 3 | 3 | 3 |
| Group 2, | Mean | 147 | 147 | 111 | 111 | 30.1 | 28.7 |
| 2.5/9.9 | SD | 1 | 0 | 2 | 2 | 2.3 | 0.9 |
| mg/kg/dose | N | 3 | 3 | 3 | 3 | 3 | 3 |
| | % Diff | 1 | 0 | 0 | 0 | 20.1 | 9.3 |
| Group 3, | Mean | 145 | 146 | 109 | 110 | 25.9 | 25.6 |
| 3.3/6.6 | SD | 1 | 1 | 1 | 1 | 2.6 | 1.0 |
| mg/kg/dose | N | 3 | 3 | 3 | 3 | 3 | 3 |
| | % Diff | 0 | −1 | −1 | −1 | 3.2 | −2.5 |
| Group 4, | Mean | 147 | 14 [symbol] | 112 | 112 | 27.3 | 2 [symbol] .7 |
| 4/4 | SD | 0 | 1 | 0 | 1 | 2.9 | 3.7 |
| mg/kg/dose | N | 3 | 3 | 3 | 3 | 3 | 3 |
| | % Diff | 1 | [symbol] | 2 | 1 | 9.0 | 9.3 |

[a] - Anova & Dunnett: * = p ≤ 0.05; ** = p ≤ 0.01; [a1] - Anova & Dunnett (Log); [a2] - Kruskal-Wallis & Dunnett on Ranks.
TCHO, total cholesterol; TG, triglyceride; K, potassium; Na, sodium; Cl, chloride; GLB, globulin.

| | | | | CK | CK |
|---|---|---|---|---|---|
| | | A/G | A/G | (U/L) | (U/L) |
| | | [a] | [a] | [a1] | [a] |
| Sex: Female | | −4 | 15 | −4 | 15 |
| Group 1, | Mean | 1.18 | 1.12 | [symbol] 83 | 293 |
| 0 | SD | 0.05 | 0.11 | 233 | 71 |
| mg/kg/dose | N | 3 | 3 | 3 | 3 |
| Group 2, | Mean | 1.01* | 0.96 | 215 | 238 |
| 2.5/9.9 | SD | 0.07 | 0.12 | 31 | 94 |
| mg/kg/dose | N | 3 | 3 | 3 | 3 |
| | % Diff | −14.44 | −13. [symbol] 4 | −44 | −19 |
| Group 3, | Mean | 1.18 | 1.14 | 221 | 162 |
| 3.3/6.6 | SD | 0.08 | 0.04 | [symbol] 1 | 28 |
| mg/kg/dose | N | 3 | 3 | 3 | 3 |
| | % Diff | 0.2 [symbol] | 1. [symbol] 9 | −42 | −45 |
| Group 4, | Mean | 1.10 | 1.01 | 233 | 200 |
| 4/4 | SD | 0.07 | 0.10 | 77 | [symbol] |
| mg/kg/dose | N | 3 | 3 | 3 | 3 |
| | % Diff | −6.39 | −9.79 | −39 | −32 |

[a] - Anova & Dunnett: * = p ≤ 0.05; [a1] - Anova & Dunnett (Log).
A/G, A/G ratio; CK, creatine kinase. [symbol]
indicates data missing or illegible when filed

In conclusion, oral administration of FL118 to beagle dogs once weekly on Day 1 and Day 8 at doses of 2.5→9.9, 3.3→6.6, or 4.4 mg/kg/dose were well tolerated and no adverse changes in the body weight, food consumption, coagulation, urinalysis gross pathology, organ weights or histopathological evaluation. The no observed adverse effect level (NOAEL) for this study was considered to be 4.4 mg/kg/dose for males.

Example 66—FL118 blood partitioning in human whole blood: As shown in Table 46, the test article FL118 has a favorable whole blood partitioning results in comparison with the positive control. So, FL118 could have much less toxicity in blood.

TABLE 46
FL118 human whole bood partitioning results

| | | RBC to Plasma | | Whole Blood to | |
|---|---|---|---|---|---|
| | | Partitioning | | Plasma Partitioning | |
| | | (K_{RBC/P}) | | (K_{WB/P}) | |
| Test | | Test | | Test | |
| Article | Species | Article | Chlorthalido | Article | Chlorthalidone |
| FL118 | Human | 1.04 | 19.7 | 1.02 | 8.73 |

Example 67—FL118 Caco-2 cell permeability results: As shown in Table 47, FL118's Caco-2 cell permeability is not affected by the Pgp inhibitor Valspodar. Therefore, FL118 could overcome P-gp expression-induced treatment resistance as an additional favorable feature for anticancer.

TABLE 47
FL118 Caco-2 cell permeability results

| | | | | | | Efflux | Absorption | P-gp |
|---|---|---|---|---|---|---|---|---|
| Test | | Recovery | Pₐₚₚ (10⁻⁶ cm/s) | | | Ratio | Potential | Substrate |
| Article | Direction | (%) | R1 | R2 | AVG | [symbol] | Classificatio [symbol] | Classificatio [symbol] |
| FL118 | A-to-B | 43 | 7.27 | 6.67 | 6.97 | 1.2 | High | Negative |
| | B-to-A | 48 | 7.35 | 8.86 | 8.10 | | | |
| FL118 + 1 μM | A-to-B | 46 | 9.26 | 7.48 | 8.37 | 1.0 | | |
| Valspodar | B-to-A | 48 | 8.29 | 8.65 | 8.47 | | | |

[symbol]

indicates data missing or illegible when filed

Absorption Potential Classification: Pₐₚₚ (A-to-B)<1.0×10⁻⁶ cm/s: Low
Pₐₚₚ (A-to-B)≥1.0×10⁻⁶ cm/s: High

### P-gp Substrate Classification:

ER≥2.0 without valspodar, and reduced by ≥50% with valspodar: Positive
ER≥2.0 without valspodar, and reduced by <50% with valspodar: Negative
ER<2.0 without and with valspodar: Negative

Example 68—FL118 human plasma protein binding studies: Interestingly, as shown in Table 48, almost all of the FL118 in blood is in a state of binding plasma protein. As shown in published studies, FL118 has shown high antitumor efficacy in human tumor animal models. Thus, the binding of FL118 to plasma proteins is not a problem and could be an advantage for FL118 such as stability.

TABLE 48
FL118 plasma protein binding experiment results

% Bound of Plasma

| | | Test Article | |
|---|---|---|---|
| Test Article | Species | (FL118) | Warfarin* |
| FL118 | Human | 99.4 | 99.3 |

*Warfarin is positive control. The Warfarin binding acceptance criteria for human plasma is ≥98.0% bound

Example 69—FL118 stability studies in human hepatocytes: As shown in Table 49a, FL118 has a long half-life in human hepatocytes in comparison with the control compound testosterone (Table 49b). This will save FL118 for circulation to tumor tissues (See FL118 oral PK studies in this invention)

TABLE 49a
FL118 half-life results in cryopreserved human hepatocytes

| | | % Remaining | | | | | | Clᵢₙₜ^{b} |
|---|---|---|---|---|---|---|---|---|
| Test | | 0 | 15 | 30 | 60 | 120 | Half- | (mL/min/ |
| Article | Species | min | min | min | min | min | life^{a} | 10⁶ cells) |
| FL118 | Human | 100 | 79 | 71 | 66 | 58 | >120 | <0.00385 |
| | | | | | | | (156 [symbol] | (0.00296) |

^{a}When the calculated half-life is longer than the duration of the experiment, the half-life is expressed as > the longest incubation time. Then, if the calculated half-life is <2x the duration of the experiment, the calculated half- life is listed in parentheses.
^{b} Intrinsic clearance (CLᵢₙₜ) was calculated based on CLᵢₙₜ = k/P, where k is the elimination rate constant and P is the cell concentration in the incubation. [symbol]
indicates data missing or illegible when filed

TABLE 49b
Half-life of testosterone results
in cryopreserved human hepatocytes

| | | Clᵢₙₜ | Half-life |
|---|---|---|---|
| | Half-life | (mL/min/10⁶ | Acceptance |
| Species | (min) | cells) | Criteria |
| Human | 2.2 | 0.212 | ≤5.0 |

Example 70—FL118 stability studies in human liver microsome: Similarly, as shown in Table 50a, FL118 has a long half-life in human liver microsome in comparison with the control compound testosterone (Table 50b).

TABLE 50a
FL118 stability results from human liver microsome

| | | % Remaining of Initial (n = 1) | | | | | | CLᵢₙ^{b} |
|---|---|---|---|---|---|---|---|---|
| Test | | 0 | 10 | 20 | 30 | 60 | Half- | (mL/min/ |
| Article | Species | min | min | min | min | min | life^{a} | [symbol] |
| FL118 | Human | 100 | 93 | 82 | 76 | 58 | >60 (75) | <0.0231 |
| | | | | | | | | (0.0186) |

^{a}When the calculated half-life is longer than the duration of the experiment, the half-life is expressed as > the longest incubation time. Then, if the calculated half-life is <2x the duration of the experiment, the calculated half- life is listed in parentheses.
^{b}Intrinsic clearance (CLᵢₙₜ) was calculated based on CLᵢₙₜ = k/P, where k is the elimination rate constant and P is the protein concentration in the incubation. [symbol]
indicates data missing or illegible when filed

TABLE 50b
Control compound testosterone results
parameters from liver microsome

| | | | CLᵢₙₜ | Acceptable |
|---|---|---|---|---|
| Control | | Half-life | (ml/min/mg | Range |
| Compound | Species | (min) | protein) | (t_{1/2}, min) |
| Testosterone | Human | 29 | 0.0478 | ≤41 |

Example 71-11.118 stability studies in human liver S9 fraction: As shown in Table 51a, FL118 has a long half-life in human liver S9 fraction in comparison with the control compounds testosterone and 7-hydroxycoumarin (Table 51b).

TABLE 51a
FL118 stability results from liver S9 fraction

| | | % Remaining of Initial (n = 1) | | | | | Half- | CLᵢₙₜ^{b} |
|---|---|---|---|---|---|---|---|---|
| Test | | 0 | 10 | 20 | 30 | 60 | life^{a} | (mL/min/mg |
| Article | Species | min | min | min | min | min | (min) | protein) |
| FL118 | Human | 100 | 82 | 73 | 69 | 52 | >60 (63) | <0.0116 (0.0111) |

^{a}When the calculated half-life is longer than the duration of the experiment, the half-life is expressed as > the longest incubation time. Then, if the calculated half-life is <2x the duration of the experiment, the calculated half-life is listed in parentheses. Similarly, if the calculated half-life is less than the shortest time point, the half-life is expressed as < that time point and the calculated half-life is also listed in parentheses.
^{b}Intrinsic clearance (CLᵢₙₜ) was calculated based on CLᵢₙₜ = k/P, where k is the elimination rate constant and P is the protein concentration in the incubation.

TABLE 51b
Results parameters from human liver S9 fraction using
control compounds testosterone and 7-hydroxycoumarin.

| | | | CLᵢₙₜ | Acceptable |
|---|---|---|---|---|
| | | Half-life | (ml/min/mg | Range |
| Control Compound | Species | (min) | protein) | (t_{1/2}, min) |
| Testosterone | Human | 33 | 0.0207 | ≤37 |
| 7-hydroxycoumarin | | 12 | 0.0573 | ≤15 |

Example 72—FL118 stability studies in human plasma and human whole blood: As shown in Table 52, FL118 has a long half-life in human plasma and human whole blood.

TABLE 52
FL118 stability results from human plasma and human whole blood

| Test | | Percent Remaining | | | | | Half-Life |
|---|---|---|---|---|---|---|---|
| Article | Matrix | 0 min | 15 min | 30 min | 60 min | 120 min | [symbol] |
| FL118 | Human Plasma | 100 | 111 | 110 | 94 | 26 | 92 |
| | Human Whole Blood | 100 | 108 | 68 | 61 | 35 | 74 |

[symbol]

indicates data missing or illegible when filed

Example 73-Studies to determine whether FL118 is a ABCG2/BCRP substrate: As shown in Table 53, in the presence or absence of the ABCG2/BCRP inhibitor Ko143, FL118 shows similar rate to pass the Caco-2 cell membrane, indicating FL118 is not a substrate of the efflux pump protein, ABCG2/BCRP.

TABLE 53
FL118 is not a substrate of the efflux pump protein, ABCG2/BCRP

| | | | | | | Efflux | BCRP |
|---|---|---|---|---|---|---|---|
| Test | | Recovery | Pₐₚₚ (10⁻⁶ cm/s) | | | Ratio | Substrate |
| Article | Direction | (%) | R1 | R2 | AVG | (ER) | Classification |
| FL118 | A-to-B | 57 | 11.2 | 10.3 | 10.7 | 0.9 | Negative |
| | B-to-A | 63 | 10.9 | 9.35 | 10.1 | | |
| FL118 | A-to-B | 57 | 14.1 | 8.43 | 11.3 | 0.6 | |
| | B-to-A | 66 | 7.70 | 6.84 | 7.27 | | |

### BCRP Substrate Classification:

ER≥2.0 without Ko143, and reduced by ≥50% with Ko143: Positive
ER≥2.0 without Ko143, and reduced by <50% with Ko143: Negative
ER<2.0 without and with Ko143 (ABCG2/BCRP inhibitor): Negative

Example 74—Studies to determine whether FL118 inhibits ABCG2/BCRP to take other substrates: As shown in Table 54, FL118 does not act like the BCRP inhibitor Kol43 that can inhibit cladribine acting as a BCRP substrate, FL118 does not inhibit cladribine acting as a BCRP substrate.

TABLE 54
FL118 does not inhibit ABCG2/BCRP to take other ABCG2/BCRP substrates

| | | | | | | | BCRP |
|---|---|---|---|---|---|---|---|
| | | | | | | Efflux | Inhibition |
| | | Recovery | Pₐₚₚ (10⁻⁶ cm/s) | | | Ratio | Potential |
| Test Article | Direction | (%) | R1 | R2 | AVG | [symbol] | Classification |
| Cladribine | A-to-B | 100 | 0.16 | 0.15 | 0.16 | 53 | — |
| | B-to-A | 104 | 8.38 | 8.13 | 8.26 | | |
| Cladribine + 1.0 | A-to-B | 101 | 0.13 | 0.10 | 0.11 | 67 | Negative |
| μM FL118 | B-to-A | 118 | 7.29 | 8.04 | 7.67 | | |
| Cladribine + | A-to-B | 99 | 0.25 | 0.19 | 0.22 | 1.1 | Positive |
| 0.5 [symbol] M K [symbol] 143 | B-to-A | 105 | 0.25 | 0.25 | 0.25 | | |

[symbol]

indicates data missing or illegible when filed

### Inhibition Potential Classification:

ER≥2.0 without inhibitor, and reduced by ≥50% with inhibitor: Positive ER≥2.0
without inhibitor, and reduced by <50 with inhibitor: Negative ER<2.0
without and with inhibitor: Negative

Example 75—Studies to determine whether FL118 inhibits P-gp to take other substrates: As shown in Table 55, FL118 does not act like the P-gp inhibitor valspodar that can inhibit digoxin acting as a P-gp substrate, FL118 does not inhibit digoxin acting as a BCRP substrate.

TABLE 55
FL118 does not inhibit P-gp to take other P-gp substrates

| | | | | | | Efflux | P-gp Inhibition |
|---|---|---|---|---|---|---|---|
| | | Recovery | Pₐₚₚ (10⁻⁶ cm/s) | | | Ratio | Potential |
| Test Article | Direction | (%) | R1 | R2 | AVG | (ER) | Classification |
| Digoxin | A-to-B | 76 | 1.59 | 1.13 | 1.36 | 8.4 | — |
| | B-to-A | 79 | 9.86 | 13.1 | 11.5 | | |
| Digoxin + | A-to-B | 80 | 0.71 | 1.18 | 0.95 | 11 | Negative |
| 1.0 μM FL118 | B-to-A | 83 | 8.76 | 11.5 | 10.1 | | |
| Digoxin + | A-to-B | 83 | 3.54 | 2.96 | 3.25 | 1.0 | Positive |
| 1.0 μM Valspodar | B-to-A | 88 | 2.78 | 3.79 | 3.29 | | |

### Inhibition Potential Classification:

ER≥2.0 without inhibitor, and reduced by ≥50% with inhibitor: Positive ER≥2.0
without inhibitor, and reduced by <50 with inhibitor: Negative ER<2.0
without and with inhibitor: Negative

Example 76-Studies to determine whether FL118 inhibits CYP P450 (CYP1A2, CYP2B6, CYP3A) activity: Table 56 shows the 3 CYP isoforms' corresponding probe substrates, metabolites and positive control inhibitor. As shown in Table 57, in comparison with the percentage inhibition by the positive controls, FL118 does not inhibit these 3 CYP isoforms that are required by the FDA for Investigational New Drug (IND) submission for clinical trials.

TABLE 56
CYP probe substrates and metabolites

| | Probe | | Positive Control |
|---|---|---|---|
| CYP | Substrate | Metabolite | Inhibitor |
| CYP1A2 | Phenacetin | Acetaminophen | α-Naphthoflavone |
| | (63 μM) | | (1 μM) |
| CYP2B6 | Bupropion | OH-bupropion | Thio-TEPA |
| | (75 μM) | | (20 μM) |
| CYP3A | Testosterone | 6β-OH | Ketoconazole |
| | (55 μM) | testosterone | (1 μM) |

TABLE 57
FL118 does not inhibit CYP P450
(CYP1A2, CYP2B6, CYP3A) activity

| CYP | % Inhibition^{a} | % Inhibition |
|---|---|---|
| Isoform | (1.0 μM FL118) | (Positive Control) |
| 1A2 | 0.1 | 92.1 |
| 2B6 | 0.0 | 71.5 |
| 3A | 0.0 | 94.5 |

^{a}Percent Inhibition values calculated as negative as reported as 0.0.

Example 77—Studies to determine whether FL118 induces CYP1A2 activity: As shown in Table 59, in comparison with the negative and positive controls, FL118 does not inhibit these induce CYP1A2 mRNA.

TABLE 59
FL118 does not induce CYP1A2 mRNA,
avoiding such potential toxicity

| | | | | | | % of |
|---|---|---|---|---|---|---|
| | | | | Fold-induction^{a} | | OME-treated |
| Donor | Test Article | Treatment | | Mean | SD | Cells^{b} |
| 1 | Control | Vehicle | | 1.0 | 0.19 | 0 |
| | | OME | | 91 | 10 | 100 |
| | FL118 | 0.01 | μM | 1.2 | 0.22 | 0.15 |
| | | 0.1 | μM | 2.0 | 0.45 | 1.1 |
| | | 1 | μM | 3.4 | 0.27 | 2.6 |
| 2 | Control | Vehicle | | 1.0 | 0.18 | 0 |
| | | OME | | 148 | 17 | 100 |
| | FL118 | 0.001 | μM | 2.3 | 0.88 | 0.88 |
| | | 0.005 | μM | 2.5 | 0.44 | 1.0 |
| | | 0.01 | μM | 4.9 | 0.38 | 2.6 |
| 3 | Control | Vehicle | | 1.0 | 0.10 | 0 |
| | | Formulation | | 1.0 | 0.07 | 0 |
| | | OME | | 67 | 4.6 | 100 |
| | FL118 | 0.025 | μM | 2.0 | 0.26 | 1.6 |
| | | 0.05 | μM | 2.1 | 0.29 | 1.6 |
| | HPβCD- | 1 | μM | 2.4 | 0.24 | 2.1 |
| | formulated | | | | | |

^{a}Fold-induction was calculated from the normalized mRNA level (2^{-ΔΔCt)} of TA- or positive inducer-treated cells relative to that of vehicle control-treated cells from triplicate measurements.
^{b}Percentage of mRNA fold-induction relative to positive inducer.

Example 78—Studies to determine whether FL118 induces CYP2B6 activity. As shown in Table 60, in comparison with the negative and positive controls, FL118 does not inhibit these induce CYP2B6 mRNA.

TABLE 60
FL118 does not induce CYP2B6 mRNA,
avoiding such potential toxicity

| | | | | | | % of |
|---|---|---|---|---|---|---|
| | | | | Fold-induction^{a} | | PB-treated |
| Donor | Test Article | Treatment | | Mean | SD | Cells^{b} |
| 1 | Control | Vehicle | | 1.0 | 0.14 | 0 |
| | | PB | | 100 | 6.3 | 100 |
| | FL118 | 0.01 | μM | 0.62 | 0.05 | 0 |
| | | 0.1 | μM | 0.45 | 0.05 | 0 |
| | | 1 | μM | 0.02 | 0.00 | 0 |
| 2 | Control | Vehicle | | 1.0 | 0.09 | 0 |
| | | PB | | 71 | 19 | 100 |
| | FL118 | 0.001 | μM | 1.5 | 0.38 | 0.70 |
| | | 0.005 | μM | 1.3 | 0.33 | 0.43 |
| | | 0.01 | μM | 1.1 | 0.15 | 0.11 |
| 3 | Control | Vehicle | | 1.0 | 0.11 | 0 |
| | | Formulation | | 1.0 | 0.12 | 0 |
| | | PB | | 355 | 43 | 100 |
| | FL118 | 0.025 | μM | 0.61 | 0.10 | 0 |
| | | 0.05 | μM | 0.71 | 0.07 | 0 |
| | HPβCD- | 1 | μM | 0.04 | 0.00 | 0 |
| | formulated | | | | | |

^{a}Fold-induction was calculated from the normalized mRNA level (2^{-ΔΔCt}) of TA- or positive inducer-treated cells relative to that of vehicle control-treated cells from triplicate measurements.
^{b}Percentage of mRNA fold-induction relative to positive inducer. Negative values are treated as zero.

Example 79—Studies to determine whether FL118 induces CYP3A activity: As shown in Table 61, FL118 only exhibits a very minor induction of CYP3A4 mRNA in one of the three hepatocyte donors. Therefore, there is no serious concern for potential toxicity

TABLE 61
FL118 induces a minor induction (5-8%) of CYP3A4
mRNA in one of the three hepatocyte donors.

| | | | | | | % of |
|---|---|---|---|---|---|---|
| | | | | Fold-induction^{a} | | RIF-treated |
| Donor | Test Article | Treatment | | Mean | SD | Cells |
| 1 | Control | Vehicle | | 1.0 | 0.37 | 0 |
| | | RIF | | 38 | 2.7 | 100 |
| | FL118 | 0.01 | μM | 2.2 | 0.46 | 3.2 |
| | | 0.1 | μM | 1.8 | 0.18 | 2.1 |
| | | 1 | μM | 0.01 | 0.00 | 0 |
| 2 | Control | Vehicle | | 1.0 | 0.20 | 0 |
| | | RIF | | 47 | 5.1 | 100 |
| | FL118 | 0.001 | μM | 3.1 | 0.33 | 4.4 |
| | | 0.005 | μM | 4.1 | 0.43 | 6.7 |
| | | 0.01 | μM | 4.6 | 0.66 | 7.7 |
| 3 | Control | Vehicle | | 1.0 | 0.26 | 0 |
| | | Formulation | | 1.0 | 0.21 | 0 |
| | | RIF | | 31 | 3.4 | 100 |
| | FL118 | 0.025 | μM | 0.83 | 0.22 | 0 |
| | | 0.05 | μM | 0.80 | 0.12 | 0 |
| | HPβCD- | 1 | μM | 0.09 | 0.02 | 0 |
| | formulated | | | | | |

^{a}Fold-induction was calculated from the normalized mRNA level (2^{-ΔΔCt}) of TA- or positive inducer-treated cells relative to that of vehicle control-treated cells from triplicate measurements.
^{b} Percentage of mRNA fold-induction relative to positive inducer. Negative values are treated as zero.

Example 80—Studies to determine cell viability after CYP Induction treatment in human hepatocytes: Table 62 shows FL118 in parallel with positive and negative controls in the cytotoxicity.

TABLE 62
Cell viability after human hepatocytes treated
with vehicle (control) and FL118

| | | | | Formazan | | % of |
|---|---|---|---|---|---|---|
| | | | | Absorbance | | Vehicle- |
| Donor | Test Article | Treatment | | Mean | SD | treated Cells |
| 1 | Control | Vehicle | | 1.64 | 0.11 | 100 |
| | | OME | | 1.60 | 0.29 | 97 |
| | | PB | | 1.34 | 0.13 | 82 |
| | | RIF | | 1.33 | 0.13 | 81 |
| | FL118 | 0.01 | μM | 1.48 | 0.25 | 91 |
| | | 0.1 | μM | 1.20 | 0.19 | 73 |
| | | 1 | μM | 0.512 | 0.03 | 31 |
| 2 | Control | Vehicle | | 1.49 | 0.06 | 100 |
| | | OME | | 1.35 | 0.02 | 91 |
| | | PB | | 1.53 | 0.04 | 102 |
| | | RIF | | 1.37 | 0.03 | 92 |
| | FL118 | 0.001 | μM | 1.53 | 0.08 | 103 |
| | | 0.005 | μM | 1.51 | 0.07 | 101 |
| | | 0.01 | μM | 1.48 | 0.06 | 99 |
| 3 | Control | Vehicle | | 1.73 | 0.08 | 100 |
| | | Formulation | | 1.71 | 0.06 | 99 |
| | | OME | | 1.39 | 0.07 | 80 |
| | | PB | | 1.44 | 0.01 | 84 |
| | | RIF | | 1.45 | 0.04 | 84 |
| | FL118 | 0.025 | μM | 1.63 | 0.06 | 94 |
| | | 0.05 | μM | 1.80 | 0.07 | 104 |
| | HPβCD- | 1 | μM | 1.19 | 0.11 | 69 |
| | formulated | | | | | |

^{a} Percentage of MTS absorbance relative to vehicle control. MTS absorbance <75% of the vehicle control is considered a positive cytotoxic result.

Conclusions for FL118's effects on CYP P450's three isoforms (CYP1A2, CYP2B6, CYP3A) are summarized blow.

In donor 1, the induction of CYP1A2, CYP2B6, and CYP3A4 mRNA expression by FL118 at 0.01, 0.1, and 1 μM was evaluated. FL118 showed dose-dependent cytotoxicity at 0.1 and 1 μM. FL118 did not increase CYP1A2 mRNA (<2-fold vs. vehicle control and <20% of positive control) at 0.01 μM; but it caused 2.0- and 3.4-fold induction at 0.1 and 1 μM, respectively. FL118 did not increase CYP2B6 mRNA at any of the three tested concentrations. FL118 caused 2.2-fold induction of CYP3A4 mRNA at 0.01 μM, but it did not show induction at 0.1 or 1 μM. The effects of 1 μM FL118 are probably underestimated due to the observed cytotoxicity at that concentration.

In donor 2, the induction of CYP1A2, CYP2B6, and CYP3A4 mRNA expression by FL118 at 0.001, 0.005, and 0.01 μM was evaluated. FL118 caused 2.3-, 2.5-, and 4.9-fold induction of CYP1A2 mRNA at 0.001, 0.005, and 0.01 μM, respectively. FL118 did not increase CYP2B6 mRNA at any of the three tested concentrations. FL118 caused 3.1-, 4.1-, and 4.6-fold induction of CYP3A4 mRNA at 0.001, 0.005, and 0.01 μM, respectively. No cytotoxicity of FL118 in the range 0.001 to 0.01 μM was observed in this donor.

In donor 3, the induction of CYP1A2, CYP2B6, and CYP3A4 mRNA expression by FL118 at 0.025 and 0.05 μM, and by HPβCD-formulated FL118 at 1 μM was evaluated. FL118 caused 2.0- and 2.1-fold induction of CYP1A2 mRNA at 0.025 and 0.05 μM, respectively. FL118 did not increase CYP2B6 and CYP3A4 mRNA at any of the two tested concentrations. HPβCD-formulated FL118 at 1 μM caused 2.4-fold induction of CYP1A2 mRNA; it did not show induction of CYP2B6 and CYP3A4 mRNA. FL118 at 0.025 and 0.05 μM did not show cytotoxicity, while HPβCD-formulated FL118 at 1 μM showed some cytotoxicity for this donor.

Example 81—FL118 showed no effects on hERG inhibition over the FL118 concentration range used. Thus, FL118 has no potential cardiovascular toxicity: The hERG potassium channel is a voltage-gated ion channel found in the heart. It is essential for cardiac re-polarization, yet many pharmacological agents can inhibit the hERG current and give rise to potentially fatal toxicity. Mammalian cells expressing the hERG potassium channel were dispensed into 384 well planar arrays and hERG tail currents measured by whole cell voltage clamping. A range of concentrations of the test compound FL118 in a wide range of FL118 concentrations in 4-replicate wells of each FL118 concentration was then added to the cells and a second recording of the hERG current was made. The percent change in hERG current was calculated and used to calculate an IC50 value (i.e., the test compound FL118 concentration which produces 50% inhibition). As shown in FIG. 75 and Table 64a, FL118 does not exhibit an inhibition of hERG activity in a wide range of FL118 concentration. The final conclusive results are summarized in Table 64b.

TABLE 64a
FL118's effects on hERG Inhibition hERG inhibition - Individual Data are shown

| Test | % Mean Inhibition | | | | | | |
|---|---|---|---|---|---|---|---|
| Article | 0 μM | 0.003 μM | 0.01 μM | 0.03 μM | 0.1 μM | 0.3 μM | 1 μM |
| FL118 | 0% | 14.8% | 18.7% | 5.6% | −5.2% | −2.9% | 19.9% |
| Control Inhibitor | 0 μM | 0.008 μM | 0.04 μM | 0.2 μM | 1 μM | 5 μM | 25 μM |
| Quinidine | 0% | −2.2% | −6.4% | 15.1% | 21.8% | 81.0% | 96.8% |

TABLE 64b
Summary of FL118's effects on hERG inhibition

| Test | Test | IC₅₀ | SE IC₅₀ | |
|---|---|---|---|---|
| Article | Concentration | (μM) | (μM) | Comment |
| FL118 | 0.003 μM-1 μM | >1 μM | N/A | No concentration- |
| | | | | dependent |
| | | | | inhibition observed |
| Quinidine | 0.008 μM-25 μM | 1.84 | 0.31 | positive control |

Example 82—FL118 Ames Bacterial Reverse Mutation Assay studies (i.e., Ames Test): The purpose of the Ames Bacterial Reverse Mutation Assay was to determine mutagenic potential of the test article (FL118) by measuring FL118's ability to induce back mutations at specific loci in five bacteria tester strains of (TA97a, TA98, TA100, TA1535, WP2-uvrA). Genotypic characterization was performed for each strain of bacteria to ensure that each strain has retained its specific genotypic markers. The tester strains must demonstrate normal results according.

Summary of the results from Ames Test: (1) A dense pack of microcolonies was observed and there was no apparent thinning or increase in the size of micro-colonies for all FL118 test article plates in comparison to the vehicle (negative) control plates; therefore, these healthy background lawns were given a score of ‘1’. No precipitate was noted on the FL118 test article plates for each strain. (2) In the assay the mean number of revertant colonies for the test article FL118 was less than 2-fold over the mean number of revertant colonies for the negative control and for all strains and conditions except for TA1535-S9 (0.32 μM, 0.10 μM, 0.010 μM concentrations). (3) In the repeat assay with TA1535, the mean number of revertant colonies for the test article was less than 2-fold over the mean number of revertant colonies for the negative control for the-S9 condition of TA1535. (4) See Tables 65-69 of the colony counts, background lawn codes, and the calculated fold increases over the negative control for each bacteria strain.

TABLE 65
Bacterial tester strain TA97A results with the Test article FL118
Bacteria: TA97a

| | | | | | | Fold | |
|---|---|---|---|---|---|---|---|
| | Colony Counts | | | | | Increase | |
| | Replicate | Replicate | Replicate | | Standard | over | Background |
| Treatment | 1 | 2 | 3 | Average | Deviation | Negative | Lawn Code |
| Positive +S9 | 1669 | 1742 | 1552 | 1654 | 95.8 | 10.7 | 1 |
| Positive −S9 | 1683 | 1763 | 1791 | 1746 | 56.0 | 18.4 | 1 |
| Negative DMSO +S9 | 150 | 160 | 151 | 154 | 5.5 | Not | 1 |
| Negative DMSO −S9 | 92 | 103 | 89 | 95 | 7.4 | Applicable | 1 |
| Test Article 1.0 μM +S9 | 140 | 146 | 139 | 142 | 3.8 | 0.9 | 1 |
| Test Article 1.0 μM −S9 | 111 | 113 | 134 | 119 | 12.7 | 1.3 | 1 |
| Test Article 0.32 μM +S9 | 125 | 142 | 130 | 132 | 8.7 | 0.9 | 1 |
| Test Article 0.32 μM −S9 | 119 | 117 | 140 | 125 | 12.7 | 1.3 | 1 |
| Test Article 0.10 μM +S9 | 189 | 150 | 178 | 172 | 20.1 | 1.1 | 1 |
| Test Article 0.10 μM −S9 | 116 | 87 | 82 | 95 | 18.4 | 1.0 | 1 |
| Test Article 0.032 μM +S9 | 158 | 150 | 143 | 150 | 7.5 | 1.0 | 1 |
| Test Article 0.032 μM −S9 | 114 | 93 | 98 | 102 | 11.0 | 1.1 | 1 |
| Test Article 0.010 μM +S9 | 123 | 140 | 161 | 141 | 19.0 | 0.9 | 1 |
| Test Article 0.010 μM −S9 | 108 | 105 | 90 | 101 | 9.6 | 1.1 | 1 |

TABLE 66
Bacterial tester strain TA98 results with the Test article FL118
Bacteria: TA98

| | | | | | | Fold | |
|---|---|---|---|---|---|---|---|
| | Colony Counts | | | | | Increase | |
| | Replicate | Replicate | Replicate | | Standard | over | Background |
| Treatment | 1 | 2 | 3 | Average | Deviation | Negative | Lawn Code |
| Positive +S9 | 1931 | 1765 | 1186 | 1627 | 391.1 | 37.0 | 1 |
| Positive −S9 | 607 | 381 | 415 | 468 | 121.9 | 15.6 | 1 |
| Negative DMSO +S9 | 51 | 28 | 54 | 44 | 14.2 | Not | 1 |
| Negative DMSO −S9 | 38 | 25 | 26 | 30 | 7.2 | Applicable | 1 |
| Test Article 1.0 μM +S9 | 35 | 45 | 52 | 44 | 8.5 | 1.0 | 1 |
| Test Article 1.0 μM −S9 | 24 | 26 | 19 | 23 | 3.6 | 0.8 | 1 |
| Test Article 0.32 μM +S9 | 28 | 43 | 25 | 32 | 9.6 | 0.7 | 1 |
| Test Article 0.32 μM −S9 | 33 | 29 | 22 | 28 | 5.6 | 0.9 | 1 |
| Test Article 0.10 μM +S9 | 45 | 41 | 46 | 44 | 2.6 | 1.0 | 1 |
| Test Article 0.10 μM −S9 | 36 | 20 | 15 | 24 | 11.0 | 0.8 | 1 |
| Test Article 0.032 μM +S9 | 36 | 45 | 32 | 38 | 6.7 | 0.9 | 1 |
| Test Article 0.032 μM −S9 | 28 | 23 | 27 | 26 | 2.6 | 0.9 | 1 |
| Test Article 0.010 μM +S9 | 32 | 37 | 45 | 38 | 6.6 | 0.9 | 1 |
| Test Article 0.010 μM −S9 | 29 | 53 | 32 | 38 | 13.1 | 1.3 | 1 |

TABLE 67
Bacterial tester strain TA100 results with the Test article FL118
Bacteria: TA100

| | | | | | | Fold | |
|---|---|---|---|---|---|---|---|
| | Colony Counts | | | | | Increase | |
| | Replicate | Replicate | Replicate | | Standard | over | Background |
| Treatment | 1 | 2 | 3 | Average | Deviation | Negative | Lawn Code |
| Positive +S9 | 2140 | 2199 | 2520 | 2286 | 204.5 | 24.1 | 1 |
| Positive −S9 | 1418 | 1395 | 1454 | 1422 | 29.7 | 14.5 | 1 |
| Negative DMSO +S9 | 88 | 105 | 92 | 95 | 8.9 | Not | 1 |
| Negative DMSO −S9 | 89 | 103 | 103 | 98 | 8.1 | Applicable | 1 |
| Test Article 1.0 μM +S9 | 105 | 106 | 87 | 99 | 10.7 | 1.0 | 1 |
| Test Article 1.0 μM −S9 | 94 | 75 | 81 | 83 | 9.7 | 0.8 | 1 |
| Test Article 0.32 μM +S9 | 109 | 93 | 85 | 96 | 12.2 | 1.0 | 1 |
| Test Article 0.32 μM −S9 | 83 | 96 | 83 | 87 | 7.5 | 0.9 | 1 |
| Test Article 0.10 μM +S9 | 96 | 74 | 98 | 89 | 13.3 | 0.9 | 1 |
| Test Article 0.10 μM −S9 | 89 | 77 | 94 | 87 | 8.7 | 0.9 | 1 |
| Test Article 0.032 μM +S9 | 83 | 87 | 92 | 87 | 4.5 | 0.9 | 1 |
| Test Article 0.032 μM −S9 | 95 | 68 | 94 | 86 | 15.3 | 0.9 | 1 |
| Test Article 0.010 μM +S9 | 101 | 96 | 79 | 92 | 11.5 | 1.0 | 1 |
| Test Article 0.010 μM −S9 | 72 | 79 | 80 | 77 | 4.4 | 0.8 | 1 |

TABLE 68a
Bacterial tester strain TA1535 results with the Test article FL118
Bacteria: TA1535

| | | | | | | Fold | |
|---|---|---|---|---|---|---|---|
| | Colony Counts | | | | | Increase | |
| | Replicate | Replicate | Replicate | | Standard | over | Background |
| Treatment | 1 | 2 | 3 | Average | Deviation | Negative | Lawn Code |
| Positive +S9 | 236 | 274 | 224 | 245 | 26.1 | 18.8 | 1 |
| Positive −S9 | 1188 | 1162 | 1170 | 1173 | 13.3 | 61.7 | 1 |
| Negative DMSO +S9 | 15 | 13 | 12 | 13 | 1.5 | Not | 1 |
| Negative DMSO −S9 | 20 | 21 | 16 | 19 | 2.6 | Applicable | 1 |
| Test Article 1.0 μM +S9 | 12 | 10 | 15 | 12 | 2.5 | 0.9 | 1 |
| Test Article 1.0 μM −S9 | 23 | 21 | 30 | 25 | 4.7 | 1.3 | 1 |
| Test Article 0.32 μM +S9 | 27 | 18 | 14 | 20 | 6.7 | 1.5 | 1 |
| Test Article 0.32 μM −S9 | 53 | 52 | 56 | 54 | 2.1 | 2.8 | 1 |
| Test Article 0.10 μM +S9 | 13 | 8 | 20 | 14 | 6.0 | 1.1 | 1 |
| Test Article 0.10 μM −S9 | 54 | 62 | 67 | 61 | 6.6 | 3.2 | 1 |
| Test Article 0.032 μM +S9 | 22 | 13 | 19 | 18 | 4.6 | 1.4 | 1 |
| Test Article 0.032 μM −S9 | 43 | 26 | 39 | 36 | 8.9 | 1.9 | 1 |
| Test Article 0.010 μM +S9 | 20 | 9 | 13 | 14 | 5.6 | 1.1 | 1 |
| Test Article 0.010 μM −S9 | 39 | 37 | 41 | 39 | 2.0 | 2.1 | 1 |

TABLE 68b
Bacterial tester strain TA1535 results with the Test article FL118 (partial repeat)
Bacteria: TA1535

| | | | | | | Fold | |
|---|---|---|---|---|---|---|---|
| | Colony Counts | | | | | Increase | |
| | Replicate | Replicate | Replicate | | Standard | over | Background |
| Treatment | 1 | 2 | 3 | Average | Deviation | Negative | Lawn Code |
| Positive −S9 | 808 | 786 | 842 | 812 | 28.2 | 81.2 | 1 |
| Negative DMSO −S9 | 6 | 13 | 11 | 10 | 3.6 | Not | 1 |
| | | | | | | Applicable | |
| Test Article 1.0 μM −S9 | 11 | 13 | 12 | 12 | 1.0 | 1.2 | 1 |
| Test Article 0.32 μM −S9 | 10 | 9 | 10 | 10 | 0.6 | 1.0 | 1 |
| Test Article 0.10 μM −S9 | 16 | 15 | 10 | 14 | 3.2 | 1.4 | 1 |
| Test Article 0.032 μM −S9 | 8 | 8 | 11 | 9 | 1.7 | 0.9 | 1 |
| Test Article 0.010 μM −S9 | 13 | 12 | 12 | 12 | 0.6 | 1.2 | 1 |

TABLE 69
Bacterial tester strain WP2-uvrA results with the Test article FL118
Bacteria: WP2-uvrA

| | | | | | | Fold | |
|---|---|---|---|---|---|---|---|
| | Colony Counts | | | | | Increase | |
| | Replicate | Replicate | Replicate | | Standard | over | Background |
| Treatment | 1 | 2 | 3 | Average | Deviation | Negative | Lawn Code |
| Positive +S9 | 146 | 141 | 142 | 143 | 2.6 | 3.4 | 1 |
| Positive −S9 | 94 | 87 | 97 | 93 | 5.1 | 2.9 | 1 |
| Negative DMSO +S9 | 45 | 31 | 49 | 42 | 9.5 | Not | 1 |
| Negative DMSO −S9 | 31 | 28 | 38 | 32 | 5.1 | Applicable | 1 |
| Test Article 1.0 μM +S9 | 35 | 38 | 43 | 39 | 4.0 | 0.9 | 1 |
| Test Article 1.0 μM −S9 | 27 | 31 | 24 | 27 | 3.5 | 0.8 | 1 |
| Test Article 0.32 μM +S9 | 41 | 45 | 43 | 43 | 2.0 | 1.0 | 1 |
| Test Article 0.32 μM −S9 | 27 | 34 | 20 | 27 | 7.0 | 0.8 | 1 |
| Test Article 0.10 μM +S9 | 48 | 38 | 54 | 47 | 8.1 | 1.1 | 1 |
| Test Article 0.10 μM −S9 | 27 | 36 | 27 | 30 | 5.2 | 0.9 | 1 |
| Test Article 0.032 μM +S9 | 40 | 45 | 39 | 41 | 3.2 | 1.0 | 1 |
| Test Article 0.032 μM −S9 | 38 | 37 | 41 | 39 | 2.1 | 1.2 | 1 |
| Test Article 0.010 μM +S9 | 38 | 47 | 34 | 40 | 6.7 | 1.0 | 1 |
| Test Article 0.010 μM −S9 | 35 | 22 | 31 | 29 | 6.7 | 0.9 | 1 |

Conclusion: FL118 passes the Ames Test and shows non-mutagenic in the Ames mutation test.

Example 83-F1.118 Mouse Lymphoma Assay (ML.A) studies: The purpose of the MLA study was to determine the genotoxic response of the test article FL118 by measuring its ability to induce gene mutations and chromosomal damage using L5178Y TK+/−mouse lymphoma cells with a range of FL118 concentrations. The corresponding detailed method was described in the section of “DETAILED DESCRIPTION OF THE DISCLOSURE” The results are now reported below in Table 70 through Table 75.

TABLE 70
Four hours (4 h) MLA suspension growth treated with vehicle (DMSO),
methyl cyclophosphamide (CP) and test article FL118
Suspension Growth Calculations

| | | | Day 1 | Day 2 | | | Relative | |
|---|---|---|---|---|---|---|---|---|
| | | | Growth | Growth | Suspension | | Suspension | |
| Treatment | | | Cell Conc. | Cell Conc. | Growth | Average | Growth (%) | SG |
| Condition | S9 | [symbol] | (Cell/mL) | (Cell/mL) | (SG) | SG | ( [symbol] SG) | Acceptance |
| Negative | +S9 | [symbol] | 7.11E+05 | 1.69E+06 | 13.4 | 13.1 | 102.3 | PASS |
| Vehicle - DMSO | +S9 | [symbol] | 6.79E+05 | 1.66E+06 | 12.7 | | 96.9 | |
| CP - 15 μg/mL | +S9 | [symbol] | 4.79E+05 | 1.29E+06 | 5.9 | 7.3 | 52.7 | |
| | +S9 | [symbol] | 5.44E+05 | 1.30E+06 | 7.7 | | 58.8 | |
| CP - 17.5 μg/mL | +S9 | 10A | 3.20E+05 | 1.14E+06 | 4.2 | 4.9 | 32.1 | |
| | +S9 | 10B | 3.86E+05 | 1.25E+06 | 5.5 | | 42.0 | |
| Test Article | +S9 | 11A | 7.55E+05 | 1.27E+06 | 10.5 | 10.7 | 80.2 | |
| Concentration 1 | +S9 | 11B | 6.56E+05 | 1.45E+06 | 10.8 | | 82.4 | |
| (1 uM) | | | | | | | | |
| Test Article | +S9 | 12A | 7.02E+05 | 1.44E+06 | 11.0 | 11.8 | 84.0 | |
| Concentration 2 | +S9 | 12B | 7.35E+05 | 1.51E+06 | 12.5 | | 95.4 | |
| (0.32 uM) | | | | | | | | |
| Test Article | +S9 | 13A | 6.71E+05 | 1.55E+06 | 11.4 | 12.9 | 87.0 | |
| Concentration 3 | +S9 | 13B | 7.82E+05 | 1.64E+06 | 14.3 | | 109.2 | |
| (0.1 uM) | | | | | | | | |
| Test Article | +S9 | 14A | 6.42E+05 | 1.45E+06 | 10.1 | 10.8 | 77.1 | |
| Concentration 4 | +S9 | 14B | 5.88E+05 | 1.50E+06 | 11.5 | | 87.8 | |
| (0.032 uM) | | | | | | | | |

SG Acceptance: Average SG of Negative Cultures ≥8 and ≤32 [symbol]
indicates data missing or illegible when filed

TABLE 71
Four hours (4 h) results from MLA cells treated with vehicle
(DMSO), cyclophosphamide (CP) and the test article FL118

| | | [symbol] Count (VC) Plate Data | | | | | | | | [symbol] Plate Data | | | | | |
|---|---|---|---|---|---|---|---|---|---|---|---|---|---|---|---|
| | | [symbol] | | | | | | | | | | | | | |
| | | | | | | Average | | | | | | | | | |
| | | | Colony Count | | | Colony | | | | Plate 1 | | Plate 2 | | Plate 3 | |
| Treatment | | | Plate | Plate | Plate | Count | | | | [symbol] | | [symbol] | | [symbol] | |
| Condition | | [symbol] | 1 | 2 | 3 | [symbol] | [symbol] | [symbol] | [symbol] | Large | Small | Large | Small | Large | Small |
| Plus S9 | Negative | [symbol] | [symbol] | [symbol] | [symbol] | [symbol] | [symbol] | [symbol] | [symbol] | [symbol] | [symbol] | [symbol] | [symbol] | [symbol] | [symbol] |
| Metabolic | Vehicle | [symbol] | [symbol] | [symbol] | [symbol] | [symbol] | [symbol] | [symbol] | [symbol] | [symbol] | [symbol] | [symbol] | [symbol] | [symbol] | [symbol] |
| Activation | [symbol] | | | | | | | | | | | | | | |
| [symbol] | CP [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] |
| | CP [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] |
| | Test Article | [symbol] | [symbol] | [symbol] | [symbol] | [symbol] | [symbol] | [symbol] | [symbol] | [symbol] | [symbol] | [symbol] | [symbol] | [symbol] | [symbol] |
| | Concen- | [symbol] | [symbol] | [symbol] | [symbol] | [symbol] | [symbol] | [symbol] | [symbol] | [symbol] | [symbol] | [symbol] | [symbol] | [symbol] | [symbol] |
| | tration 1 [symbol] | | | | | | | | | | | | | | |
| | Test Article | [symbol] | [symbol] | [symbol] | [symbol] | [symbol] | [symbol] | [symbol] | [symbol] | [symbol] | [symbol] | [symbol] | [symbol] | [symbol] | [symbol] |
| | Concen- | [symbol] | [symbol] | [symbol] | [symbol] | [symbol] | [symbol] | [symbol] | [symbol] | [symbol] | [symbol] | [symbol] | [symbol] | [symbol] | [symbol] |
| | tration 2 [symbol] | | | | | | | | | | | | | | |
| | Test Article | [symbol] | [symbol] | [symbol] | [symbol] | [symbol] | [symbol] | [symbol] | [symbol] | [symbol] | [symbol] | [symbol] | [symbol] | [symbol] | [symbol] |
| | Concen- | [symbol] | [symbol] | [symbol] | [symbol] | [symbol] | [symbol] | [symbol] | [symbol] | [symbol] | [symbol] | [symbol] | [symbol] | [symbol] | [symbol] |
| | tration 3 [symbol] | | | | | | | | | | | | | | |
| | Test Article | [symbol] | [symbol] | [symbol] | [symbol] | [symbol] | [symbol] | [symbol] | [symbol] | [symbol] | [symbol] | [symbol] | [symbol] | [symbol] | [symbol] |
| | Concen- | [symbol] | [symbol] | [symbol] | [symbol] | [symbol] | [symbol] | [symbol] | [symbol] | [symbol] | [symbol] | [symbol] | [symbol] | [symbol] | [symbol] |
| | tration 4 [symbol] | | | | | | | | | | | | | | |

| | | [symbol] Plate Data | | | [symbol] Frequency Data | | | Acceptance Criteria | | | |
|---|---|---|---|---|---|---|---|---|---|---|---|
| | | [symbol] | | | | | | | | | |
| | | | | Average | | | | | | | |
| | | | | Colony | Mutant | [symbol] | | | | | |
| Treatment | | Total | | Count | Frequency | Mutant | | | | | |
| Condition | | Count | [symbol] | [symbol] | [symbol] | Frequency | [symbol] | [symbol] | [symbol] | [symbol] | [symbol] |
| Plus S9 | Negative | [symbol] | [symbol] | [symbol] | [symbol] | | | PASS | PASS | PASS | |
| Metabolic | Vehicle | [symbol] | [symbol] | [symbol] | [symbol] | | | PASS | PASS | PASS | |
| Activation | [symbol] | | | | | | | | | | |
| [symbol] | CP | [symbol] | [symbol] | [symbol] | [symbol] | [symbol] | | PASS | | | PASS |
| | | [symbol] | [symbol] | [symbol] | [symbol] | [symbol] | | PASS | | | PASS |
| | CP | [symbol] | [symbol] | [symbol] | [symbol] | [symbol] | | FAIL | | | PASS |
| | | [symbol] | [symbol] | [symbol] | [symbol] | [symbol] | | PASS | | | PASS |
| | Test Article | [symbol] | [symbol] | [symbol] | [symbol] | [symbol] | [symbol] | PASS | | | |
| | Concen- | [symbol] | [symbol] | [symbol] | [symbol] | [symbol] | [symbol] | PASS | | | |
| | tration 1 [symbol] | | | | | | | | | | |
| | Test Article | [symbol] | [symbol] | [symbol] | [symbol] | [symbol] | [symbol] | PASS | | | |
| | Concen- | [symbol] | [symbol] | [symbol] | [symbol] | [symbol] | [symbol] | PASS | | | |
| | tration 2 [symbol] | | | | | | | | | | |
| | Test Article | [symbol] | [symbol] | [symbol] | [symbol] | [symbol] | [symbol] | PASS | | | |
| | Concen- | [symbol] | [symbol] | [symbol] | [symbol] | [symbol] | [symbol] | PASS | | | |
| | tration 3 [symbol] | | | | | | | | | | |
| | Test Article | [symbol] | [symbol] | [symbol] | [symbol] | [symbol] | [symbol] | PASS | | | |
| | Concen- | [symbol] | [symbol] | [symbol] | [symbol] | [symbol] | [symbol] | PASS | | | |
| | tration 4 [symbol] | | | | | | | | | | |

[symbol]

indicates data missing or illegible when filed

Mutant Frequency and Induced Mutant Frequency are depicted as whole numbers. These numbers represent the occurrence of mutant colonies×10⁻⁶.
Negative Control Absolute Cloning Efficiency (ACE) Acceptance: ACE Negative≥65% and ≤120%
Negative Control Acceptance: RTG ≥10% and the MF ≥35×10⁻⁶ and ≤140×10⁻⁶
Positive Control Acceptance: Induced MF (IMF) ≥300×10⁻⁶ and RTG ≥10%
Mutation Score: IMF≤89×10⁻⁶ the article is Non-Mutagenic. IMF ≥90×10⁻⁶ the article is Mutagenic
The average Negative VC is used to calculate the positive control Relative Cloning Efficiency
Only one concentration of the positive control must pass acceptance criteria for assay to be valid

TABLE 72
Four-hours (4 h) treatment - MLA results summary

| | | | | | | | | |
|---|---|---|---|---|---|---|---|---|
| Treatment | | Avg. | Avg. | Avg. | Avg. | Avg. | Avg. IMF | Avg. % of IMF |
| Condition | S9 | SG | ACE | RTG | MF | IMF | Small Colony | Small Colony |

Negative Controls

| | | | | | |
|---|---|---|---|---|---|
| Negative | +S9 | 13.1 | 81.8% | 99.4% | 63 |
| Vehicle - DMSO | | | | | |

Test Articles

| | | | | | |
|---|---|---|---|---|---|
| Test Article | +S9 | 10.7 | 62.3% | 81.5% | 231 |
| Concentration | | | | | |
| 1(1 uM) | | | | | |
| Test Article | +S9 | 11.8 | 67.5% | 95.2% | 87 |
| Concentration | | | | | |
| 2(0.32 uM) | | | | | |
| Test Article | +S9 | 12.9 | 82.5% | 98.8% | 71 |
| Concentration | | | | | |
| 3(0.1 uM) | | | | | |
| Test Article | +S9 | 10.8 | 89.5% | 89.9% | 53 |
| Concentration | | | | | |
| 4(0.032 uM) | | | | | |

Positive Controls

| | | | | | |
|---|---|---|---|---|---|
| CP - 15 μg/mL | +S9 | 21.6% | 1000 | 621 | 62.1% |
| CP - 17.5 μg/mL | +S9 | 6.7% | 1513 | 880 | 58.6% |

The averages of replicate A and B were calculated for each treatment condition. Data from Table 53 was used to calculate the average ACE, RTG, MF and IMF. Data from Table 56 was used calculate the average IMF of Small Colony and % of IMF of Small Colony.

TABLE 73
Twenty-four hours (24 h) MLA suspension growth treated with vehicle
(DMSO), methyl methanesulfonate (MMS) and the test article FL118

| | | | Day 1 | Day 2 | Day 3 | | | Relative | |
|---|---|---|---|---|---|---|---|---|---|
| | | | Growth | Growth | Growth | Suspension | | Suspension | SG |
| Treatment | | | Cell Conc. | Cell Conc. | Cell Conc. | Growth | Average | Growth | Accep- |
| Condition | S9 | [symbol] | (Cell/mL) | (Cell/mL) | (Cell/mL) | (SG) | SG | (%) ( [symbol] SG) | tance |
| Negative | [symbol] | 15A | 1.21E [symbol] | 1.38E [symbol] | 1.19E [symbol] | 73.5 | 79.1 | 93.0 | PASS |
| Vehicle - DMSO | [symbol] | 15B | 1.20E [symbol] | 1.41E [symbol] | 1.35E [symbol] | 84.6 | | 107.8 | |
| MMS - 5 μg/mL | [symbol] | 16A | 9.11E [symbol] | 1.19E [symbol] | 1.17E [symbol] | 45.8 | 48.2 | [symbol] | |
| | [symbol] | 16B | 1.03E [symbol] | 9.42E [symbol] | 1.42E [symbol] | 49.5 | | 62.5 | |
| MMS - 8 μg/mL | [symbol] | 17A | 5.51E [symbol] | 8.72E [symbol] | 8.56E [symbol] | 23.5 | 26.2 | 29.7 | |
| | [symbol] | 17B | 9.18E [symbol] | 9.23E [symbol] | 5.93E [symbol] | 28.8 | | 35.4 | |
| Test | [symbol] | 18A | 4.31E [symbol] | 1.21E [symbol] | 3.30E [symbol] | 5.2 | 4.3 | 7.8 | |
| Article(0.32 uM) | [symbol] | 18B | 4.35E [symbol] | 1.15E [symbol] | 1.07E [symbol] | 2.3 | | 2.9 | |
| Test | [symbol] | 19A | 7.95E [symbol] | 1.12E [symbol] | 8.74E [symbol] | 29.0 | 29.1 | 36.7 | |
| Article(0.1 uM) | [symbol] | 19B | 7.88E [symbol] | 9.74E [symbol] | 1.04E [symbol] | 29.1 | | 35.8 | |
| Test | [symbol] | 20A | 1.11E [symbol] | 1.08E [symbol] | 1.26E [symbol] | 55.9 | 62.0 | 70.7 | |
| Article(0.032 uM) | [symbol] | 20B | 1.11E [symbol] | 1.20E [symbol] | 1.37E [symbol] | 68.1 | | 85.1 | |
| Test | [symbol] | 21A | 9.94E [symbol] | 1.37E [symbol] | 1.02E [symbol] | 55.8 | 66.2 | 84.5 | |
| Article(0.001 uM) | [symbol] | 21B | 1.17E [symbol] | 1.20E [symbol] | 1.27E [symbol] | 65.5 | | 87.8 | |

SG Acceptance: Average SG of Negative Cultures ≥32 and ≤180 [symbol]
indicates data missing or illegible when filed

TABLE 74
Twenty-four hours (24 h) results from MLA cells treated with vehicle
(DMSO), methyl methanesulfonate (MMS) and the test article FL118

| | | [symbol] Count (VC) Plate Data | | | | | | | | | | | | | |
|---|---|---|---|---|---|---|---|---|---|---|---|---|---|---|---|
| | | | | | | Average | | | | [symbol] Plate Data | | | | | |
| | | | | | | Colony | | | | Plate 1 | | Plate 2 | | Plate 3 | |
| Treatment | | | Colony Count | | | Count | | | | [symbol] | | [symbol] | | [symbol] | |
| Condition | | [symbol] | [symbol] | [symbol] | [symbol] | [symbol] | [symbol] | [symbol] | [symbol] | Large | Small | Large | Small | Large | Small |
| [symbol] | Negative | [symbol] | [symbol] | [symbol] | [symbol] | [symbol] | [symbol] | [symbol] | [symbol] | [symbol] | [symbol] | [symbol] | [symbol] | [symbol] | [symbol] |
| Metabolic | Vehicle | [symbol] | [symbol] | [symbol] | [symbol] | [symbol] | [symbol] | [symbol] | [symbol] | [symbol] | [symbol] | [symbol] | [symbol] | [symbol] | [symbol] |
| Activation | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | MMS [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] |
| | MMS [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] |
| | Test | [symbol] | [symbol] | [symbol] | [symbol] | [symbol] | [symbol] | [symbol] | [symbol] | [symbol] | [symbol] | [symbol] | [symbol] | [symbol] | [symbol] |
| | Article [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | Test | [symbol] | [symbol] | [symbol] | [symbol] | [symbol] | [symbol] | [symbol] | [symbol] | [symbol] | [symbol] | [symbol] | [symbol] | [symbol] | [symbol] |
| | Article [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | Test | [symbol] | [symbol] | [symbol] | [symbol] | [symbol] | [symbol] | [symbol] | [symbol] | [symbol] | [symbol] | [symbol] | [symbol] | [symbol] | [symbol] |
| | Article [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | Test | [symbol] | [symbol] | [symbol] | [symbol] | [symbol] | [symbol] | [symbol] | [symbol] | [symbol] | [symbol] | [symbol] | [symbol] | [symbol] | [symbol] |
| | Article [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |

| | | [symbol] Plate Data | | | Mutant | | | | | | |
|---|---|---|---|---|---|---|---|---|---|---|---|
| | | | | Average | Frequency Data | | | | | | |
| | | | | Colony | Mutant | [symbol] | | | | | |
| Treatment | | Total | | Count | Frequency | Mutant | | Acceptance Criteria | | | |
| Condition | | Count | [symbol] | [symbol] | [symbol] | Frequency | [symbol] | [symbol] | [symbol] | [symbol] | [symbol] |
| [symbol] | Negative | [symbol] | [symbol] | [symbol] | [symbol] | | | PASS | PASS | PASS | |
| Metabolic | Vehicle | [symbol] | [symbol] | [symbol] | [symbol] | | | PASS | PASS | PASS | |
| Activation | [symbol] | [symbol] | [symbol] | [symbol] | [symbol] | [symbol] | | PASS | | | PASS |
| [symbol] | MMS [symbol] | [symbol] | [symbol] | [symbol] | [symbol] | [symbol] | | PASS | | | PASS |
| | | [symbol] | [symbol] | [symbol] | [symbol] | [symbol] | | PASS | | | PASS |
| | MMS [symbol] | [symbol] | [symbol] | [symbol] | [symbol] | [symbol] | | PASS | | | PASS |
| | | [symbol] | [symbol] | [symbol] | [symbol] | [symbol] | [symbol] | FAIL | | | |
| | Test | [symbol] | [symbol] | [symbol] | [symbol] | [symbol] | [symbol] | FAIL | | | |
| | Article [symbol] | [symbol] | [symbol] | [symbol] | [symbol] | [symbol] | [symbol] | PASS | | | |
| | Test | [symbol] | [symbol] | [symbol] | [symbol] | [symbol] | [symbol] | PASS | | | |
| | Article [symbol] | [symbol] | [symbol] | [symbol] | [symbol] | [symbol] | [symbol] | PASS | | | |
| | Test | [symbol] | [symbol] | [symbol] | [symbol] | [symbol] | [symbol] | PASS | | | |
| | Article [symbol] | [symbol] | [symbol] | [symbol] | [symbol] | [symbol] | [symbol] | PASS | | | |
| | Test | [symbol] | [symbol] | [symbol] | [symbol] | [symbol] | [symbol] | PASS | | | |
| | Article [symbol] | [symbol] | [symbol] | [symbol] | [symbol] | [symbol] | [symbol] | PASS | | | |

[symbol]

indicates data missing or illegible when filed

Mutant Frequency and Induced Mutant Frequency are depicted as whole numbers. These numbers represent the occurrence of mutant colonies×10⁻⁶.
Negative Control Absolute Cloning Efficiency (ACE) Acceptance: ACE Negative ≥65% and ≤120%
Negative Control Acceptance: RTG ≥10% and the MF ≥35×10⁶ and ≤140×10⁻⁶ Positive Control Acceptance: Induced MF (IMF) ≥300×10⁶ and RTG ≥10%
Mutation Score: IMF≤89×10⁻⁶ the article is Non-Mutagenic. IMF ≥90×10⁻⁶ the article is Mutagenic
The average Negative VC is used to calculate the positive control Relative Cloning Efficiency
The test article concentration of 0.32 μM did not pass validity criteria 7 and was therefore not used in analysis.

TABLE 75
Twenty-four-hours (24h) treatment - MLA results summary

| | | | | | | | | |
|---|---|---|---|---|---|---|---|---|
| | | Avg. | Avg. | Avg. | Avg. | Avg. | Avg. IMF | Avg. % of IMF |
| Treatment Condition | S9 | SG | ACE | RTG | MF | IMF | Small Colony | Small Colony |

Negative Controls

| | | | | | |
|---|---|---|---|---|---|
| Negative | −S9 | 79.1 | 81.5% | 100.5% | 61 |
| Vehicle - DMSO | | | | | |

Test Articles

| | | | | | |
|---|---|---|---|---|---|
| Test Article(0.32 uM) | −S9 | 4.3 | 3.0% | 0.2% | 2125 |
| Test Article(0.1 uM) | −S9 | 29.1 | 57.8% | 25.0% | 345 |
| Test Article(0.032 uM) | −S9 | 52.0 | 84.0% | 80.3% | 73 |
| Test Article(.01 uM) | −S9 | 66.2 | 84.5% | 86.7% | 125 |

Positive Controls

| | | | | | |
|---|---|---|---|---|---|
| MMS - 5 μg/mL | −S9 | 38.7% | 458 | 240 | 52.4% |
| MMS - 8 μg/mL | −S9 | 15.5% | 750 | 417 | 54.7% |

The averages of replicate A and B were calculated for each treatment condition. Data from Table 55 was used to calculate the average ACE, RTG, MF and IMF. Data from Table 56 was used calculate the average IMF of Small Colony and % of IMF of Small Colony. MF and IMF expressed as (mutants/1×10⁶ cells).

Conclusion derived from the FL118 MLA results: Given the valid negative and positive controls in parallel with the test article FL118, the Mouse Lymphoma Assay (MLA) results from FL118 are assumed to be reliable. In this regard, if the Mouse Lymphoma Assay (MLA) study was run with concentrations of ≤0.032 μM for the 4-hour-S9, ≤0.32 μM for the 4-hour+S9 and ≤0.032 μM for the 24 hour-S9 assays, the test article FL118 would have been considered unable to induce mutation in the test system. However, in the highest concentrations tested (FL118 at the concentrations of >0.1 μM for the 4-hour-S9, >1.0 μM for the 4 hour+S9 and ≥0.1 μM for the 24 hour-S9 assays), the test article FL118 was considered positive (mutagenic/clastogenic) because there was evidence of an increase in the mutant frequency above the concurrent background that was greater than the Global Evaluation Factor (the test article mutant frequency was greater than 90×10⁻⁶ of the average mutant frequency of the concurrent negative control). Additionally, the Relative Total Growth (RTG) of these test article doses were greater than 10%. Importantly, FL118 is highly potent, it does not need to have to be administered at a high dose/concentration to kill cancer cells. The PK studies indicated that a≤25 nM concentration can be reached in cancer cells. Therefore, the issue that high doses/concentrations induce mouse lymphoma cell mutation would not be an issue since such high concentration will never be reached in a physiological condition.

Example 84—FL118 oral pharmacokinetics studies: Following a single oral dose administration of FL118 to SCID mice, plasma and tumor concentrations were found to follow a monoexponential decay over time. The calculated plasma and tumor PK parameters are provided in (Table 76) with the corresponding plasma (ng/mL) and tumor (ng/g) concentrations used to calculate the PK parameters provided in Table 77.

TABLE 76
Mouse plasma and human tumor issue pharmacokinetic (PK) parameters
for a single-dose oral administration of 1.5 mg/kg FL118.

| | | | | SE | | | AUC | | |
|---|---|---|---|---|---|---|---|---|---|
| | Tₘₐₓ* | t_{1/2} | Cₘₐₓ | Cₘₐₓ | AUCₐₗₗ | AUC_{∞} | 0-8 | V_{D} | Cl |
| Matrix | (hr) | (hr) | (ng/mL) | (ng/mL) | (hr*ng/mL) | (hr*ng/mL) | (hr*ng/mL) | (mL/kg) | (mL/hr/kg) |
| Plasma | 0.500 | 5.12 | 21.4 | 3.88 | 74.1 | 88.0 | 63.0 | 126,000 | 17,000 |

| | | | | SE | | | AUC | | |
|---|---|---|---|---|---|---|---|---|---|
| | Tₘₐₓ | t_{1/2} | Cₘₐₓ | Cₘₐₓ | AUCₐₗₗ | AUC_{∞} | 0-8 | V_{D} | Cl |
| Matrix | (hr) | (hr) | (ng/g) | (ng/g) | (hr*ng/g) | (hr*ng/g) | (hr*ng/g) | (g/kg) | (g/hr/kg) |
| HT29 | 1.00 | 4.24 | 31.2 | 0.822 | 128 | 178 | 128 | 51,700 | 8,440 |
| Tumor | | | | | | | | | |
| SW620 | 1.00 | 2.99 | 60.5 | 4.74 | 188 | 224 | 188 | 28,900 | 6,710 |
| Tumor | | | | | | | | | |

*AUC, area under the concentration-time curve; AUC₀₋ₜ, area under the concentration-time curve from time zero to time t; AUC_{∞,} area under the concentration-time curve from time zero to infinity; AUC_{all,} area under the concentration-time curve from time zero to the last detected time point; Cₘₐₓ, maximum observed drug concentration; Cl, clearance; SE, standard error; t, time, time point; t_{1/2}, terminal elimination half-life in plasma; Tₘₐₓ, time of the maximum plasma drug concentration; V_{D}, apparent volume of distribution.

The AUCall for the plasma, HT29 tumor and SW620 tumor were 74.1 hr*ng/ml, and 128 and 188 hr*ng/g, respectively. The plasma and tumor exposures quantitated from 0.0-8.0 hours (AUC0-8) suggest that there was higher exposure in both SW620 and HT29 tumors compared to plasma (AUCO-8 in SW620 and HT29 tumors were 188 and 128 hr*ng/g, respectively, compared to plasma, which was 63.0 hr*ng/mL, assuming 1.0 g=1.0 ml water). The individual terminal elimination half-lives (t1/2) of FL118 for plasma, HT29 tumor, and SW620 tumor were 5.12, 4.24, and 2.99 hours, respectively. The maximum FL118 concentrations for plasma, HT29 tumor, and SW620 tumor were 21.4 ng/mL, and 31.2, and 60.5 ng/g, respectively, which correlate with the increased exposure of FL118 in the SW620 and HT29 tumors, and the increased clearance parameter calculated for plasma (Cl of plasma=17,000 mL/hr/kg compared to 8,440 g/hr/kg in HT29 and 6,710 g/hr/kg in SW620, assuming 1.0 g=1.0 mL water).

TABLE 77
Mouse plasma and human tumor tissue FL118 concentrations
(ng/mL and ng/g, respectively) over time (hours) from
a single dose oral administration of 1.5 mg/kg FL118.

| | | | Nominal Time (hours) | | | | | | | |
|---|---|---|---|---|---|---|---|---|---|---|
| Analyte | Matrix | Mouse | 0.50 | 1.0 | 2.0 | 4.0 | 8.0 | 12.0 | 24.0 | 48.0 |
| | | | FL118_Conc (ng/mL) | | | | | | | |
| FL118 | Plasma | 1 | 13.9 | NS | BLQ | NS | 2.87 | 1.59 | BLQ | BLQ |
| | | 2 | 26.7 | 14.3 | 12.7 | 5.53 | 4.70 | 1.47 | BLQ | BLQ |
| | | 3 | 23.8 | 19.1 | 9.65 | 4.78 | BLQ | 2.18 | BLQ | BLQ |
| | | n | 3 | 2 | 2 | 2 | 2 | 3 | 0 | 0 |
| | | Min | 13.9 | 14.3 | 9.65 | 4.78 | 2.87 | 1.47 | NC | NC |
| | | Max | 26.7 | 19.1 | 12.7 | 5.53 | 4.70 | 2.18 | NC | NC |
| | | Mean | 21.4 | 16.7 | 11.2 | 5.15 | 3.78 | 1.75 | NC | NC |
| | | Median | 23.8 | 16.7 | 11.2 | 5.15 | 3.78 | 1.59 | NC | NC |
| | | SD | 6.72 | 3.36 | 2.13 | 0.532 | 1.29 | 0.377 | NC | NC |
| | | CV % | 31.4 | 20.1 | 19.1 | 10.3 | 34.1 | 21.6 | NC | NC |
| | | | FL118 Concentration (ng/g tissue) | | | | | | | |
| FL118 | HT29 | 1 | 27.3 | 31.7 | 15.0 | 8.31 | 4.86 | BLQ | BLQ | BLQ |
| | Tumor | 2 | 14.2 | 29.6 | 23.4 | 16.5 | 11.6 | BLQ | BLQ | BLQ |
| | | 3 | 18.0 | 32.2 | 28.3 | 20.2 | BLQ | BLQ | BLQ | BLQ |
| | | n | 3 | 3 | 3 | 3 | 2 | 0 | 0 | 0 |
| | | Min | 14.2 | 29.6 | 15.0 | 8.31 | 4.86 | NC | NC | NC |
| | | Max | 27.3 | 32.2 | 28.3 | 20.2 | 11.6 | NC | NC | NC |
| | | Mean | 19.8 | 31.2 | 22.2 | 15.0 | 8.23 | NC | NC | NC |
| | | Median | 18.0 | 31.7 | 23.4 | 16.5 | 8.23 | NC | NC | NC |
| | | SD | 6.74 | 1.42 | 6.71 | 6.08 | 4.76 | NC | NC | NC |
| | | CV % | 34.0 | 4.57 | 30.2 | 40.6 | 57.9 | NC | NC | NC |
| FL118 | SW620 | 1 | 37.3 | 55.4 | 49.2 | 11.7 | 6.27 | BLQ | BLQ | BLQ |
| | Tumor | 2 | 37.2 | 69.9 | 25.0 | 24.2 | 15.5 | BLQ | BLQ | BLQ |
| | | 3 | 34.6 | 56.0 | 37.4 | 15.7 | 4.30 | BLQ | BLQ | BLQ |
| | | n | 3 | 3 | 3 | 3 | 3 | 0 | 0 | 0 |
| | | Min | 34.6 | 55.4 | 25.0 | 11.7 | 4.30 | NC | NC | NC |
| | | Max | 37.3 | 69.9 | 49.2 | 24.2 | 15.5 | NC | NC | NC |
| | | Mean | 36.4 | 60.5 | 37.2 | 17.2 | 8.69 | NC | NC | NC |
| | | Median | 37.2 | 56.0 | 37.4 | 15.7 | 6.27 | NC | NC | NC |
| | | SD | 1.52 | 8.21 | 12.1 | 6.37 | 5.97 | NC | NC | NC |
| | | CV % | 4.18 | 13.6 | 32.6 | 37.0 | 68.8 | NC | NC | NC |

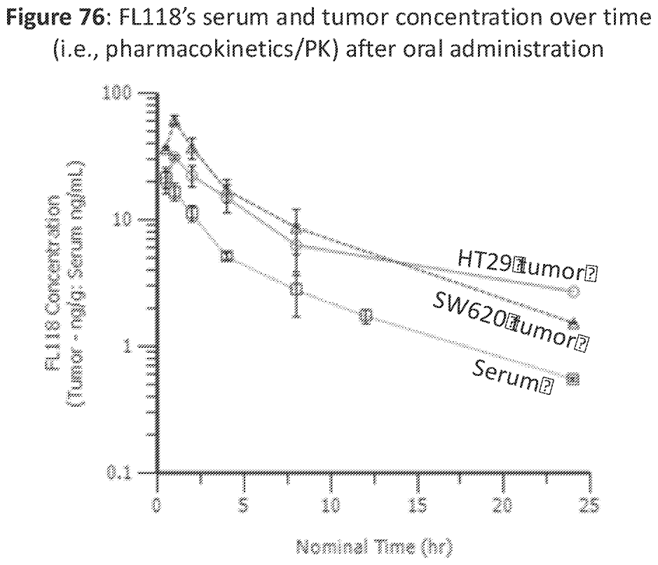

Conclusion derived from FL118 oral PK studies: After oral administration of FL118, FL118 is rapidly cleared from blood stream/circulation and accumulated in human tumor tissues (FIG. 76).

Example 85—FL118's spectral wavelength versus absorbance changes in four different solvent content buffer conditions: FL118 at 10 μM solution with or without a high NaCl salt was prepared in a PBS (pH=7.4) or a PBS containing 4% HSA (w/v, mimic of HSA concentration in a human body) and analyzed by a UV spectrometer, scanning from 310 nm to 450 nm. The data shown in FIG. 77 demonstrated that FL118 in PBS exhibited high absorbance and after additional 4% human serum albumin (HSA), FL118's absorbance decrease in comparison with FL118 in PBS, suggesting HAS can interact with FL118 to decrease FL118 UV absorbance. Furthermore, FL118 in a high NaCl salt showed significantly decreasing UV absorbance suggesting the NaCl salt can interact with FL118. After addition of 4% HSA, FL118's absorbance can further decrease suggesting that HAS can interact salted FL118 as well.

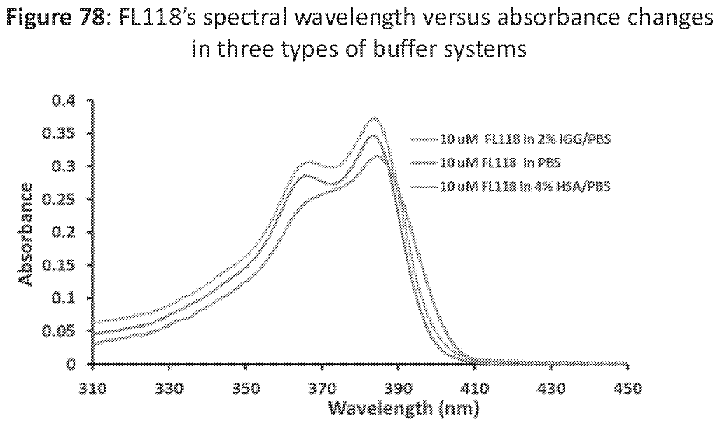

Example 86—FL118's spectral wavelength versus absorbance change in 2% IgG in PBS buffer, in PBS buffer only and in 4% HSA in PBS buffer: The same concentration (10 μM) of FL118 solutions in a PBS, or a PBS containing 4% HSA (w/v) or a PBS containing 2% IGG (w/v) were prepared and analyzed by a UV spectrometer, scanning from 310 nm to 450 nm. The data shown in FIG. 78 demonstrated that IgG has little interactions with FL118 (no UV absorbance decrease. However, HSA at 4% concentration (w/v) has a strong interaction with FL118, reflecting FL118 UV absorbance decrease (red) in comparison with FL118 in PBS.

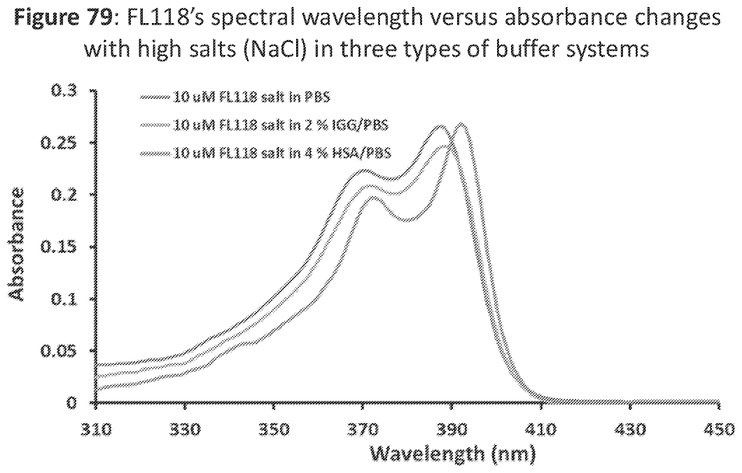

Example 87—FL118 spectral wavelength versus absorbance change with high salts (NaCl) in PBS buffer only, in 2% IgG in PBS buffer, and in 4% HSA in PBS buffer: FL118 saltwith high NaCl salt at 10 μM in a PBS, or a PBS containing 4% HSA (w/v) or a PBS containing 2% IGG (w/v) were prepared and analyzed by a UV spectrometer, scanning from 310 nm to 450 nm. The data shown in FIG. 79 demonstrated that IgG has a very weak interaction with FL118 (only slightly UV absorbance decrease). However, HSA at 4% concentration (w/v) has a strong interaction with FL118, resulted in the FL118 UV absorbance decrease in comparison with FL118 in PBS.

Example 88-Spectral wavelength versus absorbance change for FL118 and SPEFL118 in three conditions of (1) FL118 in PBS buffer only, (2) FL118 in 4% HSA in PBS buffer, and HSA-formulated FL118 (SPEFL118) in PBS buffer: FL118 at 10 μM in PBS buffer or FL118 at 10 μM in a PBS containing 4% HSA (w/v) or and SPEFL118 at 10 μM in a PBS were analyzed by a UV spectrometer, scanning from 310 nm to 450 nm. As the data shown in FIG. 80, the HAS-formulated FL118 (SPEFL118) has a tight interaction with FL118 inside the HAS protein (well encapsulation of FL 118 by a HAS. In other words, FL118 was well capsulated by HSA within the binding packet of HSA), since SPEFL118 exhibited a striking UV absorbance decrease in comparison with either the FL118 in PBS buffer only or FL118 in 4% HSA in PBS buffer.

After much experimentation, FL118 was encapsulated using single-protein-encapsulation (SPE) technology with varied ratios of drug to protein, such as, 2:1, 3:1, 4:1, 6:1, 8:1, and 12:1. In this disclosure, the antitumor efficacy and toxicity results are provided from the example using human serum albumin (HSA) to formulate FL118 into SPEFL118. In comparison with the free form (naked) FL118, the encapsulated FL118 molecule is surrounded by different environments (HSA), which causes the change of FL118 UV spectra or florescence emission spectroscopy. In other words, the carrier, binding and proximity relationships of the encapsulated FL118 molecule with the single protein HSA would exhibit a difference in absorption, fluorescence, circular dichroism spectra, compared to a corresponding un-tightly bound single protein in the presence or absence of FL118 molecules. Importantly, the lyophilized SPEFL118 is water-soluble, and the resulting solution can be stable in 2-8° C. for longer period time, such as 4 -12 weeks. Although the ddH2O-resuspended SPEFL118 can be sedimented after 1-2 weeks especially in the high concentration of FL118, gently reversing the tube can resuspend SPEFL118. The resuspended SPEFL 118 has no antitumor efficacy and toxicity property changes in up to 12 weeks.

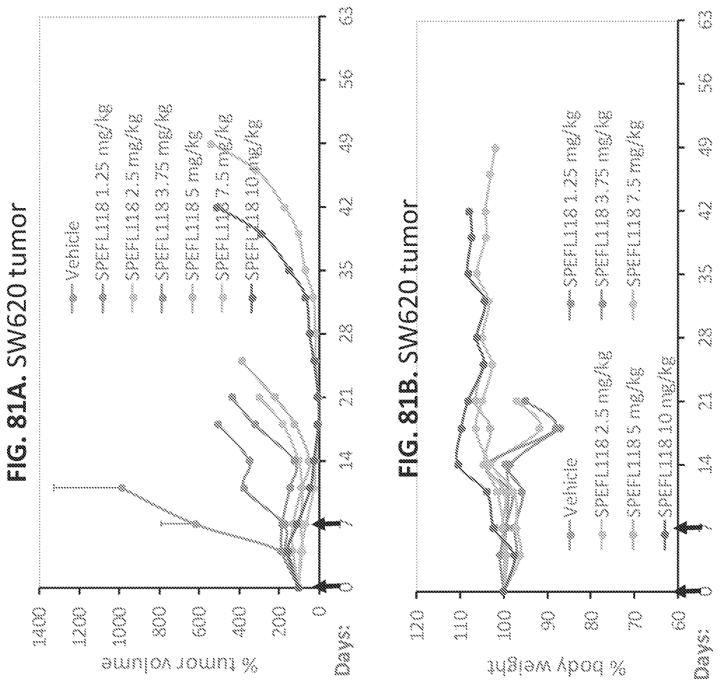

Example 89—SPEFL118's antitumor activity and toxicity in human colorectal cancer ((RC) tumor female SCID mouse models via intraperitoneal (i.p.) routes: The antitumor activity and toxicity of SPEFL118 was determined in a wide range of SPEFL 118 dosing levels using human CRC SW620 tumor animal models via i.p. routes with the schedule of weekly×2 as shown in FIG. 81 (arrowed). At many different dose levels, SPEFL118 could exhibit a high efficacy to regress SW620 tumors (FIG. 81A) without showing clear toxicity (FIG. 81B).

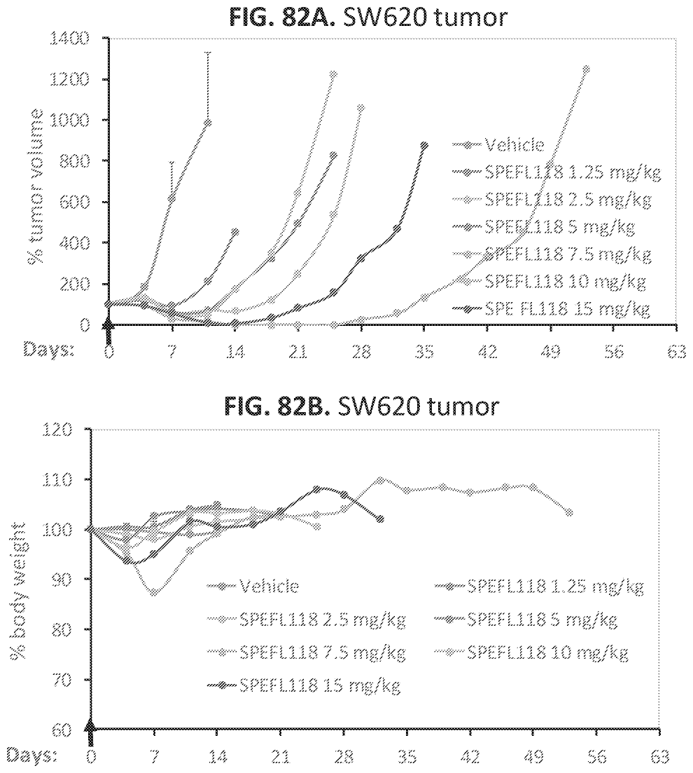

Example 90—SPEFL118's antitumor activity and toxicity in human CRC tumor female SCID mouse models via oral (per oral p.o.) routes: The SPEFL 118 antitumor activity and toxicity was determined in a wide range of SPEFL 118 dosing levels using human CRC SW620 tumor in SCID mice via p.o. routes with the schedule of only one-time SPEFL 118 administration as shown in FIG. 82 (arrowed). Surprisingly, SPEFL118 could be functionally available via p.o. routes and showed tumor regression at multiple SPEFL118 dose levels (FIG. 82A) without showing unacceptable toxicity levels (FIG. 82B).

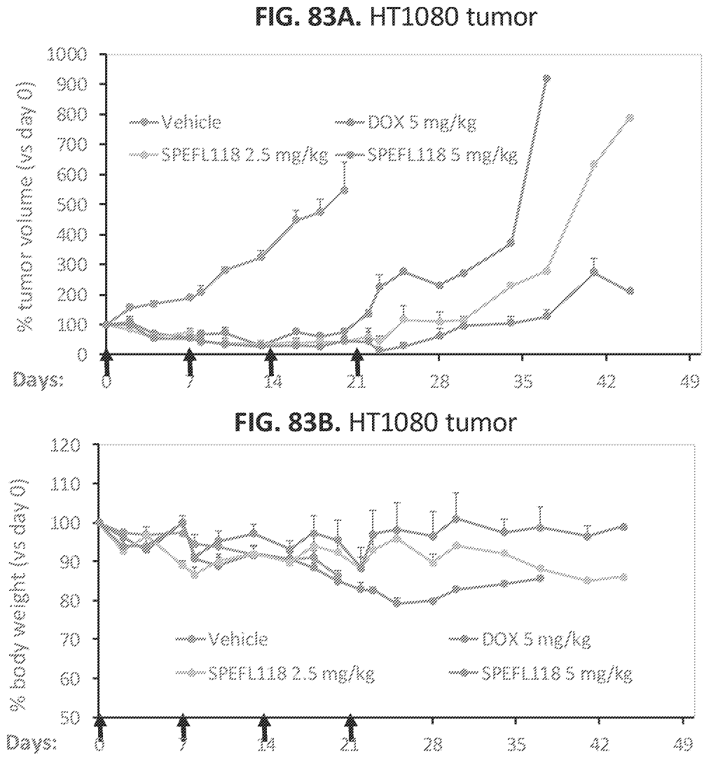

Example 91—SPEFL118's antitumor activity and toxicity in human fibrosarcoma tumor in female SCID mice via intravenous (i.v.) routes: Next, based on the data shown in FIG. 81 and FIG. 83, the anti-fibrosarcoma HT1080 tumor activity and toxicity of SPEFL118 were determined at the dose levels of 2.5 and 5 mg/kg in parallel with the doxorubicin (DOX) as a positive control (5 mg/kg, MTD) via i.v routes with the schedule of weekly×4 (arrowed) in female SCID mice. SPEFL118 exhibited better anti-HT1080 tumor efficacy than DOX in female SCID mice (FIG. 83A), while SPEFL118 showed less toxicity than DOX during and after treatment in female SCID mice (FIG. 83B).

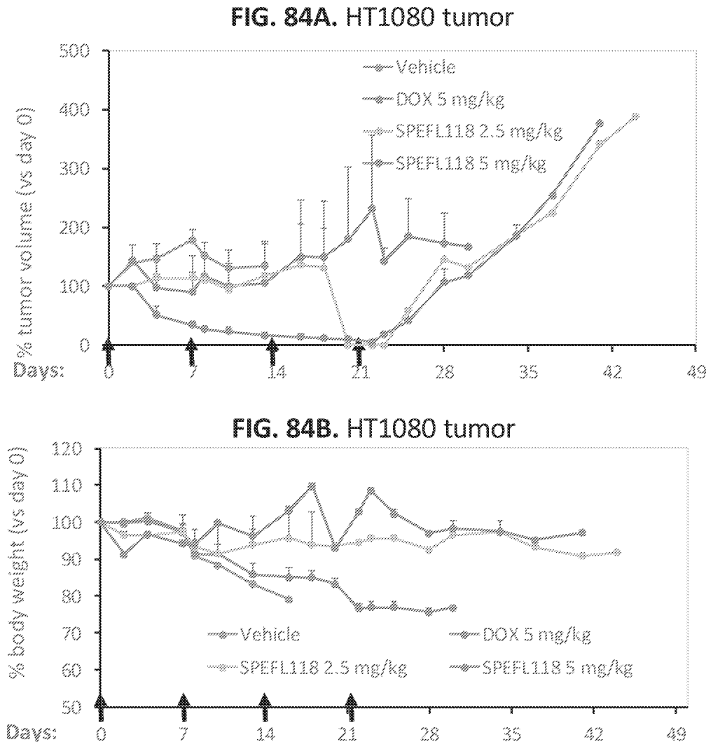

Example 92—SPEFL118's antitumor activity and toxicity in human fibrosarcoma tumor in male SCID mice via i.v. routes: The anti-fibrosarcoma HT1080 tumor activity and toxicity of SPEFL 118 were determined at the dose levels of 2.5 and 5 mg/kg in parallel with the doxorubicin (DOX) as a positive control (5 mg/kg, MTD) via i.v. routes with the schedule of weekly×4 (arrowed) in male SCID mice. Anti-HT1080 tumor results were similar to those using female SCID mice. Specifically, SPEFL118 exhibited better anti-HT1080 tumor efficacy than DOX in male SCID mice (FIG. 84A), while SPEFL118 showed less toxicity than DOX during and after treatment in female SCID mice (FIG. 84B).

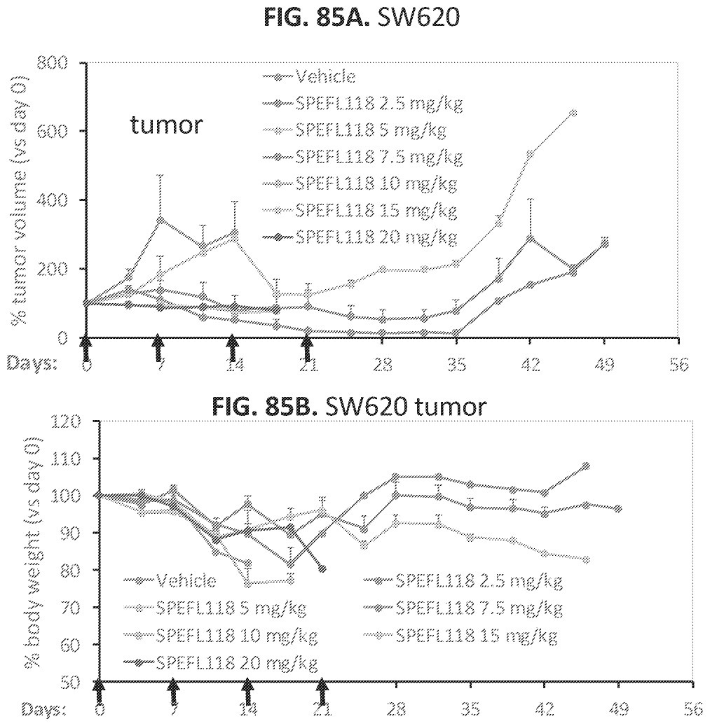

Example 93—SPEFL118's antitumor activity and toxicity in human CRC SW620 tumor in female SCID mice via 1-time i.v. (i.v.×1) and 3-time i.p. (i.p.×3) routes: Next, human CRC SW620 tumor models were used to determine SPEFL 118 efficacy and toxicity in a higher range of dosing levels with weekly×4 schedule via i.v. or i.p. routes (FIG. 85). The goal of this studies is to identify SPEFL 118 IND and antitumor efficacy with 4-time treatment. In this experimental design, SPEFL118's MTD is 5 mg/kg. SPEFL 118 at the dose 7.5 mg/kg or higher could not be successfully finished with the weekly×4 schedule due to over toxicity. SPEFL 118 at doses of 10, 15 and 20 mg/kg can be only used for 1-2-time treatment. SPEFL118 at 7.5 mg/kg after 3-time treatment of SCID mice, 2 of 3 SCID mice died. FIG. 85A shows SPEFL118 efficacy curve and FIG. 85B shows SPEFL 118's effects on SCID mouse body weight change.

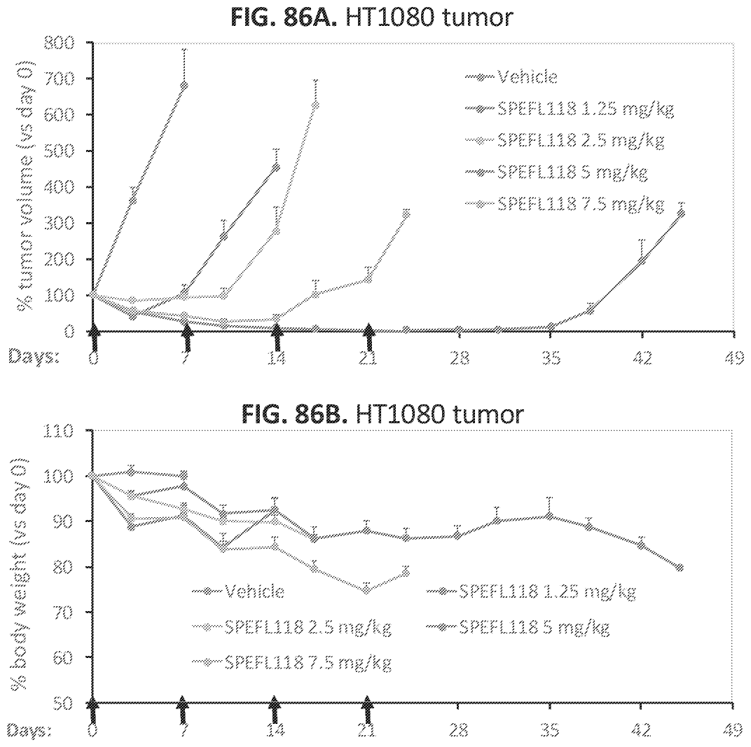

Example 94—SPEFL118's antitumor activity and toxicity in human fibrosarcoma HT1080 tumor in female SCID mice via i.v. routes: Next, based on the data obtained from the experiment shown in FIG. 85, the studies using human fibrosarcoma HT1080 tumor models were repeated (FIG. 86). The studies showed that SPEFL 118 at the dose of 5 mg/kg (roughly MTC) showed the best antitumor activity in the weekly×4 schedule (FIG. 86A), while the higher dose of SPEFL118 (7.5 mg/kg) exhibited decrease antitumor efficacy with more toxicity (FIG. 86A, FIG. 86B).

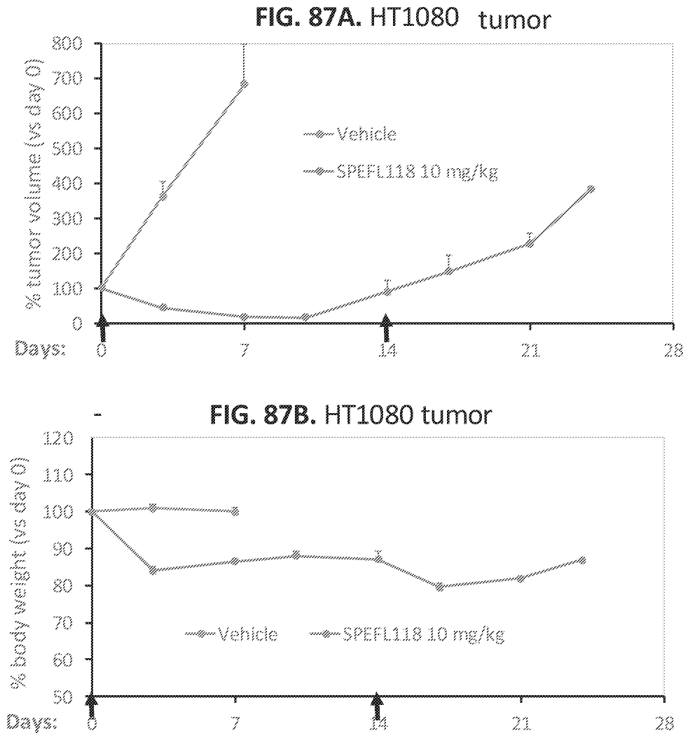

Example 95—SPEFL118's antitumor activity and toxicity in human fibrosarcoma HT1080 tumor in female SCID mice via i.v. routes with bi-weekly schedules: Due to the dose level higher than 7.5 mg/kg for SPEFL 118 inducing high toxicity, a biweekly schedule was tried for a 2-time treatment. The obtained result is presented in FIG. 87A (anti-HT1080 tumor activity) and FIG. 87B (SCID mouse body weight changes during and after treatment, arrowed).

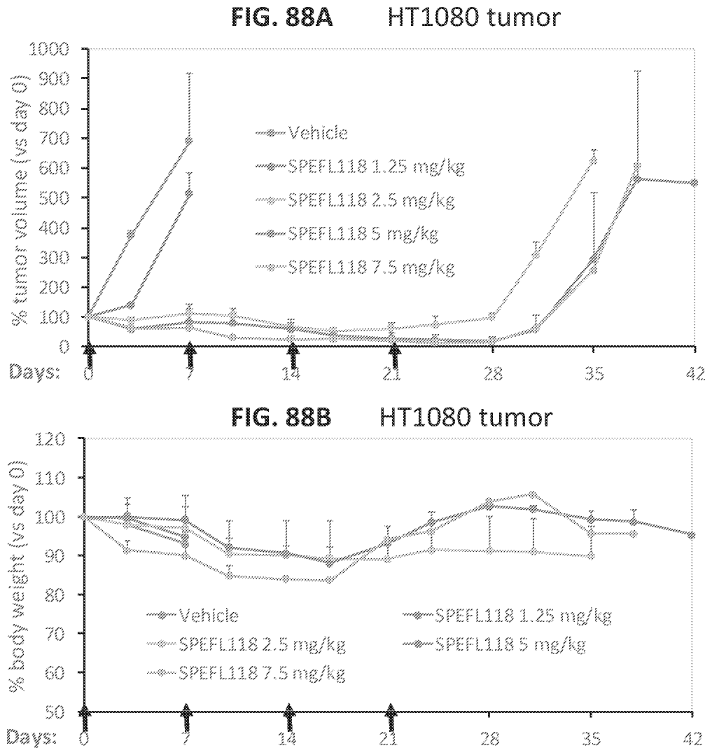

Example 96—SPEFL118's antitumor activity and toxicity in human fibrosarcoma HT1080 tumor in female SCID mice via p.o. routes: Next, based on the data obtained from the experiment shown in FIG. 85 the studies using human fibrosarcoma HT1080 tumor models were repeated via p.o. routes (FIG. 88). Surprisingly, studies indicated that SPEFL 118 showed less toxicity and better than or equivalent to the efficacy obtained using the same schedule with i.v. routes (compare the data obtained from FIG. 88 to the data obtained from FIG. 86). This is an unexpected result and suggests that SPEFL118 could be used via p.o. routes, which is a great advantage for patents who could use SPEFL118 at home without the need to go to hospital for a nurse to administrate SPEFL118 via i.v. routes.

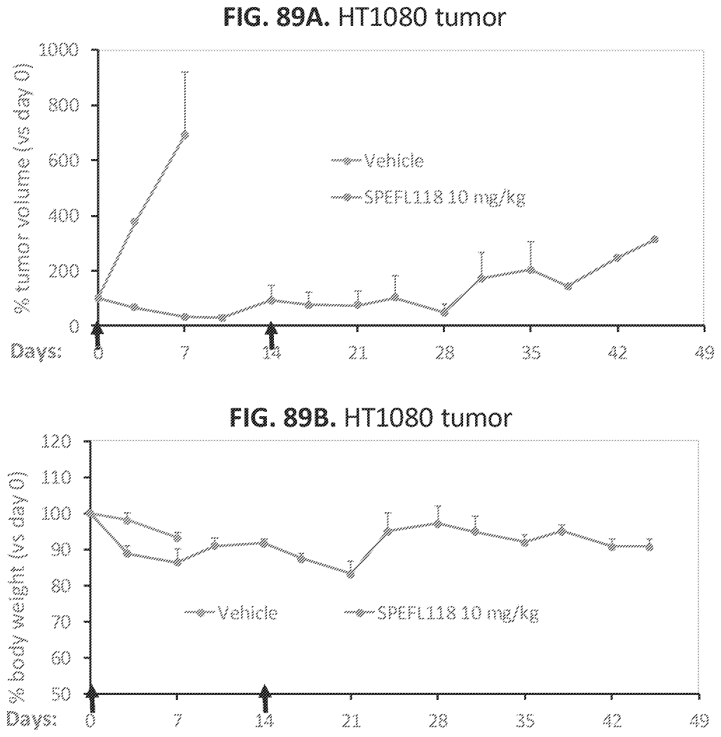

Example 97—SPEFL118's antitumor activity and toxicity in human fibrosarcoma HT1080 tumor in female SCID mice via p.o. routes with bi-weekly schedules: A biweekly schedule was tried for a 2-time treatment via p.o. routes. The obtained result is presented in FIG. 89A (anti-HT1080 tumor activity) and FIG. 89B (SCID mouse body weight changes during and after treatment, arrowed).

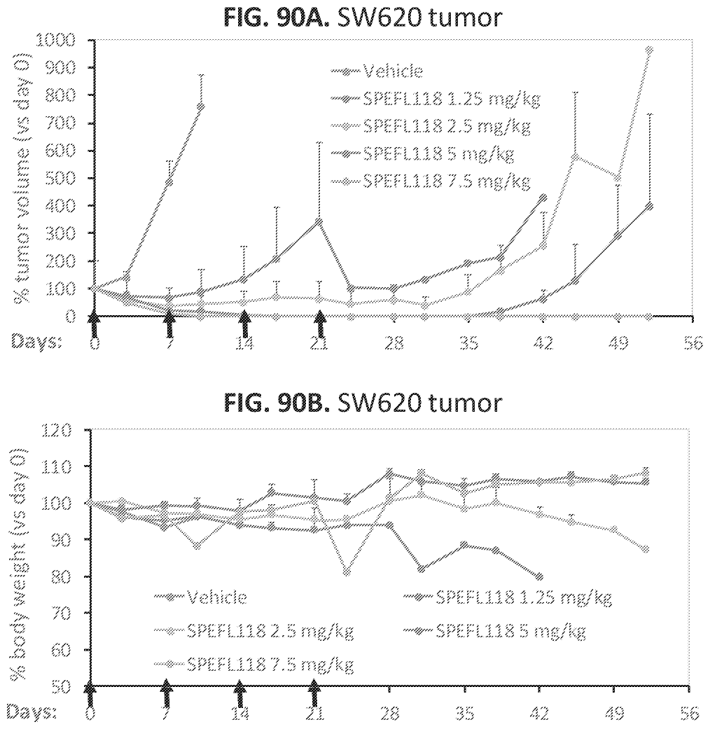

Example 98—SPEFL118's antitumor activity and toxicity in human CRC SW620 tumor in female SCID mice via p.o. routes: Next, the studies were repeated using human CRC SW620 tumor models via p.o. routes (FIG. 90). The studies indicated that SPEFL118 via p.o. routes could also exhibit excellent antitumor efficacy (FIG. 90A) with acceptable toxicity (FIG. 90B), which is better than or comparable with the anti-SW620 tumor efficacy and toxicity shown in FIG. 88. This study further confirmed that SPEFL118 could be used for p.o. administration.

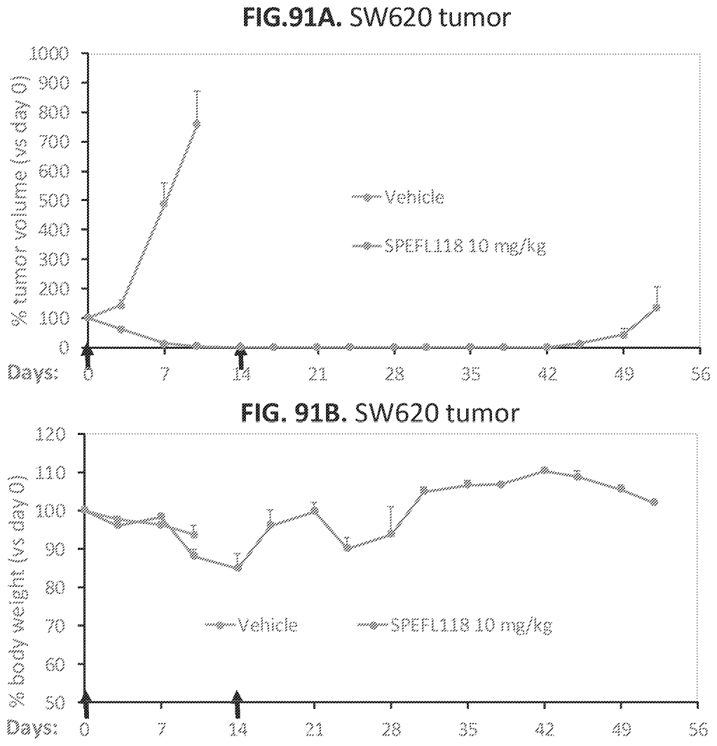

Example 99—SPEFL118's antitumor activity and toxicity in human CRC SW620 tumor in female SCID mice via p.o. routes with bi-weekly schedules for 2-time treatment: A biweekly schedule was tried for a 2-time treatment via p.o. routes in the SW620 tumor model. The obtained result is presented in FIG. 91A (anti-SW620 tumor activity) and FIG. 91B (SCID mouse body weight changes during and after treatment, arrowed).

### INCORPORATION BY REFERENCE

This application contains a sequence listing in XML format filed electronically as part of this application. The sequence listing is entitled ‘67682US02.xml’, created on Jun. 18, 2025, and is 10 kilobytes in size.

What is claimed is:

1. A method of targeting DEAD-box RNA helicase 5 (DDX5), ubiquitin-conjugating enzyme E2 T (UbE2T) and/or ubiquitin specific protease 2a (USP2a) in a subject having cancer, the method comprising administering a composition comprising an effective amount of a complex comprising a compound of the formula 1: 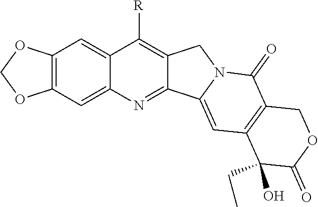
wherein R are selected from the group consisting of: H, 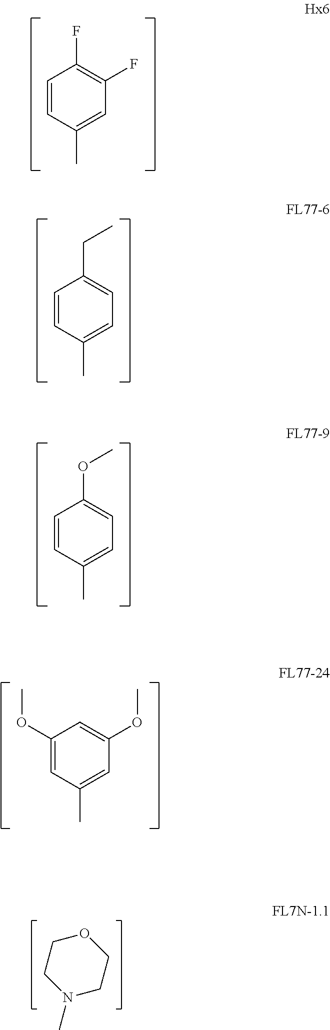 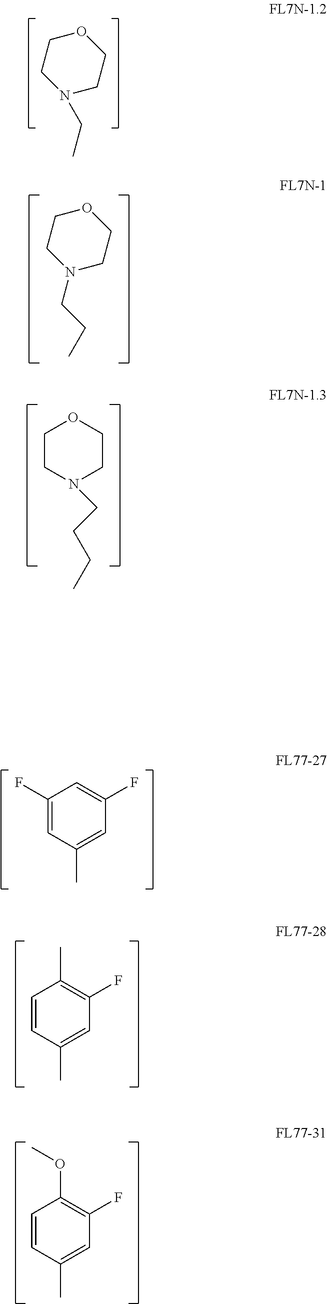 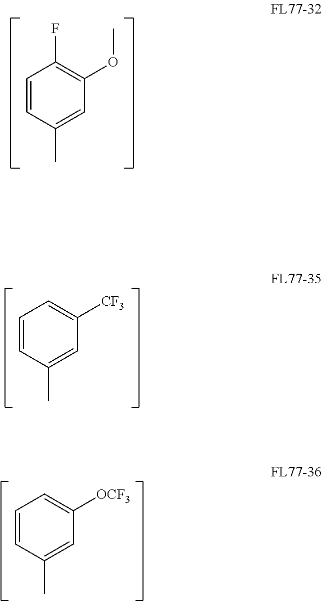
and a pharmaceutically acceptable salt.

2. The method of claim 1, wherein the complex encapsulates or partially encapsulates the compound.

3. The method of claim 1, wherein administration of the complex degrades and/or decreases the activity or expression of DDX5, UbE2T and USP2a.

4. The method of claim 1, wherein the complex further comprises a human protein or a humanized protein or a humanized protein fragment, wherein the compound is non-covalently formulated in a process with the human protein to form a non-covalent protein-compound complex.

5. The method of claim 4, wherein the human protein comprises human serum albumin (HSA), human fibrinogen, or human globulin.

6. The method of claim 5, wherein the human globulin comprises alpha globulin, beta globulin, or gamma globulin or a combination thereof.

7. The method of claim 6, wherein the human globulin comprises a human immunoglobulin (Ig).

8. The method of claim 7, wherein the human Ig comprises a human IgG antibody, a human IgA antibody, a human IgM antibody, a human IgE antibody or a human IgD antibody.

9. The method of claim 8, wherein the human Ig antibody comprises a human or humanized monoclonal antibody (mAb).

10. The method of claim 8, wherein the human Ig antibody comprises a human or humanized single-chain variable fragment fragment (scFv).

11. The method of claim 1, wherein the complex comprises a cyclodextrin selected from the group consisting of β-cyclodextrin (βCD), hydroxypropyl-β-cyclodextrin (HPβCD), sulfobutylether-β-cyclodextrin (SBEβCD), methyl-β-cyclodextrin (meβCD), and derivatives thereof.

12. The method of claim 11, wherein the complex comprises HPβCD and is formulated for oral administration.

13. The method of claim 1, wherein the complex is administered intravenously (i.v.), intraperitoneally (i.p.) or orally (per oral/p.o.) administered in a daily, weekly to biweekly dosage from about 0.1 mg/kg to about 20 mg/kg.

14. The method of the claim 1, wherein the complex is administered at a dosage between about 0.5 mg/kg/wk and about 10 mg/kg/wk of the complex, alternatively between about 1.0 mg/kg/wk and about 8 mg/kg/wk of the complex, or alternatively between about 2.5 mg/kg/wk and about 7.5 mg/kg/wk of the complex.

15. The method of claim 1, wherein the subject has drug resistant cancer.

16. The method of claim 1, wherein the subject has cancerous desmoplasia.

17. The method of claim 1, wherein the method further comprises administering the complex in combination with at least one cancer therapeutic approach.

18. The method of claim 17, wherein the at least one cancer therapeutic approach is selected from the group consisting of chemotherapy, targeted therapy, and immunotherapy.

19. The method of synthesizing a complex comprising:
dissolving a compound of formula 1 in at least one organic solvent to form a first solution;
dissolving the human protein or the humanized protein or the humanized protein fragment in an aqueous solution to form a second solution; and,
combining the first solution and the second solution to form a third solution; and,
removing the organic and aqueous components from the third solution to produce a complex.

20. The method of claim 19, wherein the organic solvent comprises dissolved cyclodextrin.

21. The method of claim 19, wherein the organic solvent includes solvents that are miscible with water or partially dissolved in water.

22. The method of the claim 19, wherein the ratio of the compound of formula 1 to the human protein or the humanized protein or the humanized protein fragment is in a ratio of about 4:1 to a ratio of about 12:1.

23. The method of manufacturing the cyclodextrin-drug complex comprising:
dissolving a cyclodextrin into at least one organic solvent forming a cyclodextrin master solution;
dissolving a compound of formula 1 into the cyclodextrin master solution forming a cyclodextrin-drug complex suspension;
homogenizing the cyclodextrin-drug complex suspension through microfluidizer;
spray-drying the cyclodextrin-drug complex suspension to produce a cyclodextrin-drug complex powder; and drying the cyclodextrin-drug complex powder.

24. The method of claim 23, wherein the organic solvent is selected from the group consisting of methanol, ethanol, formic acid (FA), a salt formate, glacial acetic acid (GAA/AcetA), a salt acetate, glyoxal, ethylene glycol (EG), propylene glycol (PG), formamide (FAD), (N,N,N′,N′)-tetramethyl-ethylenediamine (TEMED), ethanolamide (EA), 2-mercaptoethanol (MercE), and a combination thereof.

25. The method of claim 23, wherein the cyclodextrin-drug complex further comprises a cyclodextrin selected from the group consisting of β-cyclodextrin (βCD), hydroxypropyl-β-cyclodextrin (HPβCD), sulfobutylether-β-cyclodextrin (SBEβCD), methyl-β-cyclodextrin (meβCD), and derivatives thereof.

26. The method of claim 23, wherein the ratio of the compound of formula 1 to cyclodextrin is in the ratio of about 1:1 to 1:2.

27. The method of claim 23, wherein the complex comprises HPβCD and is formulated for oral administration.


SEQUENCE LISTING (9 sequences)

SEQ ID NO: 1 - protein, 520 aa, Homo sapiens
MSGYSSDRDRGRDRGFGAPRFGGSRAGPLSGKKFGNPGEKLVKKKWNLDELPKFEKNFYQEHPDLARRTAQEVETYRRSKEITVRGHNCFKPVLNFYEANFPANVMDVIARQNFTEPTAIQAQGIPVALSGLDMVGVAQTGSGKTLSYLLPAIVHINHQPFLERGDGPICLVLAPTRELAQQVQQVAAEYCRACRLKSTCIYGGAPKGPQIRDLERGVEICIATPGRLIDFLECGKTNLRRTTYLVLDEADRMLDMGFEPQIRKIVDQIRPDRQTLMJJSTWPKEVRQLAEDFLKDYIHINIGALELSANHNILQIVDVCHDVEKDEKLIRLMEEIMSEKENKTIVFVETKRRCDELTRKMRRDGWPAMGIHGDKSQQERDWVLNEFKHGKAPILIATDVASRGLDVEDVKFVINYDYPNSSEDYIHRIGRTARSTKTGTAYTFFTPNNIKQVSDLISVLREANQAINPKLLQLVEDRGSGRSRGRGGMKDDRRDRYSAGKRGGFNTFRDRENYDRGYSS

SEQ ID NO: 2 - DNA, 18 bp, unidentified
  source 1..18: mol_type = other DNA
ggcgcacagcacaagagg

SEQ ID NO: 3 - DNA, 23 bp, unidentified
  source 1..23: mol_type = other DNA
atggcaggaagcaaataagacaa

SEQ ID NO: 4 - DNA, 23 bp, unidentified
  source 1..23: mol_type = other DNA
cttctgcttcaaggagctggaag

SEQ ID NO: 5 - DNA, 22 bp, unidentified
  source 1..22: mol_type = other DNA
gcactttcttcgcagtttcctc

SEQ ID NO: 6 - DNA, 21 bp, unidentified
  source 1..21: mol_type = other DNA
ccacagcaaacctcctcacag

SEQ ID NO: 7 - DNA, 21 bp, unidentified
  source 1..21: mol_type = other DNA
gcaggatagtccttccgagtg

SEQ ID NO: 8 - DNA, 22 bp, unidentified
  source 1..22: mol_type = other DNA
gtctcctctgacttcaacagcg

SEQ ID NO: 9 - DNA, 22 bp, unidentified
  source 1..22: mol_type = other DNA
accaccctgttgctgtagccaa